SEQUENCE LISTING (878 sequences)

SEQ ID NO: 1 - protein, 20 aa, synthetic construct
  REGION 1..20: note = peptide
APDTRPAPGSTAPPAHGVTS

SEQ ID NO: 2 - protein, 19 aa, synthetic construct
  REGION 1..19: note = peptide
GVTSAPDTRPAPGSTPPAH

SEQ ID NO: 3 - protein, 4 aa, synthetic construct
  REGION 1..4: note = peptide
NRRA

SEQ ID NO: 4 - protein, 5 aa, synthetic construct
  REGION 1..5: note = peptide
NRRAC

SEQ ID NO: 5 - protein, 441 aa, synthetic construct
  REGION 1..441: note = peptide
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 12 aa, synthetic construct
  REGION 1..12: note = peptide
KDNIKHVPGGGS

SEQ ID NO: 7 - protein, 14 aa, synthetic construct
  REGION 1..14: note = peptide
FYEKLLGSDIFTGE

SEQ ID NO: 8 - protein, 22 aa, synthetic construct
  REGION 1..22: note = peptide
FYEKLLGSDIFTGEPSLLPDSP

SEQ ID NO: 9 - protein, 17 aa, synthetic construct
  REGION 1..17: note = peptide
VAQLHASLLGLSQLLQP

SEQ ID NO: 10 - protein, 27 aa, synthetic construct
  REGION 1..27: note = peptide
GEPSLLPDSPVAQLHASLLGLSQLLQP

SEQ ID NO: 11 - protein, 19 aa, synthetic construct
  REGION 1..19: note = peptide
PEGHHWETQQIPSLSPSQP

SEQ ID NO: 12 - protein, 8 aa, synthetic construct
  REGION 1..8: note = peptide
PSLLPDSP

SEQ ID NO: 13 - protein, 7 aa, synthetic construct
  REGION 1..7: note = peptide
LPDSPVA

SEQ ID NO: 14 - protein, 39 aa, synthetic construct
  REGION 1..39: note = peptide
FYEKLLGSDIFTGEPSLLPDSPVAQLHASLLGLSQLLQP

SEQ ID NO: 15 - protein, 6 aa, synthetic construct
  REGION 1..6: note = peptide
LLPDSP

SEQ ID NO: 16 - protein, 28 aa, synthetic construct
  REGION 1..28: note = peptide
LLGSDIFTGEPSLLPDSPVAQLHASLLG

SEQ ID NO: 17 - protein, 32 aa, synthetic construct
  REGION 1..32: note = peptide
FYEKLLGSDIFTGEPSLLPDSPVAQLHASLLG

SEQ ID NO: 18 - protein, 7 aa, synthetic construct
  REGION 1..7: note = peptide
QPEGHHW

SEQ ID NO: 19 - protein, 21 aa, synthetic construct
  REGION 1..21: note = peptide
LPDSPVGQLHASLLGLSQLLQ

SEQ ID NO: 20 - protein, 20 aa, synthetic construct
  REGION 1..20: note = peptide
QCQQLSQKLCTLAWSAHPLV

SEQ ID NO: 21 - protein, 14 aa, synthetic construct
  REGION 1..14: note = peptide
GHMDLREEGDEETT

SEQ ID NO: 22 - protein, 19 aa, synthetic construct
  REGION 1..19: note = peptide
LLPDSPVGQLHASLLGLSQ

SEQ ID NO: 23 - protein, 20 aa, synthetic construct
  REGION 1..20: note = peptide
LLRFKILRSLQAFVAVAARV

SEQ ID NO: 24 - protein, 189 aa, synthetic construct
  REGION 1..189: note = peptide
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 25 - protein, 16 aa, synthetic construct
  REGION 1..16: note = peptide
QPEGHHWETQQIPSLS

SEQ ID NO: 26 - protein, 21 aa, synthetic construct
  REGION 1..21: note = peptide
GHHWETQQIPSLSPSQPWQRL

SEQ ID NO: 27 - protein, 10 aa, synthetic construct
  REGION 1..10: note = peptide
QPEGHHWETQ

SEQ ID NO: 28 - protein, 11 aa, synthetic construct
  REGION 1..11: note = peptide
TQQIPSLSPSQ

SEQ ID NO: 29 - protein, 19 aa, synthetic construct
  REGION 1..19: note = peptide
QPEGHHWETQQIPSLSPSQ

SEQ ID NO: 30 - protein, 18 aa, synthetic construct
  REGION 1..18: note = peptide
QPEGHHWETQQIPSLSPS

SEQ ID NO: 31 - protein, 328 aa, synthetic construct
  REGION 1..328: note = peptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 32 - protein, 31 aa, synthetic construct
  REGION 1..31: note = peptide
LLLHKKEDGIWSTDILKDQKEPKNKTFLRCE

SEQ ID NO: 33 - protein, 11 aa, synthetic construct
  REGION 1..11: note = peptide
KSSRGSSDPQG

SEQ ID NO: 34 - protein, 335 aa, synthetic construct
  REGION 1..335: note = peptide
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 35 - protein, 21 aa, synthetic construct
  REGION 1..21: note = peptide
SVNPGLAGGSAQSQRAPDRVL

SEQ ID NO: 36 - protein, 20 aa, synthetic construct
  REGION 1..20: note = peptide
HSGQQQGLPRAAGGSVPHPR

SEQ ID NO: 37 - protein, 11 aa, synthetic construct
  REGION 1..11: note = peptide
GAGRADWPGPP

SEQ ID NO: 38 - protein, 11 aa, synthetic construct
  REGION 1..11: note = peptide
SAQSQRAPDRV

SEQ ID NO: 39 - protein, 41 aa, synthetic construct
  REGION 1..41: note = peptide
AVSVNPGLAGGSAQSQRAPDRVLCHSGQQQGLPRAAGGSVP

SEQ ID NO: 40 - protein, 11 aa, synthetic construct
  REGION 1..11: note = peptide
QQQGLPRAAGG

SEQ ID NO: 41 - protein, 11 aa, synthetic construct
  REGION 1..11: note = peptide
QQLGLPRAAGG

SEQ ID NO: 42 - protein, 11 aa, synthetic construct
  REGION 1..11: note = peptide
QQQGLPRAAEG

SEQ ID NO: 43 - protein, 11 aa, synthetic construct
  REGION 1..11: note = peptide
QQLGLPRAAEG

SEQ ID NO: 44 - protein, 10 aa, synthetic construct
  REGION 1..10: note = peptide
QQQGLPRAAG

SEQ ID NO: 45 - protein, 10 aa, synthetic construct
  REGION 1..10: note = peptide
QQLGLPRAAG

SEQ ID NO: 46 - protein, 10 aa, synthetic construct
  REGION 1..10: note = peptide
QQQGLPRAAE

SEQ ID NO: 47 - protein, 10 aa, synthetic construct
  REGION 1..10: note = peptide
QQLGLPRAAE

SEQ ID NO: 48 - protein, 14 aa, synthetic construct
  REGION 1..14: note = peptide
HSGQQQGLPRAAGG

SEQ ID NO: 49 - protein, 14 aa, synthetic construct
  REGION 1..14: note = peptide
HSGQQLGLPRAAGG

SEQ ID NO: 50 - protein, 14 aa, synthetic construct
  REGION 1..14: note = peptide
HSGQQQGLPRAAEG

SEQ ID NO: 51 - protein, 14 aa, synthetic construct
  REGION 1..14: note = peptide
HSGQQLGLPRAAEG

SEQ ID NO: 52 - protein, 14 aa, synthetic construct
  REGION 1..14: note = peptide
QSQRAPDRVLCHSG

SEQ ID NO: 53 - protein, 13 aa, synthetic construct
  REGION 1..13: note = peptide
GSAQSQRAPDRVL

SEQ ID NO: 54 - protein, 9 aa, synthetic construct
  REGION 1..9: note = peptide
WPGPPELDV

SEQ ID NO: 55 - protein, 31 aa, synthetic construct
  REGION 1..31: note = peptide
LHCPALVTYNTDTFESMPNPEGRYTFGASCV

SEQ ID NO: 56 - protein, 36 aa, synthetic construct
  REGION 1..36: note = peptide
ACPYNYLSTDVGSCTLVCPLHNQEVTAEDGTQRCEK

SEQ ID NO: 57 - protein, 19 aa, synthetic construct
  REGION 1..19: note = peptide
CPLHNQEVTAEDGTQRCEK

SEQ ID NO: 58 - protein, 18 aa, synthetic construct
  REGION 1..18: note = peptide
KLLSLIKGVIVHRLEGVE

SEQ ID NO: 59 - protein, 4 aa, synthetic construct
  REGION 1..4: note = peptide
GPSL

SEQ ID NO: 60 - protein, 1255 aa, synthetic construct [sequence omitted]

SEQ ID NO: 61 - protein, 18 aa, synthetic construct
  REGION 1..18: note = peptide
AVLDNGDPLNNTTPVTGA

SEQ ID NO: 62 - protein, 14 aa, synthetic construct
  REGION 1..14: note = peptide
LKGGVLIQRNPQLC

SEQ ID NO: 63 - protein, 21 aa, synthetic construct
  REGION 1..21: note = peptide
YNTDTFESMPNPEGRYTFGAS

SEQ ID NO: 64 - protein, 21 aa, synthetic construct
  REGION 1..21: note = peptide
PESFDGDPASNTAPLQPEQLQ

SEQ ID NO: 65 - protein, 15 aa, synthetic construct
  REGION 1..15: note = peptide
PHQALLHTANRPEDE

SEQ ID NO: 66 - protein, 17 aa, synthetic construct
  REGION 1..17: note = peptide
CRVLQGLPREYVNARHC

SEQ ID NO: 67 - protein, 14 aa, synthetic construct
  REGION 1..14: note = peptide
YMPIWKFPDEEGAC

SEQ ID NO: 68 - protein, 18 aa, synthetic construct
  REGION 1..18: note = peptide
PESFDGDPASNTAPLQPC

SEQ ID NO: 69 - protein, 16 aa, synthetic construct
  REGION 1..16: note = peptide
RVLQGLPREYVNARHC

SEQ ID NO: 70 - protein, 17 aa, synthetic construct
  REGION 1..17: note = peptide
PESFDGDPASNTAPLQP

SEQ ID NO: 71 - protein, 49 aa, synthetic construct [sequence omitted]

SEQ ID NO: 72 - protein, 47 aa, synthetic construct
  REGION 1..47: note = peptide
RVLQGLPREYVNARHSPESFDGDPASNTAPLQPYMPIWKFPDEEGAC

SEQ ID NO: 73 - protein, 12 aa, synthetic construct
  REGION 1..12: note = peptide
CQMWAPQWGPDC

SEQ ID NO: 74 - protein, 12 aa, synthetic construct
  REGION 1..12: note = peptide
CKLYWADGELTC

SEQ ID NO: 75 - protein, 12 aa, synthetic construct
  REGION 1..12: note = peptide
CVDYHYEGTITC

SEQ ID NO: 76 - protein, 12 aa, synthetic construct
  REGION 1..12: note = peptide
CKLYWADGEFTC

SEQ ID NO: 77 - protein, 12 aa, synthetic construct
  REGION 1..12: note = peptide
CVDYHYEGAITC

SEQ ID NO: 78 - protein, 15 aa, synthetic construct
  REGION 1..15: note = peptide
RLVPVGLERGTVDWV

SEQ ID NO: 79 - protein, 15 aa, synthetic construct
  REGION 1..15: note = peptide
TRWQKGLALGSGDMA

SEQ ID NO: 80 - protein, 15 aa, synthetic construct
  REGION 1..15: note = peptide
QVSHWVSGLAEGSFG

SEQ ID NO: 81 - protein, 15 aa, synthetic construct
  REGION 1..15: note = peptide
LSHTSGRVEGSVSLL

SEQ ID NO: 82 - protein, 15 aa, synthetic construct
  REGION 1..15: note = peptide
LDSTSLAGGPYEAIE

SEQ ID NO: 83 - protein, 15 aa, synthetic construct
  REGION 1..15: note = peptide
HVVMNWMREEFVEEF

SEQ ID NO: 84 - protein, 15 aa, synthetic construct
  REGION 1..15: note = peptide
SWASGMAVGSVSFEE

SEQ ID NO: 85 - protein, 20 aa, synthetic construct
  REGION 1..20: note = peptide
RSLTEILKGGVLIQRNPQLC

SEQ ID NO: 86 - protein, 20 aa, synthetic construct
  REGION 1..20: note = peptide
VLIQRNPQLCYQDTILWKDI

SEQ ID NO: 87 - protein, 20 aa, synthetic construct
  REGION 1..20: note = peptide
YQDTILWKDIFHKNNQLALT

SEQ ID NO: 88 - protein, 20 aa, synthetic construct
  REGION 1..20: note = peptide
FHKNNQLALTLIDTNRSRAC

SEQ ID NO: 89 - protein, 20 aa, synthetic construct
  REGION 1..20: note = peptide
LIDTNRSRACHPCSMPCKGS

SEQ ID NO: 90 - protein, 20 aa, synthetic construct
  REGION 1..20: note = peptide
HPCSMPCKGSRCWGESSEDC

SEQ ID NO: 91 - protein, 20 aa, synthetic construct
  REGION 1..20: note = peptide
RCWGESSEDCQSLTRTVCAG

SEQ ID NO: 92 - protein, 20 aa, synthetic construct
  REGION 1..20: note = peptide
QSLTRTVCAGGCARCKGPLP

SEQ ID NO: 93 - protein, 20 aa, synthetic construct
  REGION 1..20: note = peptide
GCARCKGPLPTDCCHEQCAA

SEQ ID NO: 94 - protein, 20 aa, synthetic construct
  REGION 1..20: note = peptide
TDCCHEQCAAGCTGPKHSDC

SEQ ID NO: 95 - protein, 20 aa, synthetic construct
  REGION 1..20: note = peptide
GCTGPKHSDCLACLHFNHSG

SEQ ID NO: 96 - protein, 20 aa, synthetic construct
  REGION 1..20: note = peptide
LACLHFNHSGICELHCPALV

SEQ ID NO: 97 - protein, 20 aa, synthetic construct
  REGION 1..20: note = peptide
ICELHCPALVTYNTDTFESM

SEQ ID NO: 98 - protein, 20 aa, synthetic construct
  REGION 1..20: note = peptide
TYNTDTFESMPNPEGRYTFG

SEQ ID NO: 99 - protein, 20 aa, synthetic construct
  REGION 1..20: note = peptide
PNPEGRYTFGASCVTACPYN

SEQ ID NO: 100 - protein, 19 aa, synthetic construct
  REGION 1..19: note = peptide
GASCVTACPYNYLSTDVGS

SEQ ID NO: 101 - protein, 22 aa, synthetic construct
  REGION 1..22: note = peptide
PYNYLSTDVGSCTLVCPLHNQE

SEQ ID NO: 102 - protein, 19 aa, synthetic construct
  REGION 1..19: note = peptide
TLVCPLHNQEVTAEDGTQR

SEQ ID NO: 103 - protein, 20 aa, synthetic construct
  REGION 1..20: note = peptide
VTAEDGTQRCEKCSKPCARV

SEQ ID NO: 104 - protein, 20 aa, synthetic construct
  REGION 1..20: note = peptide
EKCSKPCARVCYGLGMEHLR

SEQ ID NO: 105 - protein, 19 aa, synthetic construct
  REGION 1..19: note = peptide
YGLGMEHLREVRAVTSANI

SEQ ID NO: 106 - protein, 19 aa, synthetic construct
  REGION 1..19: note = peptide
EVRAVTSANIQEFAGCKKI

SEQ ID NO: 107 - protein, 9 aa, synthetic construct
  REGION 1..9: note = peptide
KIFGSLAFL

SEQ ID NO: 108 - protein, 9 aa, synthetic construct
  REGION 1..9: note = peptide
KKIFGSLAF

SEQ ID NO: 109 - protein, 9 aa, synthetic construct
  REGION 1..9: note = peptide
GSLAFLPES

SEQ ID NO: 110 - protein, 10 aa, synthetic construct
  REGION 1..10: note = peptide
FAGCKKIFGS

SEQ ID NO: 111 - protein, 10 aa, synthetic construct
  REGION 1..10: note = peptide
SLAFLPESFD

SEQ ID NO: 112 - protein, 19 aa, synthetic construct
  REGION 1..19: note = peptide
FAGCKKIFGSLAFLPESFD

SEQ ID NO: 113 - protein, 23 aa, synthetic construct
  REGION 1..23: note = peptide
QEFAGCKKIFGSLAFLPESFDGD

SEQ ID NO: 114 - protein, 19 aa, synthetic construct
  REGION 1..19: note = peptide
GAISLAPKAQIKESLRAEL

SEQ ID NO: 115 - protein, 223 aa, synthetic construct
  REGION 1..223: note = peptide
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 116 - protein, 288 aa, synthetic construct
  REGION 1..288: note = peptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 117 - protein, 290 aa, synthetic construct
  REGION 1..290: note = peptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 118 - protein, 534 aa, synthetic construct
  REGION 1..534: note = peptide
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 119 - protein, 282 aa, synthetic construct
  REGION 1..282: note = peptide
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 120 - protein, 525 aa, synthetic construct
  REGION 1..525: note = peptide
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 121 - protein, 301 aa, synthetic construct
  REGION 1..301: note = peptide
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 122 - protein, 15 aa, synthetic construct
  REGION 1..15: note = peptide
PGWFLDSPDRPWNPP

SEQ ID NO: 123 - protein, 15 aa, synthetic construct
  REGION 1..15: note = peptide
FLDSPDRPWNPPTFS

SEQ ID NO: 124 - protein, 14 aa, synthetic construct
  REGION 1..14: note = peptide
SPDRPWNPPTFSPA

SEQ ID NO: 125 - protein, 15 aa, synthetic construct
  REGION 1..15: note = peptide
ISLHPKAKIEESPGA

SEQ ID NO: 126 - protein, 19 aa, synthetic construct
  REGION 1..19: note = peptide
FMTYWHLLNAFTVTVPKDL

SEQ ID NO: 127 - protein, 40 aa, synthetic construct
  REGION 1..40: note = peptide
DAEFRHDSGYEVHHQKLVFFAEDVGSNKGAIIGLMVGGVV

SEQ ID NO: 128 - protein, 42 aa, synthetic construct [sequence omitted]

SEQ ID NO: 129 - protein, 19 aa, synthetic construct
  REGION 1..19: note = peptide
ISITEIKGVIVHRIETILF

SEQ ID NO: 130 - protein, 17 aa, synthetic construct
  REGION 1..17: note = peptide
KKKIITITRIITIITID

SEQ ID NO: 131 - protein, 20 aa, synthetic construct
  REGION 1..20: note = peptide
NYSLDKIIVDYNLQSKITLP

SEQ ID NO: 132 - protein, 20 aa, synthetic construct
  REGION 1..20: note = peptide
LINSTKIYSYFPSVISKVNQ

SEQ ID NO: 133 - protein, 20 aa, synthetic construct
  REGION 1..20: note = peptide
LEYIPEITLPVIAALSIAES

SEQ ID NO: 134 - protein, 6 aa, synthetic construct
  REGION 1..6: note = peptide
  SITE 1: note = pyroglutamic acid
EFRHDS

SEQ ID NO: 135 - protein, 6 aa, synthetic construct
  REGION 1..6: note = peptide
DAEFRH

SEQ ID NO: 136 - protein, 164 aa, synthetic construct
  REGION 1..164: note = peptide
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 137 - protein, 159 aa, synthetic construct
  REGION 1..159: note = peptide
MLSHTGPSRFALFLLCSMETLLSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 138 - protein, 159 aa, synthetic construct
  REGION 1..159: note = peptide
MLSHAGPARFALFLLCCMETLLPSHMAPAHRLQPSDVRKIILELRPMSKGLLQDYVSKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 139 - protein, 36 aa, synthetic construct
  REGION 1..36: note = peptide
SDDVQKIVEELQSLSKMLLKDVEEEKGVLVSQNYTL

SEQ ID NO: 140 - protein, 20 aa, synthetic construct
  REGION 1..20: note = peptide
DVQKIVEELQSLSKMLLKDV

SEQ ID NO: 141 - protein, 8 aa, synthetic construct
  REGION 1..8: note = peptide
EELQSLSK

SEQ ID NO: 142 - protein, 4 aa, synthetic construct
  REGION 1..4: note = peptide
DVQK

SEQ ID NO: 143 - protein, 22 aa, synthetic construct
  REGION 1..22: note = peptide
LDNKSVIDEIIEHLDKLIFQDA

SEQ ID NO: 144 - protein, 6 aa, synthetic construct
  REGION 1..6: note = peptide
DEIIEH

SEQ ID NO: 145 - protein, 27 aa, synthetic construct
  REGION 1..27: note = peptide
TDTHECKRFILTISQQFSECMDLALKS

SEQ ID NO: 146 - protein, 9 aa, synthetic construct
  REGION 1..9: note = peptide
TDTHESKRF

SEQ ID NO: 147 - protein, 15 aa, synthetic construct
  REGION 1..15: note = peptide
TDTHERKRFHESKRF

SEQ ID NO: 148 - protein, 6 aa, synthetic construct
  REGION 1..6: note = peptide
HERKRF

SEQ ID NO: 149 - protein, 6 aa, synthetic construct
  REGION 1..6: note = peptide
HECKRF

SEQ ID NO: 150 - protein, 12 aa, synthetic construct
  REGION 1..12: note = peptide
SDDVQKIVEELQ

SEQ ID NO: 151 - protein, 12 aa, synthetic construct
  REGION 1..12: note = peptide
VQKIVEELQSLS

SEQ ID NO: 152 - protein, 12 aa, synthetic construct
  REGION 1..12: note = peptide
IVEELQSLSKML

SEQ ID NO: 153 - protein, 12 aa, synthetic construct
  REGION 1..12: note = peptide
ELQSLSKMLLKD

SEQ ID NO: 154 - protein, 12 aa, synthetic construct
  REGION 1..12: note = peptide
SLSKMLLKDVEE

SEQ ID NO: 155 - protein, 12 aa, synthetic construct
  REGION 1..12: note = peptide
KMLLKDVEEEKG

SEQ ID NO: 156 - protein, 12 aa, synthetic construct
  REGION 1..12: note = peptide
LKDVEEEKGVLV

SEQ ID NO: 157 - protein, 12 aa, synthetic construct
  REGION 1..12: note = peptide
VEEEKGVLVSQN

SEQ ID NO: 158 - protein, 12 aa, synthetic construct
  REGION 1..12: note = peptide
EKGVLVSQNYTL

SEQ ID NO: 159 - protein, 12 aa, synthetic construct
  REGION 1..12: note = peptide
LDNKSVIDEIIE

SEQ ID NO: 160 - protein, 12 aa, synthetic construct
  REGION 1..12: note = peptide
KSVIDEIIEHLD

SEQ ID NO: 161 - protein, 12 aa, synthetic construct
  REGION 1..12: note = peptide
IDEIIEHLDKLI

SEQ ID NO: 162 - protein, 12 aa, synthetic construct
  REGION 1..12: note = peptide
IIEHLDKLIFQD

SEQ ID NO: 163 - protein, 12 aa, synthetic construct
  REGION 1..12: note = peptide
HLDKLIFQDAPE

SEQ ID NO: 164 - protein, 12 aa, synthetic construct
  REGION 1..12: note = peptide
KLIFQDAPETNI

SEQ ID NO: 165 - protein, 12 aa, synthetic construct
  REGION 1..12: note = peptide
FQDAPETNISVP

SEQ ID NO: 166 - protein, 12 aa, synthetic construct
  REGION 1..12: note = peptide
APETNISVPTDT

SEQ ID NO: 167 - protein, 12 aa, synthetic construct
  REGION 1..12: note = peptide
TNISVPTDTHEC

SEQ ID NO: 168 - protein, 12 aa, synthetic construct
  REGION 1..12: note = peptide
SVPTDTHESKRF

SEQ ID NO: 169 - protein, 12 aa, synthetic construct
  REGION 1..12: note = peptide
TDTHECKRFILT

SEQ ID NO: 170 - protein, 12 aa, synthetic construct
  REGION 1..12: note = peptide
TDTHESKRFILT

SEQ ID NO: 171 - protein, 12 aa, synthetic construct
  REGION 1..12: note = peptide
TDTHERKRFILT

SEQ ID NO: 172 - protein, 12 aa, synthetic construct
  REGION 1..12: note = peptide
HECKRFILTISQ

SEQ ID NO: 173 - protein, 12 aa, synthetic construct
  REGION 1..12: note = peptide
HESKRFILTISQ

SEQ ID NO: 174 - protein, 12 aa, synthetic construct
  REGION 1..12: note = peptide
HERKRFILTISQ

SEQ ID NO: 175 - protein, 12 aa, synthetic construct
  REGION 1..12: note = peptide
KRFILTISQQFS

SEQ ID NO: 176 - protein, 12 aa, synthetic construct
  REGION 1..12: note = peptide
ILTISQQFSECM

SEQ ID NO: 177 - protein, 12 aa, synthetic construct
  REGION 1..12: note = peptide
ILTISQQFSESM

SEQ ID NO: 178 - protein, 12 aa, synthetic construct
  REGION 1..12: note = peptide
ILTISQQFSERM

SEQ ID NO: 179 - protein, 12 aa, synthetic construct
  REGION 1..12: note = peptide
ISQQFSECMDLA

SEQ ID NO: 180 - protein, 12 aa, synthetic construct
  REGION 1..12: note = peptide
ISQQFSESMDLA

SEQ ID NO: 181 - protein, 12 aa, synthetic construct
  REGION 1..12: note = peptide
ISQQFSERMDLA

SEQ ID NO: 182 - protein, 12 aa, synthetic construct
  REGION 1..12: note = peptide
QFSECMDLALKS

SEQ ID NO: 183 - protein, 12 aa, synthetic construct
  REGION 1..12: note = peptide
QFSESMDLALKS

SEQ ID NO: 184 - protein, 12 aa, synthetic construct
  REGION 1..12: note = peptide
QFSERMDLALKS

SEQ ID NO: 185 - protein, 13 aa, synthetic construct
  REGION 1..13: note = peptide
SKMLLKDVEEEKG

SEQ ID NO: 186 - protein, 6 aa, synthetic construct
  REGION 1..6: note = peptide
KGVLVS

SEQ ID NO: 187 - protein, 31 aa, synthetic construct
  REGION 1..31: note = peptide
SPAIRAYLKTIRQLDNKSVIDEIIEHLDKLI

SEQ ID NO: 188 - protein, 9 aa, synthetic construct
  REGION 1..9: note = peptide
DEIIEHLDK

SEQ ID NO: 189 - protein, 14 aa, synthetic construct
  REGION 1..14: note = peptide
SVIDEIIEHLDKLI

SEQ ID NO: 190 - protein, 20 aa, synthetic construct
  REGION 1..20: note = peptide
SPAIRAYLKTIRQLDNKSVI

SEQ ID NO: 191 - protein, 9 aa, synthetic construct
  REGION 1..9: note = peptide
TDTHECKRF

SEQ ID NO: 192 - protein, 9 aa, synthetic construct
  REGION 1..9: note = peptide
HECKRFILT

SEQ ID NO: 193 - protein, 9 aa, synthetic construct
  REGION 1..9: note = peptide
HERKRFILT

SEQ ID NO: 194 - protein, 9 aa, synthetic construct
  REGION 1..9: note = peptide
HESKRFILT

SEQ ID NO: 195 - protein, 12 aa, synthetic construct
  REGION 1..12: note = peptide
SVPTDTHECKRF

SEQ ID NO: 196 - protein, 12 aa, synthetic construct
  REGION 1..12: note = peptide
SVPTDTHERKRF

SEQ ID NO: 197 - protein, 28 aa, synthetic construct
  REGION 1..28: note = peptide
SDVRKIILELQPLSRGLLEDYQKKETGV

SEQ ID NO: 198 - protein, 49 aa, synthetic construct
  REGION 1..49: note = peptide
DVRKIILELQPLSRGLLEDYELQPLSRLSDKNIIDKIIEQLDKLKFQHE

SEQ ID NO: 199 - protein, 20 aa, synthetic construct
  REGION 1..20: note = peptide
LSDKNIIDKIIEQLDKLKFQ

SEQ ID NO: 200 - protein, 7 aa, synthetic construct
  REGION 1..7: note = peptide
KLKFQHE

SEQ ID NO: 201 - protein, 6 aa, synthetic construct
  REGION 1..6: note = peptide
LSDKNI

SEQ ID NO: 202 - protein, 4 aa, synthetic construct
  REGION 1..4: note = peptide
LDKL

SEQ ID NO: 203 - protein, 5 aa, synthetic construct
  REGION 1..5: note = peptide
LSDKN

SEQ ID NO: 204 - protein, 27 aa, synthetic construct
  REGION 1..27: note = peptide
ADTFECKSFILTILQQFSACLESVFKS

SEQ ID NO: 205 - protein, 9 aa, synthetic construct
  REGION 1..9: note = peptide
ADNFERKNF

SEQ ID NO: 206 - protein, 28 aa, synthetic construct
  REGION 1..28: note = peptide
SDVRKIILELRPMSKGLLQDYVSKEIGL

SEQ ID NO: 207 - protein, 20 aa, synthetic construct
  REGION 1..20: note = peptide
DVRKIILELRPMSKGLLQDY

SEQ ID NO: 208 - protein, 22 aa, synthetic construct
  REGION 1..22: note = peptide
LSDKNTIDKIIEQLDKLKFQRE

SEQ ID NO: 209 - protein, 27 aa, synthetic construct
  REGION 1..27: note = peptide
ADNFERKNFILAVLQQFSACLEHVLQS

SEQ ID NO: 210 - protein, 24 aa, synthetic construct
  REGION 1..24: note = peptide
LQPKEIQAIIVELQNLSKKLLDDY

SEQ ID NO: 211 - protein, 20 aa, synthetic construct
  REGION 1..20: note = peptide
EIQAIIVELQNLSKKLLDDY

SEQ ID NO: 212 - protein, 20 aa, synthetic construct
  REGION 1..20: note = peptide
SLNNDKSLYIIEQLDKLNFQ

SEQ ID NO: 213 - protein, 26 aa, synthetic construct
  REGION 1..26: note = peptide
TDNFERKRFILTILRWFSNCLEHRAQ

SEQ ID NO: 214 - protein, 12 aa, synthetic construct
  REGION 1..12: note = peptide
SVPADTFECKSF

SEQ ID NO: 215 - protein, 12 aa, synthetic construct
  REGION 1..12: note = peptide
SVPADTFERKSF

SEQ ID NO: 216 - protein, 33 aa, synthetic construct
  REGION 1..33: note = peptide
NSSAILPYFRAIRPLSDKNIIDKIIEQLDKLKF

SEQ ID NO: 217 - protein, 24 aa, synthetic construct
  REGION 1..24: note = peptide
APTHQLPPSDVRKIILELQPLSRG

SEQ ID NO: 218 - protein, 6 aa, synthetic construct
  REGION 1..6: note = peptide
TGVPES

SEQ ID NO: 219 - protein, 12 aa, synthetic construct
  REGION 1..12: note = peptide
SMPADNFERKNF

SEQ ID NO: 220 - protein, 33 aa, synthetic construct
  REGION 1..33: note = peptide
NGSAILPYFRAIRPLSDKNTIDKIIEQLDKLKF

SEQ ID NO: 221 - protein, 24 aa, synthetic construct
  REGION 1..24: note = peptide
APAHRLQPSDIRKIILELRPMSKG

SEQ ID NO: 222 - protein, 6 aa, synthetic construct
  REGION 1..6: note = peptide
IGLPES

SEQ ID NO: 223 - protein, 12 aa, synthetic construct
  REGION 1..12: note = peptide
SMPTDNFERKRF

SEQ ID NO: 224 - protein, 33 aa, synthetic construct
  REGION 1..33: note = peptide
NSSAILPYFKAISPSLNNDKSLYIIEQLDKLNF

SEQ ID NO: 225 - protein, 24 aa, synthetic construct
  REGION 1..24: note = peptide
GPIYQLQPKEIQAIIVELQNLSKK

SEQ ID NO: 226 - protein, 6 aa, synthetic construct
  REGION 1..6: note = peptide
KGVQKF

SEQ ID NO: 227 - protein, 33 aa, synthetic construct
  REGION 1..33: note = peptide
HSPAIRAYLKTIRQLDNKSVIDEIIEHLDKLIF

SEQ ID NO: 228 - protein, 25 aa, synthetic construct
  REGION 1..25: note = peptide
LPVRLLRPSDDVQKIVEELQSLSKM

SEQ ID NO: 229 - protein, 20 aa, synthetic construct
  REGION 1..20: note = peptide
DVRKIILELQPLSRGLLEDY

SEQ ID NO: 230 - protein, 7 aa, synthetic construct
  REGION 1..7: note = peptide
ELQPLSR

SEQ ID NO: 231 - protein, 22 aa, synthetic construct
  REGION 1..22: note = peptide
LSDKNIIDKIIEQLDKLKFQHE

SEQ ID NO: 232 - protein, 6 aa, synthetic construct
  REGION 1..6: note = peptide
HESKRF

SEQ ID NO: 233 - protein, 14 aa, synthetic construct
  REGION 1..14: note = peptide
SKMLLKDVEEEKGC

SEQ ID NO: 234 - protein, 9 aa, synthetic construct
  REGION 1..9: note = peptide
EELQSLSKC

SEQ ID NO: 235 - protein, 7 aa, synthetic construct
  REGION 1..7: note = peptide
KGVLVSC

SEQ ID NO: 236 - protein, 15 aa, synthetic construct
  REGION 1..15: note = peptide
SVIDEIIEHLDKLIC

SEQ ID NO: 237 - protein, 21 aa, synthetic construct
  REGION 1..21: note = peptide
SPAIRAYLKTIRQLDNKSVIC

SEQ ID NO: 238 - protein, 10 aa, synthetic construct
  REGION 1..10: note = peptide
HERKRFILTC

SEQ ID NO: 239 - protein, 10 aa, synthetic construct
  REGION 1..10: note = peptide
HESKRFILTC

SEQ ID NO: 240 - protein, 13 aa, synthetic construct
  REGION 1..13: note = peptide
SVPTDTHERKRFC

SEQ ID NO: 241 - protein, 13 aa, synthetic construct
  REGION 1..13: note = peptide
SVPTDTHESKRFC

SEQ ID NO: 242 - protein, 13 aa, synthetic construct
  REGION 1..13: note = peptide
KRFILTISQQFSC

SEQ ID NO: 243 - protein, 37 aa, synthetic construct
  REGION 1..37: note = peptide
ACDTATCVTHRLAGLLSRSGGVVKNNFVPTNVGSKAF

SEQ ID NO: 244 - protein, 37 aa, synthetic construct
  REGION 1..37: note = peptide
ACNTATCVTHRLAGLLSRSGGMVKSNFVPTNVGSKAF

SEQ ID NO: 245 - protein, 10 aa, synthetic construct
  REGION 1..10: note = peptide
ACDTATCVTH

SEQ ID NO: 246 - protein, 15 aa, synthetic construct
  REGION 1..15: note = peptide
ACDTATCVTHRLAGL

SEQ ID NO: 247 - protein, 18 aa, synthetic construct
  REGION 1..18: note = peptide
ACDTATCVTHRLAGLLSR

SEQ ID NO: 248 - protein, 20 aa, synthetic construct
  REGION 1..20: note = peptide
ACDTATCVTHRLAGLLSRSG

SEQ ID NO: 249 - protein, 25 aa, synthetic construct
  REGION 1..25: note = peptide
ACDTATCVTHRLAGLLSRSGGVVKN

SEQ ID NO: 250 - protein, 13 aa, synthetic construct
  REGION 1..13: note = peptide
TATCVTHRLAGLL

SEQ ID NO: 251 - protein, 14 aa, synthetic construct
  REGION 1..14: note = peptide
ATCVTHRLAGLLSR

SEQ ID NO: 252 - protein, 8 aa, synthetic construct
  REGION 1..8: note = peptide
RLAGLLSR

SEQ ID NO: 253 - protein, 15 aa, synthetic construct
  REGION 1..15: note = peptide
RLAGLLSRSGGVVKN

SEQ ID NO: 254 - protein, 8 aa, synthetic construct
  REGION 1..8: note = peptide
RSGGVVKN

SEQ ID NO: 255 - protein, 20 aa, synthetic construct
  REGION 1..20: note = peptide
RLAGLLSRSGGVVKNNFVPT

SEQ ID NO: 256 - protein, 23 aa, synthetic construct
  REGION 1..23: note = peptide
RLAGLLSRSGGVVKNNFVPTNVG

SEQ ID NO: 257 - protein, 25 aa, synthetic construct
  REGION 1..25: note = peptide
RLAGLLSRSGGVVKNNFVPTNVGSK

SEQ ID NO: 258 - protein, 27 aa, synthetic construct
  REGION 1..27: note = peptide
RLAGLLSRSGGVVKNNFVPTNVGSKAF

SEQ ID NO: 259 - protein, 23 aa, synthetic construct
  REGION 1..23: note = peptide
LLSRSGGVVKNNFVPTNVGSKAF

SEQ ID NO: 260 - protein, 20 aa, synthetic construct
  REGION 1..20: note = peptide
RSGGVVKNNFVPTNVGSKAF

SEQ ID NO: 261 - protein, 18 aa, synthetic construct
  REGION 1..18: note = peptide
GGVVKNNFVPTNVGSKAF

SEQ ID NO: 262 - protein, 16 aa, synthetic construct
  REGION 1..16: note = peptide
VVKNNFVPTNVGSKAF

SEQ ID NO: 263 - protein, 13 aa, synthetic construct
  REGION 1..13: note = peptide
NNFVPTNVGSKAF

SEQ ID NO: 264 - protein, 10 aa, synthetic construct
  REGION 1..10: note = peptide
VPTNVGSKAF

SEQ ID NO: 265 - protein, 7 aa, synthetic construct
  REGION 1..7: note = peptide
NVGSKAF

SEQ ID NO: 266 - protein, 5 aa, synthetic construct
  REGION 1..5: note = peptide
GSKAF

SEQ ID NO: 267 - protein, 16 aa, synthetic construct
  REGION 1..16: note = peptide
  VARIANT 1: note = aliphatic amino acid selected from alanine, glycine, valine, isoleucine and leucine
XACDTATCVTHRLAGL

SEQ ID NO: 268 - protein, 19 aa, synthetic construct
  REGION 1..19: note = peptide
  VARIANT 1: note = aliphatic amino acid selected from alanine, glycine, valine, isoleucine and leucine
XACDTATCVTHRLAGLLSR

SEQ ID NO: 269 - protein, 21 aa, synthetic construct
  REGION 1..21: note = peptide
  VARIANT 1: note = aliphatic amino acid selected from alanine, glycine, valine, isoleucine and leucine
XACDTATCVTHRLAGLLSRSG

SEQ ID NO: 270 - protein, 26 aa, synthetic construct
  REGION 1..26: note = peptide
  VARIANT 1: note = aliphatic amino acid selected from alanine, glycine, valine, isoleucine and leucine
XACDTATCVTHRLAGLLSRSGGVVKN

SEQ ID NO: 271 - protein, 14 aa, synthetic construct
  REGION 1..14: note = peptide
  VARIANT 1: note = aliphatic amino acid selected from alanine, glycine, valine, isoleucine and leucine
XTATCVTHRLAGLL

SEQ ID NO: 272 - protein, 15 aa, synthetic construct
  REGION 1..15: note = peptide
  VARIANT 1: note = aliphatic amino acid selected from alanine, glycine, valine, isoleucine and leucine
XATCVTHRLAGLLSR

SEQ ID NO: 273 - protein, 9 aa, synthetic construct
  REGION 1..9: note = peptide
  VARIANT 1: note = aliphatic amino acid selected from alanine, glycine, valine, isoleucine and leucine
XRLAGLLSR

SEQ ID NO: 274 - protein, 16 aa, synthetic construct
  REGION 1..16: note = peptide
  VARIANT 1: note = aliphatic amino acid selected from alanine, glycine, valine, isoleucine and leucine
XRLAGLLSRSGGVVKN

SEQ ID NO: 275 - protein, 21 aa, synthetic construct
  REGION 1..21: note = peptide
  VARIANT 1: note = aliphatic amino acid selected from alanine, glycine, valine, isoleucine and leucine
XRLAGLLSRSGGVVKNNFVPT

SEQ ID NO: 276 - protein, 24 aa, synthetic construct
  REGION 1..24: note = peptide
  VARIANT 1: note = aliphatic amino acid selected from alanine, glycine, valine, isoleucine and leucine
XRLAGLLSRSGGVVKNNFVPTNVG

SEQ ID NO: 277 - protein, 26 aa, synthetic construct
  REGION 1..26: note = peptide
  VARIANT 1: note = aliphatic amino acid selected from alanine, glycine, valine, isoleucine and leucine
XRLAGLLSRSGGVVKNNFVPTNVGSK

SEQ ID NO: 278 - protein, 28 aa, synthetic construct
  REGION 1..28: note = peptide
  VARIANT 1: note = aliphatic amino acid selected from alanine, glycine, valine, isoleucine and leucine
XRLAGLLSRSGGVVKNNFVPTNVGSKAF

SEQ ID NO: 279 - protein, 24 aa, synthetic construct
  REGION 1..24: note = peptide
  VARIANT 1: note = aliphatic amino acid selected from alanine, glycine, valine, isoleucine and leucine
XLLSRSGGVVKNNFVPTNVGSKAF

SEQ ID NO: 280 - protein, 21 aa, synthetic construct
  REGION 1..21: note = peptide
  VARIANT 1: note = aliphatic amino acid selected from alanine, glycine, valine, isoleucine and leucine
XRSGGVVKNNFVPTNVGSKAF

SEQ ID NO: 281 - protein, 19 aa, synthetic construct
  REGION 1..19: note = peptide
  VARIANT 1: note = aliphatic amino acid selected from alanine, glycine, valine, isoleucine and leucine
XGGVVKNNFVPTNVGSKAF

SEQ ID NO: 282 - protein, 17 aa, synthetic construct
  REGION 1..17: note = peptide
  VARIANT 1: note = aliphatic amino acid selected from alanine, glycine, valine, isoleucine and leucine
XVVKNNFVPTNVGSKAF

SEQ ID NO: 283 - protein, 14 aa, synthetic construct
  REGION 1..14: note = peptide
  VARIANT 1: note = aliphatic amino acid selected from alanine, glycine, valine, isoleucine and leucine
XNNFVPTNVGSKAF

SEQ ID NO: 284 - protein, 11 aa, synthetic construct
  REGION 1..11: note = peptide
  VARIANT 1: note = aliphatic amino acid selected from alanine, glycine, valine, isoleucine and leucine
XVPTNVGSKAF

SEQ ID NO: 285 - protein, 8 aa, synthetic construct
  REGION 1..8: note = peptide
  VARIANT 1: note = aliphatic amino acid selected from alanine, glycine, valine, isoleucine and leucine
XNVGSKAF

SEQ ID NO: 286 - protein, 128 aa, synthetic construct
  REGION 1..128: note = peptide
MGFQKFSPFLALSILVLLQAGSLHAAPFRSALESSPADPATLSEDEARLLLAALVQDYVQMKASELEQEQEREGSRIIAQKRACDTATCVTHRLAGLLSRSGGVVKNNFVPTNVGSKAFGRRRRDLQA

SEQ ID NO: 287 - protein, 9 aa, synthetic construct
  REGION 1..9: note = peptide
RLAGLLSRC

SEQ ID NO: 288 - protein, 16 aa, synthetic construct
  REGION 1..16: note = peptide
RLAGLLSRSGGVVKNC

SEQ ID NO: 289 - protein, 9 aa, synthetic construct
  REGION 1..9: note = peptide
RSGGVVKNC

SEQ ID NO: 290 - protein, 14 aa, synthetic construct
  REGION 1..14: note = peptide
NNFVPTNVGSKAFC

SEQ ID NO: 291 - protein, 11 aa, synthetic construct
  REGION 1..11: note = peptide
VPTNVGSKAFC

SEQ ID NO: 292 - protein, 8 aa, synthetic construct
  REGION 1..8: note = peptide
NVGSKAFC

SEQ ID NO: 293 - protein, 5 aa, synthetic construct
  REGION 1..5: note = peptide
GGSGG

SEQ ID NO: 294 - protein, 150 aa, synthetic construct
  REGION 1..150: note = peptide
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVF

SEQ ID NO: 295 - protein, 50 aa, synthetic construct
  REGION 1..50: note = peptide
AQSIPWNLERITPPRYRADEYQPPDGGSLVEVYLLDTSIQSDHREIEGRV

SEQ ID NO: 296 - protein, 100 aa, synthetic construct
  REGION 1..100: note = peptide
MVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAA

SEQ ID NO: 297 - protein, 100 aa, synthetic construct
  REGION 1..100: note = peptide
CQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMML

SEQ ID NO: 298 - protein, 50 aa, synthetic construct
  REGION 1..50: note = peptide
SAEPELTLAELRQRLIHFSAKDVINEAWFPEDQRVLTPNLVAALPPSTHG

SEQ ID NO: 299 - protein, 242 aa, synthetic construct
  REGION 1..242: note = peptide
AGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 300 - protein, 16 aa, synthetic construct
  REGION 1..16: note = peptide
NVPEEDGTRFHRQASK

SEQ ID NO: 301 - protein, 17 aa, synthetic construct
  REGION 1..17: note = peptide
NVPEEDGTRFHRQASKC

SEQ ID NO: 302 - protein, 14 aa, synthetic construct
  REGION 1..14: note = peptide
PEEDGTRFHRQASK

SEQ ID NO: 303 - protein, 15 aa, synthetic construct
  REGION 1..15: note = peptide
CPEEDGTRFHRQASK

SEQ ID NO: 304 - protein, 15 aa, synthetic construct
  REGION 1..15: note = peptide
PEEDGTRFHRQASKC

SEQ ID NO: 305 - protein, 14 aa, synthetic construct
  REGION 1..14: note = peptide
AEEDGTRFHRQASK

SEQ ID NO: 306 - protein, 14 aa, synthetic construct
  REGION 1..14: note = peptide
TEEDGTRFHRQASK

SEQ ID NO: 307 - protein, 14 aa, synthetic construct
  REGION 1..14: note = peptide
PQEDGTRFHRQASK

SEQ ID NO: 308 - protein, 14 aa, synthetic construct
  REGION 1..14: note = peptide
PEEDGTRFHRRASK

SEQ ID NO: 309 - protein, 14 aa, synthetic construct
  REGION 1..14: note = peptide
PEEDGTRFHRKASK

SEQ ID NO: 310 - protein, 14 aa, synthetic construct
  REGION 1..14: note = peptide
PEEDGTRFHRQASR

SEQ ID NO: 311 - protein, 14 aa, synthetic construct
  REGION 1..14: note = peptide
PEEDGTRFHRTASK

SEQ ID NO: 312 - protein, 13 aa, synthetic construct
  REGION 1..13: note = peptide
SIPWNLERITPPR

SEQ ID NO: 313 - protein, 12 aa, synthetic construct
  REGION 1..12: note = peptide
PEEDGTRFHRQA

SEQ ID NO: 314 - protein, 13 aa, synthetic construct
  REGION 1..13: note = peptide
EEDGTRFHRQASK

SEQ ID NO: 315 - protein, 12 aa, synthetic construct
  REGION 1..12: note = peptide
EEDGTRFHRQAS

SEQ ID NO: 316 - protein, 11 aa, synthetic construct
  REGION 1..11: note = peptide
SIPWNLERITP

SEQ ID NO: 317 - protein, 12 aa, synthetic construct
  REGION 1..12: note = peptide
SIPWNLERITPC

SEQ ID NO: 318 - protein, 10 aa, synthetic construct
  REGION 1..10: note = peptide
SIPWNLERIT

SEQ ID NO: 319 - protein, 11 aa, synthetic construct
  REGION 1..11: note = peptide
SIPWNLERITC

SEQ ID NO: 320 - protein, 10 aa, synthetic construct
  REGION 1..10: note = peptide
LRPRGQPNQC

SEQ ID NO: 321 - protein, 9 aa, synthetic construct
  REGION 1..9: note = peptide
SRHLAQASQ

SEQ ID NO: 322 - protein, 10 aa, synthetic construct
  REGION 1..10: note = peptide
SRHLAQASQC

SEQ ID NO: 323 - protein, 10 aa, synthetic construct
  REGION 1..10: note = peptide
SRSGKRRGER

SEQ ID NO: 324 - protein, 11 aa, synthetic construct
  REGION 1..11: note = peptide
SRSGKRRGERC

SEQ ID NO: 325 - protein, 15 aa, synthetic construct
  REGION 1..15: note = peptide
IIGASSDCSTCFVSQ

SEQ ID NO: 326 - protein, 15 aa, synthetic construct
  REGION 1..15: note = peptide
IIGASSDSSTSFVSQ

SEQ ID NO: 327 - protein, 16 aa, synthetic construct
  REGION 1..16: note = peptide
IIGASSDSSTSFVSQC

SEQ ID NO: 328 - protein, 15 aa, synthetic construct
  REGION 1..15: note = peptide
CIGASSDSSTSFVSC

SEQ ID NO: 329 - protein, 14 aa, synthetic construct
  REGION 1..14: note = peptide
IGASSDSSTSFVSC

SEQ ID NO: 330 - protein, 13 aa, synthetic construct
  REGION 1..13: note = peptide
CDGTRFHRQASKC

SEQ ID NO: 331 - protein, 12 aa, synthetic construct
  REGION 1..12: note = peptide
DGTRFHRQASKC

SEQ ID NO: 332 - protein, 12 aa, synthetic construct
  REGION 1..12: note = peptide
CDGTRFHRQASK

SEQ ID NO: 333 - protein, 12 aa, synthetic construct
  REGION 1..12: note = peptide
AGRDAGVAKGAC

SEQ ID NO: 334 - protein, 8 aa, synthetic construct
  REGION 1..8: note = peptide
RDAGVAKC

SEQ ID NO: 335 - protein, 7 aa, synthetic construct
  REGION 1..7: note = peptide
RDAGVAK

SEQ ID NO: 336 - protein, 13 aa, synthetic construct
  REGION 1..13: note = peptide
SRHLAQASQLEQC

SEQ ID NO: 337 - protein, 12 aa, synthetic construct
  REGION 1..12: note = peptide
SRHLAQASQLEQ

SEQ ID NO: 338 - protein, 12 aa, synthetic construct
  REGION 1..12: note = peptide
GDYEELVLALRC

SEQ ID NO: 339 - protein, 11 aa, synthetic construct
  REGION 1..11: note = peptide
GDYEELVLALR

SEQ ID NO: 340 - protein, 11 aa, synthetic construct
  REGION 1..11: note = peptide
LVLALRSEEDC

SEQ ID NO: 341 - protein, 10 aa, synthetic construct
  REGION 1..10: note = peptide
LVLALRSEED

SEQ ID NO: 342 - protein, 9 aa, synthetic construct
  REGION 1..9: note = peptide
AKDPWRLPC

SEQ ID NO: 343 - protein, 8 aa, synthetic construct
  REGION 1..8: note = peptide
AKDPWRLP

SEQ ID NO: 344 - protein, 10 aa, synthetic construct
  REGION 1..10: note = peptide
AARRGYLTKC

SEQ ID NO: 345 - protein, 9 aa, synthetic construct
  REGION 1..9: note = peptide
AARRGYLTK

SEQ ID NO: 346 - protein, 18 aa, synthetic construct
  REGION 1..18: note = peptide
FLVKMSGDLLELALKLPC

SEQ ID NO: 347 - protein, 17 aa, synthetic construct
  REGION 1..17: note = peptide
FLVKMSGDLLELALKLP

SEQ ID NO: 348 - protein, 10 aa, synthetic construct
  REGION 1..10: note = peptide
EEDSSVFAQC

SEQ ID NO: 349 - protein, 9 aa, synthetic construct
  REGION 1..9: note = peptide
EEDSSVFAQ

SEQ ID NO: 350 - protein, 17 aa, synthetic construct
  REGION 1..17: note = peptide
CKSAQRHFRTGDEEPVN

SEQ ID NO: 351 - protein, 16 aa, synthetic construct
  REGION 1..16: note = peptide
KSAQRHFRTGDEEPVN

SEQ ID NO: 352 - protein, 11 aa, synthetic construct
  REGION 1..11: note = peptide
AGRDAGVAKGA

SEQ ID NO: 353 - protein, 140 aa, synthetic construct
  REGION 1..140: note = peptide
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 354 - protein, 7 aa, synthetic construct
  REGION 1..7: note = peptide
DQPVLPD

SEQ ID NO: 355 - protein, 8 aa, synthetic construct
  REGION 1..8: note = peptide
DQPVLPDN

SEQ ID NO: 356 - protein, 9 aa, synthetic construct
  REGION 1..9: note = peptide
DQPVLPDNE

SEQ ID NO: 357 - protein, 10 aa, synthetic construct
  REGION 1..10: note = peptide
DQPVLPDNEA

SEQ ID NO: 358 - protein, 11 aa, synthetic construct
  REGION 1..11: note = peptide
DQPVLPDNEAY

SEQ ID NO: 359 - protein, 12 aa, synthetic construct
  REGION 1..12: note = peptide
DQPVLPDNEAYE

SEQ ID NO: 360 - protein, 8 aa, synthetic construct
  REGION 1..8: note = peptide
DSPVLPDG

SEQ ID NO: 361 - protein, 8 aa, synthetic construct
  REGION 1..8: note = peptide
DHPVHPDS

SEQ ID NO: 362 - protein, 8 aa, synthetic construct
  REGION 1..8: note = peptide
DTPVLPDS

SEQ ID NO: 363 - protein, 8 aa, synthetic construct
  REGION 1..8: note = peptide
DAPVTPDT

SEQ ID NO: 364 - protein, 8 aa, synthetic construct
  REGION 1..8: note = peptide
DAPVRPDS

SEQ ID NO: 365 - protein, 9 aa, synthetic construct
  REGION 1..9: note = peptide
YDRPVQPDR

SEQ ID NO: 366 - protein, 13 aa, synthetic construct
  REGION 1..13: note = peptide
AKFVAAWTLKAAA

SEQ ID NO: 367 - protein, 12 aa, synthetic construct
  REGION 1..12: note = peptide
AKFVAAWTLKAA

SEQ ID NO: 368 - protein, 13 aa, synthetic construct
  REGION 1..13: note = peptide
  VARIANT 2: note = X can be any amino acid
  VARIANT 7: note = X can be any amino acid
  VARIANT 10: note = X can be any amino acid
  VARIANT 12: note = X can be any amino acid
AXFVAAXTLXAXA

SEQ ID NO: 369 - protein, 17 aa, synthetic construct
  REGION 1..17: note = peptide
AKFVAAWTLKAAANRRA

SEQ ID NO: 370 - protein, 15 aa, synthetic construct
  REGION 1..15: note = peptide
AKFVAAWTLKAAANC

SEQ ID NO: 371 - protein, 14 aa, synthetic construct
  REGION 1..14: note = peptide
AKFVAAWTLKAAAC

SEQ ID NO: 372 - protein, 18 aa, synthetic construct
  REGION 1..18: note = peptide
AKFVAAWTLKAAANRRAC

SEQ ID NO: 373 - protein, 15 aa, synthetic construct
  REGION 1..15: note = peptide
  VARIANT 1: note = aliphatic amino acid selected from alanine, glycine, valine, isoleucine and leucine
  VARIANT 3: note = X can be any amino acid
  VARIANT 13: note = aliphatic amino acid selected from alanine, glycine, valine, isoleucine and leucine
  VARIANT 14: note = aminocaproic acid
XKXVAAWTLKAAXXC

SEQ ID NO: 374 - protein, 19 aa, synthetic construct
  REGION 1..19: note = peptide
  VARIANT 1: note = aliphatic amino acid selected from alanine, glycine, valine, isoleucine and leucine
  VARIANT 3: note = X can be any amino acid
  VARIANT 13: note = aliphatic amino acid selected from alanine, glycine, valine, isoleucine and leucine
  VARIANT 14: note = aminocaproic acid
XKXVAAWTLKAAXXCNRRA

SEQ ID NO: 375 - protein, 13 aa, synthetic construct
  REGION 1..13: note = peptide
  VARIANT 1: note = aliphatic amino acid selected from alanine, glycine, valine, isoleucine and leucine
  VARIANT 3: note = X can be any amino acid
  VARIANT 13: note = aliphatic amino acid selected from alanine, glycine, valine, isoleucine and leucine
XKXVAAWTLKAAX

SEQ ID NO: 376 - protein, 17 aa, synthetic construct
  REGION 1..17: note = peptide
  VARIANT 1: note = aliphatic amino acid selected from alanine, glycine, valine, isoleucine and leucine
  VARIANT 3: note = X can be any amino acid
  VARIANT 13: note = aliphatic amino acid selected from alanine, glycine, valine, isoleucine and leucine
XKXVAAWTLKAAXNRRA

SEQ ID NO: 377 - protein, 13 aa, synthetic construct
  REGION 1..13: note = peptide
  VARIANT 1: note = aliphatic amino acid selected from alanine, glycine, valine, isoleucine and leucine
  VARIANT 3: note = X can be any amino acid
  VARIANT 13: note = aliphatic amino acid selected from alanine, glycine, valine, isoleucine and leucine
XAXAAAKTAAAAX

SEQ ID NO: 378 - protein, 12 aa, synthetic construct
  REGION 1..12: note = peptide
  VARIANT 1: note = aliphatic amino acid selected from alanine, glycine, valine, isoleucine and leucine
  VARIANT 3: note = X can be any amino acid
  VARIANT 12: note = aliphatic amino acid selected from alanine, glycine, valine, isoleucine and leucine
XAXAAATLKAAX

SEQ ID NO: 379 - protein, 13 aa, synthetic construct
  REGION 1..13: note = peptide
  VARIANT 1: note = aliphatic amino acid selected from alanine, glycine, valine, isoleucine and leucine
  VARIANT 3: note = X can be any amino acid
  VARIANT 13: note = aliphatic amino acid selected from alanine, glycine, valine, isoleucine and leucine
XAXVAAATLKAAX

SEQ ID NO: 380 - protein, 13 aa, synthetic construct
  REGION 1..13: note = peptide
  VARIANT 1: note = aliphatic amino acid selected from alanine, glycine, valine, isoleucine and leucine
  VARIANT 3: note = X can be any amino acid
  VARIANT 13: note = aliphatic amino acid selected from alanine, glycine, valine, isoleucine and leucine
XAXIAAATLKAAX

SEQ ID NO: 381 - protein, 12 aa, synthetic construct
  REGION 1..12: note = peptide
  VARIANT 1: note = aliphatic amino acid selected from alanine, glycine, valine, isoleucine and leucine
XKFVAAWTLKAA

SEQ ID NO: 382 - protein, 26 aa, synthetic construct
  REGION 1..26: note = peptide
VTGVTAVAQKTVEGAGNIAAATGFVK

SEQ ID NO: 383 - protein, 15 aa, synthetic construct
  REGION 1..15: note = peptide
GKTKEGVLYVGSKTK

SEQ ID NO: 384 - protein, 15 aa, synthetic construct
  REGION 1..15: note = peptide
KTKEGVLYVGSKTKE

SEQ ID NO: 385 - protein, 9 aa, synthetic construct
  REGION 1..9: note = peptide
VLYVGSKTK

SEQ ID NO: 386 - protein, 15 aa, synthetic construct
  REGION 1..15: note = peptide
MPVDPDNEAYEMPSE

SEQ ID NO: 387 - protein, 15 aa, synthetic construct
  REGION 1..15: note = peptide
DNEAYEMPSEEGYQD

SEQ ID NO: 388 - protein, 15 aa, synthetic construct
  REGION 1..15: note = peptide
EMPSEEGYQDYEPEA

SEQ ID NO: 389 - protein, 15 aa, synthetic construct
  REGION 1..15: note = peptide
EQVTNVGGAVVTGVT

SEQ ID NO: 390 - protein, 12 aa, synthetic construct
  REGION 1..12: note = peptide
DPDNEAYEMPSE

SEQ ID NO: 391 - protein, 8 aa, synthetic construct
  REGION 1..8: note = peptide
DQPVLPDC

SEQ ID NO: 392 - protein, 14 aa, synthetic construct
  REGION 1..14: note = peptide
AKFVAAWTLKAAAN

SEQ ID NO: 393 - protein, 13 aa, synthetic construct
  REGION 1..13: note = peptide
  VARIANT 1: note = aliphatic amino acid selected from alanine, glycine, valine, isoleucine and leucine
  VARIANT 13: note = aliphatic amino acid selected from alanine, glycine, valine, isoleucine and leucine
XKFVAAWTLKAAX

SEQ ID NO: 394 - protein, 6 aa, synthetic construct
  REGION 1..6: note = peptide
DKELRI

SEQ ID NO: 395 - protein, 7 aa, synthetic construct
  REGION 1..7: note = peptide
DKELRID

SEQ ID NO: 396 - protein, 8 aa, synthetic construct
  REGION 1..8: note = peptide
DKELRIDS

SEQ ID NO: 397 - protein, 9 aa, synthetic construct
  REGION 1..9: note = peptide
DKELRIDSG

SEQ ID NO: 398 - protein, 10 aa, synthetic construct
  REGION 1..10: note = peptide
DKELRIDSGY

SEQ ID NO: 399 - protein, 6 aa, synthetic construct
  REGION 1..6: note = peptide
SWEFRT

SEQ ID NO: 400 - protein, 7 aa, synthetic construct
  REGION 1..7: note = peptide
SWEFRTD

SEQ ID NO: 401 - protein, 8 aa, synthetic construct
  REGION 1..8: note = peptide
SWEFRTDS

SEQ ID NO: 402 - protein, 9 aa, synthetic construct
  REGION 1..9: note = peptide
SWEFRTDSG

SEQ ID NO: 403 - protein, 10 aa, synthetic construct
  REGION 1..10: note = peptide
SWEFRTDSGY

SEQ ID NO: 404 - protein, 7 aa, synthetic construct
  REGION 1..7: note = peptide
TLHEFRH

SEQ ID NO: 405 - protein, 7 aa, synthetic construct
  REGION 1..7: note = peptide
TLHEFKH

SEQ ID NO: 406 - protein, 7 aa, synthetic construct
  REGION 1..7: note = peptide
THTDFRH

SEQ ID NO: 407 - protein, 7 aa, synthetic construct
  REGION 1..7: note = peptide
THTDFKH

SEQ ID NO: 408 - protein, 6 aa, synthetic construct
  REGION 1..6: note = peptide
AEFKHD

SEQ ID NO: 409 - protein, 6 aa, synthetic construct
  REGION 1..6: note = peptide
AEFKHG

SEQ ID NO: 410 - protein, 6 aa, synthetic construct
  REGION 1..6: note = peptide
SEFRHD

SEQ ID NO: 411 - protein, 6 aa, synthetic construct
  REGION 1..6: note = peptide
SEFRHG

SEQ ID NO: 412 - protein, 6 aa, synthetic construct
  REGION 1..6: note = peptide
SEFKHD

SEQ ID NO: 413 - protein, 6 aa, synthetic construct
  REGION 1..6: note = peptide
SEFKHG

SEQ ID NO: 414 - protein, 6 aa, synthetic construct
  REGION 1..6: note = peptide
ILFRHG

SEQ ID NO: 415 - protein, 6 aa, synthetic construct
  REGION 1..6: note = peptide
ILFRHD

SEQ ID NO: 416 - protein, 6 aa, synthetic construct
  REGION 1..6: note = peptide
ILFKHG

SEQ ID NO: 417 - protein, 6 aa, synthetic construct
  REGION 1..6: note = peptide
ILFKHD

SEQ ID NO: 418 - protein, 6 aa, synthetic construct
  REGION 1..6: note = peptide
IRWDTP

SEQ ID NO: 419 - protein, 7 aa, synthetic construct
  REGION 1..7: note = peptide
IRYDAPL

SEQ ID NO: 420 - protein, 7 aa, synthetic construct
  REGION 1..7: note = peptide
IRYDMAG

SEQ ID NO: 421 - protein, 19 aa, synthetic construct
  REGION 1..19: note = peptide
SGYSSPGSPGTPGSRSRTP

SEQ ID NO: 422 - protein, 14 aa, synthetic construct
  REGION 1..14: note = peptide
GSRSRTPSLPTPPT

SEQ ID NO: 423 - protein, 15 aa, synthetic construct
  REGION 1..15: note = peptide
KKVAVVRTPPKSPSS

SEQ ID NO: 424 - protein, 16 aa, synthetic construct
  REGION 1..16: note = peptide
  SITE 4: note = phosphorylated serine
  SITE 12: note = phosphorylated serine
VYKSPVVSGDTSPRHL

SEQ ID NO: 425 - protein, 30 aa, synthetic construct
  REGION 1..30: note = peptide
  SITE 18: note = phosphorylated serine
  SITE 26: note = phosphorylated serine
RENAKAKTDHGAEIVYKSPVVSGDTSPRHL

SEQ ID NO: 426 - protein, 5 aa, synthetic construct
  REGION 1..5: note = peptide
  SITE 3: note = phosphorylated serine
IGSTE

SEQ ID NO: 427 - protein, 30 aa, synthetic construct
  REGION 1..30: note = peptide
PDLKNVKSKIGSTENLKHQPGGGKVQIINK

SEQ ID NO: 428 - protein, 30 aa, synthetic construct
  REGION 1..30: note = peptide
VKSKIGSTENLKHQPGGGKVQIINKKLDLS

SEQ ID NO: 429 - protein, 30 aa, synthetic construct; identical to SEQ ID NO: 428
  REGION 1..30: note = peptide
  SITE 7: note = phosphorylated serine

SEQ ID NO: 430 - protein, 30 aa, synthetic construct
  REGION 1..30: note = peptide
KIGSTENLKHQPGGGKVQIINKKLDLSNVQ

SEQ ID NO: 431 - protein, 30 aa, synthetic construct
  REGION 1..30: note = peptide
VQIINKKLDLSNVQSKCGSKDNIKHVPGGG

SEQ ID NO: 432 - protein, 30 aa, synthetic construct
  REGION 1..30: note = peptide
  SITE 17: note = phosphorylated threonine
GSPGTPGSRSRTPSLPTPPTREPKKVAVVR

SEQ ID NO: 433 - protein, 30 aa, synthetic construct
  REGION 1..30: note = peptide
  SITE 22: note = phosphorylated serine
  SITE 25: note = phosphorylated threonine
TPPSSGEPPKSGDRSGYSSPGSPGTPGSRS

SEQ ID NO: 434 - protein, 18 aa, synthetic construct
  REGION 1..18: note = peptide
VPGGGSVQIVYKPVDLSK

SEQ ID NO: 435 - protein, 7 aa, synthetic construct
  REGION 1..7: note = peptide
KHQPGGG

SEQ ID NO: 436 - protein, 7 aa, synthetic construct
  REGION 1..7: note = peptide
KHVPGGG

SEQ ID NO: 437 - protein, 7 aa, synthetic construct
  REGION 1..7: note = peptide
HHVPGGG

SEQ ID NO: 438 - protein, 7 aa, synthetic construct
  REGION 1..7: note = peptide
THVPGGG

SEQ ID NO: 439 - protein, 8 aa, synthetic construct
  REGION 1..8: note = peptide
  SITE 3: note = phosphorylated serine
  SITE 6: note = phosphorylated threonine
PGSPGTPG

SEQ ID NO: 440 - protein, 8 aa, synthetic construct
  REGION 1..8: note = peptide
PGDPGEPG

SEQ ID NO: 441 - protein, 9 aa, synthetic construct
  REGION 1..9: note = peptide
SPGDPGEPG

SEQ ID NO: 442 - protein, 9 aa, synthetic construct
  REGION 1..9: note = peptide
  SITE 3: note = phosphorylated threonine
  SITE 5: note = phosphorylated serine
SRTPSLPTP

SEQ ID NO: 443 - protein, 9 aa, synthetic construct
  REGION 1..9: note = peptide
SREPDLPTP

SEQ ID NO: 444 - protein, 7 aa, synthetic construct
  REGION 1..7: note = peptide
SREPDLP

SEQ ID NO: 445 - protein, 9 aa, synthetic construct
  REGION 1..9: note = peptide
  SITE 5: note = phosphorylated threonine
PSLPTPPTR

SEQ ID NO: 446 - protein, 9 aa, synthetic construct
  REGION 1..9: note = peptide
  SITE 2: note = phosphorylated serine
  SITE 5: note = phosphorylated threonine
PSLPTPPTR

SEQ ID NO: 447 - protein, 9 aa, synthetic construct
  REGION 1..9: note = peptide
PSLPEPPTR

SEQ ID NO: 448 - protein, 9 aa, synthetic construct
  REGION 1..9: note = peptide
PDLPEPPTR

SEQ ID NO: 449 - protein, 10 aa, synthetic construct
  REGION 1..10: note = peptide
  SITE 4: note = phosphorylated serine
VYKSPVVSGD

SEQ ID NO: 450 - protein, 9 aa, synthetic construct
  REGION 1..9: note = peptide
VYKDPVVSG

SEQ ID NO: 451 - protein, 14 aa, synthetic construct
  REGION 1..14: note = peptide
  SITE 4: note = phosphorylated serine
  SITE 12: note = phosphorylated serine
VYKSPVVSGDTSPR

SEQ ID NO: 452 - protein, 14 aa, synthetic construct
  REGION 1..14: note = peptide
VYKDPVVSGDTDPR

SEQ ID NO: 453 - protein, 11 aa, synthetic construct
  REGION 1..11: note = peptide
  SITE 6: note = phosphorylated threonine
PPAPKTPPSSG

SEQ ID NO: 454 - protein, 11 aa, synthetic construct
  REGION 1..11: note = peptide
PPAPKEPPSSG

SEQ ID NO: 455 - protein, 10 aa, synthetic construct
  REGION 1..10: note = peptide
  SITE 7: note = phosphorylated threonine
KVAVVRTPPK

SEQ ID NO: 456 - protein, 11 aa, synthetic construct
  REGION 1..11: note = peptide
KVAVVREPPKS

SEQ ID NO: 457 - protein, 30 aa, synthetic construct
  REGION 1..30: note = peptide
RENAKAKTDHGAEIVYKDPVVSGDTDPRHL

SEQ ID NO: 458 - protein, 20 aa, synthetic construct
  REGION 1..20: note = peptide
  SITE 8: note = phosphorylated serine
  SITE 16: note = phosphorylated serine
GAEIVYKSPVVSGDTSPRHL

SEQ ID NO: 459 - protein, 18 aa, synthetic construct
  REGION 1..18: note = peptide
  SITE 6: note = phosphorylated serine
  SITE 14: note = phosphorylated serine
EIVYKSPVVSGDTSPRHL

SEQ ID NO: 460 - protein, 20 aa, synthetic construct
  REGION 1..20: note = peptide
GAEIVYKDPVVSGDTDPRHL

SEQ ID NO: 461 - protein, 18 aa, synthetic construct
  REGION 1..18: note = peptide
EIVYKDPVVSGDTDPRHL

SEQ ID NO: 462 - protein, 9 aa, synthetic construct
  REGION 1..9: note = peptide
  SITE 5: note = phosphorylated serine
DMVDSPQLA

SEQ ID NO: 463 - protein, 9 aa, synthetic construct
  REGION 1..9: note = peptide
DMVDDPQLA

SEQ ID NO: 464 - protein, 7 aa, synthetic construct
  REGION 1..7: note = peptide
  SITE 3: note = phosphorylated serine
VDSPQLA

SEQ ID NO: 465 - protein, 7 aa, synthetic construct
  REGION 1..7: note = peptide
VDDPQLA

SEQ ID NO: 466 - protein, 5 aa, synthetic construct
  REGION 1..5: note = peptide
HVPGG

SEQ ID NO: 467 - protein, 16 aa, synthetic construct
  REGION 1..16: note = peptide
LDNITHVPGGGNKKIE

SEQ ID NO: 468 - protein, 12 aa, synthetic construct
  REGION 1..12: note = peptide
SPSSAKSRLQTA

SEQ ID NO: 469 - protein, 7 aa, synthetic construct
  REGION 1..7: note = peptide
DAEFRHD

SEQ ID NO: 470 - protein, 8 aa, synthetic construct
  REGION 1..8: note = peptide
DAEFRHDS

SEQ ID NO: 471 - protein, 9 aa, synthetic construct
  REGION 1..9: note = peptide
DAEFRHDSG

SEQ ID NO: 472 - protein, 10 aa, synthetic construct
  REGION 1..10: note = peptide
DAEFRHDSGY

SEQ ID NO: 473 - protein, 11 aa, synthetic construct
  REGION 1..11: note = peptide
DAEFRHDSGYE

SEQ ID NO: 474 - protein, 12 aa, synthetic construct
  REGION 1..12: note = peptide
DAEFRHDSGYEV

SEQ ID NO: 475 - protein, 13 aa, synthetic construct
  REGION 1..13: note = peptide
DAEFRHDSGYEVH

SEQ ID NO: 476 - protein, 14 aa, synthetic construct
  REGION 1..14: note = peptide
DAEFRHDSGYEVHH

SEQ ID NO: 477 - protein, 15 aa, synthetic construct
  REGION 1..15: note = peptide
DAEFRHDSGYEVHHQ

SEQ ID NO: 478 - protein, 21 aa, synthetic construct
  REGION 1..21: note = peptide
DAEFRHDSGYEVHHQKLVFFA

SEQ ID NO: 479 - protein, 6 aa, synthetic construct
  REGION 1..6: note = peptide
AEFRHD

SEQ ID NO: 480 - protein, 7 aa, synthetic construct
  REGION 1..7: note = peptide
AEFRHDS

SEQ ID NO: 481 - protein, 8 aa, synthetic construct
  REGION 1..8: note = peptide
AEFRHDSG

SEQ ID NO: 482 - protein, 9 aa, synthetic construct
  REGION 1..9: note = peptide
AEFRHDSGY

SEQ ID NO: 483 - protein, 6 aa, synthetic construct
  REGION 1..6: note = peptide
EFRHDS

SEQ ID NO: 484 - protein, 7 aa, synthetic construct
  REGION 1..7: note = peptide
EFRHDSG

SEQ ID NO: 485 - protein, 8 aa, synthetic construct
  REGION 1..8: note = peptide
EFRHDSGY

SEQ ID NO: 486 - protein, 7 aa, synthetic construct
  REGION 1..7: note = peptide
  SITE 1: note = pyroglutamic acid
EFRHDSG

SEQ ID NO: 487 - protein, 8 aa, synthetic construct
  REGION 1..8: note = peptide
  SITE 1: note = pyroglutamic acid
EFRHDSGY

SEQ ID NO: 488 - protein, 6 aa, synthetic construct
  REGION 1..6: note = peptide
EVHHQK

SEQ ID NO: 489 - protein, 7 aa, synthetic construct
  REGION 1..7: note = peptide
EVHHQKL

SEQ ID NO: 490 - protein, 8 aa, synthetic construct
  REGION 1..8: note = peptide
EVHHQKLV

SEQ ID NO: 491 - protein, 9 aa, synthetic construct
  REGION 1..9: note = peptide
EVHHQKLVF

SEQ ID NO: 492 - protein, 8 aa, synthetic construct
  REGION 1..8: note = peptide
VHHQKLVF

SEQ ID NO: 493 - protein, 7 aa, synthetic construct
  REGION 1..7: note = peptide
HHQKLVF

SEQ ID NO: 494 - protein, 6 aa, synthetic construct
  REGION 1..6: note = peptide
HQKLVF

SEQ ID NO: 495 - protein, 7 aa, synthetic construct
  REGION 1..7: note = peptide
HQKLVFF

SEQ ID NO: 496 - protein, 8 aa, synthetic construct
  REGION 1..8: note = peptide
HQKLVFFA

SEQ ID NO: 497 - protein, 9 aa, synthetic construct
  REGION 1..9: note = peptide
HQKLVFFAE

SEQ ID NO: 498 - protein, 10 aa, synthetic construct
  REGION 1..10: note = peptide
HQKLVFFAED

SEQ ID NO: 499 - protein, 11 aa, synthetic construct
  REGION 1..11: note = peptide
AIIGLMVGGVV

SEQ ID NO: 500 - protein, 10 aa, synthetic construct
  REGION 1..10: note = peptide
IIGLMVGGVV

SEQ ID NO: 501 - protein, 9 aa, synthetic construct
  REGION 1..9: note = peptide
IGLMVGGVV

SEQ ID NO: 502 - protein, 8 aa, synthetic construct
  REGION 1..8: note = peptide
GLMVGGVV

SEQ ID NO: 503 - protein, 7 aa, synthetic construct
  REGION 1..7: note = peptide
LMVGGVV

SEQ ID NO: 504 - protein, 13 aa, synthetic construct
  REGION 1..13: note = peptide
AIIGLMVGGVVIA

SEQ ID NO: 505 - protein, 18 aa, synthetic construct
  REGION 1..18: note = peptide
GMLATVAFNTFTDNVDQR

SEQ ID NO: 506 - protein, 12 aa, synthetic construct
  REGION 1..12: note = peptide
AFNKFTDNVDQR

SEQ ID NO: 507 - protein, 8 aa, synthetic construct
  REGION 1..8: note = peptide
MPRCRFGF

SEQ ID NO: 508 - protein, 11 aa, synthetic construct
  REGION 1..11: note = peptide
WRTQNDVLENG

SEQ ID NO: 509 - protein, 15 aa, synthetic construct
  REGION 1..15: note = peptide
TFTDNVDQRMPRCRH

SEQ ID NO: 510 - protein, 12 aa, synthetic construct
  REGION 1..12: note = peptide
RVVELMDWTVLH

SEQ ID NO: 511 - protein, 12 aa, synthetic construct
  REGION 1..12: note = peptide
GSAMTWGMLAAE

SEQ ID NO: 512 - protein, 12 aa, synthetic construct
  REGION 1..12: note = peptide
SYNIIATGIHPV

SEQ ID NO: 513 - protein, 12 aa, synthetic construct
  REGION 1..12: note = peptide
TMVATGLMPVLI

SEQ ID NO: 514 - protein, 7 aa, synthetic construct
  REGION 1..7: note = peptide
QDFDDIL

SEQ ID NO: 515 - protein, 9 aa, synthetic construct
  REGION 1..9: note = peptide
CLFSQGNRC

SEQ ID NO: 516 - protein, 8 aa, synthetic construct
  REGION 1..8: note = peptide
VANLKVGV

SEQ ID NO: 517 - protein, 12 aa, synthetic construct
  REGION 1..12: note = peptide
NPGGLSAAPAGS

SEQ ID NO: 518 - protein, 11 aa, synthetic construct
  REGION 1..11: note = peptide
RHASTLLGRHG

SEQ ID NO: 519 - protein, 12 aa, synthetic construct
  REGION 1..12: note = peptide
EAAESMWRVASG

SEQ ID NO: 520 - protein, 14 aa, synthetic construct
  REGION 1..14: note = peptide
QNRLNSNGNNGGSQ

SEQ ID NO: 521 - protein, 12 aa, synthetic construct
  REGION 1..12: note = peptide
DDDLQHQFDDQD

SEQ ID NO: 522 - protein, 15 aa, synthetic construct
  REGION 1..15: note = peptide
CDIKDPIRLEPGGPD

SEQ ID NO: 523 - protein, 15 aa, synthetic construct
  REGION 1..15: note = peptide
EVWREEAYHACDIKD

SEQ ID NO: 524 - protein, 15 aa, synthetic construct
  REGION 1..15: note = peptide
GKVYLVGGPELGGWK

SEQ ID NO: 525 - protein, 15 aa, synthetic construct
  REGION 1..15: note = peptide
LGGWKLQSDPRAYAL

SEQ ID NO: 526 - protein, 15 aa, synthetic construct
  REGION 1..15: note = peptide
NFTTGEDSVAEVWRE

SEQ ID NO: 527 - protein, 15 aa, synthetic construct
  REGION 1..15: note = peptide
PGGPDRFTLLTPGSH

SEQ ID NO: 528 - protein, 15 aa, synthetic construct
  REGION 1..15: note = peptide
QFKTTDVLWFNFTTG

SEQ ID NO: 529 - protein, 15 aa, synthetic construct
  REGION 1..15: note = peptide
RAYALWSARQQFKTT

SEQ ID NO: 530 - protein, 15 aa, synthetic construct
  REGION 1..15: note = peptide
TPGSHFICTKDQKFV

SEQ ID NO: 531 - protein, 7 aa, synthetic construct
  REGION 1..7: note = peptide
MISTSRK

SEQ ID NO: 532 - protein, 7 aa, synthetic construct
  REGION 1..7: note = peptide
QKRNTIT

SEQ ID NO: 533 - protein, 10 aa, synthetic construct
  REGION 1..10: note = peptide
KISEFYGRKP

SEQ ID NO: 534 - protein, 10 aa, synthetic construct
  REGION 1..10: note = peptide
VATATLPNYC

SEQ ID NO: 535 - protein, 10 aa, synthetic construct
  REGION 1..10: note = peptide
YSCGENSFMD

SEQ ID NO: 536 - protein, 10 aa, synthetic construct
  REGION 1..10: note = peptide
YYNSLGFNIK

SEQ ID NO: 537 - protein, 20 aa, synthetic construct
  REGION 1..20: note = peptide
REREREEDWRQPREDWRRPS

SEQ ID NO: 538 - protein, 20 aa, synthetic construct
  REGION 1..20: note = peptide
RTRGRQPGDYDDDRRQPRRE

SEQ ID NO: 539 - protein, 20 aa, synthetic construct
  REGION 1..20: note = peptide
DDDRRQPRREEGGRWGPAGP

SEQ ID NO: 540 - protein, 20 aa, synthetic construct
  REGION 1..20: note = peptide
TTNQRSPPGERTRGRQPGDY

SEQ ID NO: 541 - protein, 20 aa, synthetic construct
  REGION 1..20: note = peptide
QPREDWRRPSHQQPRKIRPE

SEQ ID NO: 542 - protein, 20 aa, synthetic construct
  REGION 1..20: note = peptide
GREGEQEWGTPGSHVREETS

SEQ ID NO: 543 - protein, 20 aa, synthetic construct
  REGION 1..20: note = peptide
PVNTPGQFEDFFPASSRDQS

SEQ ID NO: 544 - protein, 20 aa, synthetic construct
  REGION 1..20: note = peptide
AGGEQEERGQRRWSTRSSEN

SEQ ID NO: 545 - protein, 20 aa, synthetic construct
  REGION 1..20: note = peptide
REGEPDLSNNFGKLFEVKPD

SEQ ID NO: 546 - protein, 20 aa, synthetic construct
  REGION 1..20: note = peptide
NASSELHLLGFGINAENNHR

SEQ ID NO: 547 - protein, 20 aa, synthetic construct
  REGION 1..20: note = peptide
IDQIEKQAKDLAFPGSGEQV

SEQ ID NO: 548 - protein, 20 aa, synthetic construct
  REGION 1..20: note = peptide
LAFPGSGEQVEKLIKNQKES

SEQ ID NO: 549 - protein, 10 aa, synthetic construct
  REGION 1..10: note = peptide
AKSSPYQKKT

SEQ ID NO: 550 - protein, 10 aa, synthetic construct
  REGION 1..10: note = peptide
QEPDDLKQKA

SEQ ID NO: 551 - protein, 10 aa, synthetic construct
  REGION 1..10: note = peptide
LEYDPRLUYD

SEQ ID NO: 552 - protein, 10 aa, synthetic construct
  REGION 1..10: note = peptide
GERTRGRQPG

SEQ ID NO: 553 - protein, 10 aa, synthetic construct
  REGION 1..10: note = peptide
PGDYDDDRRQ

SEQ ID NO: 554 - protein, 10 aa, synthetic construct
  REGION 1..10: note = peptide
PRREEGGRWG

SEQ ID NO: 555 - protein, 10 aa, synthetic construct
  REGION 1..10: note = peptide
REREEDWRQP

SEQ ID NO: 556 - protein, 10 aa, synthetic construct
  REGION 1..10: note = peptide
EDWRRPSHQQ

SEQ ID NO: 557 - protein, 10 aa, synthetic construct
  REGION 1..10: note = peptide
QPKKIRPEGR

SEQ ID NO: 558 - protein, 10 aa, synthetic construct
  REGION 1..10: note = peptide
TPGQFEDFFP

SEQ ID NO: 559 - protein, 10 aa, synthetic construct
  REGION 1..10: note = peptide
SYLQEFSRNT

SEQ ID NO: 560 - protein, 10 aa, synthetic construct
  REGION 1..10: note = peptide
FNAEFNEIRR

SEQ ID NO: 561 - protein, 10 aa, synthetic construct
  REGION 1..10: note = peptide
EQEERGQRRW

SEQ ID NO: 562 - protein, 10 aa, synthetic construct
  REGION 1..10: note = peptide
DITNPINLRE

SEQ ID NO: 563 - protein, 10 aa, synthetic construct
  REGION 1..10: note = peptide
NNFGKLFEVK

SEQ ID NO: 564 - protein, 10 aa, synthetic construct
  REGION 1..10: note = peptide
GTGNLELVAV

SEQ ID NO: 565 - protein, 10 aa, synthetic construct
  REGION 1..10: note = peptide
RRYTARLKEG

SEQ ID NO: 566 - protein, 10 aa, synthetic construct
  REGION 1..10: note = peptide
ELHLLGFGIN

SEQ ID NO: 567 - protein, 10 aa, synthetic construct
  REGION 1..10: note = peptide
HRIFLAGDKD

SEQ ID NO: 568 - protein, 10 aa, synthetic construct
  REGION 1..10: note = peptide
IDOIEKOAKD

SEQ ID NO: 569 - protein, 10 aa, synthetic construct
  REGION 1..10: note = peptide
KDLAFPGSGE

SEQ ID NO: 570 - protein, 10 aa, synthetic construct
  REGION 1..10: note = peptide
KESHFVSARP

SEQ ID NO: 571 - protein, 10 aa, synthetic construct
  REGION 1..10: note = peptide
PEKESPEKED

SEQ ID NO: 572 - protein, 20 aa, synthetic construct
  REGION 1..20: note = peptide
ARQQWELQGDRRCQSQLERA

SEQ ID NO: 573 - protein, 20 aa, synthetic construct
  REGION 1..20: note = peptide
ERDPYSPSQDPYSPSPYDRR

SEQ ID NO: 574 - protein, 20 aa, synthetic construct
  REGION 1..20: note = peptide
RRCQSQLERANLRPCEQHLM

SEQ ID NO: 575 - protein, 20 aa, synthetic construct
  REGION 1..20: note = peptide
RDPYSPSPYDRRGAGSSQHQ

SEQ ID NO: 576 - protein, 20 aa, synthetic construct
  REGION 1..20: note = peptide
GRDPYSPSQDPYSPSQDPDR

SEQ ID NO: 577 - protein, 20 aa, synthetic construct
  REGION 1..20: note = peptide
PYSPSQDPDRRDPYSPSPYD

SEQ ID NO: 578 - protein, 20 aa, synthetic construct
  REGION 1..20: note = peptide
QKIQRDEDSYERDPYSPSQD

SEQ ID NO: 579 - protein, 10 aa, synthetic construct
  REGION 1..10: note = peptide
HASARQQWEL

SEQ ID NO: 580 - protein, 10 aa, synthetic construct
  REGION 1..10: note = peptide
QWELQGDRRC

SEQ ID NO: 581 - protein, 10 aa, synthetic construct
  REGION 1..10: note = peptide
DRRCQSQLER

SEQ ID NO: 582 - protein, 10 aa, synthetic construct
  REGION 1..10: note = peptide
LRPCEQHLMQ

SEQ ID NO: 583 - protein, 10 aa, synthetic construct
  REGION 1..10: note = peptide
KIQRDEDSYE

SEQ ID NO: 584 - protein, 10 aa, synthetic construct
  REGION 1..10: note = peptide
YERDPYSPSQ

SEQ ID NO: 585 - protein, 10 aa, synthetic construct
  REGION 1..10: note = peptide
SQDPYSPSPY

SEQ ID NO: 586 - protein, 10 aa, synthetic construct
  REGION 1..10: note = peptide
DRLQGRQQEQ

SEQ ID NO: 587 - protein, 10 aa, synthetic construct
  REGION 1..10: note = peptide
KRELRNLPQQ

SEQ ID NO: 588 - protein, 10 aa, synthetic construct
  REGION 1..10: note = peptide
QRCDLDVESG

SEQ ID NO: 589 - protein, 15 aa, synthetic construct
  REGION 1..15: note = peptide
ERDPYSPSQDPYSPS

SEQ ID NO: 590 - protein, 9 aa, synthetic construct
  REGION 1..9: note = peptide
RRCQSQLER

SEQ ID NO: 591 - protein, 13 aa, synthetic construct
  REGION 1..13: note = peptide
CDLEVESGGRDRY

SEQ ID NO: 592 - protein, 16 aa, synthetic construct
  REGION 1..16: note = peptide
NLRPCEQHLMQKIQRD

SEQ ID NO: 593 - protein, 7 aa, synthetic construct
  REGION 1..7: note = peptide
PQRCDLE

SEQ ID NO: 594 - protein, 17 aa, synthetic construct
  REGION 1..17: note = peptide
RQQEQQFKRELRNLPQQ

SEQ ID NO: 595 - protein, 9 aa, synthetic construct
  REGION 1..9: note = peptide
SDRLQGRQQ

SEQ ID NO: 596 - protein, 42 aa, synthetic construct
  REGION 1..42: note = peptide
RRCQSQLERANLRPCEQHLMQKIQRDEDSYGRDPYSPSQDPY

SEQ ID NO: 597 - protein, 15 aa, synthetic construct
  REGION 1..15: note = peptide
IETWNPNNQEFECAG

SEQ ID NO: 598 - protein, 15 aa, synthetic construct
  REGION 1..15: note = peptide
GNIFSGFTPEFLEQA

SEQ ID NO: 599 - protein, 15 aa, synthetic construct
  REGION 1..15: note = peptide
VTVRGGLRILSPDRK

SEQ ID NO: 600 - protein, 13 aa, synthetic construct
  REGION 1..13: note = peptide
DEDEYEYDEEDRG

SEQ ID NO: 601 - protein, 20 aa, synthetic construct
  REGION 1..20: note = peptide
EDEYEYDEEDRRRGRGSRGR

SEQ ID NO: 602 - protein, 20 aa, synthetic construct
  REGION 1..20: note = peptide
NIFSGFTPEFLEQAFQVDDR

SEQ ID NO: 603 - protein, 20 aa, synthetic construct
  REGION 1..20: note = peptide
ESEEEGAIVTVRGGLRILSP

SEQ ID NO: 604 - protein, 20 aa, synthetic construct
  REGION 1..20: note = peptide
SGFTPEFLEQAFQVDDRQIV

SEQ ID NO: 605 - protein, 20 aa, synthetic construct
  REGION 1..20: note = peptide
EEGAIVTVRGGLRILSPDRK

SEQ ID NO: 606 - protein, 20 aa, synthetic construct
  REGION 1..20: note = peptide
TYEEPAQQGRRYQSQRPPRR

SEQ ID NO: 607 - protein, 20 aa, synthetic construct
  REGION 1..20: note = peptide
RRADEEEEYDEDEYEYDEED

SEQ ID NO: 608 - protein, 20 aa, synthetic construct
  REGION 1..20: note = peptide
NHEQEFLRYQQQSRQSRRRS

SEQ ID NO: 609 - protein, 20 aa, synthetic construct
  REGION 1..20: note = peptide
QEFLRYQQQSRQSRRRSLPY

SEQ ID NO: 610 - protein, 20 aa, synthetic construct
  REGION 1..20: note = peptide
QEEREFSPRGQHSRRERAGQ

SEQ ID NO: 611 - protein, 12 aa, synthetic construct
  REGION 1..12: note = peptide
CDELNEMENTQR

SEQ ID NO: 612 - protein, 13 aa, synthetic construct
  REGION 1..13: note = peptide
CEALQQIMENQCD

SEQ ID NO: 613 - protein, 13 aa, synthetic construct
  REGION 1..13: note = peptide
CNFRAPQRCDLDV

SEQ ID NO: 614 - protein, 11 aa, synthetic construct
  REGION 1..11: note = peptide
KPCEQHIMQRI

SEQ ID NO: 615 - protein, 7 aa, synthetic construct
  REGION 1..7: note = peptide
YDSYDIR

SEQ ID NO: 616 - protein, 10 aa, synthetic construct
  REGION 1..10: note = peptide
KRELRMLPQQ

SEQ ID NO: 617 - protein, 14 aa, synthetic construct
  REGION 1..14: note = peptide
MRRERGRGGDSSSS

SEQ ID NO: 618 - protein, 31 aa, synthetic construct
  REGION 1..31: note = peptide
KAEQVKASKEMGETLLRAVESYLLAHSDAYN

SEQ ID NO: 619 - protein, 30 aa, synthetic construct
  REGION 1..30: note = peptide
GPGTIKKISFPEGFPFKYVKDRVDEVDHTN

SEQ ID NO: 620 - protein, 30 aa, synthetic construct
  REGION 1..30: note = peptide
DGGSILKISNKYHTKGDHEVKAEQVKASKE

SEQ ID NO: 621 - protein, 30 aa, synthetic construct
  REGION 1..30: note = peptide
LFPKVAPQAISSVENIEGNGGPGTIKKISF

SEQ ID NO: 622 - protein, 14 aa, synthetic construct
  REGION 1..14: note = peptide
MGETLLRAVESYLL

SEQ ID NO: 623 - protein, 24 aa, synthetic construct
  REGION 1..24: note = peptide
MGVFNYETETTSVIPAARLFKAFI

SEQ ID NO: 624 - protein, 30 aa, synthetic construct
  REGION 1..30: note = peptide
VDHTNFKYNYSVIEGGPIGDTLEKISNEIK

SEQ ID NO: 625 - protein, 10 aa, synthetic construct
  REGION 1..10: note = peptide
ISFPEGFPFK

SEQ ID NO: 626 - protein, 21 aa, synthetic construct
  REGION 1..21: note = peptide
FILDGDNLFPKVAPQAISSVE

SEQ ID NO: 627 - protein, 13 aa, synthetic construct
  REGION 1..13: note = peptide
NIEGNGGPGTIKK

SEQ ID NO: 628 - protein, 10 aa, synthetic construct
  REGION 1..10: note = peptide
DGDNLFPKVA

SEQ ID NO: 629 - protein, 16 aa, synthetic construct
  REGION 1..16: note = peptide
ILDGDNLFPKVAPQAI

SEQ ID NO: 630 - protein, 21 aa, synthetic construct
  REGION 1..21: note = peptide
SKEMGETLLRAVESYLLAHSD

SEQ ID NO: 631 - protein, 16 aa, synthetic construct
  REGION 1..16: note = peptide
EVDHTNFKYNYSVIEG

SEQ ID NO: 632 - protein, 16 aa, synthetic construct
  REGION 1..16: note = peptide
GGPIGDTLEKISNEIK

SEQ ID NO: 633 - protein, 10 aa, synthetic construct
  REGION 1..10: note = peptide
GPGTIKKISF

SEQ ID NO: 634 - protein, 23 aa, synthetic construct
  REGION 1..23: note = peptide
EQVKASKEMGETLLRAVESYLLA

SEQ ID NO: 635 - protein, 14 aa, synthetic construct
  REGION 1..14: note = peptide
GDNLFPKVAPQAIS

SEQ ID NO: 636 - protein, 11 aa, synthetic construct
  REGION 1..11: note = peptide
ISFPEGFPFKY

SEQ ID NO: 637 - protein, 14 aa, synthetic construct
  REGION 1..14: note = peptide
ISFPEGFPFKYVKD

SEQ ID NO: 638 - protein, 11 aa, synthetic construct
  REGION 1..11: note = peptide
CQQFLSVRALC

SEQ ID NO: 639 - protein, 10 aa, synthetic construct
  REGION 1..10: note = peptide
QQFLSVRALC

SEQ ID NO: 640 - protein, 10 aa, synthetic construct
  REGION 1..10: note = peptide
CQQFLSVRAL

SEQ ID NO: 641 - protein, 12 aa, synthetic construct
  REGION 1..12: note = peptide
DALTLRTATNIW

SEQ ID NO: 642 - protein, 12 aa, synthetic construct
  REGION 1..12: note = peptide
DGDALTLRTATN

SEQ ID NO: 643 - protein, 12 aa, synthetic construct
  REGION 1..12: note = peptide
DGRGAQVYIGNG

SEQ ID NO: 644 - protein, 12 aa, synthetic construct
  REGION 1..12: note = peptide
NATPQLTKNAGV

SEQ ID NO: 645 - protein, 12 aa, synthetic construct
  REGION 1..12: note = peptide
NGGPCVFIKRVS

SEQ ID NO: 646 - protein, 12 aa, synthetic construct
  REGION 1..12: note = peptide
NGNATPQLTKNA

SEQ ID NO: 647 - protein, 12 aa, synthetic construct
  REGION 1..12: note = peptide
NSDDDPVNPAPG

SEQ ID NO: 648 - protein, 12 aa, synthetic construct
  REGION 1..12: note = peptide
VENGNATPQLTK

SEQ ID NO: 649 - protein, 11 aa, synthetic construct
  REGION 1..11: note = peptide
NAGVLTCSLSK

SEQ ID NO: 650 - protein, 8 aa, synthetic construct
  REGION 1..8: note = peptide
KWVNGREI

SEQ ID NO: 651 - protein, 20 aa, synthetic construct
  REGION 1..20: note = peptide
GQCKWVNGREICNDRDRPTA

SEQ ID NO: 652 - protein, 20 aa, synthetic construct
  REGION 1..20: note = peptide
GRENSRAEVSYVHVNGAKFI

SEQ ID NO: 653 - protein, 20 aa, synthetic construct
  REGION 1..20: note = peptide
PGNKKFVVNNLFFNGPCQPH

SEQ ID NO: 654 - protein, 25 aa, synthetic construct
  REGION 1..25: note = peptide
SHIIYENVEMINSENPILINQFYCT

SEQ ID NO: 655 - protein, 20 aa, synthetic construct
  REGION 1..20: note = peptide
YCTSASACQNQRSAVQIQDV

SEQ ID NO: 656 - protein, 20 aa, synthetic construct
  REGION 1..20: note = peptide
TYKNIRGTSATAAAIQLKCS

SEQ ID NO: 657 - protein, 12 aa, synthetic construct
  REGION 1..12: note = peptide
AEVSYVHVNGAK

SEQ ID NO: 658 - protein, 9 aa, synthetic construct
  REGION 1..9: note = peptide
KGIPNTKAP

SEQ ID NO: 659 - protein, 9 aa, synthetic construct
  REGION 1..9: note = peptide
DMFQIGKYG

SEQ ID NO: 660 - protein, 9 aa, synthetic construct
  REGION 1..9: note = peptide
GIREVWPAG

SEQ ID NO: 661 - protein, 9 aa, synthetic construct
  REGION 1..9: note = peptide
SSMGAYWGG

SEQ ID NO: 662 - protein, 9 aa, synthetic construct
  REGION 1..9: note = peptide
KGTTGVRNT

SEQ ID NO: 663 - protein, 17 aa, synthetic construct
  REGION 1..17: note = peptide
CQIYPPNANKIREALAQ

SEQ ID NO: 664 - protein, 12 aa, synthetic construct
  REGION 1..12: note = peptide
GYSNAQGVDYWI

SEQ ID NO: 665 - protein, 20 aa, synthetic construct
  REGION 1..20: note = peptide
NQSLDLAEQELVDCASQHGC

SEQ ID NO: 666 - protein, 14 aa, synthetic construct
  REGION 1..14: note = peptide
VRNSWDTNWGDNGY

SEQ ID NO: 667 - protein, 15 aa, synthetic construct
  REGION 1..15: note = peptide
EQSYPCWLSGTPSTP

SEQ ID NO: 668 - protein, 15 aa, synthetic construct
  REGION 1..15: note = peptide
TPLCDYAAARVGACG

SEQ ID NO: 669 - protein, 15 aa, synthetic construct
  REGION 1..15: note = peptide
KEQLPTSYPPERAGW

SEQ ID NO: 670 - protein, 15 aa, synthetic construct
  REGION 1..15: note = peptide
NCLSSDEPLHIRWCQ

SEQ ID NO: 671 - protein, 15 aa, synthetic construct
  REGION 1..15: note = peptide
EALSEEEWPRYTSHP

SEQ ID NO: 672 - protein, 13 aa, synthetic construct
  REGION 1..13: note = peptide
SCDATQRAQGRCS

SEQ ID NO: 673 - protein, 13 aa, synthetic construct
  REGION 1..13: note = peptide
SCDSSQKKQGRCS

SEQ ID NO: 674 - protein, 13 aa, synthetic construct
  REGION 1..13: note = peptide
SCDESRRRQGRCS

SEQ ID NO: 675 - protein, 10 aa, synthetic construct
  REGION 1..10: note = peptide
CKGIPNTKAP

SEQ ID NO: 676 - protein, 10 aa, synthetic construct
  REGION 1..10: note = peptide
CDMFQIGKYG

SEQ ID NO: 677 - protein, 10 aa, synthetic construct
  REGION 1..10: note = peptide
CGIREVWPAG

SEQ ID NO: 678 - protein, 10 aa, synthetic construct
  REGION 1..10: note = peptide
CSSMGAYWGG

SEQ ID NO: 679 - protein, 10 aa, synthetic construct
  REGION 1..10: note = peptide
CKGTTGVRNT

SEQ ID NO: 680 - protein, 11 aa, synthetic construct
  REGION 1..11: note = peptide
CKGIPNTKAPC

SEQ ID NO: 681 - protein, 11 aa, synthetic construct
  REGION 1..11: note = peptide
CDMFQIGKYGC

SEQ ID NO: 682 - protein, 11 aa, synthetic construct
  REGION 1..11: note = peptide
CGIREVWPAGC

SEQ ID NO: 683 - protein, 11 aa, synthetic construct
  REGION 1..11: note = peptide
CSSMGAYWGGC

SEQ ID NO: 684 - protein, 10 aa, synthetic construct
  REGION 1..10: note = peptide
KGTTGVRNTC

SEQ ID NO: 685 - protein, 10 aa, synthetic construct
  REGION 1..10: note = peptide
KGIPNTKAPC

SEQ ID NO: 686 - protein, 10 aa, synthetic construct
  REGION 1..10: note = peptide
DMFQIGKYGC

SEQ ID NO: 687 - protein, 10 aa, synthetic construct
  REGION 1..10: note = peptide
GIREVWPAGC

SEQ ID NO: 688 - protein, 10 aa, synthetic construct
  REGION 1..10: note = peptide
SSMGAYWGGC

SEQ ID NO: 689 - protein, 9 aa, synthetic construct
  REGION 1..9: note = peptide
FVVEYTKKW

SEQ ID NO: 690 - protein, 9 aa, synthetic construct
  REGION 1..9: note = peptide
SWWNLPQIG

SEQ ID NO: 691 - protein, 9 aa, synthetic construct
  REGION 1..9: note = peptide
KGITTKWMA

SEQ ID NO: 692 - protein, 9 aa, synthetic construct
  REGION 1..9: note = peptide
AGISYTKTW

SEQ ID NO: 693 - protein, 15 aa, synthetic construct
  REGION 1..15: note = peptide
DQVDVKDCANHEIKK

SEQ ID NO: 694 - protein, 15 aa, synthetic construct
  REGION 1..15: note = peptide
VPGIDPNACHYMKCK

SEQ ID NO: 695 - protein, 32 aa, synthetic construct
  REGION 1..32: note = peptide
APKSENVVVTVKVMGDNGVLACAIATHAKIRD

SEQ ID NO: 696 - protein, 31 aa, synthetic construct
  REGION 1..31: note = peptide
CHGSEPCIIHRGKPFQLEAVFEANQNSKTAK

SEQ ID NO: 697 - protein, 33 aa, synthetic construct
  REGION 1..33: note = peptide
DQVDVKDCANHEIKKVLVPGCHGSEPCIIHRGK

SEQ ID NO: 698 - protein, 32 aa, synthetic construct
  REGION 1..32: note = peptide
EANQNSKTAKIEIKASIEGLEVDVPGIDPNAC

SEQ ID NO: 699 - protein, 42 aa, synthetic construct
  REGION 1..42: note = peptide
EVDVPGIDPNACHYMKCPLVKGQQYDIKYTWIVPKIAPKSEN

SEQ ID NO: 700 - protein, 10 aa, synthetic construct
  REGION 1..10: note = peptide
FVVEYTKKWC

SEQ ID NO: 701 - protein, 10 aa, synthetic construct
  REGION 1..10: note = peptide
SWWNLPQIGC

SEQ ID NO: 702 - protein, 10 aa, synthetic construct
  REGION 1..10: note = peptide
KGITTKWMAC

SEQ ID NO: 703 - protein, 10 aa, synthetic construct
  REGION 1..10: note = peptide
AGISYTKTWC

SEQ ID NO: 704 - protein, 11 aa, synthetic construct
  REGION 1..11: note = peptide
CFVVEYTKKWC

SEQ ID NO: 705 - protein, 11 aa, synthetic construct
  REGION 1..11: note = peptide
CSWWNLPQIGC

SEQ ID NO: 706 - protein, 11 aa, synthetic construct
  REGION 1..11: note = peptide
CKGITTKWMAC

SEQ ID NO: 707 - protein, 11 aa, synthetic construct
  REGION 1..11: note = peptide
CAGISYTKTWC

SEQ ID NO: 708 - protein, 10 aa, synthetic construct
  REGION 1..10: note = peptide
CFVVEYTKKW

SEQ ID NO: 709 - protein, 10 aa, synthetic construct
  REGION 1..10: note = peptide
CSWWNLPQIG

SEQ ID NO: 710 - protein, 10 aa, synthetic construct
  REGION 1..10: note = peptide
CKGITTKWMA

SEQ ID NO: 711 - protein, 10 aa, synthetic construct
  REGION 1..10: note = peptide
CAGISYTKTW

SEQ ID NO: 712 - protein, 11 aa, synthetic construct
  REGION 1..11: note = peptide
QEELREAFRMY

SEQ ID NO: 713 - protein, 11 aa, synthetic construct
  REGION 1..11: note = peptide
TSALREILRAL

SEQ ID NO: 714 - protein, 11 aa, synthetic construct
  REGION 1..11: note = peptide
NDELDEMIAEI

SEQ ID NO: 715 - protein, 27 aa, synthetic construct
  REGION 1..27: note = peptide
KALPVVLENARILKNCVDAKMTEEDKE

SEQ ID NO: 716 - protein, 27 aa, synthetic construct
  REGION 1..27: note = peptide
KRDVDLFLTGTPDEYVEQVAQYKALPV

SEQ ID NO: 717 - protein, 14 aa, synthetic construct
  REGION 1..14: note = peptide
DAKMTEEDKENALS

SEQ ID NO: 718 - protein, 16 aa, synthetic construct
  REGION 1..16: note = peptide
EICPAVKRDVDLFLTG

SEQ ID NO: 719 - protein, 14 aa, synthetic construct
  REGION 1..14: note = peptide
EPERTAMKKIQDCY

SEQ ID NO: 720 - protein, 14 aa, synthetic construct
  REGION 1..14: note = peptide
FAVANGNELLLDLS

SEQ ID NO: 721 - protein, 14 aa, synthetic construct
  REGION 1..14: note = peptide
KKIQDCYVENGLIS

SEQ ID NO: 722 - protein, 11 aa, synthetic construct
  REGION 1..11: note = peptide
LLDKIYTSPLC

SEQ ID NO: 723 - protein, 14 aa, synthetic construct
  REGION 1..14: note = peptide
VAQYKALPVVLENA

SEQ ID NO: 724 - protein, 14 aa, synthetic construct
  REGION 1..14: note = peptide
VKMAETCPIFYDVF

SEQ ID NO: 725 - protein, 15 aa, synthetic construct
  REGION 1..15: note = peptide
VQNTVEDLKLNTLGR

SEQ ID NO: 726 - protein, 29 aa, synthetic construct
  REGION 1..29: note = peptide
EICPAVKRDVDLFLTGTPDEYVEQVAQYK

SEQ ID NO: 727 - protein, 24 aa, synthetic construct
  REGION 1..24: note = peptide
SSKDCMGEAVQNTVEDLKLNTLGR

SEQ ID NO: 728 - protein, 34 aa, synthetic construct
  REGION 1..34: note = peptide
VKMAITCPIFYDVFFAVANGNELLLDLSLTKVNA

SEQ ID NO: 729 - protein, 13 aa, synthetic construct
  REGION 1..13: note = peptide
CPAVKRDVDLFLT

SEQ ID NO: 730 - protein, 97 aa, synthetic construct
  REGION 1..97: note = peptide
EQVAQYKALPVVLENAKALPVVLENARILNCVRILKNCVDAKMTEEDKEKENALSLLDKIYTSPLTAMKKIQDCYVENGLISRVLDGLVMTTISSSK

SEQ ID NO: 731 - protein, 12 aa, synthetic construct
  REGION 1..12: note = peptide
ENLAYTKALLTG

SEQ ID NO: 732 - protein, 11 aa, synthetic construct
  REGION 1..11: note = peptide
NNISVDGLLTS

SEQ ID NO: 733 - protein, 11 aa, synthetic construct
  REGION 1..11: note = peptide
VIRNELLLTYA

SEQ ID NO: 734 - protein, 10 aa, synthetic construct
  REGION 1..10: note = peptide
VINDLLLVLA

SEQ ID NO: 735 - protein, 6 aa, synthetic construct
  REGION 1..6: note = peptide
GDFYPL

SEQ ID NO: 736 - protein, 6 aa, synthetic construct
  REGION 1..6: note = peptide
NDFYPE

SEQ ID NO: 737 - protein, 5 aa, synthetic construct
  REGION 1..5: note = peptide
LDLNP

SEQ ID NO: 738 - protein, 10 aa, synthetic construct
  REGION 1..10: note = peptide
FAVANGNELL

SEQ ID NO: 739 - protein, 20 aa, synthetic construct
  REGION 1..20: note = peptide
LKLNTLGREICPAVKRGVDL

SEQ ID NO: 740 - protein, 5 aa, synthetic construct
  REGION 1..5: note = peptide
YVEQV

SEQ ID NO: 741 - protein, 14 aa, synthetic construct
  REGION 1..14: note = peptide
ENARILKNCVDAKM

SEQ ID NO: 742 - protein, 30 aa, synthetic construct
  REGION 1..30: note = peptide
VRYTTEGGTKTEAEDVIPEGWKADTSYESK

SEQ ID NO: 743 - protein, 15 aa, synthetic construct
  REGION 1..15: note = peptide
APYHFDLSGHAFGAM

SEQ ID NO: 744 - protein, 21 aa, synthetic construct
  REGION 1..21: note = peptide
HITDDNEEPIAPYHFDLSGHA

SEQ ID NO: 745 - protein, 18 aa, synthetic construct
  REGION 1..18: note = peptide
NEEPIAPYHFDLSGHAFG

SEQ ID NO: 746 - protein, 19 aa, synthetic construct
  REGION 1..19: note = peptide
CFEIKCTKPEACSGEPVVV

SEQ ID NO: 747 - protein, 34 aa, synthetic construct
  REGION 1..34: note = peptide
CTKPEACSGEPVVVHITDDNEEPIAPYHFDLSGH

SEQ ID NO: 748 - protein, 11 aa, synthetic construct
  REGION 1..11: note = peptide
DNEEPIAPYHF

SEQ ID NO: 749 - protein, 13 aa, synthetic construct
  REGION 1..13: note = peptide
EPIAPYHFDLSGH

SEQ ID NO: 750 - protein, 19 aa, synthetic construct
  REGION 1..19: note = peptide
EQKLRSAGELELQFRRVKC

SEQ ID NO: 751 - protein, 35 aa, synthetic construct
  REGION 1..35: note = peptide
GEPVVVHITDDNEEPIAPYHFDLSGHAFGAMAKKG

SEQ ID NO: 752 - protein, 50 aa, synthetic construct
  REGION 1..50: note = peptide
GNTPIFKSGRGCGSCFEIKCTKPEACSGEPVVVHITDDNEEPIAPYHFDL

SEQ ID NO: 753 - protein, 16 aa, synthetic construct
  REGION 1..16: note = peptide
GPFTVRYTTEGGTKTE

SEQ ID NO: 754 - protein, 23 aa, synthetic construct
  REGION 1..23: note = peptide
GTKTEAEDVIPEGWKADTSYESK

SEQ ID NO: 755 - protein, 43 aa, synthetic construct
  REGION 1..43: note = peptide
GYKDVDKPPFSGMTGCGNTPIFKSGRGCGSCFEIKCTKPEACS

SEQ ID NO: 756 - protein, 15 aa, synthetic construct
  REGION 1..15: note = peptide
HITDDNEEPIAPYHF

SEQ ID NO: 757 - protein, 24 aa, synthetic construct
  REGION 1..24: note = peptide
HITDDNEEPIAPYHFDLSGHAFGA

SEQ ID NO: 758 - protein, 27 aa, synthetic construct
  REGION 1..27: note = peptide
HVEKGSNPNYLALLVKYVNGDGDVVAV

SEQ ID NO: 759 - protein, 33 aa, synthetic construct
  REGION 1..33: note = peptide
KCTKPEACSGEPVVVHITDDNEEPIAPYHFDLS

SEQ ID NO: 760 - protein, 13 aa, synthetic construct
  REGION 1..13: note = peptide
KPPFSGMTGCGNT

SEQ ID NO: 761 - protein, 21 aa, synthetic construct
  REGION 1..21: note = peptide
KTEAEDVIPEGWKADTSYESK

SEQ ID NO: 762 - protein, 36 aa, synthetic construct
  REGION 1..36: note = peptide
LTGPFTVRYTTEGGTKTEAEDVIPEGWKADTSYESK

SEQ ID NO: 763 - protein, 8 aa, synthetic construct
  REGION 1..8: note = peptide
PIAPYHFD

SEQ ID NO: 764 - protein, 15 aa, synthetic construct
  REGION 1..15: note = peptide
PIAPYHFDLSGHAFG

SEQ ID NO: 765 - protein, 33 aa, synthetic construct
  REGION 1..33: note = peptide
VPKVTFTVEKGSNEKHLAVLVKYEGDTMAEVEL

SEQ ID NO: 766 - protein, 32 aa, synthetic construct
  REGION 1..32: note = peptide
VEKGSNEKHLAVLVKYEGDTMAEVELREHGSD

SEQ ID NO: 767 - protein, 32 aa, synthetic construct
  REGION 1..32: note = peptide
REHGSDEWVAMTKGEGGVWTFDSEEPLQGPFN

SEQ ID NO: 768 - protein, 31 aa, synthetic construct
  REGION 1..31: note = peptide
FRFLTEKGMKNVFDDVVPEKYTIGATYAPEE

SEQ ID NO: 769 - protein, 32 aa, synthetic construct
  REGION 1..32: note = peptide
CFRFLTEKGMKNVFDDVVPEKYTIGATYAPEE

SEQ ID NO: 770 - protein, 32 aa, synthetic construct
  REGION 1..32: note = peptide
FRFLTEKGMKNVFDDVVPEKYTIGATYAPEEC

SEQ ID NO: 771 - protein, 37 aa, synthetic construct
  REGION 1..37: note = peptide
AEEVKVIPAGELQVIEKVDAAFKVAATAANAAPANDK

SEQ ID NO: 772 - protein, 33 aa, synthetic construct
  REGION 1..33: note = peptide
ATTEEQKLIEKINAGFKAALAAAAGVQPADKYR

SEQ ID NO: 773 - protein, 36 aa, synthetic construct
  REGION 1..36: note = peptide
FVATFGAASNKAFAEGLSGEPKGAAESSSKAALTSK

SEQ ID NO: 774 - protein, 33 aa, synthetic construct
  REGION 1..33: note = peptide
ADLGYGPATPAAPAAGYTPATPAAPAEAAPAGK

SEQ ID NO: 775 - protein, 31 aa, synthetic construct
  REGION 1..31: note = peptide
AYKLAYKTAEGATPEAKYDAYVATLSEALRI

SEQ ID NO: 776 - protein, 30 aa, synthetic construct
  REGION 1..30: note = peptide
EAAFNDAIKASTGGAYESYKFIPALEAAVK

SEQ ID NO: 777 - protein, 32 aa, synthetic construct
  REGION 1..32: note = peptide
TVATAPEVKYTVFETALKKAITAMSEAQKAAK

SEQ ID NO: 778 - protein, 10 aa, synthetic construct
  REGION 1..10: note = peptide
SRLGRSSAWV

SEQ ID NO: 779 - protein, 10 aa, synthetic construct
  REGION 1..10: note = peptide
THWQLGERPD

SEQ ID NO: 780 - protein, 10 aa, synthetic construct
  REGION 1..10: note = peptide
PSTPGERVRH

SEQ ID NO: 781 - protein, 10 aa, synthetic construct
  REGION 1..10: note = peptide
RGGPDDLTAL

SEQ ID NO: 782 - protein, 9 aa, synthetic construct
  REGION 1..9: note = peptide
PFWVRGTTW

SEQ ID NO: 783 - protein, 10 aa, synthetic construct
  REGION 1..10: note = peptide
PSTPGSRQNM

SEQ ID NO: 784 - protein, 10 aa, synthetic construct
  REGION 1..10: note = peptide
PSTPGDNPLV

SEQ ID NO: 785 - protein, 10 aa, synthetic construct
  REGION 1..10: note = peptide
KFVVNGRWID

SEQ ID NO: 786 - protein, 10 aa, synthetic construct
  REGION 1..10: note = peptide
KFLVNGRWID

SEQ ID NO: 787 - protein, 10 aa, synthetic construct
  REGION 1..10: note = peptide
RLTENTEPLL

SEQ ID NO: 788 - protein, 10 aa, synthetic construct
  REGION 1..10: note = peptide
FTWGGLRDKS

SEQ ID NO: 789 - protein, 10 aa, synthetic construct
  REGION 1..10: note = peptide
ERAGAMERAN

SEQ ID NO: 790 - protein, 10 aa, synthetic construct
  REGION 1..10: note = peptide
RSVSKEEPGM

SEQ ID NO: 791 - protein, 10 aa, synthetic construct
  REGION 1..10: note = peptide
KLGKFGAARV

SEQ ID NO: 792 - protein, 10 aa, synthetic construct
  REGION 1..10: note = peptide
VQDLMKSSGV

SEQ ID NO: 793 - protein, 12 aa, synthetic construct
  REGION 1..12: note = peptide
CKLGKFGAARVC

SEQ ID NO: 794 - protein, 11 aa, synthetic construct
  REGION 1..11: note = peptide
CKLGKFGAARV

SEQ ID NO: 795 - protein, 11 aa, synthetic construct
  REGION 1..11: note = peptide
KLGKFGAARVC

SEQ ID NO: 796 - protein, 32 aa, synthetic construct
  REGION 1..32: note = peptide
GKATTEEQKLIEDVNASFRAAMATTANVPPAD

SEQ ID NO: 797 - protein, 35 aa, synthetic construct
  REGION 1..35: note = peptide
YKTFEAAFTVSSKRNLADAVSKAPQLVPKLDEVYN

SEQ ID NO: 798 - protein, 33 aa, synthetic construct
  REGION 1..33: note = peptide
DAVSKAPQLVPKLDEVYNAAYNAADHAAPEDKY

SEQ ID NO: 799 - protein, 33 aa, synthetic construct
  REGION 1..33: note = peptide
AADHAAPEDKYEAFVLHFSEALRIIAGTPEVHA

SEQ ID NO: 800 - protein, 17 aa, synthetic construct
  REGION 1..17: note = peptide
KAAAVKAAFWTALNRRA

SEQ ID NO: 801 - protein, 23 aa, synthetic construct
  REGION 1..23: note = peptide
DSETADNLEKTVAALSILPGHGC

SEQ ID NO: 802 - protein, 27 aa, synthetic construct
  REGION 1..27: note = peptide
DSETADNLEKTVAALSILPGHGCNRRA

SEQ ID NO: 803 - protein, 23 aa, synthetic construct
  REGION 1..23: note = peptide
ISITEIKGVIVHRIETILFNRRA

SEQ ID NO: 804 - protein, 17 aa, synthetic construct
  REGION 1..17: note = peptide
ISQAVHAAHAEINEAGR

SEQ ID NO: 805 - protein, 21 aa, synthetic construct
  REGION 1..21: note = peptide
ISQAVHAAHAEINEAGRNRRA

SEQ ID NO: 806 - protein, 20 aa, synthetic construct
  REGION 1..20: note = peptide
DSETADNLEKTTAALSILPG

SEQ ID NO: 807 - protein, 24 aa, synthetic construct
  REGION 1..24: note = peptide
DSETADNLEKTTAALSILPGNRRA

SEQ ID NO: 808 - protein, 15 aa, synthetic construct
  REGION 1..15: note = peptide
LSEIKGVIVHRLEGV

SEQ ID NO: 809 - protein, 19 aa, synthetic construct
  REGION 1..19: note = peptide
LSEIKGVIVHRLEGVNRRA

SEQ ID NO: 810 - protein, 22 aa, synthetic construct
  REGION 1..22: note = peptide
KLLSLIKGVIVHRLEGVENRRA

SEQ ID NO: 811 - protein, 15 aa, synthetic construct
  REGION 1..15: note = peptide
VSIDKFRIFCKANPK

SEQ ID NO: 812 - protein, 16 aa, synthetic construct
  REGION 1..16: note = peptide
LKFIIKRYTPNNEIDS

SEQ ID NO: 813 - protein, 15 aa, synthetic construct
  REGION 1..15: note = peptide
IREDNNTLKLDRCNN

SEQ ID NO: 814 - protein, 21 aa, synthetic construct
  REGION 1..21: note = peptide
FNNFTVSFWLRVPKVSASHLE

SEQ ID NO: 815 - protein, 14 aa, synthetic construct
  REGION 1..14: note = peptide
QYIKANSKFIGITE

SEQ ID NO: 816 - protein, 20 aa, synthetic construct
  REGION 1..20: note = peptide
PHHTALRQAILCWGELMTLA

SEQ ID NO: 817 - protein, 15 aa, synthetic construct
  REGION 1..15: note = peptide
FFLLTRILTIPQSLD

SEQ ID NO: 818 - protein, 16 aa, synthetic construct
  REGION 1..16: note = peptide
YSGPLKAEIAQRLEDV

SEQ ID NO: 819 - protein, 14 aa, synthetic construct
  REGION 1..14: note = peptide
FFLLTRILTIPQSL

SEQ ID NO: 820 - protein, 24 aa, synthetic construct
  REGION 1..24: note = peptide
GAYARCPNGTRALTVAELRGNAEL

SEQ ID NO: 821 - protein, 25 aa, synthetic construct
  REGION 1..25: note = peptide
ALNIWDRFDVFCTLGATTGYLKGNS

SEQ ID NO: 822 - protein, 15 aa, synthetic construct
  REGION 1..15: note = peptide
QYIKANSKFIGITEL

SEQ ID NO: 823 - protein, 16 aa, synthetic construct
  REGION 1..16: note = peptide
KFIIKRYTPNNEIDSF

SEQ ID NO: 824 - protein, 16 aa, synthetic construct
  REGION 1..16: note = peptide
VSIDKFRIFCKALNPK

SEQ ID NO: 825 - protein, 17 aa, synthetic construct
  REGION 1..17: note = peptide
WVRDIIDDFTNESSQKT

SEQ ID NO: 826 - protein, 19 aa, synthetic construct
  REGION 1..19: note = peptide
AGLTLSLLVICSYLFISRG

SEQ ID NO: 827 - protein, 20 aa, synthetic construct
  REGION 1..20: note = peptide
PGPLRESIVCYFMVFLQTHI

SEQ ID NO: 828 - protein, 18 aa, synthetic construct
  REGION 1..18: note = peptide
VPGLYSPCRAFFNKEELL

SEQ ID NO: 829 - protein, 15 aa, synthetic construct
  REGION 1..15: note = peptide
TGHGARTSTEPTTDY

SEQ ID NO: 830 - protein, 13 aa, synthetic construct
  REGION 1..13: note = peptide
KELKRQYEKKLRQ

SEQ ID NO: 831 - protein, 11 aa, synthetic construct
  REGION 1..11: note = peptide
TVFYNIPPMPL

SEQ ID NO: 832 - protein, 14 aa, synthetic construct
  REGION 1..14: note = peptide
DKREMWMACIKELH

SEQ ID NO: 833 - protein, 11 aa, synthetic construct
  REGION 1..11: note = peptide
FVFTLTVPSER

SEQ ID NO: 834 - protein, 13 aa, synthetic construct
  REGION 1..13: note = peptide
PKYVKQNTLKLAT

SEQ ID NO: 835 - protein, 16 aa, synthetic construct
  REGION 1..16: note = peptide
EKKIAKMEKASSVFNV

SEQ ID NO: 836 - protein, 10 aa, synthetic construct
  REGION 1..10: note = peptide
FFLLTRILTI

SEQ ID NO: 837 - protein, 19 aa, synthetic construct
  REGION 1..19: note = peptide
DQSIGDLIAEAMDKVGNEG

SEQ ID NO: 838 - protein, 14 aa, synthetic construct
  REGION 1..14: note = peptide
QVHFQPLPPAVVKL

SEQ ID NO: 839 - protein, 16 aa, synthetic construct
  REGION 1..16: note = peptide
KQIINMWQEVGKAMYA

SEQ ID NO: 840 - protein, 8 aa, synthetic construct
  REGION 1..8: note = peptide
CDQPVLPD

SEQ ID NO: 841 - protein, 7 aa, synthetic construct
  REGION 1..7: note = peptide
DMPVDPD

SEQ ID NO: 842 - protein, 8 aa, synthetic construct
  REGION 1..8: note = peptide
CDMPVDPD

SEQ ID NO: 843 - protein, 7 aa, synthetic construct
  REGION 1..7: note = peptide
DAEFRHC

SEQ ID NO: 844 - protein, 8 aa, synthetic construct
  REGION 1..8: note = peptide
MDVFMKGL

SEQ ID NO: 845 - protein, 9 aa, synthetic construct
  REGION 1..9: note = peptide
MDVFMKGLC

SEQ ID NO: 846 - protein, 10 aa, synthetic construct
  REGION 1..10: note = peptide
ATGFVKKDQL

SEQ ID NO: 847 - protein, 11 aa, synthetic construct
  REGION 1..11: note = peptide
ATGFVKKDQLC

SEQ ID NO: 848 - protein, 9 aa, synthetic construct
  REGION 1..9: note = peptide
LGKNEEGAP

SEQ ID NO: 849 - protein, 10 aa, synthetic construct
  REGION 1..10: note = peptide
EGYQDYEPEA

SEQ ID NO: 850 - protein, 11 aa, synthetic construct
  REGION 1..11: note = peptide
EGYQDYEPEAC

SEQ ID NO: 851 - protein, 7 aa, synthetic construct
  REGION 1..7: note = peptide
  SITE 1: note = pyroglutamic acid
EFRHDSC

SEQ ID NO: 852 - protein, 13 aa, synthetic construct
  REGION 1..13: note = peptide
KDNIKHVPGGGSC

SEQ ID NO: 853 - protein, 19 aa, synthetic construct
  REGION 1..19: note = peptide
WDPGMLQSELIQKEFGDYC

SEQ ID NO: 854 - protein, 18 aa, synthetic construct
  REGION 1..18: note = peptide
WDPGMLQSELIQKEFGDY

SEQ ID NO: 855 - protein, 10 aa, synthetic construct
  REGION 1..10: note = peptide
TPDAPGETVC

SEQ ID NO: 856 - protein, 9 aa, synthetic construct
  REGION 1..9: note = peptide
TPDAPGETV

SEQ ID NO: 857 - protein, 12 aa, synthetic construct
  REGION 1..12: note = peptide
TQQIPSLSPSQC

SEQ ID NO: 858 - protein, 12 aa, synthetic construct
  REGION 1..12: note = peptide
QQQGLPRAAGGC

SEQ ID NO: 859 - protein, 9 aa, synthetic construct
  REGION 1..9: note = peptide
QQFLSVRAL

SEQ ID NO: 860 - protein, 20 aa, synthetic construct
  REGION 1..20: note = peptide
GAISLAPKAQIKESLRAELC

SEQ ID NO: 861 - protein, 8 aa, synthetic construct
  REGION 1..8: note = peptide
DMPVDPDN

SEQ ID NO: 862 - protein, 9 aa, synthetic construct
  REGION 1..9: note = peptide
CDMPVDPDN

SEQ ID NO: 863 - protein, 6 aa, synthetic construct
  REGION 1..6: note = peptide
DMPVDP

SEQ ID NO: 864 - protein, 7 aa, synthetic construct
  REGION 1..7: note = peptide
CDMPVDP

SEQ ID NO: 865 - protein, 5 aa, synthetic construct
  REGION 1..5: note = peptide
DMPVD

SEQ ID NO: 866 - protein, 6 aa, synthetic construct
  REGION 1..6: note = peptide
CDMPVD

SEQ ID NO: 867 - protein, 9 aa, synthetic construct
  REGION 1..9: note = peptide
DMPVDPDNE

SEQ ID NO: 868 - protein, 10 aa, synthetic construct
  REGION 1..10: note = peptide
DMPVDPDNEC

SEQ ID NO: 869 - protein, 10 aa, synthetic construct
  REGION 1..10: note = peptide
DMPVDPDNEA

SEQ ID NO: 870 - protein, 11 aa, synthetic construct
  REGION 1..11: note = peptide
DMPVDPDNEAC

SEQ ID NO: 871 - protein, 11 aa, synthetic construct
  REGION 1..11: note = peptide
DMPVDPDNEAY

SEQ ID NO: 872 - protein, 12 aa, synthetic construct
  REGION 1..12: note = peptide
DMPVDPDNEAYC

SEQ ID NO: 873 - protein, 12 aa, synthetic construct
  REGION 1..12: note = peptide
DMPVDPDNEAYE

SEQ ID NO: 874 - protein, 13 aa, synthetic construct
  REGION 1..13: note = peptide
DMPVDPDNEAYEC

SEQ ID NO: 875 - protein, 8 aa, synthetic construct
  REGION 1..8: note = peptide
APQEGILE

SEQ ID NO: 876 - protein, 9 aa, synthetic construct
  REGION 1..9: note = peptide
APQEGILEC

SEQ ID NO: 877 - protein, 38 aa, synthetic construct
  REGION 1..38: note = peptide
WRTQNDVLENGTFTDNVDQRMPRCRHRVVELMDWTVLH

SEQ ID NO: 878 - protein, 30 aa, synthetic construct
  REGION 1..30: note = peptide
LAFPGSGEQVEKLIKNQKESAKSSPYQKKT

(19) United States
(12) Patent Application Publication
(10) Pub. No.: US 2025/0186595 A1
(43) Pub. Date: Jun. 12, 2025

(54) A CONJUGATE CONSISTING OF OR COMPRISING AT LEAST A ß-GLUCAN OR A MANNAN

(71) Applicant: TRIDEM BIOSCIENCE GMBH & CO KG, Vienna (AT)

(72) Inventors: Markus MANDLER, Vienna (AT); Sabine SCHMIDHUBER, Vienna (AT); Achim SCHNEEBERGER, Vienna (AT)

(21) Appl. No.: 18/842,260

(22) PCT Filed: Feb. 28, 2023
(86) PCT No.: PCT/EP2023/055020

§ 371 Date: Aug. 28, 2024

(30) Foreign Application Priority Data
Feb. 28, 2022 (EP) 22159191.0
Aug. 19, 2022 (EP) 22191221.5

(51) Int. Cl. A61K 47/61; A61K 47/64

(52) U.S. Cl. CPC A61K 47/61; A61K 47/64

(57) ABSTRACT

The invention relates to the use of p-glucans as C-type lectin (CLEG) polysaccharide adjuvants for B-cell or T-cell epitope polypeptides.

The present invention relates to polysaccharide adjuvants belonging to the class of C-type lectins (CLECs).

Vaccination is considered one of the most powerful means to save lives and to alleviate disease burden. By means of active immunization the vaccine is administered so that the immune system of the host develops a non-specific innate immune response as well as specific antibodies, B- and T memory cells that can act against the immunogen applied.

Polysaccharides constitute important virulence factors especially for encapsulated bacteria that present complex carbohydrate structures on their surface. Bacterial, as well as fungal or other polysaccharides are constructed from repeated monosaccharide units linked by glycosidic bonds to form polymeric linear or branched structures. It is well known that antibody responses to various bacterial polysaccharides are weak and since they do not induce immunological memory, are not boosted by subsequent immunizations.

These characteristics are based on the nature of the polysaccharides: unlike proteins polysaccharides are constituting T-cell independent (TI) antigens. Polysaccharide antigens directly activate polysaccharide-specific B-cells which then differentiate into plasma cells to produce antibodies. Memory B-cells are not formed. Proteins and peptides instead are T-cell dependent antigens (TD) antigens; following interaction with antigen-presenting cells (APCs) like Dendritic Cells (DCs), macrophages and B-cells, protein antigens are internalized and processed into small peptides which are then re-exposed and presented to T-lymphocytes in association with the major histocompatibility complex (MHC) class II molecules. Interaction with T-cells induces B-cells to differentiate into plasma cells and memory B-cells. Unlike TI antigens, TD antigens are immunogenic, responses can be boosted and enhanced by adjuvants (Peltola et al. Pediatrics November 1977, 60 (5) 730-737, Guttormsen H K et al. INFECTION AND IMMUNITY, May 1998, p. 2026-2032 and INFECTION AND IMMUNITY, December 1999, p. 6375-6384, Avci et al. Nature Medicine volume 17, pages 1602-1609 (2011)).

The limitation of pure polysaccharide vaccines has been overcome by covalent conjugation of polysaccharides to a carrier protein as source of T-cell epitopes. This concept has been successfully used in several glycoconjugate vaccines currently on the market, which have been developed against bacterial pathogens such as Neisseriae meningitidis, Streptococcus pneumoniae, Haemophilus Influenzae b and group B Streptococcus. All of these vaccines use a pathogen specific carbohydrate to induce carbohydrate specific antibodies as main protective agent.

It is well known in the field that carbohydrate-based polysaccharides from plant, bacterial, fungal and synthetic sources can act as so-called pathogen-associated molecular patterns (PAMPs). Upon contact with the immune system, PAMPs are recognized by pattern recognition receptors (PRRs) on specialized immune cells.

PRRs constitute a class of germ line-encoded receptors that upon binding/activation are crucial for the initiation of innate immunity, which plays a key role in first-line defense until more specific adaptive immunity is developed. The innate immune response is the first line of defense against infectious diseases and tissue damage. Specialized cells, i.e. mainly APCs like macrophages and DCs, as well as some non-professional cells such as epithelial cells, endothelial cells, and fibroblasts, are expressing these PRRs and play major roles in pathogen recognition during the innate immune response. Moreover, activation of APCs by innate immune signals is a key prerequisite for the generation of potent adaptive immunity, including antibody and memory responses.

Currently identified PRR families are divided into transmembrane receptors and those that reside in intracellular compartments. The transmembrane receptors include for example the Tolllike receptors (TLRs 1-9) and C-type lectin receptors (CLRs). Intracellular receptors include the nucleotide-binding oligomerization domain-(NOD-) like receptors (NLRs), retinoic acid-inducible gene-(RIG-) I-like receptors (RLRs), and AIM2-like receptors (ALR).

The PAMP character of carbohydrates, especially polysaccharides, has led to various approaches to develop polysaccharides as successful vaccine adjuvants.

One prominent example is inulin, a polysaccharide found in the roots of Compositae. It consists of linear β-D-(2, 1) polyfructofuranosyl α-D-glucoses, with up to 100 fructose moieties linked to a single terminal glucose. Inulin has no immunological activity in its native soluble form but when crystallized into different stable microcrystalline particulates (inulin alpha-delta) acquires potent adjuvant activity for protein conjugate vaccines. Delta inulin particulates are marketed as Advax™ adjuvant and show consistent spherulite-like discoid particles with 1-2 μm in diameter, which are made up of a series of lamellar sheets. Delta- and Gamma-Inulin are thought to act via alternative complement activation as the mechanism of adjuvant action of gamma inulin was shown to involve increased C3 deposition on the surface of macrophages leading to enhanced T-cell activation (Kerekes et al. J Leukoc Biol. 2001 January; 69 (1): 69-74).

Another type of polysaccharide-based adjuvant candidates are chitosan-based adjuvants. Chitosan is a linear β-(1,4)-linked copolymer of D-glucosamine and N-acetyl-D-glucosamine (GlcNAc), prepared by partial alkaline deacetylation of chitin. Soluble chitosan is also poorly immunogenic on its own. However, chitosans formulated as dry powder particles or in solution have been widely used as encapsulation agents for mucosal and systemic vaccine delivery and also in preparation of mucosal DNA vaccines. It is used in mucosal vaccine delivery as it promotes absorption and subsequent phagocytosis which enhances mucosal immune responses (Dodane et al. International Journal of Pharmaceutics 182 (1999) 21-32, Seferian et al. Vaccine 19 (2001) 661-668). The mucoadhesive property of chitosan particles is attributed to its cationic character.

An in vitro assay indicated that bovine serum albumin (BSA) or ovalbumin (OVA) encapsulated in chitosan particles could stimulate RAW264.7 macrophage and BMDC activation more efficiently compared to soluble antigens (Koppolu B, et al. The effect of antigen encapsulation in chitosan particles on uptake, activation and presentation by antigen presenting cells. Biomaterials. 2013; 34:2359-2369.).

Neimert-Andersson et al. have used ViscoGel, a hydrogel of soluble chitosan. ViscoGel together with a vaccine against Haemophilus influenza type b (Act-HIB) could induce stronger humoral and cellular response to the antigen as the non-adjuvanted vaccine. The titers of IgG1 and IgG2a in serum were significantly enhanced. Production of Th1-, Th2-, and Th17-type cytokines was elevated as well. Unfortunately, Viscogel proved non-suitable for human use (Vaccine. 2014; 32:5967-5974).

Both chitin and chitosan particles are readily phagocytosed, supporting a role for recognition via specific receptor(s) mediating phagocytosis. Receptors on myeloid cells that bind chitin or chitosan and induce a phagocytic response have yet to be definitively identified. So far, several receptors have shown binding to chitin or chitin oligosaccharides, including: FIBCD1, a homo-thetrameric 55-kDa type II transmembrane protein expressed in the gastrointestinal tract; NKR-P1, an activating receptor on rat natural killer cells; RegIIIc, a secreted C-type lectin; and galectin-3, a lectin with affinity for β-galactosides. Mannose receptors also show binding to GlcNac and are hence potential receptors for Chitosan based vaccine conjugates. ViscoGel has been reported to trigger immune response in a similar way as chitin, since chitin particles were reported to function as PAMPs and recognize TLR-2 receptor on macrophages to induce innate immune response.

Yu et al. (Mol. Pharmaceutics, 2016, DOI: 10.1021/acs.molpharmaceut. 6b00138) also demonstrated that inulin-chitosan conjugates with Mycobacterium tuberculosis CFP10-TB10.4 Fusion Protein (CT) had a significantly increased hydrodynamic volume as compared to non-adjuvanted proteins and this vaccine elicited high levels of Th1-type (IFN-γ, TNF-α, and IL-2) and Th2-type cytokines (IL4) and efficient CT-specific antibody titers, mostly in the form of IgG1 and IgG2b. Pharmacokinetic studies revealed that conjugation with inulin-Chitosans could prolong the serum exposure of CT to the immune system. (Mol. Pharmaceutics 2016, 13, 11, 3626-3635).

One of the most prominent classes of polysaccharides used as adjuvant is the class of C-type lectins (CLECs) which interact with their receptors, called C-type lectin receptors (CLRs). CLRs are considered as Ca²⁺ dependent carbohydrate recognition proteins. These receptors express single or multiple carbohydrate recognition domains (CRDs) on their C-type lectin-like domains (CLECDS) which are required for binding of CLECs. The components of the CLR family bind to different carbohydrates such as mannose, fucose, glucose, maltose, N-acetyl-D-Glucosamine or other glycans and glucans.

CLRs are classified as Transmembrane CLRs (TM-CLRs) and soluble CLRs (Collectins). The TM-CLRs are further classified as type I TM-CLRs and type II TM-CLRs. Type I TM-CLRs include mannose receptors (MRs) and ENDO180 [mannose receptor C type 2 (MRC 2)] receptors and bind to mannose, fucose, and N-acetylglucosamine. Type II™-CLRs include dendritic cell-specific intracellular adhesion molecule-3 grabbing non-integrin (DC-SIGN), langerin, and macrophage galactose-type lectin (MGL) receptors. DC-SIGN is known to bind to N-linked glycans (branched trimannose structures) for example present on HIV-I glycoprotein gp120 and other viruses such as Hepatitis C virus, Human Cytomegalovirus, Dengue or Ebola. DCSIGN also recognizes lipoarabinomannan and mannan. Langerin is the Langerhans cell (LC) related CLR which binds to mannose and fucose containing glycan residues. MGL has binding specificity towards terminal N-acetylgalactosamine (GalNAc) residues and has also been shown to possess affinity towards C. jejuni and for tumor-associated mucin MUC1 and it is involved in controlling adaptive immunity by effector T-cells. In addition, macrophage-inducible C-type lectin (Mincle, also known as CLEC4A) as well as dectin-1/dectin-2 receptor families have been described. Dectin-1 plays an important role in mediating innate immunity against fungi and has the ability to bind to β-glucans found in fungi (e.g.: Saccharomyces Cervisiae β-glucan), lichens (e.g.: pustulan, lichenan), algae (e.g.: laminarin) or barley and other grain varieties. Dectin-2 contains an EPN (Glu-Pro-Asn) amino acid motif that provides sensitivity for mannose ligands. Additionally, dectin-2 interacts with C. albicans.

Targeting antigens to the endocytic receptors on antigen presenting cells (APCs) represents an attractive approach to enhance vaccine efficacy. In particular, the mannose receptor (MR) and related C-type lectin receptor (CLR) family members such as DEC205, DC-SIGN, MGL, langerin, dectin-1, and Mincle demonstrated excellent carbohydrate antigen-capturing and processing capabilities.

Particularly immature dendritic cells (DCS) express a large panel of CLRs, linked to their important function of sensing and capturing self-, as well as non-self-antigens for processing and presentation onto MHC molecules, thus inducing antigen-specific T-cell activation. Thus, they link innate and adaptive immune responses.

CLECs have been used as unspecific stimulators of immune responses and adjuvants for immunization. For example, Vojtek et al. (Food and Agricultural Immunology, 2017, 28:6, 993-1002) could demonstrate that orally administered β-(1,3), β-(1,6) glucan applied in combination with immunization against rabies and canine parvovirus-2 in dogs lead to earlier development of protective levels of antibodies against both viruses.

Several studies have demonstrated that the activation status of APCs plays a dominant role in the type of immunity induced. For example, immunization with antigen-antibody conjugates under inflammatory conditions leads to TH1 responses, independently whether the antigen is targeted to the CLR DEC-205, langerin, Clec9A, or the Ig superfamily member Treml4.

In contrast, immunization under non-inflammatory (steady state) conditions results in tolerance induction. langerin+ migratory DCs, but not lymph node resident DCs, have been identified as major Treg inducers, independently of the source of the dendritic cell (skin vs. lung) or the targeted receptor.

Especially targeting the mannose Receptor (MR) or other mannan-sensing CLRs by using mannan as CLEC has demonstrated effective induction of cellular as well as humoral immune responses; consequently, MR- and other CLR-targeted vaccines have gained increased attention for treatment of cancer, infectious diseases, and for specific tolerance induction in autoimmune diseases.

Mannan, a polysaccharide derived from the yeast cell wall, consists of a backbone of mostly β-(1,4)-linked mannose with a small number of α-(1,6)-linked glucose and galactose side chain residues. In addition, a protein content of approximately 5% has been detected in conventional mannan preparations. As an important component of fungal cell walls, mannan has been widely used as component in carbohydrate-based vaccines for Candidiasis (Han and Rhew, Arch Pharm Res 2012, Vol 35, No 11, 2021-2027; Cassone, Nat Rev Microbiol. 2013 December; 11 (12): 884-91; Johnson and Bundle, Chem. Soc. Rev., 2013, 42, 4327). In addition, different examples for vaccines based on mannan carrier-antigen complex/conjugations have been developed, including mannan-mucin 1 (MUC1) fusion protein conjugation for tumor therapy or conjugates of mannan and model allergens like ovalbumin (OVA), Papain or Betv1.

Mucins are heavily glycosylated proteins expressed on cell surfaces. MUC1 is a prototypical mucin, which has been found to be over-expressed on a wide range of tumor cells. Along these lines, a MUC1 Fusion Protein containing 5 tandem repeats of human MUC1 (containing the immune-dominant epitope: APDTRPAPGSTAPPAHGVTS) and peptide (Cpl3-32) were produced and conjugated to mannan under either oxidative or reductive conditions leading to drastically different immunological responses: oxidized mannan-MUC1 stimulated Th1 type responses mediated by CD8+ T-cells with IFN-γ secretion and mainly IgG2a antibody response, whereas reduced mannan-MUC1 stimulated Th2 type responses with IL-4 production and a high IgG1 antibody response. The employed fusion protein represented a single protein displaying T- and B-cell epitopes.

Recently also Protein-carbohydrate/mannan complexes of papain and OVA were generated to analyse their allergenic potential. It was found that coupling mannan to the protein surface could decrease binding and crosslinking of IgE antibodies directed against Papain. Interestingly, coupling of either mannan, dextran, or maltodextrin only reduced the allergenic potential of papain, but not OVA in these experiments indicating the importance of carbohydrate selection for vaccine design (Weinberger et al. J. Control. Release 2013; 165:101-109). These experiments also showed that mannan conjugation leads to the development of elevated IgG titers against OVA following intradermal immunization.

Similar to the neoglycoconjugate vaccine used for MUC1, Ghochikyan et al. (DNA AND CELL BIOLOGY, Volume 25, Number 10, 2006, Pp. 571-580) and Petrushina et al. (Journal of Neuroinflammation 2008, 5:42) were applying Amyloid beta (AB) 28, a 28 aa residue peptide carrying combined B- and T-cell epitopes of the human Aβ42 peptide, coupled to mannan and could induce low levels of anti-AB responses in mice. These responses also proved to attenuate amyloid deposition in the cortical and hippocampal regions of APP transgenic mice following sub cutaneous immunization. The immunization also led to the induction of increased anti-mannan titers in Aβ28-mannan and BSA-mannan treated animals. The treatment however was not further developed most probably due to the occurrence of increased micro-haemorrhages in the brains of treated animals which were attributed to potential harmful effects of mannan as the trigger of adverse vascular events underlining the importance of carbohydrate selection for design of efficient and safe vaccines.

No conjugates using individual B-cell or T-cell epitope peptides coupled to mannan or other related CLECs are known so far.

β-Glucans comprise a group of β-D-glucose polysaccharides. These polysaccharides are major cell wall structural components in fungi and are also found in bacteria, yeasts, algae, lichens, and plants, such as oats and barley. Depending on the source, β-glucans vary in the type of linkage, the degree of branching, molecular weight and tertiary structure.

β-glucans are a source of soluble, fermentable fiber—also called prebiotic fiber—which provides a substrate for microbiota within the large intestine, increasing fecal bulk and producing short-chain fatty acids as by-products with wide-ranging physiological activities. For example, dietary intake of Cereal β-glucans from oat at daily amounts of at least 3 grams lowers total and low-density lipoprotein cholesterol levels by 5 to 10% in people with normal or elevated blood cholesterol levels.

Typically, β-glucans form a linear backbone with 1-3 β-glycosidic bonds but vary with respect to molecular mass, solubility, viscosity, branching structure, and gelation properties. Yeast and fungal β-glucans are usually built on a β-(1,3) backbone and contain β-(1,6) side branches, while cereal β-glucans contain both β-(1,3) and β-(1,4) backbone bonds with or without side branching.

β-Glucans are recognized by the innate immune system as pathogen-associated molecular patterns (PAMPs). The PRR dectin-1 has emerged as the primary receptor for these carbohydrates and β-glucan binding to dectin-1 induces a variety of cellular responses via the Syk/CARD9 signalling pathway, including phagocytosis, respiratory burst and secretion of cytokines. In addition, also complement receptor 3 (CR3, CD11b/CD18) has been implicated as receptor for β-glucans. It has been reported that the stimulation via dectin-1 primes Th1, Th17, and cytotoxic T lymphocyte responses.

Members of the β-glucan family include:

Beta-glucan peptide (BGP) is a high molecular weight (˜100 kDa), branched polysaccharide extracted from the fungus Trametes versicolor. BGP consists of a highly ramified glucan portion, comprising a β-(1,4) main chain and β-(1,3) side chain, with β-(1,6) side chains covalently linked to a polypeptide portion rich in aspartic, glutamic and other amino acids.

Curdlan is a high molecular weight linear polymer consisting of β-(1,3)-linked glucose residues from Agrobacterium spp.

Laminarin from the brown seaweed Laminaria digitata is a linear β-(1,3)-glucan with β-(1,6)-linkages. Laminarin is a low molecular weight (5-7 kDa), water-soluble β-glucan that can act either as a dectin-1 antagonist or agonist. It can bind to dectin-1 without stimulating downstream signalling and is able to block dectin-1 binding of particulate β-(1,3)-glucans, such as zymosan.

Pustulan is a median molecular weight (20 kDa), linear β-(1,6) linked β-D-glucan from lichen Lasallia pustulata which is also able to bind to dectin-1 as major receptor and activate signalling via dectin-1.

Lichenan is a high molecular weight (ca 22-245 kDa) linear, β-(1,3) β-(1,4)-β-D glucan from Cetraria islandica with a structure similar to that of barley and oat β-glucans. Lichenan has a much higher proportion of 1,3- to 1,4-β-D linkages than do the other two glucans. The ratio of β-(1,4)-to β-(1,3)-β-D linkages is approximately 2:1.

β-Glucan from oat and barley are linear, β-(1,3) β-(1,4)-β-D glucans and are commercially available with different molecular weights (medium molecular weight fractions of 35.6 kDa to high molecular weight fractions of up to 650 kDa).

Schizophyllan (SPG) is a gel-forming β-glucan from the fungus Schizophyllum commune. SPG is a high molecular weight (450 kDa) β-(1,3)-D-glucan that has a β-(1,6) monoglucosyl branch in every three β-(1,3)-glucosyl residues on the main chain.

Scleroglucan is a high molecular weight (>1000 kDa) polysaccharide produced by fermentation of the filamentous fungus Sclerotium rolfsii. Scleroglucan consists of a linear β-(1,3) D-glucose backbone with one β-(1,6) D-glucose side chain every three main residues.

Whole glucan particles (WGP) are beta-glucans notable for their ability to modulate the immune response. WGP Dispersible (WGP® Dispersible from Biothera) is a particulate Saccharomyces cerevisiae β-glucan preparation. It consists of hollow yeast cell wall “ghosts” composed primarily of long polymers of β-(1,3) glucose obtained after a series of alkaline and acid extractions from S. cerevisiae cell wall. In contrast to other dectin-1 ligands such as Zymosan, WGP Dispersible lacks TLR-stimulating activity. In contrast, soluble WGP binds dectin-1 without activating this receptor. And it can significantly block the binding of WGP Dispersible to macrophages and its immunostimulatory effect.

Zymosan, an insoluble preparation of yeast cell and activates macrophages via TLR2. TLR2 cooperates with TLR6 and CD14 in response to zymosan. Zymosan is also recognized by dectin-1, a phagocytic receptor expressed on macrophages and dendritic cells, which collaborates with TLR2 and TLR6 enhancing the immune responses triggered by the recognition of zymosan by each receptor.

As a major component of fungal cell walls, different β-glucans have been used as antigens for generating anti-glucan antibodies against fungal infections (e.g.: Torosantucci et al. J Exp Med. 2005 Sep. 5; 202 (5): 597-606., Bromuro et al., Vaccine 28 (2010) 2615-2623, Liao et al., Bioconjug Chem. 2015 Mar. 18; 26 (3): 466-76).

Torosantucci et al. (2005) and Bromuro, et al. (2010) disclose conjugates of the branched β-glucan laminarin, and the linear β-glucan Curdlan coupled to the diphtheria toxoid CRM197. These conjugate vaccines induced high IgG titers against the β-glucan and conferred protection against fungal infections in mice. In addition, also high titers against CRM197 can be detected using such conjugates (Donadei et al., Mol Pharm. 2015 May 4; 12 (5): 1662-72). The authors have also generated β-glucan-CRM197 vaccines, with synthetic linear β-(1,3)-oligosaccharides or β-(1,6)-branched β-(1,3)-oligosaccharides, formulated with the human-acceptable adjuvant MF59. All conjugates induced high titers of anti-β-(1,3)glucan IgG and/or also anti-β-(1,6)-glucan antibodies in addition to the anti-β-(1,3)-glucan IgG demonstrating the immunogenicity of different glucans in combination with classical carrier proteins. Interestingly, Torosantucci et al. failed to demonstrate superior anti-CRM titers following immunization using CRM-glucan conjugates as compared to non-conjugated CRM alone.

Donadei et al. (2015) also analysed conjugates of the diphtheria toxoid CRM197 coupled to linear β-(1,3) glucan Curdlan or to synthetic β-(1,3) oligosaccharides. The conjugates were immunogenic, mounting comparable antibody responses against CRM197. Interestingly, the authors showed that CRM Curdlan conjugates when delivered intradermally resulted in higher antibody titers in comparison to intramuscular (i.m.) immunization. However, intradermal application of CRM-Curdlan did not show different immunogenicity as compared to sub cutaneous application. In addition, in vivo effects were comparable between CRM-Curdlan and non-Curdlan coupled CRM adjuvanted with Alum. Thus, no added benefit of the CLEC coupling on overall immune responses could be detectable in this system.

Liao et al. (2015) disclosed a series of linear β-(1,3)-β-glucan oligosaccharides (hexa-, octa-, deca-, and dodeca-β-glucans) which have been coupled to KLH to generate glycoconjugates. These conjugates were shown to elicit robust T-cell responses and were highly immunogenic inducing high anti-glucan antibody levels. Mice immunized with such vaccines were also eliciting protective immune responses against the deadly pathogen, C. albicans. No comparison of anti-KLH titers with non-conjugated KLH has been performed, hence no information on a potential benefit of the Bglucan is available in this experimental setting.

These findings are highly important for the applicability of glucan-based neoglycoconjugates as novel vaccines: potential antiglucan antibodies induced upon an initial glucan-conjugate immunization could lead to quick elimination of either the same β-glucan vaccines in subsequent booster immunisations or could attenuate immune responses against novel neoglycoconjugate vaccines directed against other indications, an effect well known from vector vaccines. The presence or even (re) stimulation of high-level anti-glucan antibodies, as demonstrated above for mannan and β-glucans (Petrushina et al. 2008, Torosantucci et al. 2005, Bromuro et al., 2010, Liao et al., 2015), could thereby reduce or eliminate potential immune reactions elicited by conjugate vaccines. Thus, it would be crucial for a novel and sustainable platform using CLECs, especially β-glucans, as backbone for immunization to guarantee for a very low or absent glucan antibody inducing capacity of the poly/oligosaccharide used.

Glucan particles (GPs) are highly purified 2-4 μm hollow porous cell wall microspheres composed primarily of β-(1,3)-D-glucans, with low amounts of β-(1,6)-D-glucans and chitin, typically isolated from Saccharomyces cerevisiae, using a series of hot alkaline, acid and organic extractions. They interact with their receptors dectin-1 and CR3 (there is also evidence implying interaction with toll-like receptors and CD5 as additional factors for GP function) and upregulate cell surface presentation of MHC molecules, lead to altered expression of co-stimulation molecules as well as induce the production of inflammatory cytokines. Due to their immunomodulatory properties, GPs have been explored for vaccine delivery.

There are three general approaches for applying GPs in vaccines: (i) as a co-administered adjuvant with antigen(s) to enhance T- and B-cell-mediated immune responses, (ii) chemically crosslinked with antigens and most frequently used (iii) as a physical delivery vehicle of antigens trapped inside the hollow GP cavity, to provide targeted antigen delivery to APCs.

Ad (i): Antigen-specific adaptive immune responses can be enhanced by co-administering GPs together with antigens. In this conventional adjuvant strategy, both innate as well as adaptive immune responses are activated to exert protective responses against pathogens. Williams et al. (Int J Immunopharmacol. 1989; 11(4):403-10) for example adjuvanted a killed Trypanosoma cruzi vaccine by co-administering GPs. The immune response elicited using this formulation resulted in 85% survival of mice challenged with T. cruzi. In contrast, controls that received dextrose, glucan or vaccine alone had 100% mortality.

Ad (ii): The carbohydrate surface of GPs can also be covalently modified using NaIO₄ oxidation, carbodiimide cross-linking or 1-cyano-4-dimethylaminopyridinium tetrafluoroborate-mediated conjugation of antigens to the GP shell. Using this approach, coupling efficacies are very low (approx. 20%, e.g. as described in Pan et al. Sci Rep 5, 10687 (2015)), which limits applicability and the number of vaccine candidates significantly compared to i.e. antigen encapsulation in GPs or the proposed platform technology provided in this application. Such covalently linked antigen-GP conjugates were used in studies for cancer immunotherapy and infectious diseases. For example, Pan et al. (2015) used OVA cross-linked to periodate-oxidized GPs and subcutaneously immunized mice with this vaccine. When mice were challenged with OVA-expressing E.G7 lymphoma cells, a significant reduction in tumor size was observed. GP-OVA was detectable in DCs (CD11c⁺MHC-II⁺) in lymph nodes 12 and 36 h post-subcutaneous injection. Tumor protection was associated with an increase in total anti-Ova immunoglobulin (Ig) G titer, enhanced MHC-II and co-stimulatory molecule (CD80, CD86) expression and heightened cytotoxic lymphocyte responses.

Ad (iii): the most effective approach for applying GPs in vaccines is to employ them for encapsulation of vaccines/antigens into the hollow core. GPs can encapsulate one or more antigens/DNA/RNA/adjuvants/drugs/combinations thereof with high loading efficiency, which is dictated by the type of payload and the mode of delivery intended.

Antigens can be encapsulated in the hollow cavity of the GPS using polymer nano-complexation methods like loading and complexation of the payload using bovine or murine serum albumin and yeast RNA/tRNA or the addition of alginate-calcium or alginate-calcium-chitosan mixtures. Using these strategies, for example Huang et al. (Clin. Vaccine Immunol. 2013; 20:1585-91) reported that mice vaccinated with GP-OVA showed strong CD4+ T-cell lymphoproliferation, a Th1 and Th17 skewed T-cell-mediated immune response together with high IgG1- and IgG2c-specific antibody responses against ovalbumin. The non-covalent encapsulation strategy elicited stronger immune responses compared to GPs co-administered with antigen.

Examples for GP-encapsulated subunit vaccines are GPs encasing soluble alkaline extracts of Cryptococcus neoformans acapsular strain (cap59) which protected mice challenged with lethal doses of highly virulent C. neoformans (60% survival) by inducing an antigen-specific CD4+ T-cell response (positive for IFN-γ, IL-17A) that reduced the fungal colony-forming units (CFU) more than 100-fold from the initial challenge dose (Specht C A et al. Mbio 2015; 6: e01905-e1915, and Specht C A et al., mBio 2017; 8: e01872-e1917.). Additionally, vaccinating mice with GP encapsulating antigens proved efficacious against Histoplasma capsulatum (Deepe G S et al., Vaccine 2018; 36:3359-67), F. tularensis (Whelan A O et al., PLOS ONE 2018; 13: e0200213), Blastomyces dermatitidis (Wuthrich M et al., Cell Host Microbe 2015; 17:452-65) and C. posadasii (Hurtgen B J et al., Infect. Immun. 2012; 80:3960-74).

Beside cancer and infectious disease applications, also a limited number of studies using self-antigens has been performed using GPs as encapsulation agent for vaccine delivery. Along these lines, Rockenstein et al. (J. Neurosci., Jan. 24, 2018·38(4):1000-1014) describe the application of GPs loaded with recombinant human aSynuclein protein (containing both, B- and T-cell epitopes suitable for induction of anti-aSyn immune responses) and Rapamycin which is known to induce antigen-specific regulatory T-cells (Tregs) in a murine model for Synucleinopathies. As expected from previous studies using full length aSynuclein as immunogen, application of the GPs containing aSyn leads to induction of robust anti-aSynuclein antibody titers and alleviates aSynuclein triggered pathologic alteration in the animals to a similar extent as previously published. Addition of Rapamycin efficiently induced the formation of iTregs (CD25 and FOXP3+) cells as the number of such Treg cells was significantly increased following Rapamycin exposure. GPs loaded with antigen aSynuclein and Rapamycin were thus triggering both neuroprotective humoral and iTreg responses in mouse models of synucleinopathy with the combination vaccine (aSyn+Rapamycin) being more effective than either humoral (GP aSyn) or cellular immunization (GP rapamycin) alone. No information on comparability of the effects to conventional, non-GP containing aSynuclein immunization have been reported.

β-glucan neoglycoconjugates efficiently target dendritic cells via the C-type lectin receptor dectin-1, boosting their immunogenicity. Specifically, certain β-glucans have also been used as potential carriers for vaccination using model antigens like OVA (Xie et al., Biochemical and Biophysical Research Communications 391 (2010) 958-962; Korotchenko et al., Allergy. 2021; 76:210-222.) or fusion proteins based on MUC1 (Wang et al., Chem. Commun., 2019, 55, 253).

Xie et al. and Korotchenko et al. were using the branched β-glucan laminarin as backbone for OVA conjugation. These gluconeoconjugates were then applied to mice either epictuaneously or via the subcutaneous route. Xie et al. showed that laminarin/OVA conjugates but not non-conjugated mixing of the compounds was inducing increased anti-OVA CD4+ T-cell responses as compared to ovalbumin alone. Importantly, co-injection of unconjugated laminarin blocked this enhancement supporting the function of laminarin mediated APC targeting. As expected, native OVA and the mixture of OVA and laminarin stimulated low level of anti-OVA antibody production. On the contrary, OVA/laminarin conjugate significantly enhanced antibody responses. Similarly, Korotchenko et al. demonstrated that laminarin conjugation to OVA significantly increased uptake and induced activation of BMDCs and secretion of pro-inflammatory cytokines. These properties of LamOVA conjugate also resulted in enhanced stimulation of OVA-specific naïve T-cells co-cultured with BMDCs. In a prophylactic immunization experiment the authors could confirm that immunization with LamOVA reduced its allergenicity and induced ˜threefold higher IgG1 antibody titers compared with OVA after two immunizations. However, this effect was lost in all groups treated after the third immunization when all groups displayed similar antibody titers. Lam/OVA conjugates and OVA/alum conjugates showed comparable therapeutic efficacy in a murine model of allergic asthma. Thus, these experiments could not provide a clearly superior effect of glucan-based conjugates compared to conventional vaccines.

Wang et al. (2019) analysed the effects of a β-glucan based MUC1 cancer vaccine candidate. Again, the MUC1 tandem repeat sequence GVTSAPDTRPAPGSTPPAH, a well-studied cancer biomarker, was chosen as the peptide antigen providing T- and B-cell epitopes within the repeat sequence. An ethylene glycol (i.e. PEG) spacer was used to link β-glucan and the MUC1 peptide with yeast β-(1,3)-β-glucan polysaccharide applying 1,1′-carbonyl-diimidazole (CDI)-mediated conditions. Size of the β-glucan-MUC1 nanoparticles have been in the range of 150 nm (actual average 162 nm) while unmodified β-glucan was forming particles of approx. 540 nm. The β-glucan-MUC1 conjugate elicited high titers of anti-MUC1 IgG antibodies, significantly higher compared to the control groups. Further analysis of the isotypes and subtypes of the antibodies generated showed that IgG2b is the major subtype, indicating the activation of Th1-type response as a ratio of IgG2b/IgG1 is >1. The observed substantial amount of IgM antibodies indicates the involvement of the C3 component of the complement system, which often induces cytotoxicity and could be problematic for use of such backbones for vaccines which should avoid the development of cytotoxicity, e.g. for chronic or degenerative diseases.

US 2013/171187 A1 discloses an immunogenic composition comprising a glucan and a pharmaceutically acceptable carrier to elicit protective anti-glucan antibodies. Metwali et al. (Am. J. Respir. Crit. Care Med. 185 (2012), A4152; poster session C31 Regulation of Lung Inflammation) investigate into the immunomodulatory effect of a glucan derivative in lung inflammation. WO 2021/236809 A2 discloses a multi-epitope vaccine comprising amyloid-beta and tau peptides for the treatment of Alzheimer's disease (AD). US 2017/369570 A1 discloses β-(1,6)-glucan linked to an antibody directed to a cell present in a tumor microenvironment. US 2002/077288 A1 discloses synthetic immunogenic but non-amyloidogenic peptides homologous to amyloid-beta alone or conjugated for the treatment of AD. US 2013/171187 A1 discloses anti-glucan antibodies used as protective agents against fungal infections with C. albicans. WO 2004/012657 A2 discloses a microparticulate β-glucan as a vaccine adjuvant. CN 113616799 A discloses a vaccine vector consisting of oxidized mannan and a cationic polymer. CN 111514286 A discloses a Zika virus E protein conjugate vaccine with a glucan. U.S. Pat. No. 4,590,181 A discloses a viral antigen mixed in solution with pustulan or mycodextran. Lang et al. (Front. Chem. 8 (2020): 284) reviews carbohydrate conjugates in vaccine developments. Larsen et al. (Vaccines 8 (2020): 226) report that pustulan activated chicken bone marrow-derived dendritic cells in vitro and promotes ex vivo CD4⁺ T-cell recall response to infectious bronchitis virus. US 2010/266626 A1 discloses glucans, preferably laminarin and curdlan, as antigens conjugated to an adjuvant for immunising against fungi. Mandler et al. (Alzh. Dement. 15 (2019), 1133-1148) report on the effects of single and combined immunotherapy approach targeting amyloid-beta protein and alpha synuclein in a dementia with Lewy bodies-like model. Mandler et al. (Acta Neuropathol. 127 (2014), 861-879) reports a next-generation active immunization approach for synucleinopathies using short, immunogenic (B-cell response) peptides that are too short for inducing a T-cell response (autoimmunity) and do not carry the native epitope, but rather a sequence that mimics the original epitope (e.g., oligomeric alpha synuclein) and its implications for Parkinson's disease (PD) clinical trials. Mandler et al. (Mol. Neurodegen. 10 (2015), 10) report that active immunization against alpha synuclein ameliorates the degenerative pathology and prevents demyelination in a model of multiple system atrophy (MSA). Jin et al. (Vaccine 36 (2018), 5235-5244) review β-glucans as potential immunoadjuvants, mainly on the adjuvanticity, structure-activity relationship and receptor recognition properties. WO 2022/060487 A1 discloses a vaccine comprising specific alpha synuclein peptides for the treatment of neurodegenerative diseases. WO 2022/060488 A1 discloses a multi-epitope vaccine comprising amyloid-beta and alpha synuclein peptides for the treatment of AD. US 2009/169549 A1 discloses conformational isomers of modified versions of alpha synuclein produced by introducing cysteines into the alpha synuclein polypeptide and scrambling the disulphide bonds to form stable and immunogenic isomers. WO 2009/103105 A2 discloses vaccines with mimotopes of the alpha synuclein epitope extending from amino acid D115 to amino acid N122 in the native alpha synuclein sequence.

So far, no reports have been published demonstrating the construction or use of individual B-cell or T-cell epitope peptides which were coupled to β-glucans, especially linear β-glucans and/or pustulan with high binding specificity/ability to dectin-1, thereby forming novel gluconeoconjugates as those proposed in this application.

It is therefore an object of the present invention to provide improved vaccines as conjugate vaccines made of the vaccination antigen conjugated to carbohydrate-based CLEC adjuvants, especially to provide vaccines which provide an improved immune response in the vaccinated individual compared to current state of the art conjugate vaccines, especially carbohydrate-based CLEC-peptide/protein conjugate vaccines.

It is a further object of the present invention to provide vaccine compositions which confer immunity to short, easily interchangeable, highly specific B/T-cell epitopes using a CLEC backbone with previously unmet efficacy, specificity and affinity by conventional vaccines.

A specific object of the present invention is the provision of vaccines with improved selectivity and/or specificity of a CLEC-based vaccine for the dermal compartment.

Another object of the present invention is to provide vaccines which—as exclusively as possible—induce target-specific immune responses while inducing no or only very limited CLEC- or carrier protein-specific antibody responses.

It is a further object of the present invention to provide vaccine compositions which confer immunity to short, easily interchangeable, highly specific B/T-cell epitopes of alpha synuclein using a CLEC backbone with previously unmet efficacy, specificity and affinity by conventional vaccines for appropriate prevention and treatment of synucleopathies.

A specific object of the present invention is the provision of alpha synuclein vaccines with improved selectivity and/or specificity of a CLEC-based vaccine for the dermal compartment.

Another object of the present invention is to provide vaccines which—as exclusively as possible—induce alpha synuclein—specific immune responses while inducing no or only very limited CLEC- or carrier protein-specific antibody responses.

Another object of the present invention is to provide peptide immunogen constructs of the alpha synuclein protein (aSyn) and formulations thereof for treatment of synucleinopathies.

Therefore, the present invention provides a β-glucan for use as a C-type lectin (CLEC) polysaccharide adjuvant for B-cell and/or T-cell epitope polypeptides, preferably, wherein the β-glucan is covalently conjugated to the B-cell and/or T-cell epitope polypeptide to form a conjugate of the β-glucan and the B-cell and/or T-cell epitope polypeptide, wherein the β-glucan is a predominantly linear β-(1,6)-glucan with a ratio of β-(1,6)-coupled monosaccharide moieties to non-β-(1,6)-coupled monosaccharide moieties of at least 1:1, preferably at least 2:1, more preferred, at least 5:1, especially at least 10:1.

With the present invention one or more objects listed above are successfully solved. This was unexpected for a person skilled in the art, because until now, no reports have been published in the present field of technology demonstrating the construction and applicability or efficacy of compounds similar to the novel, small, modular gluconeoconjugates according to the present invention.

Surprisingly, it was shown with the present invention that by conjugation (i.e. by covalently coupling; used synonymously herein) of peptides/proteins to the selected CLEC-carrier according to the present invention, wherein the conjugation may be based on state-of-the-art chemistry, superior pharmaceutical formulations for effecting immune responses were obtained. In the present field of technology, a significant number of different coupling methods is available. In the course of establishing the present invention, hydrazone formation or coupling via heterobifunctional linkers have been identified as specifically preferred methods. In general, activation of the CLEC prior to conjugation (e.g.: formation of reactive aldehydes on vicinal OH groups of the sugar moieties) and presence of reactive groups on the peptide/protein of choice (e.g. N- or C-terminal hydrazide residues, SH groups (e.g.: via N- or C-terminal cysteines)) is required. The reaction can be a single step reaction (e.g. mixing of activated CLECs with Hydrazide-peptides leading to hydrazone formation or a multistep process (e.g.: activated CLEC is reacted with a hydrazide from a heterobifunctional linker and subsequently the peptide/protein is coupled via respective reactive groups).

Accordingly, the components of the conjugates according to the present invention may be directly coupled to each other, e.g. by coupling the B-cell epitope and/or the T-cell epitope to the β-glucan and/or to a carrier protein or by coupling the β-glucan to a carrier protein (in all possible orientations). Referring to a “B-cell epitope polypeptide” or a “T-cell epitope polypeptide” herein means by default the B-cell or T-cell epitope of the “B-cell epitope polypeptide” or the “T-cell epitope polypeptide” and not to a B-cell or T-cell epitope of the carrier protein (if present), except if its explicitly referred to a B-cell or T-cell epitope of the carrier protein. According to a preferred embodiment, the B-cell epitope and/or the T-cell epitope is preferably linked to the β-glucan or mannan and/or to a carrier protein by a linker, more preferred a cysteine residue or a linker comprising a cysteine or glycine residue, a linker resulting from hydrazide-mediated coupling, from coupling via heterobifunctional linkers, such as N-β-maleimidopropionic acid hydrazide (BMPH), 4-[4-N-maleimidophenyl] butyric acid hydrazide (MPBH), N-[ε-Maleimidocaproic acid) hydrazide (EMCH) or N-[κ-maleimidoundecanoic acid] hydrazide (KMUH), from imidazole mediated coupling, from reductive amination, from carbodiimide coupling a —NH—NH₂ linker; an NRRA, NRRA-C or NRRA-NH—NH₂ linker, peptidic linkers, such as bi-, tri-, tetra-(or longer)-meric peptide groups, such as CG or CG, or cleavage sites, such as a cathepsin cleavage site; or combinations thereof, especially by a cysteine or NRRA-NH—NH₂ linker. It is clear that “a linker resulting from (e.g.) hydrazide-mediated coupling” refers to the resulting chemical structure in the conjugate after conjugations, i.e. as present in the resulting conjugate after conjugation. Amino acid linkers may be present in the conjugated form either with a peptidic bond (e.g. with glycine containing linkers) or via a functional group of the amino acid (such as the disulfide bond for cysteine linkers).

The novel class of conjugates according to the present invention turned out to confer immunity to short, easily interchangeable, highly specific B/T-cell epitopes by using the CLEC backbone of the present invention showing efficacy, specificity and affinity previously unmet by conventional vaccines: In fact, the conjugates according to the present invention are the first examples for use of short B-cell/T-cell epitopes in a CLEC based vaccine avoiding the need for presenting the epitopes in the form of fusion proteins including formation of tandem repeats of epitopes or fusion of different tandem repeats to form a stable and effective immunogen (as e.g. necessary for the MUC1 approach with mannan referred to above).

With the present invention, also the necessity to use full length proteins for use in CLEC vaccines (i.e. payload in glucan particles (GPS)) can be avoided. Moreover, the problem of autoimmune-reactions especially induced by (unwanted) T-cell epitopes present in immunogens like self-proteins (e.g.: T-cell epitopes in aSyn, amyloid β etc.) or mixed self-epitopes (e.g.: the MUC1-tandem repeat used as immunogen) when using CLECs can also be avoided.

According to the present invention, for the first-time short epitopes (B- and/or T-cell epitopes, mainly peptides, modified peptides) can be united with a functional CLEC-based backbone using covalent coupling based on well-established chemistry wherein the possible methods for conjugation can be adapted to the requirements of the specific epitope based on methods well known in the field.

The presentation of the short peptide(s) according to the present invention can be made as individually conjugated moieties in combination with an individual foreign T-cell epitope (as short peptide or long protein) or as a complex/conjugate with a larger carrier molecule providing the T-cell epitope for inducing a sustainable immune response. The design of the vaccines according to the present invention allows for preparation of multivalent conjugates as a prerequisite for efficient immune response induction by highly efficient B-cell receptor (BCR)-crosslinking.

Moreover, with the present invention a CLEC based vaccine can be provided with an excellent selectivity/specificity for the dermal compartment. In fact, the conjugate design according to the present invention builds on CLECs as carrier for the target specific epitopes which display high binding specificity for PRRs on dermal APCs/DCs, especially on dectin-1 (or MR and DC-SIGN in the case of mannan) to allow for skin selective/specific- and receptor mediated uptake (=targeted vaccine delivery).

The CLEC polysaccharide used as carrier according to the present invention is used to focus the carrier-peptide conjugate into preferably dermal/cutaneous DCs and to initiate an immune response. This is i.a. due to an epidermal or dermal (not subcutaneous) specificity. The CLEC backbone and the efficient dermal immune response initiation according to the present invention also helps to avoid the compulsory use of adjuvants, typical for conventional vaccines and also used in exemplary CLEC based vaccines (e.g.: use of Alum, MF59, CFA, PolyI:C or other adjuvants). According to a preferred embodiment of the present invention, the use of adjuvants may be significantly reduced or omitted, e.g. in circumstances wherein addition of adjuvants is not indicated.

Several CLECs have been used in previous applications however none of the proposed conjugate structures could confer skin selectivity (i.e. high dectin-1 binding ability, highly efficient dermal DC targeting and superior immunogenicity for dermal application as compared to all other routes (i.e. subcutaneous, intramuscular and i.p.).

The selection of the CLEC according to the present invention has been made as to provide a novel solution to target skin specific DCs and skin specific immunization with high efficacy. The conjugates according to the present invention also exert limited activity in other classical tissues for immunization like muscle or sub-cutaneous tissue which is in contrast to previous CLEC-based vaccines/vaccine candidates described which have been applied i.m. or s.c. As a result of the experiments conducted in the course of the present invention, vaccines according to the present invention, especially those which use pustulan as CLEC were identified as being surprisingly selective for skin immunization.

The present invention is drawn to any B-cell and/or T-cell epitope polypeptide and any predominantly linear β-(1,6)-glucan with a ratio of (1,6)-coupled monosaccharide moieties to non-(1,6)-coupled monosaccharide moieties of at least 1:1. As also shown in the example section below, the present teaching enables and provides support for any B-cell and/or T-cell epitope polypeptide and has not revealed any limitation with respect to such epitopes, especially if the epitopes are already part of the prior art and/or established epitopes. The specific B-cell and/or T-cell epitope polypeptides as shown and referred to herein are preferred epitopes but the present invention is not limited thereto. In the course of the present invention and after the numerous epitopes tested so far (see the functionally and structurally very diverse group of epitopes (including a significant number of model epitopes) investigated and confirmed experimentally in the example section), no limitation with respect to the nature and structure of the B-cell and/or T-cell epitope appeared (linear polypeptides, self-peptides, polypeptides with posttranslational modifications, such as sugar structures or pyro-glutamate, mimotopes, allergens, structural epitopes, conformational epitopes, etc.), especially for pustulan as the β-(1,6)-glucan. In each of the cases it was experimentally shown that it is the β-glucan according to the present invention and the covalent conjugation to the epitope polypeptide which is responsible for the immunological performance and not the concrete structural characteristics of the individual epitope.

The terms “B-cell and/or T-cell epitope polypeptide” as used herein is an accepted functional term in the present field of technology: T-cell epitopes are presented on the surface of an antigen-presenting cell, where they are bound to major histocompatibility complex (MHC) molecules. In humans, professional antigen-presenting cells are specialized to present MHC class II peptides, whereas most nucleated somatic cells present MHC class I peptides. T-cell epitopes presented by MHC class I molecules are typically peptides between 8 and 11 amino acids in length, whereas MHC class II molecules present longer peptides, 13-17 amino acids in length, and non-classical MHC molecules also present non-peptidic epitopes such as glycolipids. A B-cell epitope is the part of the antigen that immunoglobulin or antibodies bind to. B-cell epitopes can be e.g. conformational or linear.

According to a preferred embodiment of the present invention, the conjugate according to the present invention comprises polypeptides with at least one B-cell and at least one T-cell epitope, preferably a B-cell epitope+CRM197 conjugate covalently linked to β-glucan, especially a peptide+CRM197+linear β-(1,6)-glucan or a peptide+CRM197+linear pustulan conjugate. Preferred glucan to peptide ratios, especially pustulan to peptide ratios, are ranging from 10 to 1 (w/w) to 0.1 to 1 (w/w), preferably 8 to 1 (w/w) to 2 to 1 (w/w), especially 4 to 1 (w/w), with the proviso if the conjugate comprises a carrier protein, the preferred ratio of β-glucan or mannan to B-cell-epitope-carrier polypeptide is from 50:1 (w/w), to 0.1:1 (w/w), especially 10:1 to 0.1:1.

With the present invention, it becomes possible to focus on the induction of target specific immune responses while inducing no or only very limited CLEC- or carrier-protein specific antibody responses. The conjugates according to the present invention thereby solve the problem posed by classical conjugate vaccines, which have to rely on the use of foreign carrier proteins to induce a sustainable immune response. Current state of the art conjugate vaccine development is strongly built on carrier molecules like KLH, CRM197, Tetanus Toxoid or other suitable proteins, which are complexed with target specific short antigens delivering the substrate for immune reactions against the different target diseases like infectious, degenerative or neoplastic diseases, including for example Her2-neu positive cancer, aSynuclein for synucleinopathies like Parkinson's disease, amyloid β peptides for amyloidosis like Alzheimer's disease, Tau for treatment of tauopathies including Alzheimer's disease, PCSK9 for hypercholesteremia, IL23 for psoriasis, TDP43 and FUS for Frontotemporal lobar degeneration (FTLD) and Amyotrophic lateral sclerosis (ALS), (mutant) Huntingtin for Huntington's disease, Immunoglobulin light and heavy chain amyloidosis (AL, AH, AA), Islet amyloid polypeptide (IAPP) and amylin for diabetes type 2, (mutant) Transthyretin for ATTR/Transthyretin amyloidosis, and others.

The immunological performance and efficiency of the conjugates according to the present invention and the vaccines comprising these conjugates are also unexpected and surprising in view of the guidance of the prior art wherein β-glucans, especially predominantly linear β-(1,6)-glucans, have mainly been used as antigens themselves for eliciting specific immune responses against fungi in which such β-glucans are present (see e.g. US 2013/171187 A1; Metwali et al., Am. J. Respir. Crit. Care Med. 185 (2012), A4152; poster session C31; US 2013/171187 A1, US 2010/266626 A1, Jin et al. (Vaccine 36 (2018), 5235-5244)). However, with the present invention it was demonstrated that the conjugates according to the present invention are not able to elicit a significant immune response to the β-glucans, but that-due to the architecture of the present conjugates—the immune response is shifted to the B-cell and/or T-cell epitope polypeptide covalently conjugated to the β-glucans. Conjugating these B-cell and/or T-cell epitope polypeptides to the linear β-glucans seems to hide the immune response eliciting ability of the β-glucans but to expose and significantly improve the presentation of the covalently coupled B-cell and/or T-cell epitope polypeptides to the immune system. This teaching was neither disclosed in the prior art nor made obvious with such prior art:

US 2017/369570 A1 disclosing β-(1,6)-glucan linked to an antibody directed to a cell present in a tumor microenvironment is based on a completely different concept and mechanism (tumor treatment).

On the other hand, glucans were used as components in vaccines (mostly as “(liposomal) glucan (nano)particles”) but not with covalent coupling of a B-cell and/or T-cell epitope polypeptide to the glucan (e.g. WO 2004/012657 A2, CN 113616799 A, U.S. Pat. No. 4,590,181 A, Lang et al., Front. Chem. 8 (2020): 284; Larsen et al., Vaccines 8 (2020): 226).

Finally, the improved effect of the predominantly linear β-(1,6)-glucans according to the present invention over the constructs and compositions according to WO 2022/060487 A1, WO 2022/060488 A1, US 2009/169549 A1, WO 2009/103105 and CN 111514286 A (such as β-(1,2)-glucans or β-(1,3)-glucans) have been demonstrated in the example section below.

In view of these advantageous properties of the conjugates of the present invention, it follows that the conjugates and vaccines according to the present invention are specifically useable for active anti-Tau protein vaccination, also including variants, undergoing truncation, (hyper) phosphorylation, nitration, glycosylation and/or ubiquitination, for the treatment and prevention of Tauopathies, especially Alzheimer's Disease and Down Syndrome or other tauopathies including Pick disease, progressive supranuclear palsy (PSP), corticobasal degeneration, Frontotemporal dementia and parkinsonism linked to chromosome 17 (FTDP-17) and argyrophilic grain disease. Emerging other entities and pathologies include globular glial tauopathies, primary age-related tauopathy (PART), which includes neurofibrillary tangle dementia, chronic traumatic encephalopathy (CTE), and aging-related tau astrogliopathy. In addition, also other entities are included like vacuolar tauopathy, ganglioglioma and gangliocytoma, lytico-bodig disease (Parkinson-dementia complex of Guam), meningioangiomatosis, postencephalitic parkinsonism and subacute sclerosing panencephalitis (SSPE).

Tauopathies are often overlapped with synucleinopathies, possibly due to interaction between the synuclein and tau proteins. Hence, anti-Tau conjugates according to the present invention are specifically useable for active anti-Tau protein vaccination against synucleinopathies, especially Parkinson's disease (PD), Dementia with Lewy bodies (DLB) and Parkinson's disease dementia (PDD).

The anti-Tau vaccines may be highly effective when used alone or in combination with pre-existing peptide vaccines directed against other pathologic molecules involved in B-amyloidoses, tauopathies or synucleopathies, especially with mixed pathology (i.e. the presence of AB-pathology with Tau-pathology and/or aSyn pathology). Therefore, it is a preferred embodiment to provide a combination of anti-Tau vaccines with anti-AB and/or anti-aSyn peptide vaccines to treat degenerative disease like Alzheimer's disease, dementia in Down syndrome, dementia with Lewy bodies, Parkinson's disease dementia, Parkinson's disease.

According to a preferred embodiment, the Tau protein derived polypeptide is selected from native human Tau (441 aa isoform; GenBank entry >AAC04279.1; Seq ID No

MAEPRQEFEV MEDHAGTYGL GDRKDQGGYT
MHQDQEGDTD AGLKESPLQT PTEDGSEEPG
SETSDAKSTP TAEDVTAPLV DEGAPGKQAA
AQPHTEIPEG TTAEEAGIGD TPSLEDEAAG
HVTQARMVSK SKDGTGSDDK KAKGADGKTK
IATPRGAAPP GQKGQANATR IPAKTPPAPK
TPPSSGEPPK SGDRSGYSSP GSPGTPGSRS
RTPSLPTPPT REPKKVAVVR TPPKSPSSAK
SRLQTAPVPM PDLKNVKSKI GSTENLKHQP
GGGKVQIINK KLDLSNVQSK CGSKDNIKHV
PGGGSVQIVY KPVDLSKVTS KCGSLGNIHH
KPGGGQVEVK SEKLDFKDRV QSKIGSLDNI
THVPGGGNKK IETHKLTFRE NAKAKTDHGA
EIVYKSPVVS GDTSPRHLSN VSSTGSIDMV
DSPQLATLAD EVSASLAKQG L

or a polypeptide comprising or consisting of amino acid residues derived from human Tau including post-translationally modified, phosphorylated, double-phosphorylated, hyperphosphorylated, nitrated, glycosylated and/or ubiquitinated) amino acids including Tau2-18, Tau 176-186, Tau 181-210, Tau 200-207, Tau 201-230, Tau 210-218, Tau 213-221, Tau 225-234, Tau 235-246, Tau 251-280, Tau 256-285, Tau 259-288, Tau 275-304, Tau260-264, Tau 267-273, Tau294-305, Tau 298-304, Tau 300-317, Tau 329-335, Tau 361-367, Tau 362-366, Tau379-408, Tau 389-408, Tau 391-408, Tau 393-402, Tau 393-406, Tau393-408, Tau 418-426, Tau 420-426.

According to a preferred embodiment, the Tau protein derived polypeptide is selected from mimics of the above-mentioned Tau derived polypeptides including mimotopes and peptides containing amino acid substitutions mimicking phosphorylated amino acids including substitution of phosphorylated S by D and phosphorylated T by E, respectively including Tau176-186, Tau200-207, Tau210-218, Tau213-221, Tau225-234, Tau379-408, Tau389-408, Tau391-408, Tau393-402, Tau393-406, Tau418-426, Tau420-426.

US 2008/050383 A1 as well as Asuni et al. (Journal of Neuroscience 34:9115-9129) disclose that antibodies directed to Tau379-408 with two phosphorylated aas: pS396 and pS404 as suitable for immunotherapy against Tau pathology and Boutajangout et al. (J. Neurosci., Dec. 8, 2010 30 (49): 16559-16566) disclose use of the same epitope: double phosphorylated polypeptide Tau379-408 with pSp396 and pS404 in combination with the adjuvant AdjuPhos as effective as active immunotherapeutic preventing cognitive decline in several tests in the htau/PS1 model that was associated with reduction in pathological tau within the brain. Bi et al. (2011, PLOS One 12: e26860.) also show that Tau-targeted immunization using a 10-mer polypeptide derived from double phosphorylated sequence Tau395-406 (with ps396 and ps404) conjugated to KLH and adjuvanted with with either complete or incomplete Freund's adjuvant impedes progression of neurofibrillary histopathology in aged P301L Tau transgenic mice.

Boimel M et al. (2010; Exp Neurol 2:472-485) showed that use of the double phosphorylated peptides Tau195-213 [ps202/pT205], Tau207-220 [pT212/pS214] and Tau224-238 [pT231] emulsified in complete Freund's adjuvant (CFA) and pertussis toxin leads to alleviation of Tau associated pathology in animals.

Troquier et al. (2012 Curr Alzheimer Res 4:397-405) show that targeting Tau by active Tau immunotherapy using artificial peptide constructs consisting of a N-terminal YGG linker fused to a 7-(Tau418-426) or 11-mer (Tau417-427) peptide derived from human Tau carrying ps422 coupled to KLH and adjuvanted with CFA in the THYTau22 Mouse Model can be a suitable therapeutic approach as a decrease in insoluble Tau species (AT100- and pS422 immunoreactive) correlating with a significant cognitive improvement using the Y-maze was detectable.

US 2015/0232524 A1 as well as Davtyan H et al. (Sci Rep. 2016; 6:28912, Vaccine. 2017; 35:2015-24 and Alzheimer's Research & Therapy (2019) 11:107) and Joly-Amado et al. (Neurobiol Dis. 2020 February; 134:104636) disclose peptide immunogens and show that the vaccine AV-1980R and AV-1980D both based on the MultiTEP platform consisting of 3 repeats of Tau2-18 fused to several promiscuous T-cell epitopes as recombinant polypeptide or as DNA vaccine, respectively, induces strong immune responses and reduces tau pathology in in different tauopathy models.

EP 3 097 925 B1 discloses peptide immunogens consisting from phospho-peptides derived from human Tau441 and Theunis et al. (2013, PLOS ONE 8 (8): e72301) show, based on EP 3 097 925 B1 a liposomal vaccine carrying Tau peptide Tau 393-408 (carrying pS396 and pS402) which is able to elicit anti-phospho Tau antibodies which was accompanied by improvement in the clinical condition and reduced indices of tauopathy in the brain of the Tau. P301L mice.

Sun et al. (Signal Transduction and Targeted Therapy (2021) 6:61) disclose various immunogens based on Norovirus P particles. The vaccine pTau31 (consisting of particles containing fusion peptides of Tau195-213 with ps202 and pT205 and Tau395-406 with ps396 and pS404) generated robust pTau antibodies and could significantly reduce tau pathology and improve behavioral deficits in a Tau Tg animal model.

EP 2 758 433 B1 discloses peptide based immunogens for interfering with Tau pathology. The invention discloses use as peptide conjugate vaccines (e.g.: as peptide KLH vaccines). Kontsekova et al. (Alzheimer's Research & Therapy 2014, 6:44) disclose such peptide vaccines (i.e. Axon peptide 108 (Tau294-305; KDNIKHVPGGGS) conjugated to KLH and adjuvanted with Alum; also known as AADvac1) induced a robust protective humoral immune response, with antibodies discriminating between pathological and physiological tau. Active immunotherapy reduced the levels of tau oligomers and the extent of neurofibrillary pathology in the brains of transgenic rats.

Although in principle, the present invention is able to improve all suggested Tau vaccination polypeptides, selected epitopes were specifically assessed with respect to their suitability with the present platform. For example, Tau294-305, SeqID35+36 was shown to be superior to a KLH based vaccine as suggested in EP2 758 433 B1 and Kontsekova et al.

Further preferred target sequences include:

| Tau-position | Sequence |
|---|---|
| 195-213 | SGYSSPGSPGTPGSRSRTP |
| 207-220 | GSRSRTPSLPTPPT |
| 224-238 | KKVAVVRTPPKSPSS |
| 393-408 | VYKpSPVVSGDTpSPRHL |
| 379-408 [P-Ser396, 404] | RENAKAKTDHGAEI- |
| | VYKpSPVVSGDTpSPRHL |
| Tau260-264 [P-Ser262] | IGpSTE |
| 294-305 | KDNIKHVPGGGS |
| 251-280 | PDLKNVKSKIGSTENLKHQPGGGKVQIINK |
| 256-285 | VKSKIGSTENLKHQPGGGKVQIINKKLDLS |
| 256-285-pS262 | VKSKIGpSTENLKHQPGGGKVQIINKKLDLS |
| 259-288 | KIGSTENLKHQPGGGKVQIINKKLDLSNVQ |
| 275-304 | VQIINKKLDLSNVQSKCGSKDNIKHVPGGG |
| 201-230-pT217 | GSPGTPGSRSRTPSLPpTPPTREPKKVAVVR |
| 379-408-pS396pS404 | RENAKAKTDHGAEI- |
| | VYKpSPVVSGDTpSPRHL |
| 181-210-pS202pT205 | TPPSSGEPPKSGDRSGYSSPGPSPGpT- |
| | PGSRS |
| 300-317 | VPGGGSVQIVYKPVDLSK |
| 267-273 | KHQPGGG |
| 298-304 | KHVPGGG |
| 329-335 | HHVPGGG |
| 361-367 | THVPGGG |
| 200-207 (pS202/pT205) | PGpSPGpTPG |
| 200-207 (pS202/pT205) | PGDPGEPG |
| phospho mimic | |
| 200-207 (pS202/pT205) | SPGDPGEPG |
| phospho mimic | |
| 210-218 (pT212/pS214) | SRpTPpSLPTP |
| 210-218 (pT212/pS214) | SREPDLPTP |
| phospho mimic | |
| 210-218 (pT212/pS214) | SREPDLP |
| phospho mimic | |
| 213-221 (pT217) | PSLPpTPPTR |
| 213-221 (pS214/pT217) | PpSLPpTPPTR |
| 213-221 (pT217) | PSLPEPPTR |
| phospho mimic | |
| 213-221 (ps214/pT217 | PDLPEPPTR |
| phospho mimic | |
| 393-402 (pSer396) | VYKpSPVVSGD |
| 393-402 (pSer396) | VYKDPVVSG |
| phospho mimic | |
| 393-406 (pSer396, pSer404) | VYKpSPVVSGDTpSPR |
| 393-406 (pSer396, pSer404) | VYKDPVVSGDTDPR |
| phospho mimic | |
| 176-186 (pT181) | PPAPKpTPPSSG |
| 176-186 (pT181) | PPAPKEPPSSG |
| phospho mimic | |
| 225-234 (pT231) | KVAVVRpTPPK |
| 225-234 (pT231) | KVAVVREPPKS |
| phospho mimic | |
| Tau379-408 [P-Ser396, 404] | RENAKAKTDHGAEI- |
| | VYKpSPVVSGDTpSPRHL |
| Tau379-408 [P-Ser396, 404] | RENAKAKTDHGAEIVYKD |
| phospho mimic | PVVSGDTDPRHL |
| 389-408 | GAEIVYKpSPVVSGDTpSPRHL |
| 391-408 | EIVYKpSPVVSGDTpSPRHL |
| 389-408 phospho mimic | GAEIVYKDPVVSGDTDPRHL |
| 391-408 phospho mimic | EIVYKDPVVSGDTDPRHL |
| 418-426 | DMVDpSPQLA |
| 418-426 phospho mimic | DMVDDPQLA |
| 420-426 | VDpSPQLA |
| 420-426 phospho mimic | VDDPQLA |
| 362-366 | HVPGG (LDNITHVPGGGNKKIE) |
| 235-246 | SPSSAKSRLQTA |

In view of these advantageous properties of the conjugates of the present invention, it follows that the conjugates and vaccines according to the present invention are specifically useable for active immunotherapy for IL12/IL23 related disease and autoimmune inflammatory diseases. IL-23 related disease is selected from the group psoriasis, psoriatic arthritis, rheumatoid arthritis, systemic lupus erythematosus, diabetes, preferably type 1 diabetes, atherosclerosis, inflammatory bowel disease (IBD)/M. Crohn, multiple sclerosis, Behcet disease, ankylosing spondylitis, Vogt-Koyanagi-Harada disease, chronic granulomatous disease, hidratenitis suppurtiva, anti-neutrophil cytoplasmic antibodies (ANCA-) associated vasculitides, neurodegenerative diseases, preferably M. Alzheimer or multiple sclerosis, atopic dermatitis, graft-versushost disease, cancer, preferably Oesophagal carcinoma, colorectal carcinoma, lung adenocarcinoma, small cell carcinoma, and squamous cell carcinoma of the oral cavity, especially psoriasis, neurodegenerative diseases or IBD. Furthermore, the IL-12/23-directed vaccines can be used together/in combination with vaccines against other targets, as recent data suggest that IL-23-driven inflammation can exacerbate other diseases, such as Alzheimer's disease or possibly diabetes.

According to a preferred embodiment the IL12/IL23 protein derived polypeptide is derived from native human IL12/IL23 or is a mimic with one or more aa exchanges forming a mimotope of the respective native sequence.

According to a preferred embodiment, the IL12/IL23 protein derived polypeptide is selected from the subunit of the heterodimeric protein IL23, native human IL-23p19 or a polypeptide comprising or consisting of amino acid residues derived from this subunit or of mimotopes. In WO 2005/108425 A1, peptides FYEKLLGSDIFTGE, FYEKLLGSDIFTGEPSLLPDSP, VAQLHASLLGLSQLLQP, GEPSLLPDSPVAQLHASLLGLSQLLOP, PEGHHWETQQIPSLSPSQP, PSLLPDSP, LPDSPVA, FYEKLLGSDIFTGEPSLLPDSPVAQLHASLLGLSQLLOP, LLPDSP, LLGSDIFTGEPSLLPDSPVAQLHASLLG, FYEKLLGSDIFTGEPSLLPDSPVAQLHASLLG, QPEGHHW, LPDSPVGQLHASLLGLSOLLQ and QCQOLSOKLCTLAWSAHPLV derived from IL23p19 were proposed as vaccination peptides for IL-23. In WO 03/084979 A2, GHMDLREEGDEETT, LLPDSPVGOLHASLLGLSQ and LLRFKILRSLQAFVAVAARV from IL-23p19 were mentioned as possible anti-cytokine vaccines. WO 2016/193405 A1 discloses peptide immunogens derived from IL12/23 p19 subunit (accession number: Q9NPF7) with the amino acid sequence

MLGSRAVMLL LLLPWTAQGR AVPGGSSPAW
TQCQQLSQKL CTLAWSAHPL VGHMDLREEG
DEETTNDVPH IQCGDGCDPQ GLRDNSQFCL
QRIHQGLIFY EKLLGSDIFT GEPSLLPDSP
VGQLHASLLG LSQLLQPEGH HWETQQIPSL
SPSQPWQRLL LRFKILRSLQ AFVAVAARVE
AHGAATLSP

as possible anti-cytokine vaccines especially aa136-145, aa136-143, aa 136-151, aa137-146, aa144-154, aa144-155 thereof and others, especially sequences: QPEGHHWETQQIPSLS, GHHWETQQIPSLSPSQPWQRL, QPEGHHWETQ, TQQIPSLSPSQ, QPEGHHWETQQIPSLSPSQ, QPEGHHWETQQIPSLSPS.

According to a preferred embodiment, the IL12/IL23 protein derived polypeptide is selected from the subunit of the heterodimeric protein IL23, native human IL12/23p40 or a polypeptide comprising or consisting of amino acid residues aa15-66, aa38-46, aa53-71, aa119-130, aa160-177, aa236-253, aa274-285, aa315-330 of native human IL12/23p40 (accession number: P29460.1) having the following amino acid sequence:

MCHQQLVISW FSLVFLASPL VAIWELKKDV
YVVELDWYPD APGEMVVLTC DTPEEDGITW
TLDQSSEVLG SGKTLTIQVK EFGDAGQYTC
HKGGEVLSHS LLLLHKKEDG IWSTDILKDQ
KEPKNKTFLR CEAKNYSGRF TCWWLTTIST
DLTFSVKSSR GSSDPQGVTC GAATLSAERV
RGDNKEYEYS VECQEDSACP AAEESLPIEV
MVDAVHKLKY ENYTSSFFIR DIIKPDPPKN
LQLKPLKNSR QVEVSWEYPD TWSTPHSYFS
LTFCVQVQGK SKREKKDRVF TDKTSATVIC
RKNASISVRA QDRYYSSSWS EWASVPCS

In WO 03/084979 A2, peptides LLLHKKEDGIWSTDILKDQKEPKNKTFLRCE and KSSRGSSDPQG from the IL-12/23 p40 subunit were mentioned as possible anti-cytokine vaccines.

Luo et al. J Mol Biol 2010 Oct. 8; 402 (5): 797-812. disclose the conformational epitope of the anti-IL12/IL23p40 specific antibody Ustekinumab—aa15-66 which is efficiently reducing IL12 (IL23 related disease. Guan et al. (Vaccine 27 (2009) 7096-7104) disclose immunogens aa38-46, aa53-71, aa119-130, aa160-177, aa236-253, aa274-285, aa315-330 of murine IL12/23 accession numbers: P43432 (p40) and Q9EQ14 (p19)) which has the following amino acid sequence: P43432 (p40):

MCPQKLTISW FAIVLLVSPL MAMWELEKDV
YVVEVDWTPD APGETVNLTC DTPEEDDITW
TSDQRHGVIG SGKTLTITVK EFLDAGQYTC
HKGGETLSHS HLLLHKKENG IWSTEILKNF
KNKTFLKCEA PNYSGRFTCS WLVQRNMDLK
FNIKSSSSSP DSRAVTCGMA SLSAEKVTLD
QRDYEKYSVS CQEDVTCPTA EETLPIELAL
EARQQNKYEN YSTSFFIRDI IKPDPPKNLQ
MKPLKNSQVE VSWEYPDSWS TPHSYFSLKF
FVRIQRKKEK MKETEEGCNQ KGAFLVEKTS
TEVQCKGGNV CVQAQDRYYN SSCSKWACVP
CRVRS

recombinantly joined to HBcAg.

Although in principle, the present invention is able to improve all suggested IL12/IL23 related disease vaccination polypeptides, selected epitopes were specifically assessed with respect to their suitability with the present platform. For example, SeqID37/38 and SeqID41/42 WISIT vaccines were shown to be superior to a KLH based vaccine. The murine sequence SeqID39/40 showed similar efficacy as KLH based conjugates in mice and was also active in IL12/23 recognition.

In view of these advantageous properties of the conjugates of the present invention, it follows that the conjugates and vaccines according to the present invention are specifically useable for active anti-EMPD (Extra Membrane Proximal Domain, as part of the membrane IgE-BCR) vaccination for the treatment and prevention of IgE related diseases. Exclusive targeting and crosslinking of membrane IgE-BCR has been achieved by addressing the membrane anchoring region that is only found on membrane-IgE but not on soluble serum IgE—the extracellular membrane proximal domain of IgE (EMPD IgE). IgE-related disease include allergic diseases such as seasonal, food, pollen, mold spores, poison plants, medication/drug, insect-, scorpion- or spider-venom, latex or dust allergies, pet allergies, allergic asthma bronchiale, non-allergic asthma, ChurgStrauss Syndrome, allergic rhinitis and -conjunctivitis, atopic dermatitis, nasal polyposis, Kimura's disease, contact dermatitis to adhesives, antimicrobials, fragrances, hair dye, metals, rubber components, topical medicaments, rosins, waxes, polishes, cement and leather, chronic rhinosinusitis, atopic eczema, autoimmune diseases where IgE plays a role (“autoallergies”), chronic (idiopathic) and autoimmune urticaria, cholinergic urticaria, mastocytosis, especially cutaneous mastocytosis, allergic bronchopulmonary aspergillosis, chronic or recurrent idiopathic angioedema, interstitial cystitis, anaphylaxis, especially idiopathic and exercise-induced anaphylaxis, immunotherapy, eosinophil-associated diseases such as eosinophilic asthma, eosinophilic gastroenteritis, eosinophilic otitis media and eosinophilic oesophagitis (see e.g. Holgate 2014 World Allergy Organ. J. 7:17., U.S. Pat. No. 8,741,294 B2). Furthermore, the vaccines or conjugates according to the present invention are used for the treatment of lymphomas or the prevention of sensibilisation side effects of an anti-acidic treatment, especially for gastric or duodenal ulcer or reflux. For the present invention, the term “IgE-related disease” includes or is used synonymously to the terms “IgE-dependent disease” or “IgE-mediated disease”.

According to a preferred embodiment the EMPD protein derived polypeptide is derived from native human IgE-BCR or is a mimic with one or more aa exchanges forming a mimotope of the respective native sequence.

Development of dedicated antibodies that specifically target human or mouse EMPD IgE allowed for clinical and preclinical validation of this targeting strategy in vitro and in vivo (Liour et al., 2016 Pediatr Allergy Immunol August; 27 (5): 446-51). The IgE-BCR crosslinking concept was first demonstrated in vivo by passive administration of anti-EMPD IgE antibodies in wild type mice (Feichtner et al., 2008 J. Immunol. 180:5499-5505) and a dedicated mouse model with a partially humanized IgE EMPD region (Brightbill et al., 2010 J. Clin. Invest. 120:2218-2229.). Chen at al (2010 Journal of Immunology 184, 1748-1756) showed that mAbs specific for the N-terminal or middle segment of CemX can bind to mIgE-expressing B-cells and induce their apoptosis and ADCC effectively. CemX refers to human membrane-bound e chain. This isoform contains an extra domain of 52 aa residues, located between the CH4 domain and the C-terminal membrane-anchor peptide and is referred to as CemX or M1′ peptide. This is specifically shown for antibodies to CemX N-terminal segment P1 (SVNPGLAGGSAQSQRAPDRVL, in which SVNP represents the C-terminal 4 aa residues of the CH4 domain of and m) the middle segment segment P2 (HSGQQQGLPRAAGGSVPHPR) whereas C-terminal P3 (GAGRADWPGPP) was not successful.

In addition, antibodies generated by active immunization against the human EMPD IgE region were able to mediate apoptosis and ADCC in vitro (Lin et al., Mol. Immunol., 52 (2012), pp. 190-199). Lin et al. disclose immunogens using HBcAg carrying inserts of CemX or its P1, P2, and P1-P2 parts as anti-EMPD vaccines.

The first clinical anti-human EMPD IgE monoclonal antibody Quilizumab showed selective IgE suppression in healthy volunteers combined with clinical benefit in allergic rhinitis and mild asthmatic patients in phase I and II studies, respectively (Scheerens et al., 2012 Asthma Therapy: Novel Approaches: p. A6791; Gauvreau et al., 2014 Sci. Transl. Med. 6, 243ra85.), but failed to improve the clinical outcome in patients with severe asthma bronchiale (Harris et al., 2016 Respir. Res. 17:29.). The epitope of Quilizumab also serves as potential immunogen and is located within a 11-residue segment SAQSQRAPDRV of CemX.

WO 2017/005851 A1 and Vig1 et al. (Journal of Immunological Methods 449 (2017) 28-36) disclose peptides as active anti-EMPD immunogens in combination with a suitable protein carrier located in the membrane proximal domain of EMPD. Sequence disclosed comprise AVSVNPGLAGGSAQSQRAPDRVLCHSGQQQGLPRAAGGSVP, QQQGLPRAAGG, QQLGLPRAAGG, QQQGLPRAAEG, QQLGLPRAAEG, QQQGLPRAAG, QQLGLPRAAG, QQQGLPRAAE, QQLGLPRAAE, HSGQQQGLPRAAGG, HSGQQLGLPRAAGG, HSGQQQGLPRAAEG, HSGQQLGLPRAAEG, QSQRAPDRVLCHSG, GSAQSQRAPDRVL, and WPGPPELDV.

Although in principle, the present invention is able to improve all suggested IgE-related disease vaccination polypeptides, selected epitopes were specifically assessed with respect to their suitability with the present platform. For example, SeqID43/44 (QQQGLPRAAGG) was shown to be superior to a KLH based vaccine.

In view of these advantageous properties of the conjugates of the present invention, it follows that the conjugates and vaccines according to the present invention are specifically useable for active anti-Human Epidermal Growth Factor Receptor 2 (anti-Her2) vaccination for the treatment and prevention of Her2 positive neoplastic diseases. Amplification or overexpression of Her2 occurs in approximately 15-30% of breast cancers and 10-30% of gastric/gastroesophageal cancers and serves as a prognostic and predictive biomarker. Her2 overexpression has also been seen in other cancers like ovary, endometrium and uterine serous endometrial carcinoma, uterine cervix, bladder, lung, colon, and head and neck. According to a preferred embodiment the Her2 protein derived polypeptide is derived from native human Her2 or is a mimic with one or more aa exchanges forming a mimotope of the respective native sequence.

Dakappagari et al. (JBC (2005) 280, 1, 54-63) disclose conformational epitope aa626-649 synthesized co-linearly with a promiscuous TH epitope derived from the measles virus fusion protein MVF (amino acids 288-302) and cyclisised by disulfide bridges. Peptides were formulated with muramyl dipeptide adjuvant, nor-MDP (N-acetylglucosamine-3yl-acetyl-L-alanyl-D-isoglutamine) and emulsified in Montanide ISA 720. Vaccines have been immunogenic and immunization with the vaccines reduced tumor burden in a tumor model.

EP 1 912 680 B1 and Allen et al. (J Immunol 2007; 179:472-482) disclose immunogens using three conformational peptide constructs (aa266-296 (LHCPALVTYNTDTFESMPNPEGRYTEGASCV), aa298-333 (ACPYNYLSTDVGSCTLVCPLHNQEVTAEDGTORCEK), and aa315-333 (CPLHNQEVTAEDGTORCEK) to mimic regions of the dimerization loop of the receptor. Vaccine candidates also contained MVF T-cell epitope (aa 288-302) KLLSLIKGVIVHRLEGVE and GPSL-linker. All peptides elicited high anti-Her2 immune responses and constructs using peptide aa266-296 have been equally effective as compared with Herceptin. The aa266-296 peptide of the Her2 sequence (accession number P04626):

MELAALCRWG LLLALLPPGA ASTQVCTGTD
MKLRLPASPE THLDMLRHLY QGCQVVQGNL
ELTYLPTNAS LSFLQDIQEV QGYVLIAHNQ
VRQVPLQRLR IVRGTQLFED NYALAVLDNG
DPLNNTTPVT GASPGGLREL QLRSLTEILK
GGVLIQRNPQ LCYQDTILWK DIFHKNNQLA
LTLIDTNRSR ACHPCSPMCK GSRCWGESSE
DCQSLTRTVC AGGCARCKGP LPTDCCHEQC
AAGCTGPKHS DCLACLHENH SGICELHCPA
LVTYNTDTFE SMPNPEGRYT FGASCVTACP
YNYLSTDVGS CTLVCPLHNQ EVTAEDGTQR
CEKCSKPCAR VCYGLGMEHL REVRAVTSAN
IQEFAGCKKI FGSLAFLPES FDGDPASNTA
PLQPEQLQVF ETLEEITGYL YISAWPDSLP
DLSVFQNLQV IRGRILHNGA YSLTLQGLGI
SWLGLRSLRE LGSGLALIHH NTHLCFVHTV
PWDQLFRNPH QALLHTANRP EDECVGEGLA
CHQLCARGHC WGPGPTQCVN CSQFLRGQEC
VEECRVLQGL PREYVNARHC LPCHPECQPQ
NGSVTCFGPE ADQCVACAHY KDPPFCVARC
PSGVKPDLSY MPIWKFPDEE GACQPCPINC
THSCVDLDDK GCPAEQRASP LTSIISAVVG
ILLVVVLGVV FGILIKRRQQ KIRKYTMRRL
LQETELVEPL TPSGAMPNQA QMRILKETEL
RKVKVLGSGA FGTVYKGIWI PDGENVKIPV
AIKVLRENTS PKANKEILDE AYVMAGVGSP
YVSRLLGICL TSTVQLVTQL MPYGCLLDHV
RENRGRLGSQ DLLNWCMQIA KGMSYLEDVR
LVHRDLAARN VLVKSPNHVK ITDEGLARLL
DIDETEYHAD GGKVPIKWMA LESILRRRFT
HQSDVWSYGV TVWELMTFGA KPYDGIPARE
IPDLLEKGER LPQPPICTID VYMIMVKCWM
IDSECRPRER ELVSEFSRMA RDPQREVVIQ
NEDLGPASPL DSTFYRSLLE DDDMGDLVDA
EEYLVPQQGF FCPDPAPGAG GMVHHRHRSS
STRSGGGDLT LGLEPSEEEA PRSPLAPSEG
AGSDVEDGDL GMGAAKGLQS LPTHDPSPLQ
RYSEDPTVPL PSETDGYVAP LTCSPQPEYV
NQPDVRPQPP SPREGPLPAA RPAGATLERP
KTLSPGKNGV VKDVFAFGGA VENPEYLTPQ
GGAAPQPHPP PAFSPAFDNL YYWDQDPPER
GAPPSTEKGT PTAENPEYLG LDVPV

as a vaccine had statistically reduced tumor onset in both transplantable tumor models and significant reduction in tumor development in two transgenic mouse tumor models.

Garret et al. (J Immunol 2007; 178:7120-7131) disclose Her2 peptides as immunogens aa563-598, aa585-598, aa597-626, and aa613-626 were synthesized colinearly with a promiscuous Th epitope derived from the measles virus fusion protein (aa 288-302) and applied in combination with Montanide ISA 720. Vaccines have been immunogenic and immunization with the vaccines carrying the aa597-626 epitope significantly reduced tumor burden in a tumor model.

Jasinska et al, (Int. J. Cancer: 107, 976-983 (2003)) disclose 7 peptides from the extracellular domain of Her2 as potential antigens for immunotherapy: cancer aa115-132 P1 AVLDNGDPLNNTTPVTGA, P2 aa149-162 LKGGVLIQRNPQLC, P3 aa274-295 YNTDTFESMPNPEGRYTEGAS, P4 aa378-398 PESFDGDPASNTAPLOPEQLQ, P5 aa489-504 PHOALLHTANRPEDE, P6 aa544-560 CRVLQGLPREYVNARHC, P7 aa610-623 YMPIWKFPDEEGAC which were coupled to tetanus toxoid and adjuvanted using Gerbu and induced humoral immune response with anti-tumor activity in an animal model. Similarly, Wagner et al. (2007 Breast Cancer Res Treat. 2007; 106:29-38) disclose peptide immunogens for immunization studies, applying the single peptides P4 (aa378-394: PESFDGDPASNTAPLQPC), P6 (aa545-560: RVLQGLPREYVNARHC) and P7 (aa610-623: YMPIWKFPDEEGAC) coupled to tetanus toxoid and adjuvanted with Gerbu. Vaccination was performed with or without IL12 addition and resulted in anti-tumor efficacy in preclinical models. Tobias et al. 2017 (BMC Cancer (2017) 17:118) disclose peptide immunogens for immunization studies, applying the single peptides P4 (aa378-394: PESFDGDPASNTAPLQP), P6 (aa545-560: RVLQGLPREYVNARHC) and P7 (aa610-623: YMPIWKFPDEEGAC) combined as hybrid peptides P467 (PESFDGDPASNTAPLQPRVLQGLPREYVNARHSLPYMPIWKFPDEEGAC) and P647 (RVLQGLPREYVNARHSPESEDGDPASNTAPLQPYMPIWKFPDEEGAC). The Cysteine (C) of P6 was replaced by ‘SLP’ or ‘S’, respectively. Both constructs were either coupled to virosomes or to diphtheria toxoid CRM197 (CRM) in combination with either Montanide or Aluminium hydroxide (Alum) as adjuvant and antibodies induced exhibited anti-tumor properties.

Riemer et al. (J Immunol 2004; 173:394-401) report the generation of peptide mimics of the epitope recognized by Trastuzumab on Her-2/neu by using a constrained 10 mer phage display library. Peptide mimotopes were coupled to the immunogenic carrier, tetanus toxoid (TT) and adjuvanted with Aluminium-hydroxide. Sequences comprise: C-QMWAPQWGPD-C, C-KLYWADGELT-C, C-VDYHYEGTIT-C, C-QMWAPQWGPD-C, C-KLYWADGELT-C, C-KLYWADGEFT-C, C-VDYHYEGTIT-C, CVDYHYEGAIT-C. Similarly, Singer et al. (ONCOIMMUNOLOGY 2016, VOL. 5, NO. 7, e1171446) disclose mimotopes to the trastuzumab epitope deduced from an AAV-mimotope library platform. Mimotope sequences tested comprise RLVPVGLERGTVDWV, TRWQKGLALGSGDMA, QVSHWVSGLAEGSFG, LSHTSGRVEGSVSLL, LDSTSLAGGPYEAIE, HVVMNWMREEFVEEF, SWASGMAVGSVSFEE. QVSHWVSGLAEGSFG and LSHTSGRVEGSVSLL proved to be immunogenic and effective in a tumor model.

Miyako et al. (ANTICANCER RESEARCH 31:3361-3368 (2011)) disclose peptides especially from the Her-2/neu extracellular domain (aa167-175) presented in the form of Her-2/neu-related multiple antigen peptides (MAP). Her-2/neu peptide contained epitopes for CD4+ and CD8+ T-cells, which contributes to the suppressive effect on Her-2/neu-expressing tumor cell growth. Sequences disclosed comprise:

Peptide Sequence (B; t-Butoxycarbonyl Residue (Boc)).

| | |
|---|---|
| N: 143-162 | (RSLTEILKGGVLIQRNPQLC-BBB)8-K4K2KB |
| N: 153-172 | (VLIQRNPQLCYQDTILWKDI-BBB)8-K4K2KB |
| N: 163-182 | (YQDTILWKDIFHKNNQLALT-BBB)8-K4K2KB |
| N: 173-192 | (FHKNNQLALTLIDTNRSRAC-BBB)8-K4K2KB |
| N: 183-202 | (LIDTNRSRACHPCSMPCKGS-BBB)8-K4K2KB |
| N: 193-212 | (HPCSMPCKGSRCWGESSEDC-BBB)8-K4K2KB |
| N: 203-222 | (RCWGESSEDCQSLTRTVCAG-BBB)8-K4K2KB |
| N: 213-232 | (QSLTRTVCAGGCARCKGPLP-BBB)8-K4K2KB |
| N: 223-242 | (GCARCKGPLPTDCCHEQCAA-BBB)8-K4K2KB |
| N: 233-252 | (TDCCHEQCAAGCTGPKHSDC-BBB)8-K4K2KB |
| N: 243-263 | (GCTGPKHSDCLACLHFNHSG-BBB)8-K4K2KB |
| N: 253-272 | (LACLHFNHSGICELHCPALV-BBB)8-K4K2KB |
| N: 263-282 | (ICELHCPALVTYNTDTFESM-BBB)8-K4K2KB |
| N: 273-292 | (TYNTDTFESMPNPEGRYTFG-BBB)8-K4K2KB |
| N: 283-302 | (PNPEGRYTFGASCVTACPYN-BBB)8-K4K2KB |
| N: 292-310 | (GASCVTACPYNYLSTDVGS-BBB)8-K4K2KB |
| N: 300-321 | (PYNYLSTDVGSCTLVCPLHNQE-BBB)8-K4K2KB |
| N: 312-330 | (TLVCPLHNQEVTAEDGTQR-BBB)8-K4K2KB |
| N: 322-341 | (VTAEDGTQRCEKCSKPCARV-BBB)8-K4K2KB |
| N: 332-351 | (EKCSKPCARVCYGLGMEHLR-BBB)8-K4K2KB |
| N: 343-361 | (YGLGMEHLREVRAVTSANI-BBB)8-K4K2KB |
| N: 352-370 | (EVRAVTSANIQEFAGCKKI-BBB)8-K4K2KB |

Humoral immune responses were induced, tumor growth in immunized mice was suppressed and tumor-infiltrating lymphocytes comprised more CD8+ T-cells, which secreted larger amounts of interleukin-2 after the peptide restimulation.

Henle et al. (J Immunol. 2013 Jan. 1; 190 (1): 479-488) disclose peptide epitopes derived from Her2 that generate cross-reactive T-cells. For HER-2/neu HLA-A2 binding peptide aa369-377 (KIFGSLAFL), it has been shown that cytotoxic T lymphocytes (CTLs) specific for this epitope can directly kill HER-2/neu overexpressing breast cancer cells. Other epitopes disclosed comprise HER-2/neu peptides p368-376, KKIFGSLAF; p372-380, GSLAFLPES; p364-373, FAGCKKIFGS; p373-382, SLAFLPESFD; p364-382, FAGCKKIFGSLAFLPESFD; and p362-384, QEFAGCKKIFGSLAFLPESEDGD. One of these sequences, p373-382 (SLAFLPESFD), bound HLA-A2 stronger than p369-377 and identified as potential epitope for vaccination.

Kaumaya et al. (ONCOIMMUNOLOGY 2020, VOL. 9, NO. 1, e1818437) disclose the combination of a Her2 targeting vaccine (aa266-296 and aa597-626 in combination with measles virus fusion peptide (MVF) amino acid 288-302 via a four amino acid residue (GPSL) emulsified in Montanide ISA 720VG) and a novel PD1 immune checkpoint targeting vaccine (PD-1 B-cell peptide epitope (aa92-110; GAISLAPKAQIKESLRAEL) in combination with virus fusion peptide (MVF) amino acid 288-302 via a four amino acid residue (GPSL) emulsified in Montanide ISA 720VG) for the combined treatment of Her2 positive disease. Thus, it is also a preferred embodiment to provide combination of anti-neoplastic disease vaccines, especially of a cancer target specific vaccine and an immune checkpoint targeting vaccine.

Although in principle, the present invention is able to improve all suggested Her2-related disease vaccination polypeptides, selected epitopes were specifically assessed with respect to their suitability with the present platform. For example, SeqID No47/48 (aa610-623: YMPIWKFPDEEGAC) was shown to be superior to a CRM based vaccine.

In view of these advantageous properties of the conjugates of the present invention, it follows that the conjugates and vaccines according to the present invention are specifically useable in individualized neoantigen specific therapy, preferably with NYESO-1, MAGE-A1, MAGE-A3, MAGE-C1, MAGE-C2, MAGE-C3, Survivin, gp100, tyrosinase, CT7, WT1, PSA, PSCA, PSMA, STEAP1, PAP, MUC1, 5 T4, KRAS, or Her2.

In view of these advantageous properties of the conjugates of the present invention, it follows that the conjugates and vaccines according to the present invention are specifically useable for active anti-immune checkpoint vaccination for controlling the cancer microenvironment, for the treatment and prevention of neoplastic diseases and for treatment and prevention of T-cell dysfunction in cancer/neoplastic disease (e.g. avoiding exhaustion of CD8 T-cells infiltrating cancer tissues) and chronic degenerative diseases including diseases with reduced T-cell activity like Parkinson's Disease.

It is well accepted in the field that PD patients suffer from different changes in their T-cell compartment as compared to healthy controls (e.g.: Bas et al., J Neuroimmunol 2001; 113:146-52 or Gruden et al., J Neuroimmunol 2011; 233:221-7). Such phenotypic changes of T-cells in PD are for example: reduced absolute lymphocyte counts, decreased absolute and relative counts of total T-cells, decreased absolute and relative counts of CD4+, and sometimes also CD8+ lymphocytes, increased Th1/Th2 and Th17/Treg ratios and increased expression of inflammatory cytokines. However, most of these changes are also found during healthy aging, making it difficult to discern the impact of a disease, such as PD, which presents with a very broad range of onset (˜30-90 years) and variable progression rate. Regarding absolute cell numbers, there appears to be consensus of a net reduction in CD3+CD4+ T-cells in PD. This CD4 reduction is supported by the altered CD4:CD8 ratio described.

Along these lines, for example, Bhatia et al. (J Neuroinflammation (2021) 18:250) show an overall decrease of total CD3+ T-cells in PD associated with disease severity (e.g. measured using H+Y stages). This suggests a progressing generalized T-cell dysfunction with ongoing disease, probably reflecting the combined effect of ongoing inflammation, medication and lifestyle change. Also, Lindestam Arlehamn et al. (2020) show that highest T-cell activity is detectable in PD patients at prodromal or early clinical stages (<10 years duration and H+Y stages 0-2).

Thus, it is a preferred embodiment of the present invention to provide a treatment for augmenting or preserving T-cell numbers, especially T-effector cell numbers, and T-cell function in a PD patient. This preferably includes a combination of checkpoint inhibitors or vaccines using anti-immune check point inhibitor epitopes to induce an anti-immune checkpoint inhibitor immune response in combination with target specific vaccines of the current invention to augment or preserve T-cell numbers, especially T-effector cell numbers and T-cell function in a PD patient.

Patients amenable to/suitable for the treatment are characterized by an overall reduction of CD3+ cells, especially of CD3+CD4+ cells typical for PD patients at all stages of the disease. The preferred stages of disease defining the suitable patient groups for this combination are H+Y stages 1-4, preferred H+Y1-3, most preferred H+Y 2-3, respectively.

Examples for such immune checkpoints targeting vaccines are vaccines providing epitopes to cytotoxic T lymphocyte-associated antigen 4 (CTLA-4, accession number P16410) and programmed cell death protein 1 (PD-1, accession number Q15116) or its ligand, programmed cell death ligand 1 (PD-L1 or PD1-L1, accession number Q9NZQ7), CD276 (accession number Q5ZPR3), VTCN1 (accession number Q7Z7D3), LAG3 (accession number P18627) or Tim3 (accession number Q8TDQ0); having the following amino acid sequences:

Human CTLA4: >sp|P16410|CTLA4 Uniprot
MACLGFQRHK AQLNLATRTW PCTLLEFLLF IPVFCKAMHV AQPAVVLASS RGIASFVCEY
ASPGKATEVR VTVLRQADSQ VTEVCAATYM MGNELTFLDD SICTGTSSGN QVNLTIQGLR
AMDTGLYICK VELMYPPPYY LGIGNGTQIY VIDPEPCPDS DELLWILAAV SSGLFFYSFL
LTAVSLSKML KKRSPLTTGV YVKMPPTEPE CEKQFQPYFI PIN
Human PD1: >sp|Q15116|PDCD1 Uniprot
MQIPQAPWPV VWAVLQLGWR PGWELDSPDR PWNPPTESPA LLVVTEGDNA TFTCSESNTS
ESFVLNWYRM SPSNQTDKLA AFPEDRSQPG QDCRERVTQL PNGRDFHMSV VRARRNDSGT
YLCGAISLAP KAQIKESLRA ELRVTERRAE VPTAHPSPSP RPAGQFQTLV VGVVGGLLGS
LVLLVWVLAV ICSRAARGTI GARRTGQPLK EDPSAVPVFS VDYGELDFQW REKTPEPPVP
CVPEQTEYAT IVFPSGMGTS SPARRGSADG PRSAQPLRPE DGHCSWPL
Human PD1-L1 >sp|Q9NZQ7|PD1L1 Uniprot
MRIFAVFIFM TYWHLLNAFT VTVPKDLYVV EYGSNMTIEC KFPVEKQLDL AALIVYWEME
DKNIIQFVHG EEDLKVQHSS YRQRARLLKD QLSIGNAALQ ITDVKLQDAG VYRCMISYGG
ADYKRITVKV NAPYNKINQR ILVVDPVTSE HELTCQAEGY PKAEVIWTSS DHQVLSGKTT
TTNSKREEKL FNVTSTLRIN TTTNEIFYCT FRRLDPEENH TAELVIPELP LAHPPNERTH
LVILGAILLC LGVALTFIFR LRKGRMMDVK KCGIQDTNSK KQSDTHLEET
Human B7-H3-CD276 >sp|Q5ZPR3|CD276 Uniprot
MLRRRGSPGM GVHVGAALGA LWFCLTGALE VQVPEDPVVA LVGTDATLCC SFSPEPGESL
AQLNLIWQLT DTKQLVHSFA EGQDQGSAYA NRTALEPDLL AQGNASLRLQ RVRVADEGSF
TCFVSIRDFG SAAVSLQVAA PYSKPSMTLE PNKDLRPGDT VTITCSSYQG YPEAEVFWQD
GQGVPLTGNV TTSQMANEQG LFDVHSILRV VLGANGTYSC LVRNPVLQQD AHSSVTITPQ
RSPTGAVEVQ VPEDPVVALV GTDATLRCSF SPEPGESLAQ LNLIWQLTDT KQLVHSFTEG
RDQGSAYANR TALFPDLLAQ GNASLRLQRV RVADEGSFTC FVSIRDFGSA AVSLQVAAPY
SKPSMTLEPN KDLRPGDTVT ITCSSYRGYP EAEVFWQDGQ GVPLTGNVTT SQMANEQGLE
DVHSVLRVVL GANGTYSCLV RNPVLQQDAH GSVTITGQPM TFPPEALWVT VGLSVCLIAL
LVALAFVCWR KIKQSCEEEN AGAEDQDGEG EGSKTALQPL KHSDSKEDDG QEIA
Human B7-H4-VTCN1 >sp|Q7Z7D3|VTCN1 Uniprot
MASLGQILFW SIISIIIILA GAIALIIGFG ISGRHSITVT TVASAGNIGE DGILSCTFEP
DIKLSDIVIQ WLKEGVLGLV HEFKEGKDEL SEQDEMERGR TAVFADQVIV GNASLRLKNV
QLTDAGTYKC YIITSKGKGN ANLEYKTGAF SMPEVNVDYN ASSETLRCEA PRWFPQPTVV
WASQVDQGAN FSEVSNTSFE LNSENVTMKV VSVLYNVTIN NTYSCMIEND IAKATGDIKV
TESEIKRRSH LQLLNSKASL CVSSFFAISW ALLPLSPYLM LK
Human LAG3: >sp|P18627|LAG3 Uniprot
MWEAQFLGLL FLQPLWVAPV KPLQPGAEV PVVWAQEGAP AQLPCSPTIP LQDLSLLRRA
GVTWQHQPDS GPPAAAPGHP LAPGPHPAAP SSWGPRPRRY TVLSVGPGGL RSGRLPLQPR
VQLDERGRQR GDFSLWLRPA RRADAGEYRA AVHLRDRALS CRLRLRLGQA SMTASPPGSL
RASDWVILNC SFSRPDRPAS VHWERNRGQG RVPVRESPHH HLAESFLFLP QVSPMDSGPW
GCILTYRDGF NVSIMYNLTV LGLEPPTPLT VYAGAGSRVG LPCRLPAGVG TRSFLTAKWT
PPGGGPDLLV TGDNGDFTLR LEDVSQAQAG TYTCHIHLQE QQLNATVTLA IITVTPKSFG
SPGSLGKLLC EVTPVSGQER FVWSSLDTPS QRSFSGPWLE AQEAQLLSQP WQCQLYQGER
LLGAAVYFTE LSSPGAQRSG RAPGALPAGH LLLFLILGVL SLLLLVTGAF GFHLWRRQWR
PRRESALEQG IHPPQAQSKI EELEQEPEPE PEPEPEPEPE PEPEQL
Human Tim3: >sp|Q8TDQ0|HAVR2
Uniprot
MFSHLPFDCV LLLLLLLLT RSSEVEYRAE VGQNAYLPCF YTPAAPGNLV PVCWGKGACP
VFECGNVVLR TDERDVNYW TSRYWLNGDF RKGDVSLTIE NVTLADSGIY CCRIQIPGIM
NDEKFNLKLV IKPAKVTPA PTRQRDETAA FPRMLTTRGH GPAETQTLGS LPDINLTQIS
TLANELRDSR LANDLRDSG ATIRIGIYIG AGICAGLALA LIFGALIFKW YSHSKEKIQN
LSLISLANLP PSGLANAVA EGIRSEENIY TIEENVYEVE EPNEYYCYVS SRQQPSQPLG
CRFAMP

Antibodies targeting CTLA-4 inhibit an immune response in several ways, including hindering autoreactive T-cell activation at a proximal step in the immune response, typically in lymph nodes. In contrast, the PD-1 pathway regulates T-cells at a later stage of the immune response, typically in peripheral tissues. Thus, two main directions of intervention are now clinically available for manipulating immune checkpoints by either targeting CTLA-4 or PD-1/PD-L1: Anti-CTLA-4 is involved in the lymphocyte proliferation process after antigen specific T-cell receptor activation while anti-PD-1/PD-L1 act predominantly in peripheral tissues during the effector step. However, CTLA-4 is also expressed on regulatory T lymphocytes and is thus involved in peripheral inhibition of T-cell proliferation.

Today, several immune checkpoint-blocking antibodies such as Ipilimumab (anti-CTLA-4 antibody), nivolumab and pembrolizumab (both anti-PD-1 antibodies), avelumab (anti-PD-L1 antibody) or atezolizumab and durvalumab (both anti-B7-H1/PD-L1 antibodies) can induce high anti-cancer immunity and low side effects.

According to a preferred embodiment the CTLA4 protein derived polypeptide is derived from native human CTLA4 or is a mimic with one or more aa exchanges forming a mimotope of the respective native sequence.

According to a preferred embodiment the PD1 protein derived polypeptide is derived from native human PD1 or is a mimic with one or more aa exchanges forming a mimotope of the respective native sequence. Protein sequences corresponding to the extracellular domains of murine PD1 (Q02242; Uniprot) and Human PD1 (Q15116; Uniprot).

According to a preferred embodiment the PD-L1 protein derived polypeptide is derived from native human PD-L1 or is a mimic with one or more aa exchanges forming a mimotope of the respective native sequence.

Guo et al. (Br J Cancer. 2021; 125:152-154) and Kaumaya et al. (Oncoimmunology. 2020; 9:1818437) disclose a PD1 derived peptide (aa92-110: GAISLAPKAQIKESLRAEL) which induces antibodies reducing tumor growth in a syngeneic BALB/c model with CT26 colon carcinoma cells. Furthermore, the combination of the disclosed PD1-epitope vaccine with a HER-2 peptide vaccine showed enhanced inhibition of tumor growth in colon carcinoma.

Tobias et al. (Front Immunol. 2020; 11:895.) disclose the peptides/mimotopes (=epitopes of anti-human PD1 mAb Nivolumab and anti-murine mAb clone 29F.1A12) from murine and human PD-1. The peptides comprise the human PD1-derived sequences PGWFLDSPDRPWNPP, FLDSPDRPWNPPTFS, and SPDRPWNPPTFSPA, corresponding to the positions aa21-35, aa24-38, and aa27-41 on human PD1, designated as JT-N1, JT-N2, and JT-N3, respectively. Furthermore, the mimotopes to murine PD1 comprise ISLHPKAKIEESPGA (JT-mPD1) corresponding to amino acid residues aa126-140 of mPD1. The antitumor effect by mimotope JT-mPD1 was shown to be associated with a significant reduction of proliferation and increased apoptotic rates in the tumors in the employed Her-2/neu-expressing syngeneic tumor mouse model. Further, the antitumor effect of a Her-2/neu vaccine was shown to be potentiated when combined with JT-mPD1.

Chen et al. (Cancers 2019, 11, 1909) disclose PDL1-Vax, a fusion protein of human PD-L1 (aa19-220 of human PD-L1) linked to a T helper epitope sequence and a human IgG1 Fc sequence as novel PD-L1 targeting vaccine. Jorgensen et al. (Front Immunol. 2020; 11:595035.) disclose a 19-amino-acid peptide (FMTYWHLLNAFTVTVPKDL) derived from the signal peptide of human PD-L1 as novel PD-L1 targeting vaccine. Tian et al. (Cancer Letters 476 (2020) 170-182) disclose truncated murine PDL1 extracellular domain (aa19-239) fused to the NitraTh epitope, hPDL1-NitraTh was also constructed by fusing the truncated human PDL1 extracellular domain (aa19-238) to the NitraTh epitope as novel PD-L1 targeting vaccine.

These anti-immune checkpoint vaccines may be highly effective when used alone or in combination with pre-existing peptide vaccines. Therefore, it is a preferred embodiment to provide a combination of anti-immune checkpoint vaccines with pre-existing peptide vaccines to treat neoplastic or degenerative disease like Parkinson's disease.

Although in principle, the present invention is able to improve all suggested PD1 and PD-L1-related vaccination polypeptides, selected epitopes were specifically assessed with respect to their suitability with the present platform. For example, SeqID No 49/50 (GAISLAPKAQIKESLRAEL) were shown to be superior to a KLH based vaccine.

In view of these advantageous properties of the conjugates of the present invention, it follows that the conjugates and vaccines according to the present invention are specifically useable for active anti-Aβ immunotherapy for use in the prevention, treatment and diagnosis of diseases associated with β-amyloid formation and/or aggregation. The most prominent form of β-Amyloidoses is Alzheimer's disease (AD). Other examples include familial and sporadic AD, familial and sporadic Aβ cerebral amyloid angiopathies, Hereditary cerebral hemorrhage with amyloidosis (HCHWA), Dementia with Lewy bodies and Dementia in Down syndrome, Retinal ganglion cell degeneration in glaucoma, Inclusion body myositis/myopathy,

The Aβ peptide exists in several forms, including full-length Aβ1-42 and Aβ1-40 various modified forms of Aβ including truncated, N-terminally truncated or C terminally truncated, nitrated, acetylated and the N-truncated species, pyroglutamate Aβ3-40/42 (i.e. AβpE3-40 and AβpE3-42) and Aβ4-42, which appear to play a major role in neurodegeneration.

According to a preferred embodiment, the Aβ peptide derived polypeptide is selected from native human Aβ1-40 and/or Aβ1-42 with the following amino acid sequence:
Aβ 1-40: DAEFRHDSGY EVHHQKLVFF AEDVGSNKGA IIGLMVGGVV
Aβ 1-42: DAEFRHDSGY EVHHQKLVFF AEDVGSNKGA IIGLMVGGVV IA
or a polypeptide comprising or consisting of amino acid residues derived from human Aβ1-40 and/or Aβ1-42 including truncated, especially N-terminally truncated, C terminally truncated, posttranslationally modified, nitrated, glycosylated, acetylated, ubiquitinated peptides amino acids or peptides carrying a pyroglutamate residue at aa3 or aa11 including Aβ aa1-6, aa1-7, aa1-8, aa1-9, aa1-10, aa1-11, aa1-12, aa1-13, aa1-14, aa1-15, aa1-21, aa2-7, aa2-8, aa2-9, aa2-10, aa3-8, aa3-9, aa3-10, aa pE3-8, aa pE3-9, aa pE3-10, aa11-16, aa11-17, aa11-18, aa11-19, aa12-19, aa13-19, aa14-19, aa14-20, aa14-21, aa14-22, aa14-23, aa30-40, aa31-40, aa32-40, aa33-40, aa34-40, aa30-42, aa37-42.

According to a preferred embodiment, the Aβ 1-40 or Aβ1-42 derived polypeptide is selected from mimics of the above mentioned Aβ derived polypeptides including mimotopes and peptides containing amino acid substitutions mimicking pyroglutamate amino acids. Schenk et al. (Nature. 1999 Jul. 8; 400 (6740):173-7.) disclose Aβ1-42 as immunogen for anti-Aβ immunotherapy, Pride et al. (Neurodegenerative Dis 2008; 5:194-196) disclose peptide epitopes of Aβ1-6 coupled to CRM197 adjuvanted with QS21 and Wiesner et al. (J Neurosci. 2011 Jun. 22; 31 (25): 9323-31) disclose Aβ1-6 peptide coupled to a Qβ virus-like particle as efficient immunotherapeutic.

Wang et al. (Alzheimer's & Dementia: Translational Research & Clinical Interventions 3 (2017) 262-272) and US 2018/0244739 A1 disclose Aβ 1-42 peptide immunogens and especially UB311, comprising two Aβ immunogens, the cationic Aβ1-14-εK-KKK-MvF5 Th [ISITEIKGVIVHRIETILF] and Aβ1-14-εK-HBsAg3 Th [KKKIITITRIITIITID] peptides, in equimolar ratio; they were mixed with polyanionic CpG oligodeoxynucleotide (ODN) to form stable immunostimulatory complexes of micron-size particulates, to which aluminum mineral salt (Adju-Phos), was added to the final formulation.

Illouz et al. (Vaccine Volume 39, Issue 34, 9 Aug. 2021, Pages 4817-4829) disclose Aβ1-11 fused to HBsAg as vaccine in aged mice.

Davtyan H et al. (J Neurosci. 2013 Mar. 13; 33 (11): 4923-4934) and Petrushina et al. (Molecular Therapy Vol. 25 No 1 153-164) disclose vaccines comprising two foreign Th-cell epitopes from Tetanus Toxin, P30, and P2 and three copies of the B-cell epitopes of Aβ1-12 adjuvanted with QuilA. Similarly, Davtyan H et al. (Alzheimer's & Dementia 10 (2014) 271-283) disclose DNA based vaccines building on protein coding regions consisting either of the immunoglobulin (Ig) k-chain signal sequence, 3 copies of the Aβ1-11 B-cell epitope, 1 synthetic peptide (PADRE), and a string of 8 non-self, promiscuous Th epitopes from tetanus toxin (TT) (P2, P21, P23, P30, and P32), hepatitis B virus (HBsAg, HBVnc), and influenza (MT) or also comprising 3 additional Th epitopes from TT (P7 (NYSLDKIIVDYNLQSKITLP); P17 (LINSTKIYSYFPSVISKVNQ); and P28 (LEYIPEITLPVIAALSIAES)).

Petrushina et al. (Journal of Neuroinflammation 2008, 5:42) disclose Aβ1-28 with an N-terminal linker (n-CAGA) coupled to bro-moacetylated S. cerevisiae mannan as potential vaccine although with severe side effects.

US 2011/0002949 A1 discloses multivalent vaccine construct (Aβ3-10/Aβ21-28) (MVC) and the monovalent vaccine construct Aβ1-8 (MoVC1-8) conjugated to a carrier (KLH) and administered with a saponin-based adjuvant, ISCOMATRIX.

Muhs et al. (Proc Natl Acad Sci USA. 2007 Jun. 5; 104(23):9810-5), Hickman et al. (J Biol Chem. 2011 Apr. 22; 286(16):13966-76) and Belichenko et al. (PLOS One. 2016; 11(3):e0152471) disclose Aβ1-15 as array of Aβ1-15 sequences, sandwiched between palmitoylated lysines at either end, which are anchored into the surface of liposomes for the peptides to adopt an aggregated β-sheet structure, forming a conformational epitope.

Ding et al. (Neuroscience Letters, Volume 634, 10 Nov. 2016, Pages 1-6) disclose peptides by coupling Aβ3-10 to the immunogenic carrier protein keyhole limpet hemocyanin (KLH) or by joining 5 Aβ3-10 epitopes linearly in tandem.

Bakrania et al. (Mol Psychiatry (2021). https://doi.org/10.1038/s41380-021-01385-7) disclose cyclised Aβ1-14 (thioacetal bridged Aβ peptide 1-14-KLH conjugate; DAC*FRHDSGYEC*HH[Cys]-amide emulsified in complete Freund's adjuvant (CFA), followed by booster doses of protein emulsified in incomplete Freund's adjuvant (IFA) as suitable immunogens.

Lacosta et al. (Alzheimers Res Ther. 2018 Jan. 29; 10 (1):12.) disclose Aβ peptide immunogens comprising multiple repeats of a short C-terminal fragment of Aβ1-40. To generate an immune response, the repeats are conjugated to the keyhole limpet cyanine (KHL) carrier protein and formulated with the adjuvant alum hydroxide

Axelsen et al. (Vaccine Volume 29, Issue 17, 12 Apr. 2011, Pages 3260-3269) discloses Aβ37-42 coupled to Keyhole limpet hemocyanin.

WO 2004/062556 A2, WO 2006/005707 A2, WO 2009/149486 A2 and WO 2009/149485 A2 disclose mimotopes of epitopes of AB. It is shown that these mimotopes are able to induce the in vivo formation of antibodies directed to non-truncated Aβ1-40/42, and N-terminally truncated forms AβpE3-40/42, Aβ3-40/42, Aβ11-40/42, AβpE11-40/42 and Aβ14-40/42, respectively.

According to a preferred embodiment, the Aβ peptide derived polypeptide is selected from:

| native Aβ peptides | |
|---|---|
| Aβ - position | Sequence |
| 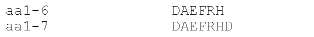 | |
| aa1-8 | DAEFRHDS |
| aa1-9 | DAEFRHDSG |
| aa1-10 | DAEFRHDSGY |
| aa1-11 | DAEFRHDSGYE |
| 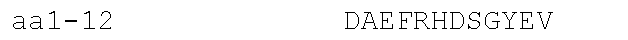 | |
| aa1-13 | DAEFRHDSGYEVH |
| 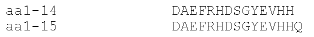 | |
| aa1-21 | DAEFRHDSGYEVHHQKLVFFA |
| 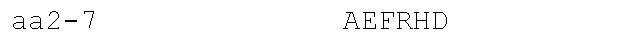 | |
| aa2-8 | AEFRHDS |
| aa2-9 | AEFRHDSG |
| aa2-10 | AEFRHDSGY |
| aa3-8 | EFRHDS |
| aa3-9 | EFRHDSG |
| aa3-10 | EFRHDSGY |
| aa pE3-8 | p (E) FRHDS |
| aa pE3-9 | p (E) FRHDSG |
| aa pE3-10 | p (E) FRHDSGY |
| aa11-16 | EVHHQK |
| aa11-17 | EVHHQKL |
| aa11-18 | EVHHQKLV |
| aa11-19 | EVHHQKLVF |
| aa12-19 | VHHQKLVF |
| aa13-19 | HHQKLVF |
| aa14- 19 | HQKLVF |
| aa14- 20 | HQKLVFF |
| aa14- 21 | HQKLVFFA |
| aa14- 22 | HQKLVFFAE |
| aa14- 23 | HQKLVFFAED |

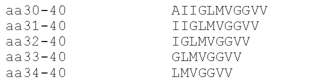

mimotope Aβ peptides

| Aβ-position | Sequence |
|---|---|
| aa1-6 | DKELRI |
| aa1-7 | DKELRID |
| aa1-8 | DKELRIDS |
| aa1-9 | DKELRIDSG |
| aa1-10 | DKELRIDSGY |
| aa1-6 | SWEFRT |
| aa1-7 | SWEFRTD |
| aa1-8 | SWEFRTDS |
| aa1-9 | SWEFRTDSG |
| aa1-10 | SWEFRTDSGY |
| aa1-7 | TLHEFRH |
| aa1-7 | TLHEFKH |
| aa1-7 | THTDFRH |
| aa1-7 | THTDFKH |
| aa2-7 | AEFKHD |
| aa2-7 | AEFKHG |
| aa2-7 | SEFRHD |
| aa2-7 | SEFRHG |
| aa2-7 | SEFKHD |
| aa2-7 | SEFKHG |
| aa2-7 | ILFRHG |
| aa2-7 | ILFRHD |
| aa2-7 | ILFKHG |
| aa2-7 | ILFKHD |
| aa pE3-8 | IRWDTP |
| aa pE3-8 | IRYDAPL |
| aa pE3-8 | IRYDMAG |

These anti-Aβ vaccines are highly effective when used alone or in combination with pre-existing peptide vaccines directed against other pathologic molecules involved in β-amyloidoses, tauopathies or synucleopathies, especially with mixed pathology (i.e. the presence of Aβ-pathology with Tau-pathology and/or aSyn pathology). Therefore, it is a preferred embodiment to provide a combination of anti-Aβ vaccines with anti-Tau and/or anti-aSyn peptide vaccines to treat degenerative disease like Alzheimer's disease, dementia in Down syndrome, dementia with Lewy bodies, Parkinson's disease dementia, Parkinson's disease or Tauopathies.

Although in principle, the present invention is able to improve all suggested Aβ and Aβ-related vaccination polypeptides, selected epitopes were specifically assessed with respect to their suitability with the present platform. For example, SeqID32/33 (AβpE3-8; pEFRHDS) were shown to be superior to a KLH based vaccine and SeqID10 (Aβ1-6; DAEFRH) proved to be immunogenic in combination with different CLECs.

In view of these advantageous properties of the conjugates of the present invention, it follows that the conjugates and vaccines according to the present invention are specifically useable for active anti-IL31 vaccination for the treatment and prevention of IL31 related diseases and autoimmune inflammatory diseases.

IL31-related diseases include pruritus-causing allergic diseases, pruritus-causing inflammatory diseases and pruritus-causing autoimmune diseases in mammals, including humans, dogs, cats and horses. These diseases include atopic dermatitis, prurigo nodularis, psoriasis, cutaneous T-Cell lymphoma (CTCL), and other pruritic disorders, such as uremic pruritus, cholestatic pruritus, bullous pemphigoid and chronic urticaria, allergic contact dermatitis (ACD), dermatomyositis, chronic pruritus of unknown origin (CPUO), primary localized cutaneous amyloidosis (PLCA), mastocytosis, chronic spontaneous urticaria, bullous pemphigoid, dermatitis herpetiformis and other dermatologic conditions including lichen planus, cutaneous amyloidosis, statis dermatitis, scleroderma, itch associated with wound healing and non-pruritic diseases such as allergic asthma, allergic rhinitis, inflammatory bowel disease (IBD), osteoporosis, follicular Lymphoma, Hodgkin lymphoma and chronic myeloid leukemia.

According to a preferred embodiment single IL31 epitopes may be used to trigger an immune response against different domains of IL31. In another preferred embodiment a combination of IL31 epitopes may be used to trigger an immune response against different domains of IL31, in particular involving helix C or A, and further involving helix D, thereby preventing IL31 binding to both of the IL31 receptors, interleukin 31 receptor alpha (IL-31RA) and oncostatin M receptor (OSMR).

The anti-IL31 vaccines may be highly effective when used alone or in combination with peptide vaccines directed against other pathologic molecules involved in pruritus-causing allergic diseases, pruritus-causing inflammatory diseases and pruritus-causing autoimmune diseases. Therefore, it is a preferred embodiment to provide a combination of anti-IL31 vaccines with anti-IL4 and/or anti-IL13 peptide vaccines to treat pruritus-causing allergic diseases, pruritus-causing inflammatory diseases and pruritus-causing autoimmune diseases.

According to a preferred embodiment, the IL31 protein derived polypeptide is a fragment of the IL-31 protein, and/or is preferably selected from native human IL31 (Genbank: AAS86448.1; MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIE HLDKLIFQDAPETNISVPTDTHECKRFILTISQQFSECMDLALKSLTSGAQQATT); native canine IL31 (Genbank: BAH97742.1; MLSHTGPSRFALFLLCSMETLLSSHMAPTHQLPPSDVRKIILELQPLSRGLLEDYQKKETGVPESNRTLLLCLTSDSQPPRINSSAILPYFRAIRPLSDKNIIDKIIEQLDKL KFQHEPETEISVPADTFECKSFILTILQQFSACLESVEKSLNSGPQ); native feline IL31 (UNIPROT: A0A2I2UKP7 MLSHAGPARFALFLLCCMETLLPSHMAPAHRLOPSDVRKIILELRPMSKGLLODYVSKEIGLPESNHSSLPCLSSDSQLPHINGSAILPYFRAIRPLSDKNTIDKIIEQLDKLKFOREPEAKVS MPADNFERKNFILAVLQQFSACLEHVLQSLNSGPQ);
or native equine IL31 (UNIPROT F7AHG9 MVSHIGSTRFALFLLCCLGTLMFSHTGPIYQLQPKEIQAIIVELQNLSKKLLDDYVSALETSILSCFFKTDLPSCFTSDSOAPGNINSSAILPYFKAISPSLNNDKSLYIIEQLDKLNFQNAP ETEVSMPTDNFERKRFILTILRWFSNCLEHRAQ) or any peptide sequence which has at least 70, 75, 80, 85, 90 or 95% sequence identity to any of the foregoing, or which differs from the naturally occurring sequence by a number of point mutations of surface exposed amino acids, wherein the number of point mutations is 1, 2, or 3.

According to a preferred embodiment, the IL31 protein derived polypeptide is selected from mimics of the above-mentioned IL31 derived polypeptides including mimotopes and peptides containing amino acid substitutions.

Further preferred target sequences include (presented as linear or constrained peptides e.g. cyclisized or peptides joint by a suitable aa linker, e.g.: ggsgg or similar):
for human IL31: peptides derived for sequences aa98-145, aa87-150, aa105-113, aa85-115, aa84-114, aa86-117, aa87-116; or fragments thereof and peptides SDDVQKIVEELQSLSKMLLKDVEEEKGVLVSQNYTL; DVQKIVEELQSLSKMLLKDV, EELQSLSK and DVOK, LDNKSVIDEIIEHLDKLIFQDA; and DEIIEH, TDTHECKRFILTISQQFSECMDLALKS, TDTHESKRE, TDTHERKRF HESKRF, HERKRF, HECKRF; SDDVOKIVEELQ, VOKIVEELQSLS, IVEELQSLSKML, ELQSLSKMLLKD, SLSKMLLKDVEE, KMLLKDVEEEKG, LKDVEEEKGVLV, VEEEKGVLVSQN, EKGVLVSQNYTL, LDNKSVIDEIIE, KSVIDEIIEHLD, IDEIIEHLDKLI, IIEHLDKLIFQD, HLDKLIFQDAPE, KLIFQDAPETNI, FQDAPETNISVP, APETNISVPTDT, TNISVPTDTHEC, SVPTDTHESKRF, TDTHECKRFILT, TDTHESKRFILT, TDTHERKRFILT, HECKRFILTISQ, HESKRFILTISQ, HERKRFILTISQ, KRFILTISQQFS, ILTISQQFSECM, ILTISQOFSESM, ILTISQQFSERM, ISQQFSECMDLA, ISQQFSESMDLA, ISQQFSERMDLA, OFSECMDLALKS, QFSESMDLALKS, QFSERMDLALKS, SKMLLKDVEEEKG, EELQSLSK, KGVLVS, SPAIRAYLKTIRQLDNKSVIDEIIEHLDKLI, DEIIEHLDK, SVIDEIIEHLDKLI, SPAIRAYLKTIRQLDNKSVI, TDTHECKRF, HECKRFILT, HERKRFILT, HESKRFILT, SVPTDTHECKRF, SVPTDTHESKRF, and SVPTDTHERKRF for canine IL31: peptides consisting of aa97-144, aa97-133, aa97-122, aa97-114, aa90-110, aa90-144, aa86-144, aa97-149, aa90-149, aa86-149, aa 124-135 or fragments thereof and peptides: SDVRKIILELQPLSRGLLEDYQKKETGV, DVRKIILELQPLSRGLLEDY ELQPLSR LSDKNIIDKIIEQLDKLKFQHE, LSDKNIIDKIIEQLDKLKFQ, KLKFQHE, LSDKNI, LDKL, LSDKN, ADTFECKSFILTILQQFSACLESVEKS and ADNFERKNF for feline IL31: aa124-135 of a feline IL-31 sequence and peptides SDVRKIILELRPMSKGLLODYVSKEIGL and DVRKIILELRPMSKGLLQDY, LSDKNTIDKIIEQLDKLKFQRE, ADNFERKNFILAVLOOFSACLEHVLOS and ADNFERKNF
for equine IL31: aa118-129 of an equine IL-31 sequence and peptides: LOPKEIQAIIVELQNLSKKLLDDY, EIQAIIVELQNLSKKLLDDY, SLNNDKSLYIIEQLDKLNFQ and TDNFERKRFILTILRWFSNCLEHRAQ for mimotopes:
canine IL-31 mimotopes comprises the amino acid sequence SVPADTFECKSF, SVPADTFERKSF, NSSAILPYFRAIRPLSDKNIIDKIIEQLDKLKF, APTHOLPPSDVRKIILELQPLSRG, TGVPES or variants thereof.
feline IL-31 mimotopes comprises the amino acid sequences SMPADNFERKNF, NGSAILPYFRAIRPLSDKNTIDKIIEQLDKLKF, APAHRLOPSDIRKIILELRPMSKG, IGLPES or variants thereof. equine IL-31 mimotopes comprise the amino acid sequences SMPTDNFERKRF, NSSAILPYFKAISPSLNNDKSLYIIEQLDKLNF, GPIYQLQPKEIQAIIVELONLSKK, KGVQKF or variants thereof.
human IL-31 mimotopes comprise the amino acid sequences SVPTDTHECKRF, SVPTDTHERKRF, HSPAIRAYLKTIRQLDNKSVIDEIIEHLDKLIF, LPVRLLRPSDDVQKIVEELQSLSKM, KGVLVS or variants thereof that retain anti-IL-31 binding.

According to a preferred embodiment, the IL31 epitope can be a conformational epitope comprising at least two amino acids or amino acid sequences, which are spatially distinct from each other, but in close proximity such as to form a respective paratope. The paratope is typically bound by an anti-IL31 antibody e.g., a polyclonal anti-IL31 antibody obtained upon vaccinating a mammal with the vaccine and specifically recognizing the naturally occurring IL31.

IL31 is a protein with 4 helix bundle structure as found in the gp 30/IL-6 cytokine family. The receptor for IL-31 is a heterodimer of the interleukin 31 receptor alpha (IL-31 RA, also referred to as GPL or gp130-like receptor) and oncostatin M receptor (OSMR). Both structures of the heterodimer are referred to as IL-31 receptor or IL-31 co-receptor. The putative interaction sites between human IL-31 and its receptors have been described by Saux et al. (J Biol Chem 2010, 285, 3470-34). Targeting of IL31 may be achieved by antibodies targeting IL-31 and/or its receptor. Development of dedicated monoclonal antibodies that specifically target IL31 allowed for clinical and preclinical validation of this targeting strategy in vitro and in vivo (Front Med (Lausanne. 2021 Feb. 12; 8:638325)).

BMS-981164 is an anti-IL-31 monoclonal antibody targeting circulating IL-31 being developed by Bristol-Myers Squibb. A two-part, phase I, single-dose, dose-escalation study was conducted between 2012 and 2015 to explore the safety and pharmacokinetic profile of BMS-981164 (NCT01614756). The study design was randomized, double-blind, placebo-controlled, and the drug was administered as both SC and IV formulations (0.01 to 3 mg/kg) to healthy volunteers (part 1) and adults with atopic dermatitis (part 2). Adult subjects in part 2 were required to have at least moderate atopic dermatitis (assessed by Physician Global Assessment rating of _3 on a scale of 0 to 5) and pruritus severity of at least 7 of 10 on a visual analog scale. To date, no results from this study have been released. As of 2016, BMS-981164 was no longer listed in the development pipeline of Bristol-Myers Squibb, and no new trials have been announced.

U.S. Pat. No. 8,790,651 B2 describes monoclonal antibodies binding to IL-31 for treatment of immunological disorders, such as atopic dermatitis. A monoclonal antibody against canine IL-31 (Lokivetmab, Zoetis) is available on the market for the treatment of canine atopic dermatitis. Lokivetmab is putatively interfering with the binding of IL-31 to the co-receptor GPL.

EP 4 019 546 A1 discloses mono- and multi-specific antibodies where the antibody variable domain blocks the binding of IL-31 to the interleukin 31 receptor alpha (IL-31RA)/oncostatin M receptor (OSMR) complex (IL-31RA/OS-MR complex.

Bachmann et al. disclose a vaccine utilizing complete canine IL-31 coupled to virus like particles for immunization of dogs for the treatment of atopic dermatitis. (Bachmann, M. F.; Zeltins, A.; Kalnins, G.; Balke, I.; Fischer, N.; Rostaher, A.; Tars, K.; Favrot, C. Vaccination against IL-31 for the Treatment of Atopic Dermatitis in Dogs. J. Allergy Clin. Immunol. 2018, 142, 279-281. el). Similarly, U.S. Pat. No. 11,324,836 B2, U.S. Pat. No. 11,207,390 B2 and U.S. Pat. No. 10,556,003 and Fettelschloss et al (doi: 10.1111/eve.13408) disclose VLP based immunogens for targeting IL31 and IL31 related diseases from different species including human, canine, equine or porcine IL31. These VLP based immunogens are characterised by anti IL31 immunogens with full length, native as well as full length modified IL31-derived sequences, respectively.

US2021/0079054A1 discloses peptide-based immunogens building on the UbiTh platform technology targeting IL31 for the treatment and/or prevention of a pruritic condition or an allergic condition such as atopic dermatitis. Along these lines, B-cell epitope based immunogens derived from canine IL31 (Genbank: BAH97742.1; MLSHTGPSRFALFLLCSMETLLSSHMAPTHOLPPSDVRKIILELQPLSRGLLEDYQKKETGVPESNRTLLLCLTSDSQPPRINSSAILPYFRAIRPLSDKNIIDKIIEQLDKL KFQHEPETEISVPADTFECKSFILTILQQFSACLESVFKSLNSGPQ) and human IL31 (Genbank: AAS86448.1; MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIE HLDKLIFQDAPETNISVPTDTHECKRFILTISQOFSECMDLALKSLTSGAQQATT are presented including:
for canine IL31: peptides consisting of aa97-144, aa97-133, aa97-122, aa97-114, aa90-110, aa90-144, aa86-144, aa97-149, aa90-149, aa86-149; for human IL31: peptides derived for sequences aa98-145, aa87-150, aa105-113, aa85-115, aa84-114, aa86-117, aa87-116 with modifications if suitable, e.g.: Serine and Cysteine replacement. B-cell epitopes are linear or constrained and fused to promiscuous T-helper epitopes and formulated in the presence of adjuvants (e.g.: different CpG molecules, Alhydrogel, AdjuPhos, Montanides like ISA50V2, ISA51, ISA720).

US2019/0282704 A1 discloses vaccine compositions for immunizing and/or protecting a mammal against an IL-31 mediated disorder, wherein the composition includes the combination of a carrier polypeptide (e.g. CRM197) and at least one mimotope to an IL31 derived epitope selected from a feline IL-31 mimotope, a canine IL-31 mimotope, a horse IL-31 mimotope, or a human IL-31 mimotope; and an adjuvant. The mimotopes can be linear or constrained (e.g.: cyclisised).

The canine IL-31 mimotopes comprises the amino acid sequence SVPADTFECKSF, SVPADTFERKSF, NSSAILPYFRAIRPLSDKNIIDKIIEQLDKLKF, APTHOLPPSDVRKIILELQPLSRG, TGVPES or variants thereof.

The feline IL-31 mimotopes comprises the amino acid sequences SMPADNFERKNF, NGSAILPYFRAIRPLSDKNTIDKIIEQLDKLKF, APAHRLOPSDIRKIILELRPMSKG, IGLPES or variants thereof.

The equine IL-31 mimotopes comprise the amino acid sequences SMPTDNFERKRF, NS SAILPYFKAISPSLNNDKSLYIIEQLDKLNF, GPIYQLQPKEIQAIIVELONLS KK, KGVQKF or variants thereof.

The human IL-31 mimotopes comprise the amino acid sequences SVPTDTHECKRF, SVPTDTHERKRF, HSPAIRAYLKTIRQLDNKSVIDEIIEHLDKLIF, LPVRLLRPSDDVQKIVEELQSLSKM, KGVLVS or variants thereof that retain anti-IL-31 binding.

In addition, a region between about amino acid residues 124 and 135 of a feline IL-31 sequence represented by (UNIPROT: A0A2I2UKP7); and a region between about amino acid residues 124 and 135 of a canine IL-31 sequence represented by (Genbank: BAH97742.1); and a region between about amino acid residues 118 and 129 of an equine IL-31 sequence represented by (UNIPROT F7AHG9) are disclosed as suitable epitopes.

WO 2019/086694 A1 discloses peptide-based immunogens targeting IL31 achieved by an IL31 antigen comprising an unpacked IL31 helix peptide, or an epitope contained therein from canine, human, feline, equine, porcine, bovine or camelid IL31. The antigen is coupled to a conventional carrier molecule (e.g.: KLH) and adjuvanted with Imject Alum or can be coupled to anti-CD32 scFv constructs potentially containing the TLR9 agonist CpG or the TLR7/8 agonist Imidazoquinoline. Specifically, the IL31 peptide comprises or consists of the amino acid sequence identified as any one of

Helix A:
human:
SDDVQKIVEELQSLSKMLLKDVEEEKGVLVSQNYTL;
and
DVQKIVEEL-QSLSKMLLKDV, EELQSLSK
and
DVQK
canine:
SDVRKIILELQPLSRGLLEDYQKKETGV,
and
DVRKIILELQPLSRGLLEDY
and
ELQPLSR
feline:
SDVRKIILELRPMSKGLLQDYVSKEIGL
and
DVRKIILELRPMSKGLLQDY
equine:
LQPKEIQAIIVELQNLSKKLLDDY
and
EIQAIIVELQNLSKKLLDDY
Helix C
human:
LDNKSVIDEIIEHLDKLIFQDA;
and
DEIIEH
canine:
LSDKNIIDKIIEQLDKLKFQHE,
LSDKNIIDKIIEQLDKLKFQ,
KLKFQHE, LSDKNI, LDKL, LSDKN,
feline:
LSDKNTIDKIIEQLDKLKFQRE
equine:
SLNNDKSLYIIEQLDKLNFQ
and/or
Helix D:
human:
TDTHECKRFILTISQQFSECMDLALKS, TDTHESKRF
and
HESKRF
canine:
ADTFECKSFILTILQQFSACLESVEKS
and
ADNFERKNF
feline:
ADNFERKNFILAVLQQFSACLEHVLQS
and
ADNFERKNF
equine:
TDNFERKRFILTILRWESNCLEHRAQ

either alone or in combination, also fused using linker sequences as disclosed.

WO 2022/131820 A1 discloses immunomodulatory or anti-inflammatory IL31 derived peptides as an active ingredient for preventing or treating atopic dermatitis as pharmaceutical or cosmetic. It also discloses conjugates in which a IL31 peptide or a fragment thereof is conjugated with a biocompatible polymer, eg.: pullulan, chondroitin sulfate, hyaluronic acid (HA), glycol chitosan, starch, chitosan, dextran, pectin, curdlan, poly-L-lysine, polyaspartic acid (PAA), polylactic acid (PLA), polyglycol Ride (polyglycolide, PGA), polycaprolactone (poly (E-caprolactone), PCL), poly (caprolactone-lactide) random copolymer (PCLA), poly (caprolactone-glycolide) random copolymer (PCGA), poly (lactide-glycolicolide) random copolymer (PLGA), polyethylene glycol (PEG), pluronic F-68 and pluronic F-127 (pluronic F-127) or a fatty acid, e.g.: hexanoic acid (hexanoic acid), caprylic acid (caprylic acid, C8), capric acid (capric acid, C10), lauric acid (lauric acid, C12), myristic acid (myristic acid, C14), palmitic acid (C16), stearic acid (C18) and cholesterol (cholesterol) to increase stability and skin permeability of the peptide. Neither peptides nor conjugates are suggested as immunogens in this disclosure.

Although in principle, the present invention is able to improve all suggested IL31 related disease vaccination polypeptides, selected epitopes (see SeqIDs) were specifically assessed with respect to their suitability with the present platform in comparison to a CRM197 based vaccine.

### Selected Sequences

SeqID132
SKMLLKDVEEEKG-NHNH2
SeqID133
SKMLLKDVEEEKG-C
SeqID134
EELQSLSK-NHNH2;
SeqID135
EELQSLSK-C;
SeqID136
KGVLVS-NHNH2;
SeqID137
KGVLVS-C;
SeqID138
SVIDEIIEHLDKLI-NHNH2;
SeqID139
SVIDEIIEHLDKLI-C;
SeqID140
SPAIRAYLKTIRQLDNKSVI-NHNH2;
SeqID141
SPAIRAY-LKTIRQLDNKSVI-C;
SeqID142
HERKRFILT-NHNH2;
SeqID143
HERKRFILT-C;
SeqID144
HESKRFILT-NHNH2;
SeqID145
HESKRFILT-C;
SeqID146
SVPTDTHERKRF-NHNH2,
SeqID147
SVPTDTHERKRF-C
SeqID148
SVPTDTHESKRF-NHNH2,
SeqID149
SVPTDTHESKRF-C
SeqID150
KRFILTISQQFS-NHNH2
SeqID151
KRFILTISQQFS-C

In view of these advantageous properties of the conjugates of the present invention, it follows that the conjugates and vaccines according to the present invention are specifically useable for active immunotherapy for calcitonin gene related peptide (CGRP) related disease.

CGRP related disease is selected from the group episodic and chronic migraine and cluster headache, hyperalgesia, hyperalgesia in dysfunctional pain states, such as for example rheumatoid arthritis, osteoarthritis, visceral pain hypersensitivity syndromes, fibromyalgia, inflammatory bowel syndrome, neuropathic pain, chronic inflammatory pain and headaches.

According to a preferred embodiment the CGRP derived polypeptide is derived from native human CGRP a (ACDTATCVTHRLAGLLSRSGGVVKNNFVPTNVGSKAF; a 37 aa peptide fragment of aa83-119 of calcitonin i isoform alpha-CGRP preproprotein, accession number NP_001365879.1) or of aa82-228 of native human CGRP beta (ACNTATCVTHRLAGLLSRSGGMVKSNFVPTNVGSKAF; a 37 aa peptide fragment of aa82-118 of calcitonin gene-related peptide 2 precursor, accession number NP_000719.1) or its precursor molecules (NP_001365879.1 and NP_000719.1). The CGRP derived polypeptide can also be a mimic with one or more aa exchanges forming a mimotope of the respective native sequence.

According to a preferred embodiment, the CGRP derived polypeptide is selected from functional sites of native human CGRP including the central region of CGRP (e.g. aa8-35) or fragments thereof, the C-terminal CGRP receptor binding region (e.g.: aa11-37) or fragments thereof or the N-terminal region potentially also containing the cyclic C2-C7 loop within CGRP (e.g. aa1-20) or fragments thereof consisting of amino acid residues derived from these sites or of mimotopes.

Further preferred target sequences include ACDTATCVTH; ACDTATCVTHRLAGL; ACDTATCVTHRLAGLLSR; ACDTATCVTHRLAGLLSRSG; ACDTATCVTHRLAGLLSRSGGVVKN; TATCVTHRLAGLL; ATCVTHRLAGLLSR; RLAGLLSR; RLAGLLSRSGGVVKN; RSGGVVKN; RLAGLLSRSGGVVKNNFVPT; RLAGLLSRSGGVVKNNFVPTNVG; RLAGLLSRSGGVVKNNFVPTNVGSK; RLAGLLSRSGGVVKNNFVPTNVGSKAF; LLSRSGGVVKNNFVPTNVGSKAF; RSGGVVKNNFVPTNVGSKAF; GGVVKNNFVPTNVGSKAF; VVKNNFVPTNVGSKAF; NNFVPTNVGSKAF; VPTNVGSKAF; NVGSKAF; GSKAF

In US 2022/0073582 A1 polypeptide constructs containing CGRP derived peptides aa1-10 ACDTATCVTH; aa 1-15 ACDTATCVTHRLAGL; aa 1-18 ACDTATCVTHRLAGLLSR; aa 1-20 ACDTATCVTHRLAGLLSRSG; aa 1-25 ACDTATCVTHRLAGLLSRSGGVVKN; aa 4-16 TATCVTHRLAGLL; aa 5-18 ATCVTHRLAGLLSR; aa 11-18 RLAGLLSR; aa 11-25 RLAGLLSRSGGVVKN; aa 11-30 RLAGLLSRSGGVVKNNFVPT; aa 11-33 RLAGLLSRSGGVVKNNFVPTNVG; aa 11-35 RLAGLLSRSGGVVKNNFVPTNVGSK; aa 11-37 RLAGLLSRSGGVVKNNFVPTNVGSKAF; aa 15-37 LLSRSGGVVKNNFVPTNVGSKAF; aa 18-37 RSGGVVKNNFVPTNVGSKAF; aa 20-37 GGVVKNNFVPTNVGSKAF; aa 22-37 VVKNNFVPTNVGSKAF; aa 25-37 NNFVPTNVGSKAF; aa 28-37 VPTNVGSKAF; aa 31-37 NVGSKAF; of native human CGRP (accession number: NP_001365879.1) having the following amino acid sequence: MGFQKFSPFLALSILVLLQAGSLHAAPFRSALESSPADPATLSEDEAR-LLLAALVODYVOMKASELEQEQEREGSRIIAQKRACDTATCVTHRLAGLLSRSGGVVKNNFVPT NVGSKAFGRRRRDLQA were disclosed. Peptide immunogen constructs disclosed in US 2022/0073582 A1 require a CGRP derived B-cell epitopes coupled to one or more promiscuous T-cell epitopes to be functional as peptide immunogen constructs for targeting GCRP.

In addition to active immunotherapeutics, humanized anti-calcitonin gene-related peptide (CGRP) monoclonal antibodies have been suggested as anti CGRP targeting paradigm. Antibodies have been found to be effective in reducing the frequency of chronic migraine (Dodick D W et al. (2014) Lancet Neurol. 13:1100-1107; Dodick D W et al. (2014) Lancet Neurol. 13:885-892; Bigal M E et al. (2015) Lancet Neurol. 14:1081-1090; Bigal M E et al. (2015) Lancet Neurol. 14:1091-1100; and Sun H et al. (2016) Lancet Neurol. 15:382-390).

Along these lines, U.S. Pat. No. 8,597,649 B2, EP 1957106 B1 and U.S. Pat. No. 9,266,951 B2 disclose clinically used monoclonal antibodies targeting aa25-37 and/or aa33-37 within human CGRP to treat migraine, cluster headache and tension headache. US20120294797 A1 discloses clinically used CGRP targeting monoclonal antibodies which are also specific for a C-terminal epitope aa26-37 (https://doi.org/10.1080/21655979.2021.2006977) according to co-cristallization results indicating that this epitope is suitable for immunotherapy. U.S. Pat. No. 9,505,838 B2 also discloses clinically used monoclonal antibody directed against CGRP, binding to the C-terminal fragment having amino acids 25-37 of CGRP or a C-terminal epitope within amino acids 25-37 of CGRP

Although in principle, the present invention is able to improve all suggested CGRP related disease vaccination polypeptides, selected epitopes (see SeqID 152 to SeqID162) were specifically assessed with respect to their suitability with the present platform in comparison to a CRM197 based vaccine.

Selected sequences for experiments:

SeqID152
RLAGLLSR-NHNH2,
SeqID153
RLAGLLSR-C
SeqID154
RLAGLLSRSGGVVKN-NHNH2,
SeqID155
RLAGLLSRSGGVVKN-C
SeqID156
RSGGVVKN-NHNH2,
SeqID157
RSGGVVKN-C
SeqID158
NNFVPTNVGSKAF-NHNH2,
SeqID159
NNFVPTNVGSKAF-C
SeqID160
VPTNVGSKAF-NHNH2,
SeqID161
VPTNVGSKAF-C
SeqID162
NVGSKAF-NHNH2,
SeqID163
NVGSKAF-C

In view of these advantageous properties of the conjugates of the present invention, it follows that the CLEC based conjugates and CLEC based vaccines according to the present invention are specifically useable for specific allergen immunotherapy (AIT) for the treatment of IgE mediated type I allergic disease. Allergic disease typically refers to a number of conditions caused by the hypersensitivity of the immune system to typically harmless substances in the environment. These diseases include but are not limited to hay fever, seasonal-, food-, pollen-, mold spores-, poison plants-, medication/drug-, insect-, scorpion- or spider-venom, latex- or dust allergies, pet allergies, allergic asthma bronchiale, allergic rhinitis and -conjunctivitis, atopic dermatitis, contact dermatitis to adhesives, antimicrobials, fragrances, hair dye, metals, rubber components, topical medicaments, rosins, waxes, polishes, cement and leather, chronic rhinosinusitis, atopic eczema, autoimmune diseases where IgE plays a role (“autoallergies”), chronic (idiopathic) and autoimmune urticaria, anaphylaxis, especially idiopathic and exercise-induced anaphylaxis.

To date, specific AIT is the only curative approach for allergy and is mediated by repeated injections of allergen containing extracts of different sources such as food, pollen, animal dander, mites, or insect venoms. The specific AIT paradigms currently used in clinical practice however are characterized by very long treatment periods, the need for frequent injections, and a limited efficacy, which together result in low patient compliance (Musa et al. Hum Vaccin Immunother. 2017 March; 13(3): 514-517. doi: 10.1080/21645515.2016.1243632).

The primary mechanism of AIT is the induction of so-called blocking antibodies, preferably of the IgG4 isotype but also other isotypes (e.g. IgG1 or IgA). It has been shown that naturally occurring IgA and IgG target epitopes on the surface of an allergen that differ from epitopes specifically recognized by IgE (so-called IgE epitopes) (Shamji, Valenta et al. 2021; Allergy 76 (12): 3627-3641). The latter epitopes however are responsible for crosslinking IgE bound to mast cells via the high affinity FcεRI receptor and thus the induction of the immediate type allergic immune response.

In contrast, AIT induced blocking antibodies (pre-dominantly of the IgG- and IgA-type) are directed against said IgE epitopes. Their binding to the allergen interferes with cross-linking of cell bound IgE thereby inhibiting the initiation of the allergic response. IgG4 exhibits favorable characteristics as blocking antibody as it is unable to cross-link allergens and shows low affinity for activating Fc receptor for IgG (FcγR) while retaining high affinity for the FcγRIIb. These characteristics enable IgG4 to be an efficient inhibitor of IgE-dependent reactions without untoward inflammation associated with IgG immune complex formation and complement activation (Shamji, Valenta et al. 2021). However, the blocking capacity of IgG4 is not necessarily superior to other IgG subclasses {Ejrnaes et al. 2004; Molecular Immunology Vol. 41, Issue 5, 2004, P. 471-478}, and particularly early in AIT blocking activity is also conferred by other IgG types, especially IgG1 (Strobl, Demir et al. 2023, Journal of Allergy and Clinical Immunology doi: 10.1016/j.jaci.2023.01.005).

According to a preferred embodiment, single allergen epitopes may be used to trigger an immune response against the respective allergens (e.g. IgE epitopes mentioned in Table A and B). In another preferred embodiment a combination of epitopes from one allergen may be used to trigger an immune response against different domains of an allergen.

These anti-single allergen vaccines are highly effective when used alone or in combination with peptide vaccines directed against other allergen molecules involved in allergic diseases. Therefore, it is a preferred embodiment to provide a combination of epitopes of different allergens to trigger an immune response against different allergens.

According to a preferred embodiment, the allergen derived polypeptide is a fragment of one allergen protein, especially of one described in Table A and B and/or is preferably selected from native proteins, especially those listed in Table A and B.

According to a preferred embodiment, the allergen derived polypeptide is a linear fragment of one allergen protein, including those described in Table A and B.

According to a preferred embodiment, the allergen derived polypeptide is selected from mimics of the above-mentioned allergen derived polypeptides including mimotopes and peptides containing amino acid substitutions.

According to a preferred embodiment the allergen derived polypeptide is derived from native allergens or is a mimic with one or more aa exchanges forming a mimotope of the respective native sequence.

According to a preferred embodiment, the allergen epitope can be a conformational epitope comprising at least two amino acids or amino acid sequences, which are spatially distinct from each other, but in close proximity such as to form a respective paratope. The paratope is typically bound by an anti-allergen antibody e.g., a polyclonal anti-allergen antibody obtained upon vaccinating a mammal with the vaccine and specifically recognizing the naturally occurring allergen.

According to a preferred embodiment respective conformational epitopes or mimotopes can be acquired from the literature or identified using predictive algorithms (as disclosed in: Dall′Antonia and Keller 2019, Nucleic Acids Research 47(W1): W496-W501) or publicly available databases (e.g.: https://www.iedb.org/). Selected examples of potential target antigens and their respective epitopes/mimotopes to be used with the current invention are summarized in Table A and B.

According to a preferred embodiment further preferred target sequences include constrained peptides e.g. cyclisized peptides or peptides joint by a suitable aa linker known to a man skilled in the art, e.g.: (G)n linkers, (K)n linkers, GGSGG or similar.

TABLE A
Preferred allergens for use in CLEC based vaccines

| Allergen source | Allergens | UNIPROT |
|---|---|---|
| Alnus glutinosa | Aln g 1 | P38948 |
| Alternaria alternata | Alt a 1 | P79085 |
| | Alt a 3 | P78983 |
| | Alt a 4 | Q00002 |
| | Alt a 5 | P42037 |
| | Alt a 6 | Q9HDT3 |
| | Alt a 7 | P42058 |
| | Alt a 8 | P0C0Y4 |
| | Alt a 10 | P42041 |
| | Alt a 12 | P49148 |
| | Alt a 13 | Q6R4B4 |
| Ambrosia | Amb a 1 | P27761 |
| artemisiifolia | | |
| | Amb a 1.05 | P27762 |
| | Amb a 3 | P00304 |
| | Amb a 5 | P02878 |
| | Amb a 6 | O04004 |
| | Amb a 7 | no full sequence available |
| | Amb a 8 | Q2KN24 |
| | Amb a 9 | Q2KN27 |
| | Amb a 10 | Q2KN25 |
| Apis mellifera | Api m 1 | P00630 |
| | Api m 2 | Q08169 |
| | Api m 3 | Q4TUB9 |
| | Api m 4 | 01501 |
| | Api m 5 | B2D0J4 |
| | Api m 6 | Q27SJ8 |
| | Api m 7 | Q8MQS8 |
| | Api m 8 | B2D0J5 |
| | Api m 9 | C9WMM5 |
| | Api m 10 | Q1HHN7 |
| | Api m 11 | B3GM11 |
| | Api m 12 | Q868N5 |
| Apium graveolens | Api g 1 | P49372 |
| | Api g 2 | E6Y8S8 |
| | Api g 3 | P92919 |
| | Api g 4 | Q9XF37 |
| | Api g 5 | P81943 |
| Arachis hypogae | Ara h 1 | P43238 |
| | Ara h 2 | Q6PSU2-1 |
| | Ara h 3 | O82580 |
| | Ara h 3.02 | Q9SQH7 |
| | Ara h 5 | Q9SQI9 |
| | Ara h 6 | Q647G9 |
| | Ara h 7 | Q9SQH1 |
| | Ara h 8 | Q6VT83 |
| Betula verrucosa | Bet v 1 | P15494 |
| | Bet v 2 | P25816 |
| | Bet v 3 | P43187 |
| | Bet v 4 | Q39419 |
| | Bet v 6 | O65002 |
| | Bet v 7 | P81531 |
| Canis familiaris | Can f 1 | O18873 |
| | Can f 2 | O18874 |
| | Can f 3 | P49822 |
| | Can f 4 | D7PBH4 |
| | Can f 5 | P09582 |
| | Can f 6 | H2B3G5 |
| | Can f 7 | Q28895 |
| | Can f 8 | F1PHB6 |
| Carpinus betulus | Car b 1 | P38949 |
| Castanea sativa | Cas s 1 | B7TWE3 |
| | Cas s 5 | Q42428 |
| | Cas s 8 | sequence unclear |
| Cladosporium | Cla h 2 | no full sequence available |
| herbarum | | |
| | CIa h 5 | P42039 |
| | CIa h 6 | P42040 |
| | CIa h 7 | P42059 |
| | CIa h 8 | P0C0Y5 |
| | CIa h 9 | B7ZK61 |
| | CIa h 10 | P40108 |
| | CIa h 12 | P50344 |
| Corylus avellana | Cor a 1 | Q08407 |
| | Cor a 2 | Q9AXH5 |
| | Cor a 6 | A0A0U1VZC8 |
| | Cor a 8 | Q9ATH2 |
| | Cor a 9 | Q8W1C2 |
| | Cor a 10 | Q9FSY7 |
| | Cor a 11 | Q8S4P9 |
| Cryptomeria japonica | Cry j 1 | P18632 |
| | Cry j 2 | P43212 |
| Cyprinus carpio | Cyp c 1 | Q8UUS3 |
| Daucus carota | Dau c 1 | O04298 |
| | Dau c 4 | Q8SAE6 |
| Dermatophagoides | Der p 1 | P08176 (variant) |
| pteronyssinus | | |
| | Der p 2 | P49278 |
| | Der p 3 | P39675 |
| | Der p 4 | Q9Y197 |
| | Der p 5 | P14004 |
| | Der p 6 | P49277 |
| | Der p 7 | P49273 |
| | Der p 8 | P46419 |
| | Der p 9 | Q7Z163 |
| | Der p 10 | O18416 |
| | Der p 11 | Q6Y2F9 |
| | Der p 14 | Q8N0N0 |
| | Der p 20 | B2ZSY4 |
| | Der p 21 | Q2L7C5 |
| | Der p39 | XP_027203190.1 |
| Fagus sylvatica | Fag s 1 | B7TWE6 |
| Felis domesticus | Fel d 1 | P30438 (chain 1), P30440 |
| | | (chain2) |
| | Fel d 2 | P49064 |
| | Fel d 3 | Q8WNR9 |
| | Fel d 4 | Q5VFH6 |
| | Fel d 5 | no full sequence available |
| | Fel d 6 | no full sequence available |
| | Fel d 7 | E5D2Z5 |
| | Fel d 8 | F6K0R4 |
| Hevea brasiliensis | Hev b 1 | P15252 |
| | Hev b 2 | P52407 |
| | Hev b 3 | O82803 |
| | Hev b 4 | Q6T4P0 |
| | Hev b 5 | Q39967 |
| | Hev b 6 | P02877 |
| | Hev b 7.01 | O04008 |
| | Hev b 7.02 | O65811 |
| | Hev b 8 | O65812 |
| | Hev b 9 | Q9LEJ0 |
| | Hev b 10 | P35017 |
| | Hev b 11 | Q949H3 |
| | Hev b 12 | Q8RYA8 |
| | Hev b 13 | Q7Y1X1 |
| Juniperus ashei | Jun a 1 | P81294 |
| | Jun a 2 | Q9FY19 |
| | Jun a 3 | P81295 |
| Malus domestica | Mal d 1 | P43211 |
| | Mal d 2 | Q9FSG7 |
| | Mal d 3.0101w | Q5J026 |
| | Mal d 4 | Q9XF42 |
| Quercus alba | Que a 1 | B6RQS1 |
| Phleum pratense | PhI p 1 | Q40967 |
| | PhI p 2 | P43214 |
| | PhI p 4 | Q5ZQK5 |
| | PhI p 5 | Q40960 |
| | PhI p 6 | P43215 |
| | PhI p 7 | O82040 |
| | PhI p 11 | Q8H6L7 |
| | PhI p 12 | P35079 |
| | PhI p 13 | Q9XG86 |
| Polistes annularis | Pol a 1 | Q9U6W0 |
| | Pol a 5 | Q05109 |
| Polistes dominulus | Pol d 1 | Q6Q252 |
| | Pol d 4 | Q7Z269 |
| | Pol d 5 | Q68KJ8 |
| Polistes exclamans | Pol e 1 | no full sequence available |
| | Pol e 4 | no full sequence available |
| | Pol e 5 | Q68KJ9 |
| Polistes fuscatus | Pol f 5 | P35780 |
| Polistes gallicus | Pol g 1 | P83542 |
| | Pol g 5 | P83377 |
| Polistes metricus | Pol m 5 | P35780 |

TABLE B
Preferred allergen epitopes for use in CLEC based vaccines

| Allergens | UNIPROT | EPITOPES/MIMOTOPES |
|---|---|---|
| Amb a 1 | P27761 | GMLATVAFNT FTDNVDQR, (Mol Immunol. 1988 |
| | | Apr.; 25 (4): 355-65), AFNKFTDNVDQR, MPRCRFGF, |
| | | WRTQNDVLENG (Mol Immunol. 2009 Feb.; 46(5): |
| | | 873-83.), TFTDNVDQRMPRCRH (Peptides. 2021 |
| | | Nov.; 145: 170628.), RVVELMDWTVLH, GSAMTWGMLAAE, |
| | | SYNIIATGIHPV, TMVATGLMPVLI, QDEDDIL, |
| | | CLFSQGNRC, VANLKVGV, NPGGLSAAPAGS, RHAST- |
| | | LLGRHG, EAAESMWRVASG, QNRLNSNGNNGGSQ, |
| | | DDDLQHQFDDQD (Acta Chim. Slov. 2019, 66, |
| | | 37-44) |
| Amb a 3 | P00304 | CDIKDPIRLEPGGPD, EVWREEAYHACDIKD, GKVYLVGG- |
| | | PELGGWK, LGGWKLQSDPRAYAL, NFTTGEDSVAEVWRE, |
| | | PGGPDRFTLLTPGSH, PGGPDRFTLLTPGSH, QFKTT- |
| | | DVLWENETTG, RAYALWSARQQFKTT, TPGSHFICTKDQKFV |
| | | (Eur J Immunol. 1986 Mar.; 16(3): 229-35) |
| Alt a1 | P79085 | MISTSRK, QKRNTIT, (Li et al. J Immunol (2019) |
| | | 203 (1): 31-38.), KISEFYGRKP, VATATLPNYC, |
| | | YSCGENSFMD, YYNSLGENIK (Kurup et al., Pep- |
| | | tides. 2003 Feb; 24 (2): 179-85.) |
| Ara h 1 | P43238 | REREREEDWRQPREDWRRPS, RTRGRQPGDYDDDRRQPRRE, |
| | | DDDRRQPRREEGGRWGPAGP, TTNQRSPPGERTRGRQPGDY, |
| | | QPREDWRRPSHQQPRKIRPE, GREGEQEWGTPGSHVREETS, |
| | | PVNTPGQFEDFFPASSRDQS, AGGEQEERGQRRWSTRSSEN, |
| | | REGEPDLSNNFGKLFEVKPD, NASSELHLLGFGINAENNHR, |
| | | IDQIEKQAKDLAFPGSGEQV, LAFPGSGEQVEKLIKNQKES |
| | | (Cell Rep Med 2 (10): 100410.), AKSSPYQKKT, |
| | | QEPDDLKQKA, LEYDPRLUYD, GERTRGRQPG, |
| | | PGDYDDDRRQ, PRREEGGRWG, REREEDWRQP, ED- |
| | | WRRPSHQQ, QPKKIRPEGR, TPGQFEDFFP, SYLQEFSRNT, |
| | | FNAEFNEIRR, EQEERGQRRW, DITNPINLRE, |
| | | NNFGKLFEVK, GTGNLELVAV, RRYTARLKEG, |
| | | ELHLLGFGIN, HRIFLAGDKD, IDQIEKQAKD, |
| | | KDLAFPGSGE, KESHFVSARP, PEKESPEKED |
| | | (Eur J Biochem 245 (2): 334-339) |
| Ara h 2 | Q6PSU2-1 | ARQQWELQGDRRCQSQLERA, ERDPYSPSQDPYSPSPYDRR, |
| | | RRCQSQLERANLRPCEQHLM, RDPYSPSPYDRRGAGSSQHQ, |
| | | GRDPYSPSQDPYSPSQDPDR, PYSPSQDPDRRDPYSPSPYD, |
| | | QKIQRDEDSYERDPYSPSQD (Cell Rep Med 2 (10): |
| | | 100410.), HASARQQWEL, QWELQGDRRC, DRRCQSQLER, |
| | | LRPCEQHLMQ, KIQRDEDSYE, YERDPYSPSQ, |
| | | SQDPYSPSPY, DRLQGRQQEQ, KRELRNLPQQ, QRCDLDVESG |
| | | (Arch Biochem Biophys 342(2): 244-253), |
| | | HASARQQWEL, ERDPYSPSQDPYSPS, RRCQSQLER, |
| | | CDLEVESGGRDRY, ERDPYSPSQDPYSPS, |
| | | NLRPCEQHLMQKIQRD, PQRCDLE, RQQEQQFKRELRNLPQQ, |
| | | SDRLQGRQQ (Deak, Vrabel et al. 2017), |
| | | RRCQSQLERANLRPCEQHLMQKIQRDEDSYGRDPYSPSQDPY |
| | | (Mol Immunol 85: 81-88) |
| Ara h 3 | O82580 | IETWNPNNQEFECAG, GNIFSGFTPEFLEQA, VTVRGGL- |
| | | RILSPDRK, DEDEYEYDEEDRG (J Clin Invest 103 (4): |
| | | 535-542), EDEYEYDEEDRRRGRGSRGR, NIFSGFTPE- |
| | | FLEQAFQVDDR, ESEEEGAIVTVRGGLRILSP, SGFTPE- |
| | | FLEQAFQVDDRQIV, EEGAIVTVRGGLRILSPDRK, |
| | | TYEEPAQQGRRYQSQRPPRR, RRADEEEEYDEDEYEYDEED, |
| | | NHEQEFLRYQQQSRQSRRRS, QEFLRYQQQSRQSRRRSLPY, |
| | | QEEREFSPRGQHSRRERAGQ (Cell Rep Med 2 (10): |
| | | 100410) |
| Ara h 6 | Q647G9 | CDELNEMENTQR, CEALQQIMENQCD, CNFRAPQRCDLDV, |
| | | KPCEQHIMQRI, YDSYDIR, KRELRMLPQQ, MRRERGRGG- |
| | | DSSSS (Sci Rep 7(1): 3981) |
| Bet v 1 | P15494 | KAEQVKASKEMGETLLRAVESYLLAHSDAYN, GPGTIK- |
| | | KISFPEGFPFKYVKDRVDEVDHTN, DGGSILKISNKYHT- |
| | | KGDHEVKAEQVKASKE, LFPKVAPQAISSVENIEGNGGPGTIK- |
| | | KISE, MGETLLRAVESYLL, MGVENYE- |
| | | TETTSVIPAARLFKAFI, VDHTNFKYNYSVIEGGPIGD- |
| | | TLEKISNEIK (Clin Exp Allergy 34 (10): 1525- |
| | | 1533), ISFPEGFPFK, FILDGDNLFPKVAPQAISSVE, |
| | | NIEGNGGPGTIKK (Anal Chem 90 (19): 11315-11323), |
| | | DGDNLFPKVA (Int Arch Allergy Appl Immunol |
| | | 89(4): 410-415), ILDGDNLFPKVAPQAI Int Arch Al- |
| | | lergy Immunol 101 (1): 89-94), SKEMGETLLRAVE- |
| | | SYLLAHSD (J Allergy Clin Immunol 116(2): 370- |
| | | 376), EVDHTNFKYNYSVIEG, GGPIGDTLEKISNEIK, |
| | | GPGTIKKISF (J Immunol 186(9): 5333-5344), EQV- |
| | | KASKEMGETLLRAVESYLLA (Allergy 67 (12): 1530- |
| | | 1537), GDNLFPKVAPQAIS, ISFPEGFPFKY, |
| | | ISFPEGFPFKYVKD (Clin Exp Allergy 44 (2): 288- |
| | | 299), |
| | | aa 32-37, aa 42-50, aa 138-153 (BIOLOGICS AND |
| | | IMMUNOTHERAPY, VOLUME 149, ISSUE 1, p200-211, |
| | | JANUARY 2022), Mimotopes: CQQFLSVRALC, |
| | | QQFLSVRALC, COQFLSVRAL |
| Cry j 1 | P18632 | DALTLRTATNIW, DGDALTLRTATN, DGRGAQVYIGNG, |
| | | NATPQLTKNAGV, NGGPCVFIKRVS, NGNATPQLTKNA, |
| | | NSDDDPVNPAPG, VENGNATPQLTK (Biol Pharm Bull |
| | | 28(8): 1496-1499), NAGVLTCSLSK (Mol Immunol |
| | | 30 (2): 183-189) |
| Cry j 2 | P43212 | KWVNGREI, GQCKWVNGREICNDRDRPTA, GRENSRAEVSYV- |
| | | HVNGAKFI, PGNKKFVVNNLFENGPCQPH, SHIIYENVEMIN- |
| | | SENPILINQFYCT, YCTSASACQNORSAVQIQDV (Clin Exp |
| | | Allergy 33(2): 211-217), TYKNIRGTSATAAAIQLKCS |
| | | (J Clin Immunol 33(5): 977-983), AEVSYVHVNGAK |
| | | (Eur J Immunol 32 (6): 1631-1639) |
| Der p 1 | P08176 | KGIPNTKAP, DMFQIGKYG, GIREVWPAG, SSMGAYWGG, |
| | (variant) | KGTTGVRNT (Molecular Immunology 45 (5): 1308- |
| | | 1317), CQIYPPNANKIREALAQ, GYSNAQGVDYWI, |
| | | NOSLDLAEQELVDCASQHGC, VRNSWDTNWGDNGY (Mol Im- |
| | | munol 29(6): 739-749), EQSYPCWLSGTPSTP, TPLC- |
| | | DYAAARVGACG, KEQLPTSYPPERAGW, NCLSSDEPLHIRWCQ, |
| | | EALSEEEWPRYTSHP, SCDATQRAQGRCS, SCDSSQKKOGRCS, |
| | | SCDESRRRQGRCS (Clin Exp Allergy 29(11): 1563- |
| | | 1571), Mimotopes: KGIPNTKAP, DMFQIGKYG, GI- |
| | | REVWPAG, SSMGAYWGG, KGTTGVRNT, CKGIP- |
| | | NTKAP, CDMFQIGKYG, CGIREVWPAG, CSSMGAYWGG, |
| | | CKGTTGVRNT, CKGIPNTKAPC, CDMFQIGKYGC, CGIREVW- |
| | | PAGC, CSSMGAYWGGC, KGTTGVRNTC, KGIPNTKAPC, |
| | | DMFQIGKYGC, GIREVWPAGC, SSMGAYWGGC, KGTT- |
| | | GVRNTC, |
| Der p 2 | P49278 | FVVEYTKKW, SWWNLPQIG, KGITTKWMA, AGISYTKTW |
| | | (Molecular Immunology 45 (5): 1308-1317), |
| | | DQVDVKDCANHEIKK, VPGIDPNACHYMKCK (Mol Immunol |
| | | 28 (11): 1225-1232), APKSENVVVTVKVMGDNGVLACAI- |
| | | ATHAKIRD, CHGSEPCIIHRGKPFQLEAVFEANQNSKTAK, |
| | | DQVDVKDCANHEIKKVLVPGCHGSEPCIIHRGK, |
| | | EANQNSKTAKIEIKASIEGLEVDVPGIDPNAC, |
| | | EVDVPGIDPNACHYMKCPLVKGQQYDIKYTWIVPKIAPKSEN |
| | | (Allergy 67 (5): 609-621), Mimotopes: FVVEY- |
| | | TKKW, SWWNLPQIG, KGITTKWMA, AGISYTKTW, FVVEY- |
| | | TKKWC, SWWNLPQIGC, KGITTKWMAC, AGISYTKTWC, C |
| | | FVVEYTKKWC, CSWWNLPQIGC, CKGITTKWMAC, CAG- |
| | | ISYTKTWC, CFVVEYTKKW, CSWWNLPQIG, CKGITTKWMA, |
| | | CAGISYTKTW |
| Der p39 | XP_ | QEELREAFRMY, TSALREILRAL, NDELDEMIAEI (World |
| | 027203190.1 | Allergy Organization Journal (2022) 15: 100651) |
| Fel d 1 | P30438 | KALPVVLENARILKNCVDAKMTEEDKE, |
| | (chain 1), | KRDVDLELTGTPDEYVEQVAQYKALPV (Proc Natl Acad |
| | P30440 | Sci U S A 90 (16): 7608-7612), DAKMTEEDKENALS, |
| | (chain2) | EICPAVKRDVDLELTG, EPERTAMKKIQDCY, FA- |
| | | VANGNELLLDLS, KKIQDCYVENGLIS, LLDKIYTSPLC, |
| | | VAQYKALPVVLENA, VKMAETCPIFYDVE, VQNTVED- |
| | | LKLNTLGR (J Allergy Clin Immunol 93(1 Pt 1): |
| | | 34-43), EICPAVKRDVDLELTGTPDEYVEQVAQYK, |
| | | SSKDCMGEAVQNTVEDLKLNTLGR, VKMAITCPIFYDVFFA- |
| | | VANGNELLLDLSLTKVNA (Clin Exp Allergy 44 (6): |
| | | 882-894), CPAVKRDVDLELT, EQVAQYKALPVVLENA KAL- |
| | | PVVLENARILNCV RILKNCVDAKMTEEDKE KE- |
| | | NALSLLDKIYTSPL TAMKKIQDCY VENGLI SRVLD- |
| | | GLVMTTISSSK (J ALLERGY CLIN IMMUNOL VOLUME 131, |
| | | NUMBER 1, 109.e4), ENLAYTKALLTG; NNISVDGLLTS; |
| | | VIRNELLLTYA; VINDLLLVLA; GDFYPL; NDFYPE; LDLNP |
| | | (Molecular Immunology 71 (2016) 176-183), FA- |
| | | VANGNELL; LKLNTLGREICPAVKRGVDL ; YVEQV; |
| | | ENARILKNCVDAKM; aa 21-31, aa 21-47 (J ALLERGY |
| | | CLIN IMMUNOL, JANUARY 2013, 109.e1) |
| PhI p 1 | Q40967 | VRYTTEGGTKTEAEDVIPEGWKADTSYESK, APYHEDLSGHAF- |
| | | GAM, HITDDNEEPIAPYHEDLSGHA, NEEPIAPY- |
| | | HFDLSGHAFG, CFEIKCTKPEACSGEPVVV, CTK- |
| | | PEACSGEPVVVHITDDNEEPIAPYHEDLSGH, DNEEPIAPYHE, |
| | | EPIAPYHEDLSGH, EQKLRSAGELELQFRRVKC, |
| | | GEPVVVHITDDNEEPIAPYHEDLSGHAFGAMAKKG, |
| | | GNTPIFKSGRGCGSCFEIKCTK- |
| | | PEACSGEPVVVHITDDNEEPIAPYHEDL, GPFTVRYTTE- |
| | | GGTKTE, GTKTEAEDVIPEGWKADTSYESK, |
| | | GYKDVDKPPFSGMTGCGNTPIFKSGRGCGSCFEIKCTKPEACS, |
| | | HITDDNEEPIAPYHE, HITDDNEEPIAPYHFDLSGHAFGA, |
| | | HVEKGSNPNYLALLVKYVNGDGDVVAV, KCTK- |
| | | PEACSGEPVVVHITDDNEEPIAPYHEDLS, KPPFSGMTGCGNT, |
| | | KTEAEDVIPEGWKADTSYESK, LTGPFTVRYTTEGGTKTE- |
| | | AEDVIPEGWKADTSYESK, PIAPYHED, PIAPYHEDLSGHAFG |
| | | (Faseb j 13(11): 1277-1290) |
| PhI p 2 | P43214 | VPKVTFTVEKGSNEKHLAVLVKYEGDTMAEVEL, |
| | | VEKGSNEKHLAVLVKYEGDTMAEVELREHGSD, RE- |
| | | HGSDEWVAMTKGEGGVWTEDSEEPLQGPEN, |
| | | FRFLTEKGMKNVEDDVVPEKYTIGATYAPEE (J Allergy |
| | | Clin Immunol 135 (5): 1207-1207. e1201-1211), |
| | | CFRFLTEKGMKNVEDDVVPEKYTIGATYAPEE |
| | | (EBioMedicine 11 (2016) 43-57), |
| | | FRFLTEKGMKNVEDDVVPEKYTIGATYAPEEC |
| PhI p 5 | Q40960 | AEEVKVIPAGELQVIEKVDAAFKVAATAANAAPANDK,AT- |
| | | TEEQKLIEKINAGFKAALAAAAGVQPADKYR, FVAT- |
| | | FGAASNKAFAEGLSGEPKGAAESSSKAALTSK, |
| | | ADLGYGPATPAAPAAGYTPATPAAPAEAAPAGK, |
| | | AYKLAYKTAEGATPEAKYDAYVATLSEALRI, |
| | | EAAFNDAIKASTGGAYESYKFIPALEAAVK, |
| | | TVATAPEVKYTVFETALKKAITAMSEAQKAAK (J Allergy |
| | | Clin Immunol 133(3): 836-845.e811), SRLGRS- |
| | | SAWV, THWQLGERPD, PSTPGERVRH, RGGPDDLTAL, |
| | | PFWVRGTTW, PSTPGSRQNM, PSTPGDNPLV, KFVVNGRWID, |
| | | KFLVNGRWID, RLTENTEPLL, FTWGGLRDKS, ERAGAM- |
| | | ERAN, RSVSKEEPGM, KLGKFGAARV, VQDLMKSSGV (J |
| | | Allergy Clin Immunol 114 (6): 1294-1300), Mimo- |
| | | topes: KLGKFGAARV, CKLGKFGAARVC, CKLGKFGAARV, |
| | | KLGKFGAARVC, |
| PhI p 6 | P43215 | GKATTEEQKLIEDVNASFRAAMATTANVPPAD, |
| | | YKTFEAAFTVSSKRNLADAVSKAPQLVPKLDEVYN, |
| | | DAVSKAPQLVPKLDEVYNAAYNAADHAAPEDKY, |
| | | AADHAAPEDKYEAFVLHESEALRIIAGTPEVHA (J Allergy |
| | | Clin Immunol 135 (5): 1207-1207.e1201-1211) |

Positive outcome of AIT has been associated with the induction of high affinity IgG antibodies which are capable of neutralizing allergen (Svenson, Jacobi et al. 2003, Molecular Immunology 39(10): 603-612; Zha, Leoratti et al. 2018, Journal of Allergy and Clinical Immunology 142 (5): 1529-1536.e1526.). However, during classical AIT the initial avidity of the induced blocking IgG does not further increase over time (Strobl et al. 2023; Jakobsen C G et al, 2005, Clinical & Experimental Allergy, 35:193-198. doi: 10.1111/j.1365-2222.2005.02160.x) supporting the idea that AIT-induced inhibition of allergen binding to IgE can be explained mainly or solely by induction of increased amounts of specific IgG (Svenson et al, 2003, Molecular Immunology 39 (10): 603-612; Jakobsen et al, 2005). It is thus believed that the rather limited success of conventional AIT could be mainly due to the low immunogenicity of existing AIT compounds and lack of further avidity maturation upon prolonged AIT application.

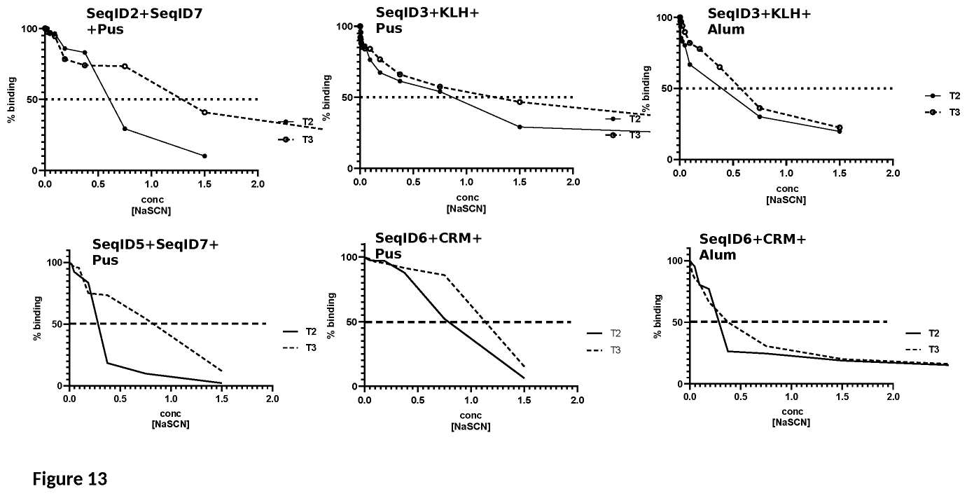

In contrast, the vaccines or conjugates according to the present invention are especially suited for AIT and the required induction of high avidity IgG as they induce IgE-epitope specific immune responses with higher antibody levels (as conventional vaccines) which display a prolonged affinity maturation after repeated immunization (see for example FIG. 13 and FIG. 21). This results in higher avidity immune sera as compared to classical vaccines including Alum adjuvanted vaccines and conjugate vaccines (with and without adjuvantation).

Currently, AIT exclusively uses allergen extracts from natural sources which represent complex heterogenous mixtures of allergenic and nonallergenic proteins, glycoproteins and polysaccharides (Cox et al 2005, Expert Review of Clinical Immunology 1(4): 579-588.). The resulting products are difficult to standardize and can induce unwanted side effects including anaphylaxis and T-cell based late phase responses (Mellerup, Hahn et al. 2000, Experimental Allergy 30(10): 1423-1429).

Novel vaccine concepts in clinical development therefore make use of platforms providing universal T-cell help (virus like particles {Shamji, 2022 #14} or carrier proteins such as KLH or hepatitis preS fusion protein (Marth et al. 2013, The Journal of Immunology 190(7): 3068-3078) and recombinant allergenic proteins or peptides (comprising allergenic epitopes or mimotopes thereof) to increase immunogenicity and affinity maturation (Bachmann et al, 2020, Trends in Molecular Medicine 26(4): 357-368).

The latter approach of applying peptide-carrier conjugates comprising allergenic epitopes or mimotopes thereof, would be especially favorable for a novel AIT paradigm in patients as it focuses the immune response on the desired target epitope(s) (i.e. the IgE epitopes) and completely avoids immediate (i.e. crosslinking of cell bound IgE by the vaccine) as well as late phase side effects (i.e. activation of allergen specific T-cell responses).

Marth et al (2013) disclose an AIT compound based on fusion proteins of two nonallergenic peptides, PA and PB, derived from the IgE-reactive areas of the major birch pollen allergen Bet v 1 which were fused to the hepatitis B surface protein, Pres, in four recombinant fusion proteins containing different numbers and combinations of the peptides. Similarly, the clinically tested AIT vaccine BM32 used 4 fusion proteins consisting of peptides from the 4 major timothy grass pollen allergens (Phl p 1, Phl p 2, Phl p 5, and Phl p 6) fused to the Pres carrier protein from hepatitis B. Weber et al. (2017; doi: 10.1016/j.jaci.2017.03.048) could demonstrate similar immunogenicity of Alum adjuvanted BM32 and conventional extract-mediated AIT in rabbits. However, despite initially promising clinical results (Eckl-Dorna, 2019 EBioMedicine. 2019 December; 50:421-432. doi: 10.1016/j.ebiom.2019.11.006.), the further development of the BM32 approach was abandoned after a Phase IIb study. So far no peptide-carrier conjugate or fusion protein AIT approach, nor any other novel recombinant vaccines for AIT have been licensed (Pavón-Romero, 2022, Cells. 2022 Jan. 8; 11 (2): 212. doi: 10.3390/cells 11020212).

Along these lines, it has been shown that a single injection of allergic patients with two monoclonal antibodies directed against two epitopes within the major cat allergen Fel d 1 is equally effective compared to years of conventional AIT (Orengo, Radin et al. 2018, Nature Communications 9(1): 1421) indicating that a small number of target epitopes within a given allergen may be sufficient to provide full protection from allergic immune responses. The vaccines or conjugates according to the present invention are especially suited to combine universal T-cell epitopes with such IgE epitopes or mimotopes on CLEC backbones to treat allergies.

Although in principle, the present invention is able to improve all suggested allergic disease vaccination polypeptides, selected epitopes (see Table A and B and SeqID45/46) are specifically preferred. For example, SeqID45/46 was shown to be superior to a KLH based vaccine.

In view of these advantageous properties of the conjugates of the present invention, it follows that the CLEC based conjugates and CLEC based vaccines according to the present invention are specifically useable for enhancing immunogenicity of marketed peptide/glyco-conjugate vaccines, especially also glycoconjugate vaccines used for the prevention of infectious diseases. Such diseases are for example microbial infections or viral infections, for example caused by Haemophilus influenzae type b (Hib), Streptococcus pneumoniae, Neisseria meningitidis and Salmonella Typhi or other infectious agents including those causing Hepatitis A or B, Human Papilloma Virus infections, Influenza, Thyphoid Fever, Measles, Mumps and Rubella. In addition, infections caused by meningococcal group B bacteria, Cytomegalovirus (CMV), Respiratory Syncytial Virus (RSV), Clostridioides Difficile, Extraintestinal Pathogenic Escherichia Coli (Expec), Klebsiella Pneumoniae, Shigella, Staphylococcus Aureus, Plasmodium falciparum, P. vivax, P. ovale, and P. malariae, Coronavirus (SARS-COV, MERS-COV, SARS-COV-2), Ebola Virus, Borrelia burgdorferi, HIV and others.

To date, several carrier proteins have been used in licensed conjugate vaccines: a genetically modified cross-reacting material (CRM197) of diphtheria toxin, tetanus toxoid (TT), meningococcal outer membrane protein complex (OMPC), diphtheria toxoid (DT), H. influenzae protein D (HiD), and recombinant Pseudomonas aeruginosa exotoxin A (rEPA). Clinical trials have demonstrated the efficacy of these conjugate vaccines in preventing infectious diseases and altering the spread of Haemophilus influenzae type b, Streptococcus pneumoniae, and Neisseria meningitidis and Typhoid fever. All carrier proteins have been effective in increasing vaccine immunogenicity but differ in the quantity and avidity of antibody they elicit, ability to carry multiple polysaccharides in the same product and to be given concurrently with other vaccines.

According to a preferred embodiment, the conjugate vaccines amenable for CLEC modification and immunogenicity enhancement include but are not limited to currently available vaccines including Haemophilus b Conjugate Vaccines (e.g.: PedvaxHIB®, ActHIB®, Hiberix®), recombinant Hepatitis B Vaccines (e.g.: Recombivax HB®, PREHEVBRIOR, Engerix-B, HEPLISAV-BR), Human Papillomavirus vaccines (e.g.: Gardasil®, Gardasil 9®, Cervarix®), Meningococcal (Groups A, C, Y, and W-135) Oligosaccharide Diphtheria CRM197 Conjugate Vaccines (e.g. Menveo®), Meningococcal (Groups A, C, Y and W-135) Polysaccharide Diphtheria Toxoid Conjugate Vaccine (e.g.: Menactra®), Meningococcal (Groups A, C, Y, W) TT-Conjugate Vaccine (e.g.: MenQuadfi®), multivalent Pneumococcal Conjugate Vaccine (e.g.: Prevnar-13®, Prevnar 20®, Pneumovax-23®, Vaxneuvance®), anti-typhoid vaccines (e.g.: Typhim V®, Typhim VI®, Typherix®, Vi polysaccharide bound to a non-toxic recombinant Pseudomonas aeruginosa exotoxin A, or Vi-rEPA or the Polysaccharide Tetanus Toxoid Conjugate Vaccine Typbar-TCV®), Varizella-Zoster-Virus vaccine (e.g.: Shingrix®) as well as other anti-infective conjugate vaccines carrying genetically modified cross-reacting material (CRM197) of diphtheria toxin, or tetanus toxoid (TT), or meningococcal outer membrane protein complex (OMPC), or diphtheria toxoid (DT), or H. influenzae protein D (HiD) or recombinant Pseudomonas aeruginosa exotoxin A (rEPA) as carrier molecule.

According to a further aspect, the novel conjugates according to the present invention can be used for the prevention of infectious diseases. Such diseases are for example microbial infections or viral infections, for example caused by Haemophilus influenzae type b (Hib), Streptococcus pneumoniae, Neisseria meningitidis and Salmonella Typhi or other infectious agents including those causing Hepatitis A or B, Human Papilloma Virus infections, Influenza, Thyphoid Fever, Measles, Mumps and Rubella. In addition infections caused by meningococcal group B bacteria, Cytomegalovirus (CMV), Respiratory Syncytial Virus (RSV), Clostridioides Difficile, Extraintestinal Pathogenic Escherichia Coli (Expec), Klebsiella Pneumoniae, Shigella, Staphylococcus Aureus, Plasmodium Sp., Coronavirus (SARS-COV, MERS-COV, SARS-COV-2), Ebola Virus, Borrelia burgdorferi, HIV and others.

Although in principle, the present invention can improve all suggested anti-infective conjugate vaccines, selected vaccines were specifically analysed. For example, the CLEC-modified Meningococcal (Groups A, C, Y, and W-135) Oligosaccharide Diphtheria CRM197 Conjugate Vaccines (i.e. Menveo®) and the Haemophilus b Conjugate Vaccine ActHIB® were shown to be superior to commercially available Menveo® and ActHIB® vaccine.

In view of these advantageous properties of the conjugates of the present invention, it follows that the conjugates and vaccines according to the present invention are specifically useable for active immunotherapy for Proprotein convertase subtilisin/kexin type 9 (PCSK9) related disease including but not limited to hyperlipidemia, hypercholesteremia, atherosclerosis, increased serum level of low-density lipoprotein cholesterol (LDL-C) and cardiovascular events, stroke or various forms of cancer.

According to a preferred embodiment the PCSK9 protein derived polypeptide is derived from native human PCSK9 (accession number: Q8NBP7) with the amino acid sequence:

MGTVSSRRSW WPLPLLLLLL LLLGPAGARA QEDEDGDYEE
LVLALRSEED GLAEAPEHGT TATFHRCAKD PWRLPGTYVV
VLKEETHLSQ SERTARRLQA QAARRGYLTK ILHVFHGLLP
GFLVKMSGDL LELALKLPHV DYIEEDSSVE AQSIPWNLER
ITPPRYRADE YQPPDGGSLV EVYLLDTSIQ SDHREIEGRV
MVTDFENVPE EDGTRFHRQA SKCDSHGTHL AGVVSGRDAG
VAKGASMRSL RVLNCQGKGT VSGTLIGLEF IRKSQLVQPV
GPLVVLLPLA GGYSRVLNAA CQRLARAGVV LVTAAGNERD
DACLYSPASA PEVITVGATN AQDQPVTLGT LGTNFGRCVD
LFAPGEDIIG ASSDCSTCFV SQSGTSQAAA HVAGIAAMML
SAEPELTLAE LRQRLIHFSA KDVINEAWFP EDQRVLTPNL
VAALPPSTHG AGWQLFCRTV WSAHSGPTRM ATAVARCAPD
EELLSCSSFS RSGKRRGERM EAQGGKLVCR AHNAFGGEGV
YAIARCCLLP QANCSVHTAP PAEASMGTRV HCHQQGHVLT
GCSSHWEVED LGTHKPPVLR PRGQPNQCVG HREASIHASC
CHAPGLECKV KEHGIPAPQE QVIVACEEGW TLTGCSALPG
TSHVLGAYAV DNTCVVRSRD VSTTGSTSEG AVTAVAICCR
SRHLAQASQE LQ

or fragments thereof, or is a mimic with one or more aa exchanges forming a mimotope of the respective native sequence. Further preferred target sequences include linear or constrained peptides (e.g. cyclisized) or peptides joint by a suitable aa linker (e.g.: ggsgg or similar).

According to a preferred embodiment, the PCSK9 protein derived polypeptide is selected from the region: aa150 to 170, aa153-162, aa205 to 225, aa211-223, aa368-382, or a polypeptide comprising or consisting of amino acid residues derived from these subunits or of mimotopes.

According to a preferred embodiment, the PCSK9 protein derived polypeptide is selected from derived sequences:

NVPEEDGTRFHRQASK, NVPEEDGTRFHRQASKC,
PEEDGTRFHRQASK, CPEEDGTRFHR-QASK,
PEEDGTRFHRQASKC, AEEDGTRFHRQASK,
TEEDGTRFHRQASK, PQEDGTRFHRQASK,
PEEDGTRFHRRASK, PEEDGTRFHRKASK,
PEEDGTRFHRQASR, PEEDGTRFHRTASK,
SIPWNLERITPPR, PEEDGTRFHRQASK,
PEEDGTRFHRQA, EEDGTRFHRQASK,
EEDGTRFHRQAS, SIPWNLERITP,
SIPWNLERITPC, SIPWN-LERIT,
SIPWNLERITC, LRPRGQPNQC, SRHLAQASQ,
SRHLAQASQC, SRSGKRRGER, SRSGKRRGERC,
IIGASSDCSTCFVSQ, IIGASSDSSTSFVSQ,
IIGASSDSSTSFVSQC, CIGASSDSSTSFVSC,
IGASSDSSTSFVSC, CDGTRFHRQASKC,
DGTRFHRQASKC, CDGTRFHRQASK,
AGRDAGVAKGAC, RDAGVAKC, RDAGVAK,
SRHLAQASQLEQC; SRHLAQASQLEQ, GDYEELVLALRC;
GDYEELVLALR, LVLALRSEEDC; LVLALRSEED,
AKDPWRLPC; AKDPWRLP, AARRGYLTKC, AARRGYLTK,
FLVKMSGDLLELALKLPC; FLVKMSGDLLELALKLP,
EEDSSVFAQC, EEDSSVFAQ, NVPEEDGTRFHRQASKC,
NVPEEDGTRFHRQASK, CKSAQRHERT-GDEEPVN,
KSAQRHFRTGDEEPVN,

According to a preferred embodiment single PCSK9 derived epitopes may be used to trigger an immune response against different regions within the 3 different domains of PCSK9 (i.e. inhibitory pro-domain (aa1-152), catalytic domain (aa153-448) and the C-terminal domain (449-692)). In another preferred embodiment a combination of PCSK9 derived epitopes may be used to trigger an immune response against different epitopes within the domains of PCSK9, in particular involving the catalytic domain (aa153-449), and further involving the inhibitory pro-domain (aa1-152) and/or the C-terminal domain (449-692).

Vascular disorders such as hyperlipidemia, hypercholesteremia, atherosclerosis, coronary heart disease and stroke are one of the main cause of death worldwide and elevated levels of LDL-C are playing key role in their pathogenesis. Therefore, LDL-C management is a very important element for a successful treatment of hyperlipidemia, hypercholesteremia, atherosclerosis. Accordingly, PCSK9 plays a crucial role in LDL catabolism through direct action on LDLR. Inhibition of PCSK9 turns out to be beneficial for the LDL-C levels. Therefore, anti-PCSK9 therapies are a promising approach in terms of beneficial modulation of LDL-C levels and treatment of PCSK9 related diseases.

WO2015128287A1 and EP2570135A1 disclose PCSK9 mimotope carrier conjugate vaccines (e.g.: KLH or CRM197 as carrier) and disclose the sequences PEEDGTRFHRQASK, AEEDGTRFHRQASK, TEEDGTRFHRQASK, PQEDGTRFHRQASK, PEEDGTRFHRRASK, PEEDGTRFHRKASK, PEEDGTRFHRQASR, PEEDGTRFHRTASK and aa150 to 170 and/or aa205 to 225 of PCSK9, especially SIPWNLERITPPR, PEEDGTRFHRQASK, PEEDGTRFHRQA, EEDGTRFHRQASK, EEDGTRFHRQAS, SIPWNLERITP and SIPWNLERIT.

CN105085684A discloses recombinant vaccine comprising an PCSK9 epitope and the DTT of diphtheria toxin. The epitope peptide is ligated to the C-terminus of the transmembrane domain DTT of the carrier protein diphtheria toxin. CN106822881A discloses protein vaccines characterized by recombinant PCSK9 protein fragment polypeptides (catalytic domain and C-terminal domain).

WO2022150661A2 discloses a virus (including a bacteriophage virus or a plant virus) or virus-like particle(s) for PCSK9 immunotherapy, especially comprising the PCSK9 derived sequence NVPEEDGTRFHRQASKC.

EP3434279A1 discloses an OSK-1-PCSK9 conjugate vaccine; using PCSK9 derived sequences LRPRGQPNQC, SRHLAQASQ and SRSGKRRGER. WO2021/154947 A1; discloses anti PCSK9 immunogens building on the Ubith technology, i.e. conjugate vaccines comprising PCSK9 epitopes fused to promiscuous T-cell epitopes. Sequences disclosed include aa153-162, aa368-382, aa211-223 and SIPWNLERIT, CIGASSDSSTSFVSC, CDGTRFHRQASKC.

WO2011/027257 A2, and WO 2012/131504 A1: disclose PCSK9 derived peptide-VLP and PCSK9 derived peptide-Carrier vaccines targeting PCSK9 including sequences SIPWNLERITPC, SIPWNLERITC, SIPWNLERITP, AGRDAGVAKGA, RDAGVAK; SRHLAQASQLEQ; GDYEELVLALR; LVLALRSEED; AKDPWRLP-; AARRGYLTK; FLVKMSGDLLELALKLP; EEDSSVFAQ. WO2015/123291 A1: discloses peptide-VLP (Qb) targeting PCSK9 vaccines comprising sequences: NVPEEDGTRFHRQASKC and CKSAQRHERTGDEEPVN and WO2018/189705 discloses peptide-carrier conjugates targeting PCSK9 based on sequence SIPWNLERITPC and modified derivatives thereof.

Preferred polypeptide immunogen constructs according to the present invention contain a B-cell epitope from alpha synuclein and a heterologous T helper cell (Th) epitope coupled to a CLEC. The present invention delivers surprisingly superior new conjugates which are surpassing conventional vaccines in immunogenicity, cross reactivity against alpha synuclein, selectivity for alpha synuclein species/aggregates, affinity, affinity maturation and inhibition capacity as compared to conventional vaccines.

The covalent conjugation of the alpha synuclein polypeptide to the β-glucan or mannan according to the present invention enables a surprisingly and unexpectable enhancement of the immune response for such polypeptides. This is specifically impressive in direct comparison with traditional vaccine formulations, such as the ones described by Rockenstein et al. (J. Neurosci., Jan. 24, 2018·38 (4): 1000-1014), as also demonstrated in the example section below.

Rockenstein et al. (2018) disclose the application of yeast whole glucan particles (GPs) non-covalently complexed with aSyn and rapamycin as immunotherapeutic for Parkinson's disease. These GPs have been created following a series of hot alkali, organic, and aqueous extraction steps from Saccharomyces cerevisiae leading to the final product consisting of a highly purified 3- to 4-μm-diameter yeast cell wall preparations devoid of cytoplasmic content and bounded by a porous, insoluble shell of β-glucans (mainly ß1-3 β-glucans).

Importantly, the vaccine composition disclosed by Rockenstein et al. (2018) consisted of GPs which were non-covalently complexed with either ovalbumin and mouse serum albumin (MSA), human aSyn and MSA or human aSyn, MSA and rapamycin. This complexation method relies on co-incubation of the different payloads with GPs and the subsequent diffusion into the hollow GP cavity without covalent attachment and is therefore similar to a set of vaccines disclosed in Example 28 provided within this application where only a mixing but no covalent attachment of components was used to formulate a vaccine and which proved inefficient and unsuitable as compared to the vaccines according to the present invention.
1) Rockenstein et al. show that non-covalent mixing of aSyn and GPs leads to a detectable immune response against aSyn hence showing that GPs can act as adjuvants. However, Rockenstein et al. also show that the non-covalent addition/co-complexation of rapamycin is required to induce significantly enhanced functionality of such a vaccine as compared to controls. In this view a mix of various adjuvants (GPs as well as the mTOR inhibitor rapamycin) is required to provide a fully functional vaccine, such as the vaccines disclosed by the present invention.
2) The vaccine disclosed by Rockenstein et al. is active in this aSyn overexpression model as it provides aSyn specific T-cell epitopes (among other T-cell epitopes like MSA-derived epitopes) in order to exert its full functionality namely induction of a neuroprotective, anti-aSyn directed cellular (i.e.: T-cell mediated) and humoral (i.e. antibody/B-cell based) immune response. This is in direct contrast to the teachings of the present invention, where it is already sufficient if only aSyn specific B-cell responses are elicited by the vaccines selected.
3) The use of full length aSyn is also posing the danger of inducing/augmenting autoreactive, aSyn specific T-cells which have the potential to exacerbate the underlying neuropathology in PD and other synucleopathies. Hence the GP-aSyn-rapamycin vaccine proposed by Rockenstein et al. is also with respect to this issue not preferred for human use.
4) As shown in Example 5 non-covalent mixing of aSyn derived peptides (e.g.: SeqID2 i.e. B-cell epitopes) and promiscuous T-cell epitopes (e.g.: SeqID7) with a β-Glucan particle (e.g.: non-oxidised pustulan), similar to Rockenstein et al., is also able to induce a low level antibody response against aSyn. However, vaccines according to the present invention, which build on covalent linkage of such peptides to a suitable glucan exert a significantly different and superior immune response (see also FIG. 5).

In addition, and also disclosed in Example 6 and FIG. 7, such covalently linked vaccines also show a highly beneficial lack of anti-glucan antibody responses as compared to non-covalently mixed vaccines building on glucan particles and peptides as disclosed by the present invention.

Hence, the prior art disclosure by Rockenstein et al. does not suggest the claimed subject matter disclosed by the present invention.

Specifically preferred aSyn polypeptides to be conjugated in the present invention are selected from native alpha synuclein or a polypeptide comprising or consisting of amino acid residues 1 to 5, 1 to 8, 1 to 10, 60 to 100, 70 to 140, 85 to 99, 91 to 100, 100 to 108, 102 to 108, 102 to 109, 103 to 129, 103 to 135, 107 to 130, 109 to 126, 110 to 130, 111 to 121, 111 to 135, 115 to 121, 115 to 122, 115 to 123, 115 to 124, 115 to 125, 115 to 126, 118 to 126, 121 to 127, 121 to 140, or 126 to 135, of the amino acid sequence of native human alpha synuclein:

MDVFMKGLSK AKEGVVAAAE KTKQGVAEAA
GKTKEGVLYV GSKTKEGVVH GVATVAEKTK
EQVTNVGGAV VTGVTAVAQK TVEGAGSIAA
ATGFVKKDOL GKNEEGAPQE GILEDMPVDP
DNEAYEMPSE EGYQDYEPEA
(human aSyn (1-140): UNIPROT accession
number P37840),

preferably a polypeptide comprising or consisting of amino acid residues 1 to 8, 91 to 100, 100 to 108, 103 to 135, 107 to 130, 110 to 130, 115 to 121, 115 to 122, 115 to 123, 115 to 124, 115 to 125, 115 to 126, 118 to 126, 121 to 127, or 121 to 140; or mimotopes selected from the group DQPVLPD, DQPVLPDN, DQPVLPDNE, DQPVLPDNEA, DQPVLPDNEAY, DQPVLPDNEAYE, DSPVLPDG, DHPVHPDS, DTPVLPDS, DAPVTPDT, DAPVRPDS, and YDRPVQPDR.

The current state of the art CLEC vaccines all induce high titers against the carrier proteins used (e.g.: CRM197 or OVA). However, this high immunogenicity as well as the structural complexity and heterogeneity of the carrier protein component may lead to the induction of high levels of carrier/protein specific antibodies at the expense of target specific responses which therefore might be underrepresented in comparison to the carrier response induced.

Also affinity maturation of target specific responses induced upon repeated immunization using carrier conjugates is compromised due to overrepresentation of carrier specific epitopes in the conjugates. Affinity maturation in immunology, as used and understood herein, is the process by which T_{FH} cell-activated B-cells produce antibodies with increased affinity for antigen during the course of an immune response. With repeated exposures to the same antigen, a host will produce antibodies of successively greater affinities. A secondary response can elicit antibodies with several fold greater affinity than in a primary response. Affinity maturation primarily occurs on surface immunoglobulin of germinal center B-cells and as a direct result of somatic hypermutation (SHM) and selection by T_{FH} Cells (see also: https://en.wikipedia.org/wiki/Affinity_maturation). Affinity Maturation according to the Segen's Medical Dictionary (https://medical-dictionary.thefreedictionary.com/affinity+maturation “>affinity maturation</a>) is the increased average affinity of antibodies to an antigen, which follows immunisation. Affinity maturation results from an increase of specific and more homogeneous IgG antibodies, and follows a less specific and more heterogeneous early response by IgM molecules.

Furthermore, high anti-carrier responses also pose the risk of immunological rejection and associated safety issues.

Thus, the identification of effective constructs with high immunogenicity, high target specificity and high tolerability/safety with low or absent carrier reactivity (i.e. against the protein carrier) according to the present invention successfully addresses this challenge by innovative solutions. In addition, it is crucial for the novel vaccines according to the present invention to provide immunotherapeutic agents which are inducing no/very weak immune responses against the sugar backbone. This is especially important as high anti-CLEC antibody levels induced upon immunization could inhibit or lower the efficacy of repeated immunization using the same CLEC-based vaccine due to vaccine neutralization or could also negatively impact the use of this type of vaccines for consecutive immunization against various different targets.

The vaccine platform according to the present invention also fulfils the need to combine various epitopes directed to one or several targets within one formulation without posing the risk to reduce efficacy due to unintended epitope spreading as reported for classical vaccines. The modular design of the platform according to the present invention allows for easy exchange of B- and T-cell epitopes without negative effects of a carrier induced response.

The present invention is based on a CLEC which exerts high specific binding to the cognate receptor. This binding is crucial and only strong binders are efficient as vaccine carriers/backbones.

According to the present invention, CLEC-conjugation enables an efficient immune response with novel characteristics. The conjugation according to the present invention precludes formation of anti-CLEC antibodies, especially for pustulan, such preclusion could be impressively shown in the course of the present invention. This lack of elicitation of anti-CLEC antibodies is very important for reusability and for reboostability of individual vaccines designed with the platform according to the present invention-be it with the same or different antigens.

In contrast to the conjugated embodiment of the present invention, a mere mixing of the CLEC polysaccharide adjuvant and the B-cell or T-cell epitope peptides does not lead to comparable effects in vivo. If conjugated, however, orientation of the peptide does not significantly influence the performance of the compounds according to the present invention; CLEC conjugation is therefore substantially independent from peptide orientation in the construct. In the course of the present invention, it could be shown that CLEC conjugation, especially to pustulan, leads to improvement of novel as well as existing peptide immunogens/antigens: This improvement is effected by higher, more target specific and more affine antibody reactions (as can be shown by antibody selectivity and functionality). This effect is most pronounced for pustulan or similar β-glucans which are predominantly linear β-(1,6)-glucans with a ratio of β-(1,6)-coupled monosaccharide moieties to non-β-(1,6)-coupled monosaccharide moieties of at least 1:1, preferably at least 2:1, more preferred, at least 5:1, especially at least 10:1, which performed surprisingly even considerably better than KLH or CRM in direct comparison and even better than mannan or lichenan conjugates or conjugates comprising barley β-glucans.

As used herein, the term “predominantly linear” β-(1,6)-glucans refers to β-(1,6)-D-glucans where no or only few cross-linking sugar monomer entities are present, i.e. wherein less than 1%, preferably less than 0.1%, especially less than 0.01%, of the monosaccharide moieties have more than two covalently attached monosaccharide moieties.

As already stated above, pustulan is the most preferred CLEC according to the present invention. Pustulan is usually free of cross-linking sugar moieties and predominantly β-(1,6)-coupled so that usual pustulan preparations to be used in the preparation of the conjugates according to the present invention contain less than 1%, preferably less than 0.1%, especially less than 0.01%, monosaccharide moieties with more than two covalently attached monosaccharide moieties, and contains maximally 10% impurities with β-(1,3)- or β-(1,4)-coupled monosaccharides.

The fact that pustulan turned out to be the most effective CLEC in the course of the present invention was unexpected, because various references show that Pustulan should be less effective in Dectin-1 binding (e.g. Adams et al., J Pharmacol Exp Ther. 2008 April; 325(1):115-23); in the literature, linear 1,3 and branched (1,3 main chain and 1,6 side branch) have been reported to be the most effective Dectin-1 binders. For example, Adams et al., 2008, have reported that murine recombinant Dectin-1 only recognized and interacted with polymers that contained a β-(1,3)-linked glucose backbone. Dectin-1 did not interact with a glucan that was exclusively composed of a β-(1,6)-glucose backbone (pustulan), nor did it interact with non-glucan carbohydrate polymers, such as mannan.

Therefore, according to a preferred embodiment of the present invention, the β-glucan of the present conjugate is a dectin-1 binding β-glucan. The ability of any compound, especially glucans, to bind to dectin-1 can easily be determined with the methods as disclosed herein, especially in the example section. In case of doubt, a “dectin-1 binding β-glucan” is a β-glucan which binds to the soluble murine Fc-dectin-1a receptor with an IC50 value lower than 10 mg/ml, as determined by a competitive ELISA, e.g. as disclosed in the examples.

Dectin-1 binding β-glucans according to the present invention (such as linear β-(1,6)-glucans) are advantageous compared to other glucans, e.g. DC-SIGN β-glucans (such as β-(1,2)-glucans), because with such dectin-1 binding glucans a broader range of DCs may be addressed (immature, mature, myeloid, plasmacytoid; in addition: APCs) which significantly increases the potential to elicit an effective immune response in vivo compared to non-dectin-1 binding glucans (immature DCs, myeloid DCS) which limits applicability.

WO 2022/060487 A1 and WO 2022/060488 A1 disclose conjugates linking peptide immunogens to an immunostimulatory polymer molecule (e.g. β-(1,2) glucans). β-(1,2) glucans including cyclic variants have previously been implied as potential adjuvants (Martirosyan A et al., doi: 10.1371/journal.ppat.1002983). They are a class of glucans which are predominantly binding to a specific PRR, DC-SIGN (Zhang H et al. doi: 10.1093/glycob/cww041), specifically binding to N-linked high-mannose oligosaccharides and branched fucosylated structures. Importantly, β-1,2 glucans fail to bind to dectin 1 (Zhang H et al., doi: 10.1093/glycob/cww041) thereby limiting their activity to DC-SIGN positive cells.

DC-SIGN (CD209) was the first SIGN molecule identified and found to be highly expressed on a restricted subset of DCs only, including immature (CD83-negative) DCs, as well as on specialized macrophages in the placenta and lung (Soilleux E J et al., doi: 10.1189/jlb.71.3.445). In the periphery, eg. in skin or at mucosal sites, expression and hence the potential to be biologically active as receptor according to this invention is only detectable in subsets of immature DCs. Mature, plasmacytoid DCs and other APCs like epithelial DC-like Langerhans cells do not express DC-SIGN (Engering A, et al., doi: 10.4049/jimmunol.168.5.2118)

In contrast thereto, the target receptor of the β-glucan based immunogens as provided in the present invention is dectin-1. Dectin-1 is expressed on a variety of different DC types, including not only immature DCs, myeloid DCs but also plasmacytoid DCs, which express dectin-1 in both mRNA and protein levels as well as DC-like Langerhans cells in the skin (Patente et al., doi: 10.3389/fimmu.2018.03176; Joo et al. doi: 10.4049/jimmunol.1402276).

Hence, biological activity of DC-SIGN targeting polymers like β-(1,2) glucans is limited to specific DC target cell populations whereas dectin-1 targeting polymers as applied in this present invention can exert their function in a variety of different additional DC-types. Therefore, these novel conjugates can exert a significantly different and superior immune response as compared to other conjugates. The prior art disclosure therefore does not suggest the claimed subject matter disclosed by the present invention.

According to a specifically preferred embodiment, the conjugates of the present invention comprise a strong dectin-1 binding β-glucan, preferably a β-glucan which binds to the soluble murine Fc-dectin-1a receptor with an IC50 value lower than 10 mg/ml, more preferred with an IC50 value lower than 1 mg/ml, even more preferred with an IC50 value lower than 500 μg/ml, especially with an IC50 value lower than 200 μg/ml, as determined by a competitive ELISA, e.g. as disclosed in the examples. Specifically preferred are conjugates which bind to the soluble murine Fc-dectin-1a receptor with an IC50 value lower than 1 mg/ml, more preferred with an IC50 value lower than 500 μg/ml, even more preferred with an IC50 value lower than 200 μg/ml, especially with an IC50 value lower than 100 μg/ml, as determined by a competitive ELISA; and/or—a β-glucan which binds to the soluble human Fc-dectin-1a receptor with an IC50 value lower than 10 mg/ml, more preferred with an IC50 value lower than 1 mg/ml, even more preferred with an IC50 value lower than 500 μg/ml, especially with an IC50 value lower than 200 μg/ml, as determined by a competitive ELISA; and/or—wherein the conjugates bind to the soluble human Fc-dectin-1a receptor with an IC50 value lower than 1 mg/ml, more preferred with an IC50 value lower than 500 μg/ml, even more preferred with an IC50 value lower than 200 μg/ml, especially with an IC50 value lower than 100 μg/ml, as determined by a competitive ELISA, e.g. as disclosed in the examples.

Moreover, the conjugates according to the present invention also showed a proportionally highly increased ratio of antibodies reacting to target polypeptide than to carrier molecules as in non-CLEC, especially non-pustulan containing vaccines. This significantly increases the specific focus of the antibody immune response to the target rather than the carrier which then results in an increased efficacy and specificity of the response.

The CLEC conjugation according to the present invention, especially to pustulan, also leads to increased affinity maturation (AM) towards target proteins (AM is increased strongly, whereas KLH/CRM conjugates only show limited AM upon repeated immunization).

In the field of vaccines, suitable vaccines have been disclosed with only B-cell epitopes or only T-cell epitopes. There are specific circumstances where it is appropriate and preferred to have vaccines with exclusively T-cell epitopes or exclusively B-cell epitopes. However, most of the vaccines on the market contain both kinds of epitopes, i.e. T-cell epitopes and B-cell epitopes.

For example, vaccines containing only B-cell epitopes are in most cases not very effective, even though they do lead to a detectable antibody immune response. In most cases, however, this immune response is usually much less effective compared to a vaccine containing B- and T-cell epitopes. This is also in line with the examples given in the example section of the present invention wherein a lower level of response was detectable.

On the other hand, vaccines which only contain T-cell epitopes (e.g. in vaccines where a specific T-cell response would be the active component of the response), are specifically interesting for certain applications, especially for cancer, where cancer specific cytotoxic T lymphocyte and T-helper cell epitopes or only CTL epitopes are combined with the vaccine platform according to the present invention. In this case a T-cell epitope with the CLEC polysaccharide adjuvant according to the present invention is provided with the T-cell epitope only. This is specifically preferred e.g. in cases where somatic mutations in cancers affect protein coding genes which can give rise to potentially therapeutic neoepitopes. These neoepitopes can guide adoptive cell therapies and peptide-(and RNA-based) neoepitope vaccines to selectively target tumor cells using autologous patient cytotoxic T-cells. This can be used according to the present invention for general antigens and for individualized neoantigen specific therapy (for example with NY-ESO-1, MAGE-A1, MAGE-A3, MAGE-C1, MAGE-C2, MAGE-C3, Survivin, gp100, tyrosinase, CT7, WT1, PSA, PSCA, PSMA, STEAP1, PAP, MUC1, 5 T4, KRAS, Her2, and others. Using a vaccine with only T-cell epitopes may also preferred with respect to specific autoimmune diseases. The treatment effect of the respective T-cell epitope only conjugate is associated with a reduction of effector T-cells and the development of regulatory T-cell (T_{reg}-cell) populations which leads to the dampening of the respective autoimmune disease (e.g.: multiple sclerosis or similar diseases).

Since most of the usual vaccine set-ups contain both, B-cell and T-cell epitopes, also the CLEC conjugates according to the present invention therefore preferably comprise both, individual B- and T-cell epitopes (at minimum: at least one B-cell epitope and at least one T-cell epitope) for a sustained B-cell immune response. However, a weak effect may demonstrate T-cell independent immunity if required.

The conjugates according to the present invention are therefore not limited with respect to possible vaccine antigens. It is, however, preferred that the vaccine antigens (i.e. B-cell and/or T-cell epitope polypeptides) have a length of 6 to 50 amino acid residues, preferably of 7 to 40 amino acid residues, especially of 8 to 30 amino acid residues.

A cross-linking of B-cell receptors is also possible using the vaccines according to the present invention. According to a specific embodiment, the conjugates according to the present invention are used for a T-cell independent immunization. T-cell independent responses are well known for polysaccharide vaccines. These vaccines/the polysaccharide produces an immune response by direct stimulation of B-cells, without the assistance of T-cells. The T-cell independent antibody response is short-lived. Antibody concentrations for pneumococcal capsule polysaccharides decline to baseline in typically 3-8 years, depending on serotype. Usually, additional doses cannot be used to enhance the vaccine response, as the polysaccharide vaccine does not constitute immunological memory. In children under two years of age, the polysaccharide vaccine is poorly immunogenic. Here the reason for direct stimulation could be that B-cells express a molecule called CR3 (complement receptor type 3). Macrophage-1 antigen or CR3 is a human cell surface receptor found on B- and T-lymphocytes, polymorphonuclear leukocytes (mostly neutrophils), NK cells, and mononuclear phagocytes like macrophages. CR3 also recognizes iC3b when bound to the surface of foreign cells and β-Glucan which means that direct uptake of the vaccine by B-cells via Pus-CR3 interaction could lead to the stimulation of the cells and the development of a low level TI immune response.

The adjuvants, conjugates and vaccines according to the present invention could fix complement and may be opsonized. Opsonized conjugates according to the present invention could have an increased B-cell activating ability which could lead to higher antibody titers and antibody affinity. This effect is known for C3d conjugates (Green et al., J. Virol. 77 (2003), 2046-2055) and is unexpectedly also useable in the course of the present invention.

Another unexpected advantage of the present invention is that the CLEC architecture of the present invention allows a modular design of the vaccine. For example, epitopes can be combined at will and the platform is independent from conventional carrier molecules. Although the major emphasis of the present invention is on peptide-only vaccines, it also works with independent coupling of proteins and peptides as well as with coupling of peptide-protein conjugates to the CLEC backbones according to the present invention, especially to pustulan. As shown in the example section with pustulan a significant superior immune response as compared to classical vaccines is obtained according to the present invention.

As already outlined above, the conjugates according to the present invention, if provided in a pharmaceutical preparation (e.g. as a vaccine intended to be administered to a (human) subject to elicit an immune response to a specific polypeptide epitope conjugated to the CLEC backbone, to which epitope the immune response should be elicited), can be administered without the need to use (by co-administration) a (further) adjuvant in this preparation. According to a preferred embodiment, the pharmaceutical formulation comprising the conjugate according to the present invention is free of adjuvants.

A specifically preferred class of CLEC polysaccharide adjuvants according to the present invention are β-glucans, especially pustulan. Another preferred CLEC polysaccharide adjuvant is mannan. In contrast to the present invention, pustulan has only been used in the prior art for anti-fungal vaccines (where pustulan was used as antigen and not as carrier as in the present invention). Pustulan is also displaying a different main chain as it only consists of β-(1,6)-linked sugar moieties.

Pustulan is a medium sized linear β-(1,6) glucan. Pustulan as well as synthetic forms of linear β(1,6) glucan are different from all other glucans used as β-glucans usually consist of branched glucan chains (preferably β-(1,3) main chains with 3-(1,6) side chains like yeast extracts, GPS, laminarin, schizophyllan, scleroglucan) or linear glucans only relying on β-(1,3) glucans like synthetic β-Glucan, curdlan, S. cerevisiae β-glucan (150 kDa) or linear β-(1,3:1,4) glucans like barley- and oat β-glucan as well as Lichenan.

As shown for the first time with the present invention, the binding of glucan conjugates to the dectin-1 receptor in vitro is a surrogate for subsequent in vivo efficacy: low binding molecules can only exert low immune responses, medium binders are better whereas highly efficient binders induce highly efficient responses (oat/barley BG<lichenan<pustulan).

According to the present invention, the CLECs are coupled (e.g. by standard techniques) to individual polypeptides to create small nanoparticles with low polydispersity (range of the hydrodynamic radius (HDR): 5-15 nm) which are not crosslinked and do not aggregate to form larger particulates similar to conventional CLEC vaccines such as glucan particles (2-4 μm) or β-glucan particles as disclosed in the literature, usually characterized by a size range of >100 nm (typical range (diameter; 150-500 nm, e.g. Wang et al. (2019) provide particles with a diameter of 160 nm (assessed by DLS) and a size of ca. 150 nm as assessed by TEM; Jin et al. (Acta Biomater. 2018 Sep. 15; 78:211-223) provide β-glucan particles (nanoparticles of aminated β-glucan-ovalbumin) with 180-215 nm size (as assessed by DLS and SEM, respectively).

By definition, the DLS measured hydrodynamic radius is the radius of a hypothetical hard sphere that diffuses with the same speed as the particle under examination. The radius is calculated from the diffusion coefficient assuming globular shape of your molecule/particle and a given viscosity of a buffer. The HDR is also called Stokes radius and is calculated from the diffusion coefficient using the Stokes-Einstein equation (see https://en.wikipedia.org/wiki/Stokes radius).

Preferred size ranges of the nanoparticles according to the present invention may be those typically provided in the prior art, i.e. with a size of 1 to 5000 nm, preferably of 1 to 200 nm, especially of 2 to 160 nm, determined as hydrodynamic radius (HDR) by dynamic light scattering (DLS). According to a preferred embodiment of the present invention, the particle size is smaller, e.g. from 1 to 50 nm, preferably from 1 to 25 nm, especially from 2 to 15 nm, determined as HDR by DLS. These preferred particles are therefore smaller, including the peptide only conjugates (about 5 nm average HDR) and CRM-pustulan conjugates (about 10-15 nm average HDR). Accordingly, preferred particles according to the present invention are smaller than 100 nm, this would separate us from Wang et al.

Accordingly, the present invention also relates to a vaccine product designed for vaccinating an individual against a specific antigen, wherein the product comprises a compound comprising a Bglucan or mannan as a C-type lectin (CLEC) polysaccharide adjuvant covalently coupled to the specific antigen.

Preferably, the vaccine product according to the present invention comprises a conjugate as disclosed herein or obtainable or obtained by a method according to the present invention.

According to a preferred embodiment, the vaccine product according to the present invention comprises an antigen comprising at least one B-cell epitope and at least one T-cell epitope, preferably wherein the antigen is a polypeptide comprising one or more B-cell and T-cell epitopes.

According to a preferred embodiment, the covalently coupled antigen and CLEC polysaccharide adjuvant in the vaccine product according to the present invention are present as particles with a size of 1 to 5000 nm, preferably of 1 to 200 nm, especially of 2 to 160 nm, determined as hydrodynamic radius (HDR) by dynamic light scattering (DLS). As used herein, all particle sizes are median particle sizes, wherein the median is the value separating the half of the particles with a higher size from the half of the particles with lower size. It is the determined particle size from which half of the particles are smaller and half are larger.

According to a preferred embodiment, the covalently coupled antigen and CLEC polysaccharide adjuvant in the vaccine product according to the present invention are present as particles with a size of 1 to 50 nm, preferably of 1 to 25 nm, especially of 2 to 15 nm, determined as HDR by DLS.

Preferably, the covalently coupled antigen and CLEC polysaccharide adjuvant in the vaccine product according to the present invention are present as particles with a size smaller than 100 nm, 50 nm, preferably smaller than 70 nm, especially smaller than 50 nm, determined as HDR by DLS.

The vaccine products according to the present invention show a high storage stability. Virtually no aggregation takes place upon storage as liquid or frozen material (storage temperature:80° C., −20° C., 2-8° C. or at room temperature over extended time periods, at least 3 months) as can be determined that the particle size does not significantly (i.e. more than 10%) increase over storage.

The extremely high efficacy of such small particles produced by using the medium molecular weight component pustulan according to the present invention is surprising: For example, according to Adams et al. (J Pharmacol Exp Ther. 2008 April; 325 (1): 115-23) the best dectin-1 substrates are linear β(1,3) glucan phosphate (ca. 150 kda) and branched glucans (containing a β(1,3) main chain and B (1,6) side chains) like Scleroglucans or glucans from C. albicans or Laminarin. In addition, the data of Adams et al., Palma et al. (J Biol Chem. 281(9) (2006) 5771-5779) and Willment et al. (J Biol Chem. 276(47) (2001), 43818-23) imply that dectin-1 does not or only weakly interact with pustulan, nor that it interacts with non-glucan carbohydrate polymers, such as mannan. In fact, various references report pustulan as being less effective in dectin-1 binding. In general, however, linear 1,3 and branched (1,3 main chain and 1,6 side branch) are the most effective dectin-1 binders; Adams et al. (2008) show that murine recombinant dectin-1 only recognized and interacted with polymers that contained a β(1,3)-linked glucose backbone. Dectin-1 did not interact with a glucan that was exclusively composed of a β(1,6)-glucose backbone (such as pustulan), nor did it interact with non-glucan carbohydrate polymers, such as mannan.

In contrast to these findings, it was shown in the course of the present invention that pustulan based conjugates are able to strongly bind to dectin-1 and to elicit cellular responses in vitro.

According to a preferred embodiment of the present invention, a β-(1,6)-glucan is used. Usually large particulates are reported in the prior art to be more effective in activating PRRs than small (“soluble”) monomeric formulations, so particles containing large glucans are superior (and therefore preferred) and small, soluble glucans can be used to block activation of DCs thereby interfering with the intended effect. It is well accepted that particulate β-glucans, such as the widely used yeast cell-wall fraction zymosan, bind to and activate dectin-1 thereby inducing cellular responses. In contrast, the interaction of soluble β-glucans with dectin-1 is subject to debate. The general consensus, though, is that soluble β-glucans, such as the small, branched glucan laminarin (β-(1,3) and β-(1,6) side chains), bind to dectin-1 but are unable to initiate signaling and induce cellular responses in the DCs (Willment et al., J Biol Chem. 276(47) (2001), 43818-23, Goodridge et al. Nature. 2011, 472(7344): 471-475.).

According to the present invention, it could be shown that conjugates using high mol. weight glucans (10x the size of pustulan; e.g.: oat/barley 229 kDa/lichenan 245 kDa) perform less effective than pustulan particles (20 kDa). Korotchenko et al. show that OVA/Lam conjugates have a ca 10 nm diameter, bind dectin-1 and induce DC activation in vitro but are branched glucans, not skin specific and regarding the effect in vivo not superior compared to OVA applied into the skin or OVA/alum applied s.c. Wang et al. provide β-glucan particles with >100 nm size (average size: 160 nm). Jin et al. (2018) show aminated β-glucan-ovalbumin nanoparticles with 180-215 nm size.

According to the present invention, it was shown that pustulan-based particles are strong dectin-1 binders, activate DCs in vitro (changes in surface marker expression) and elicit a very strong immune response, superior to a) other routes and b) comparable to KLH/CRM conjugate vaccines (usually also much bigger particles) and C) larger glucans and also mannan. This is true for Pep+Padre+pustulan (size of 5 nm) and for Pep+CRM+pustulan (size of 11 nm).

For optimal immune responses, the degree of activation of the CLEC, esp. pustulan, and the peptide/sugar ratio resulting from this degree of activation is decisive. Activation of the respective CLEC is achieved by mild periodate oxidation. Thus, the degree of oxidation is determined based on adding the periodate solution at a defined molar ratio: i.e. periodate: sugar subunit; 100%=1 Mol periodate per Mol sugar monomers.

According to a preferred embodiment, the conjugates according to the present invention comprise a CLEC activated with a ratio of periodate to β-glucan or mannan (monomer) moiety of ⅕ (i.e. 20% activation) to 2, 6/1 (i.e. 260% activation), preferably of 60% to 140%, especially 70% to 100%.

The optimal range of oxidation degree (which will be directly proportional to the number of epitope polypeptides in the final conjugate) between a low/middle oxidation degree and a high degree of oxidation can be defined as the reactivity with Schiff's fuchsin-reagent similar to that of an equal amount of the given carbohydrate (e.g. pustulan) oxidized with periodate at a molar ratio (sugar monomer:periodate) of 0.2-0.6 (low/middle), 0.6-1.4 (optimal range) and 1.4-2.6 (high), respectively.

Preferred glucan to peptide ratios are ranging from 10 to 1 (w/w) to 1 to 1 (w/w), preferably 8 to 1 (w/w) to 2 to 1 (w/w), especially 4 to 1 (w/w); i.e. 24 to 1 molar ratio of sugar monomer to peptide, with the proviso if the conjugate comprises a carrier protein, the preferred ratio of β-glucan or mannan to B-cell-epitope-carrier polypeptide is from 50:1 (w/w) to 0.1:1 (w/w), especially 10:1 to 0.1:1; which are lower than effective vaccines reported elsewhere (e.g. Liang et al., Bromuro et al.).

The degree of oxidation and the amount of reactive aldehydes available for coupling of the sugar is determined using state of the art methods like: 1) gravimetric measurement allowing for determination of the total mass of the sample; 2) the anthrone method (according to Laurentin et al. 2003)—for concentration determination of intact, non-oxidized sugars in the sample; in this case glucans are dehydrated with concentrated H₂SO₄ to form Furfural, which condenses with anthrone (0.2% in H₂SO₄) to form a green color complex which can be measured colorimetrically at 620 nm) or 3) Schiff's assay: Oxidation status of carbohydrates used for conjugation is assessed using Schiff's fuchsin-sulfite reagent. Briefly, fuchsin dye is decolorized by Sulphur dioxide. Reaction with aliphatic aldehydes (on Glucan) restores the purple color of fuchsin, which can then be measured at 570-600 nm. Resulting color reaction is proportional to the oxidation degree (the amount of aldehyde groups) of the carbohydrate. Other suitable analytical methods are possible as well. Peptide ratios can be assessed using suitable methods including UV analysis (205 nm/280 nm) and amino acid analysis (aa hydrolysis, derivatization and RP-HPLC analysis).

The conjugates according to the present invention can further be used for the induction of target specific immune responses while inducing no or only very limited CLEC- or carrier-protein specific antibody responses. As also shown in the example section below, the present invention also enables an improvement and focusing to the target-specific immune response because it triggers the immune response away from reactions to the carrier protein or the CLEC (as e.g. in conventional peptide-carrier conjugates or non-conjugated comparative set-ups, especially also applying non-oxidised CLECs, such as pustulan).

Unless indicated to the contrary, “peptides” as used herein refer to shorter polypeptide chains (of 2 to 50 amino acid residues) whereas “proteins” refer to longer polypeptide chains (of more than 50 amino acid residues). Both are referred to as “polypeptides”. The B-cell and/or T-cell epitope polypeptides conjugated to the CLECs according to the present invention comprise besides the polypeptides with the naturally used amino acid residues of normal gene expression and protein translation also all other forms of such polypeptide-based B-cell and/or T-cell epitopes, especially naturally or artificially modified forms thereof, such as glycopolypeptides und all other post-translationally modified forms thereof (e.g. the pyro-Glu forms of Aβ as disclosed in the examples). Moreover, the CLECs according to the present invention are specifically suitable for presenting conformational epitopes, for example conformational epitopes which are part of larger native polypeptides, mimotopes, cyclic polypeptides or surface-bound constructs.

According to a preferred embodiment, the conjugate according to the present invention comprises a CLEC polysaccharide backbone and a B-cell epitope. A “B-cell epitope” is the part of the antigen that immunoglobulin or antibodies bind. B-cell epitopes can be divided into two groups: conformational or linear. There are two main methods of epitope mapping: either structural or functional studies. Methods for structurally mapping epitopes include X-ray crystallography, nuclear magnetic resonance, and electron microscopy. Methods for functionally mapping epitopes often use binding assays such as western blot, dot blot, and/or ELISA to determine antibody binding. Competition methods determine if two monoclonal antibodies (mAbs) can bind to an antigen at the same time or compete with each other to bind at the same site. Another technique involves high-throughput mutagenesis, an epitope mapping strategy developed to improve rapid mapping of conformational epitopes on structurally complex proteins. Mutagenesis uses randomly/site-directed mutations at individual residues to map epitopes. B-cell epitope mapping can be used for the development of antibody therapeutics, peptide-based vaccines, and immunodiagnostic tools (Sanchez-Trincado et al., J. Immunol. Res. 2017-2680160). For many antigens, B-cell epitopes are known and may be used in the present CLEC platform.

According to a specifically preferred embodiment, the conjugate according to the present invention comprises a CLEC polysaccharide backbone and one or more T-cell epitopes, preferably comprises a promiscuous T-cell epitope and/or a MHCII epitope which are known to work with several/al MHC alleles of a given species as well as in other species.

According to another aspect, the present invention also relates to the use of the present CLEC technology to improve known T-cell epitopes. Accordingly, the present invention also encompasses a β-glucan or mannan for use as a C-type lectin (CLEC) polysaccharide adjuvant for T-cell epitope polypeptides, wherein the β-glucan or mannan is covalently conjugated to the T-cell epitope polypeptide to form a conjugate of the β-glucan or mannan and the T-cell epitope polypeptide.

A single T-cell epitope which binds to more than one HLA allele is referred to as “promiscuous T-cell epitope”. Preferred promiscuous T-cell epitopes bind to 5 or more, preferably 10 or more, especially 15 or more, HLA alleles. Promiscuous T-cell epitopes are suitable for different species and most importantly for several MHC/HLA haplotypes (referring to both, MHCI and MHCII epitopes which are known to work with several/all MHC alleles) of a given species as well as in other species. For example, the MHCII epitope PADRE (=nonnatural pan DR epitope (PADRE)), as referred to in the example section, works in several human MHC alleles and in mouse (C57/B16, although it is less effective in Balb/c). For example, the MHCII epitope PADRE (=nonnatural pan DR epitope (PADRE)), as referred to in the example section, works in several human MHC alleles and in mouse (C57/B16, although it is less effective in Balb/c). According to a preferred embodiment, the conjugate of the present invention comprises a T-cell epitope, preferably a T-cell epitope comprising the amino acid sequence AKFVAAWTLKAAA (“PADRE (polypeptide)”) or a PADRE (polypeptide) variant.

Preferred PADRE polypeptides or PADRE polypeptide variants include a linker (as also preferred for other polypeptides epitopes used herein), such as a cysteine residue or a linker comprising a cysteine reside (“—C” or “C—”; specifically for maleimide coupling), an NRRA, NRRA-C or NRRA-NH—NH₂ linker. Preferred PADRE polypeptide variants include the variants disclosed in the prior art (e.g. in Alexander et al., Immunity 1 (1994), 751-761; U.S. Pat. No. 9,249,187 B2, or), preferably a shortened variant without the C-terminal A residue (AKFVAAWTLKAA), variants wherein the first residue alanine is replaced by an aliphatic amino acid residue (e.g. glycine, valine, isoleucine and leucine), variants wherein the third residue phenylalanine is replaced with L-cyclohexylalanine, variants wherein the thirteenth (last) amino acid residue alanine is replaced by an aliphatic amino acid residue (e.g. glycine, valine, isoleucine and leucine), variants comprising aminocaproic acid, preferably coupled to the C-terminus of the PADRE variant, or variants with the amino acid sequence AX₁FVAAX₂TLX₃AX₄A, wherein X₁ is selected from the group consisting of W, F, Y, H, D, E, N, Q, I, and K; X₂ is selected from the group consisting of F, N, Y, and W, X₃ is selected from the group consisting of H and K, and X₄ is selected from the group consisting of A, D, and E (with the proviso that the oligopeptide sequence is not AKFVAAWTLKAAA; U.S. Pat. No. 9,249,187 B2); especially wherein the T-cell epitope is selected from AKFVAAWTLKAAANRRA-(NH—NH₂), AKFVAAWTLKAAAN-C, AKFVAAWTLKAAAC, AKFVAAWTLKAAANRRA-C, aKXVAAWTLKAAaZC, aKXVAAWTLKAAaZCNRRA (SeqID7, 8, 87, 88, 89, 90, 91, 92), aKXVAAWTLKAAa, aKXVAAWTLKAAaNRRA, aA(X)AAAKTAAAAa, aA(X)AAATLKAAa, aA(X)VAAATLKAAa, aA(X)IAAATLKAAa, aK(X)VAAWTLKAAa, and aKFVAAWTLKAAa (sequences 760.5, 760.57, 906.09, 906.11, 965.10, 1024.03 from Alexander et al., 1994), wherein X is L-cyclohexylalanine, Z is aminocaproic acid and a is an aliphatic amino acid residue selected from alanine, glycine, valine, isoleucine and leucine.

T-cell epitopes are presented on the surface of an antigen-presenting cell, where they are bound to major histocompatibility complex (MHC) molecules. In humans, professional antigen-presenting cells are specialized to present MHC class II peptides, whereas most nucleated somatic cells present MHC class I peptides. T-cell epitopes presented by MHC class I molecules are typically peptides between 8 and 11 amino acids in length, whereas MHC class II molecules present longer peptides, 13-17 amino acids in length; non-classical MHC molecules also present non-peptidic epitopes such as glycolipids. MHC class I and II epitopes can be reliably predicted by computational means alone, although not all in-silico T-cell epitope prediction algorithms are equivalent in their accuracy. There are two main methods of predicting peptide-MHC binding: data-driven and structure-based. Structure based methods model the peptide-MHC structure and require great computational power. Data-driven methods have higher predictive performance than structure-based methods. Data-driven methods predict peptide-MHC binding based on peptide sequences that bind MHC molecules (Sanchez-Trincado et al., 2017). By identifying T-cell epitopes, scientists can track, phenotype, and stimulate T-cells. For many antigens, T-cell epitopes are known and may be used in the present CLEC platform.

Interestingly, recent breakthrough studies have demonstrated that alpha-synuclein-specific T-cells are increased in PD patients, probably in association with risk haplotypes of HLA, and suggest an autoimmune involvement of T-cells in PD [Sulzer et al., Nature 2017; 546:656-661 and Lindestamn Arlehamn et al., Nat Commun. 1875; 2020:11]. A causal role of alpha-synuclein reactive T-cells was recently reinforced also by an animal model study [Williams et al., Brain. 2021; 144:2047-2059). The occurrence of alpha-synuclein-reactive T-cells was increased years before motor onset in a case study and their frequency was highest around and shortly after motor onset in a larger cross-sectional cohort of PD patients (Lindestam Arlehamn et al.). After motor onset, the T-cell response to alpha-synuclein declined with increasing disease duration. Thus, anti aSyn T-cell responses are highest before or shortly after diagnosis of motor PD and wane thereafter (i.e. maximum activity detectable less than 10 years after diagnosis; and Hoehn and Yahr (H+Y) stages 1 and 2 are preferred) (Lindestamn Arlehamn et al. 2020).

Accordingly, there are commonly known T-cell epitopes contained within the sequence of human alpha synuclein. Examples are provided in Benner et al. (PLOS ONE 3(1): e1376.60), Sulzer et al., (2017) and Lindestam Arlehamn et al. (2020).

Benner et al (Benner et al., (2008) PLOS ONE 3(1): e1376.) use a 60 aa long nitrated (at Y-residues) polypeptide comprising the C-terminal part of aSyn emulsified in an equal volume of CFA containing 1 mg/ml Mycobacterium tuberculosis as immunogen in a PD model and disclose the alpha synuclein T-cell epitope aa71-86 (VTGVTAVAQKTVEGAGNIAAATGFVK).

Sulzer et al. (Nature 2017; 546:656-661) identified two T-cell antigenic regions at the N-terminal and C-terminal regions in alpha synuclein in human PD patients. The first region is located near the N terminus, composed of the MHCII epitopes aa31-45 (GKTKEGVLYVGSKTK) and aa32-46 (KTKEGVLYVGSKTKE) also containing the 9 mer polypeptide aa37-45 (VLYVGSKTK) as potential MHCI class epitope. The second antigenic region disclosed by Sulzer et al. is near the C terminus (aa116-140) and required phosphorylation of amino acid residue S129. The three phosphorylated aas129 epitopes aa116-130 (MPVDPDNEAYEMPSE), aa121-135 (DNEAYEMPSEEGYQD), and aa126-140 (EMPSEEGYQDYEPEA) produced markedly higher responses in PD patients than in healthy controls. The authors also demonstrate that the naturally occurring immune responses to alpha synuclein associated with PD have both MHC class I and II restricted components.

In addition, Lindestam Arlehamn et al. (Nat Commun. 1875; 2020:11) also disclose the alpha synuclein peptide aa61-75 (EQVTNVGGAVVTGVT) as T-cell epitope (MHCII) in PD patients.

Accordingly, preferred T-cell epitopes according to the present invention include the alpha synuclein polypeptides GKTKEGVLYVGSKTK (aa31-45), KTKEGVLYVGSKTKE (aa32-46), EQVTNVGGAVVTGVT (aa61-75), VTGVTAVAQKTVEGAGNIAAATGFVK (aa71-86), DPDNEAYEMPSE (aa116-130), DNEAYEMPSEEGYQD (aa121-135), and EMPSEEGYQDYEPEA (aa126-140).

The regulatory T-cells (“Treg cells” or “Tregs”) are a subpopulation of T-cells that modulate the immune system, maintain tolerance to self-antigens, and prevent autoimmune disease. Treg cells are immunosuppressive and generally suppress or downregulate induction and proliferation of effector T-cells. Tregs produced by a normal thymus are termed “natural”. The selection of natural Tregs occurs on radio-resistant haematopoietically-derived MHC class II-expressing cells in the medulla or Hassal's corpuscles in the thymus. The process of Treg selection is determined by the affinity of interaction with the self-peptide MHC complex. Selection to become a Treg is a “Goldilocks” process—i.e. not too high, not too low, but just right, a T-cell that receives very strong signals will undergo apoptotic death; a cell that receives a weak signal will survive and be selected to become an effector cell. If a T-cell receives an intermediate signal, then it will become a regulatory cell. Due to the stochastic nature of the process of T-cell activation, all T-cell populations with a given TCR will end up with a mixture of Teff and Treg—the relative proportions determined by the affinities of the T-cell for the self-peptide-MHC. Treg formed by differentiation of naïve T-cells outside the thymus, i.e. the periphery, or in cell culture are called “adaptive” or “induced” (i.e. iTregs).

Natural Treg are characterised as expressing both the CD4 T-cell co-receptor and CD25, which is a component of the IL-2 receptor. Treg are thus CD4+ CD25+. Expression of the nuclear transcription factor Forkhead box P3 (FoxP3) is the defining property which determines natural Treg development and function. Tregs suppress activation, proliferation and cytokine production of CD4+ T-cells and CD8+ T-cells, and are thought to suppress B-cells and dendritic cells thereby dampening autoimmune reactions.

Along these lines several studies indicate that Treg number and function is reduced in PD patients. E.g: Hutter Saunders et al. (J Neuroimmune Pharmacol (2012) 7:927-938) and Chen et al. (MOLECULAR MEDICINE REPORTS 12:6105-6111, 2015) show impaired abilities of regulatory T-cells (Treg) from PD patients to suppress effector T-cell function and that the proportion of Th1 and Th17 cells was increased, while that of Th2 and Treg cells was decreased. Thome et al. (npj Parkinson's Disease (2021) 7:41) showed that declining PD Treg function correlates with increasing proinflammatory T-cell activation which can directly result in the subsequent increase in pro-inflammatory signaling by other immune cell populations. Treg suppression of T-cell proliferation significantly correlated with peripheral pro-inflammatory immune cell phenotypes. The suppressive capacity of PD Tregs on T-effector cells (e.g.: CD4+) proliferation decreased with increasing PD disease burden using the H&Y disease scale with highest activity at stages H+Y 1 and 2. Importantly, Lindestam Arlehamn et al. (2020) showed that anti aSyn T-cell responses are highest before or shortly after diagnosis of motor PD and wane thereafter (i.e. maximum activity detectable less than 10 years after diagnosis; and Hoehn and Yahr (H+Y) stages 1 and 2 are preferred) (Lindestamn Arlehamn et al., 2020).

Thus, the combination of the vaccines according to the present invention with
1) vaccines containing an alpha synuclein specific Treg epitope (e.g. a CD4 epitope like those disclosed by Brenner et al, Sulzer et al. and Lindestam Arlehamn et al. (aa31-45 (GKTKEGVLYVGSKTK), aa32-46 (KTKEGVLYVGSKTKE), aa61-75 (EQVTNVGGAVVTGVT), aa71-86 (VTGVTAVAQKTVEGAGNIAAATGFVK), aa116-130 (MPVDPDNEAYEMPSE), aa121-135 (DNEAYEMPSEEGYQD), and aa126-140 (EMPSEEGYQDYEPEA));
and/or
2) with Treg inducing agents like rapamycin, low-dose IL-2, TNF receptor 2 (TNFR2) agonist, anti-CD20 antibodies (e.g.: rituximab), prednisolone, inosine pranobex, glatiramer acetate, sodium butyrate
is preferred at early stages of the disease (i.e. less than 10 years after diagnosis; and Hoehn and Yahr stages 1 and 2 are preferred) to augment waning/reduced Treg number and activity and thereby reduce autoimmune reactivity of aSyn specific T-effector cells and dampen autoimmune responses in PD patients.

More to this, Tregs are found to be decreased and/or dysfunctional in a number of diseases, especially chronic degenerative or autoimmune diseases such as (active) systemic lupus erythematosus (SLE, aSLE), type 1 diabetes (T1D), autoimmune diabetes (AID), multiple sclerosis (MS), amyotrophic lateral sclerosis (ALS), and Alzheimer's disease (AD) among other degenerative diseases (ALS: Beers et al., JCI Insight 2, e89530 (2017); AD: Faridar et al., Brain Commun. 2, fcaa112 (2020); ALS: Beers et al., JAMA Neurol. 75, 656-658 (2018); MS: Haas et al., Eur. J. Immunol. 35, 3343-3352 (2005); T1D: Lindley et al., Diabetes 54, 92-99 (2005): AID: Putnam et al., J. Autoimmun. 24, 55-62 (2005); autoimmune diseases: Ryba-Stanislawowska et al., Expert Rev. Clin. Immunol. 15, 777-789 (2019); aSLE: Valencia et al., J. Immunol. 178, 2579-2588 (2007); MS: Viglietta et al., J. Exp. Med. 199, 971-979 (2004); SLE: Zhang et al., Clin. Exp. Immunol. 153, 182-187 (2008); AD+MS: Ciccocioppo et al., Sci. Rep. 9, 8788 (2019)).

It is therefore also preferred to provide T-cell epitopes suitable as Treg epitopes or Treg inducing agents in diseases with reduced or dysfunctional Treg populations as a combination with the vaccines according to present invention to augment waning/reduced Treg number and activity and thereby reduce autoimmune reactivity of disease specific T-effector cells and dampen autoimmune responses in patients. Whereas suitable Treg epitopes are defined as self MHC epitopes (MHCII type) which are characterized by the ability to induce intermediate signals during T-cell selection processes.

According to a preferred embodiment, the conjugate according to the present invention comprises a polypeptide comprising or consisting of the amino acid sequences SeqID7, 8, 22-29, 87-131, GKTKEGVLYVGSKTK, KTKEGVLYVGSKTKE, EQVTNVGGAVVTGVT, VTGVTAVAQKTVEGAGNIAAATGFVK, MPVDPDNEAYEMPSE), DNEAYEMPSEEGYQD, EMPSEEGYQDYEPEA, or combinations thereof.

Preferred T-cell epitopes are therefore:

| | | |
|---|---|---|
| SeqID7 | AKFVAAWTLKAAANRRA-(NH-NH2) | PADRE |
| SeqID8 | AKFVAAWTLKAAAN-C | PADRE |
| SeqID22 | AKFVAAWTLKAAA-(NH-NH2) | PADRE-original |
| SeqID23 | KAAAVKAAFWTAL-NRRA-(NH-NH2) | alternative synthetic peptide |
| SeqID24 | DSETADNLEKTVAALSILPGHGC-(NH- | Toxin Diphtheria (TV-TT ex- |
| | NH2) | change) |
| SeqID25 | DSETADNLEKTVAALSILPGHGCNRRA- | Toxin Diphtheria (TV-TT ex- |
| | (NH-NH2) | change) |
| SeqID26 | ISITEIKGVIVHRIETILF-(NH-NH2) | MvF5 Th (UBITh®1) |
| SeqID27 | ISITEIKGVIVHRIETILENRRA-(NH- | MvF5 Th (UBITh®1) |
| | NH2) | |
| SeqID28 | ISQAVHAAHAEINEAGR-(NH-NH2) | chicken Ova (323-339) |
| SeqID29 | ISQAVHAAHAEINEAGRNRRA-(NH- | chicken Ova (323-339) |
| | NH2) | |
| SeqID87 | AKFVAAWTLKAAA-C | Padre (original) for Malei- |
| | | midecoupling |
| SeqID88 | AKFVAAWTLKAAANRRA-C | Tridem' Padre (original) for |
| | | Maleimidecoupling |
| SeqID89 | aKXVAAWTLKAAaZC | PADRE, alternative aas |
| SeqID90 | aKXVAAWTLKAAaZCNRRA | PADRE, alternative aas |
| SeqID91 | aKXVAAWTLKAAa | PADRE, alternative aas |
| SeqID92 | aKXVAAWTLKAAaNRRA | PADRE, alternative aas |
| SeqID93 | DSETADNLEKTTAALSILPG | Diphteria |
| SeqID94 | DSETADNLEKTTAALSILPGNRRA | Diphteria |
| SeqID95 | LSEIKGVIVHRLEGV | MvF |
| SeqID96 | LSEIKGVIVHRLEGVNRRA | MvF |
| SeqID97 | KLLSLIKGVIVHRLEGVE | MvF |
| SeqID98 | KLLSLIKGVIVHRLEGVENRRA | MvF |
| SeqID99 | VSIDKFRIFCKANPK | P23-TT |
| Se- | LKFIIKRYTPNNEIDS | P32-TT |
| qID100 | | |
| Se- | IREDNNTLKLDRCNN | P21-TT |
| qID101 | | |
| Se- | FNNFTVSFWLRVPKVSASHLE | P30-TT |
| qID102 | | |
| Se- | QYIKANSKFIGITE | P2-TT |
| qID103 | | |
| Se- | LEYIPEITLPVIAALSIAES | TT |
| qID104 | | |
| Se- | LINSTKIYSY FPSVISKVNQ | TT |
| qID105 | | |
| Se- | NYSLDKIIVDYNLQSKITLP | TT |
| qID106 | | |
| Se- | PHHTALRQAILCWGELMTLA | HBV nuclear capsid |
| qID107 | | |
| Se- | FELLTRILTIPQSLD | HBV surface AG |
| qID108 | | |
| Se- | YSGPLKAEIAQRLEDV | MT Influenza matrix epitope |
| qID109 | | |
| Se- | FELLTRILTIPQSL | HBsAg |
| qID110 | | |
| Se- | GAYARCPNGTRALTVAELRGNAEL | Bordetella pertussis |
| qID111 | | |
| Se- | ALNIWDRFDVFCTLGATTGYLKGNS | Cholera Toxin |
| qID112 | | |
| Se- | QYIKANSKFIGITEL | Clostridium tetani TT1 |
| qID113 | | |
| Se- | FNNFTVSFWLRVPKVSASHLE | Clostridium tetani TT2 |
| qID114 | | |
| Se- | KFIIKRYTPNNEIDSE | Clostridium tetani TT3 |
| qID115 | | |
| Se- | VSIDKFRIFCKALNPK | Clostridium tetani TT4 |
| qID116 | | |
| Se- | WVRDIIDDFTNESSQKT | Clostridium tetani2 |
| qID117 | | |
| Se- | AGLTLSLLVICSYLFISRG | EBV BHRF1 |
| qID118 | | |
| Se- | PGPLRESIVCYEMVELQTHI | EBV EBNA-1 |
| qID119 | | |
| Se- | VPGLYSPCRAFENKEELL | EBV CP |
| qID120 | | |
| Se- | TGHGARTSTEPTTDY | EBV GP340 |
| qID121 | | |
| Se- | KELKRQYEKKLRQ | EBV BPLF1 |
| qID122 | | |
| Se- | TVFYNIPPMPL | EBV EBNA-2 |
| qID123 | | |
| Se- | DKREMWMACIKELH | HCMV IE1 |
| qID124 | | |
| Se- | FVFTLTVPSER | Influenza MP1-1 |
| qID125 | | |
| Se- | PKYVKQNTLKLAT | Influenza Hemagluttinin |
| qID126 | | |
| Se- | EKKIAKMEKASSVENV | Malaria T3 epitope |
| qID127 | | |
| Se- | FELLTRILTI | Hepatitis B surface antigen |
| qID128 | | |
| Se- | DQSIGDLIAEAMDKVGNEG | Heat Shock Protein 65 |
| qID129 | | |
| Se- | QVHFQPLPPAVVKL | bacille CalmetteGuerin |
| qID130 | | |
| Se- | KQIINMWQEVGKAMYA | HIV gp120 |
| qID131 | | |

wherein X is L-cyclohexylalanine, Z is aminocaproic acid and a is an aliphatic amino acid selected from alanine, glycine, valine, isoleucine and leucine.

According to another preferred embodiment, the conjugate according to the present invention comprises a B-cell epitope and a T-cell epitope, preferably a pan-specific/promiscuous T-cell epitope, independently coupled to the CLEC polysaccharide backbone according to the present invention, especially to pustulan.

According to another preferred embodiment, the conjugate according to the present invention comprises a B-cell epitope coupled to a “classic” carrier protein, such as CRM197, wherein this construct is further coupled to a CLEC carrier according to the present invention, especially to pustulan.

For example, in a first step, CRM conjugate formation may be performed by activation of CRM via GMBS or sulfo-GMBS etc.; then the maleimide-groups of the activated CRM are reacted with SH groups of the peptide (cysteine). CRM conjugates are then treated with DTT to reduce disulphide bonds and generate SH-groups on cysteins. Subsequently, a one pot reaction mixing reduced CRM-conjugate with BMPH (N-β-maleimid-propionic acid hydrazide) and activated pustulan (oxidised) may be done to create the CLEC-based vaccine. The mechanism in the one pot reaction may be (with respect to pustulan) that oxidised pustulan is reacted with BMPH (has the hydrazide residues) and to form a BMPH-hydrazone. The reduced CRM conjugate is then reacting via SH groups on CRM-conjugate with the maleimide of the BMPH activated pustulan.

According to another preferred embodiment, the conjugates according to the present invention comprise a “classical” carrier protein, such as CRM197, containing multiple T-cell epitopes. The conjugate according to the present invention also comprises a B-cell epitope covalently coupled to the polysaccharide moiety. In this embodiment, both polypeptides (B-cell epitope and carrier molecule) are coupled independently to a CLEC carrier according to the present invention, especially to pustulan.

According to another preferred embodiment, the conjugates according to the present invention also comprise a “classical” carrier protein, such as CRM197, containing multiple T-cell epitopes. The conjugate according to the present invention also comprises a B-cell epitope covalently coupled to the “classical” carrier protein. The peptide-carrier conjugate according to the present invention is also covalently coupled to the polysaccharide moiety. In this embodiment, both polypeptides (B-cell epitope and carrier molecule) are coupled as one conjugate to a CLEC carrier according to the present invention, especially to pustulan. The carrier protein then represents a link between the β-glucan or mannan and the B-cell and/or T-cell epitope polypeptide(s) in the conjugate according to the present invention. The covalent conjugation between the β-glucan or mannan and the B-cell and/or T-cell epitope polypeptides is then made by the carrier protein (as a functional linking moiety).

Preferred conjugates according to the present invention may comprise a B-cell epitope coupled to CRM197, wherein this construct is further coupled to a CLEC polymer according to the present invention especially to a β-glucan wherein the β-glucan is pustulan, lichenan, laminarin, curdlan, β-glucan peptide (BGP), schizophyllan, scleroglucan, whole glucan particles (WGP), zymosan, or lentinan, preferably pustulan, laminarin, lichenan, lentinan, schizophyllan, or scleroglucan, especially pustulan.

According to the present invention, it was shown that novel B-cell epitope-CRM197 conjugates coupled to pustulan are strong dectin-1 binders and elicit a very strong immune response, superior to conventional CRM conjugate vaccines.

According to the present invention it was shown that CLEC conjugation to novel B-cell epitope-CRM197 conjugates, especially creating B-cell epitope-CRM197-glucan, more preferably B-cell epitope-CRM197-linear β-(1,6)-glucan or B-cell epitope-CRM197-pustulan conjugates is indispensable for induction of the superior immunogenicity described for various peptide-CRM197-CLEC, especially peptide-CRM197-β-glucan, more preferably peptide-CRM197-linear β-(1,6)-glucan or peptide-CRM197-linear pustulan conjugates as compared to conventional CRM conjugate vaccines with or without adjuvantation by mixing with β-glucan/pustulan.

According to a preferred embodiment of the present invention, the CLEC conjugates according to the present invention comprise oligo-/polysaccharides as B-cell epitope(s) coupled to a carrier protein as source of T-cell epitopes (e.g.: CRM197, KLH, diphtheria toxoid (DT), tetanus toxoid (TT), Haemophilus influenzae protein D (HipD), and the outer membrane protein com-plex of serogroup B meningococcus (OMPC), recombinant non-toxic form of Pseudomonas aeruginosa exotoxin A (rEPA), flagellin, Escherichia coli heat labile enterotoxin (LT), cholera toxin (CT), mutant toxins (e.g., LTK63 and LTR72)) wherein this construct is further coupled to a CLEC polymer according to the present invention, especially to a β-glucan wherein the β-glucan is pustulan, lichenan, laminarin, curdlan, β-glucan peptide (BGP), schizophyllan, scleroglucan, whole glucan particles (WGP), zymosan, or lentinan, preferably pustulan, laminarin, lichenan, lentinan, schizophyllan, or scleroglucan, especially pustulan. If the conjugate comprises a carrier protein, it is a preferred embodiment of the present invention that the conjugate according to the present invention comprises at least a further, independently conjugated T-cell or B-cell epitope. This preferred embodiment further clarifies that the present invention is not about eliciting specific antibodies against the predominantly linear β-(1,6)-glucan with a ratio of (1,6)-coupled monosaccharide moieties to non-β-(1,6)-coupled monosaccharide moieties of at least 1:1, such as pustulan. Therefore, conjugated containing the predominantly linear β-(1,6)-glucan with a ratio of (1,6)-coupled monosaccharide moieties to non-β-(1,6)coupled monosaccharide moieties of at least 1:1 which only contain the saccharide as antigen and the carrier protein are excluded from the present invention, because the conjugates according to the present invention significantly reduce or eliminate the induction of a strong de novo immune responses directed against the glucan backbone in vivo, if the conjugate contains an additional T-cell or B-cell epitope (see e.g. Example 7 and FIG. 7, below). In contrast, repeated application of unconjugated glucan (or glucan conjugated only to a carrier protein) leads to the induction of a strong anti-glucan immune response by boosting antibody levels against the glucan polysaccharide. This shows that it is necessary for the conjugates according to the present invention to have a further T-cell or B-cell epitope polypeptide being covalently conjugated to the conjugate of the predominantly linear β-(1,6)-glucan with the carrier protein. This also explains that the conjugates according to the present invention are not encompassing the prevention or treatment of diseases caused directly or indirectly by fungi, especially by C. albicans, by providing the predominantly linear β-(1,6)-glucan with a ratio of (1,6)-coupled monosaccharide moieties to non-β-(1,6)-coupled monosaccharide moieties of at least 1:1 as an antigen (eventually coupled to a carrier protein).

According to the present invention, it is shown that such oligo-/polysaccharide conjugate vaccines coupled to pustulan are strong dectin-1 binders and elicit a beneficial/efficient immune response if applied in vivo.

Accordingly, the present invention also relates to the improvement and/or optimisation of carrier proteins by covalently coupling the carrier protein (already containing one or more T-cell antigens (as part of its polypeptide sequence, optionally in post-translationally-modified form)) to the CLEC polysaccharide adjuvant according to the present invention, i.e. the β-glucan or mannan, preferably to pustulan, lichenan, laminarin, curdlan, β-glucan peptide (BGP), schizophyllan, scleroglucan, whole glucan particles (WGP), zymosan, or lentinan. The present invention therefore relates to a β-glucan or mannan for use as a C-type lectin (CLEC) polysaccharide adjuvant for B-cell and/or T-cell epitope polypeptides, wherein the β-glucan or mannan is covalently conjugated to the B-cell and/or T-cell epitope polypeptide to form a conjugate of the β-glucan or mannan and the B-cell and/or T-cell epitope polypeptide, wherein a carrier protein is covalently coupled to the β-glucan or mannan.

This improvement/optimization leads to a significant reduction or elimination of the B-cell response to the CLEC and/or to the carrier protein and/or an enhancement (or at least preservation) of the T-cell response to the T-cell epitopes of the carrier protein. This enables a reduction or elimination of an antibody-response to the CLEC and/or the carrier (which then only delivers a T-cell response) and a specific enhancement of the antibody-response to the actual target polypeptide which is conjugated to the carrier and/or the CLEC.

Accordingly, a specifically preferred embodiment of the present invention is a conjugate consisting of or comprising (a) a β-glucan
(b) at least a B-cell or T-cell epitope polypeptide, and
(c) a carrier protein, wherein the three components (a), (b) and (c) are covalently conjugated with each other.

This combination of these three components can be provided in any orientation or sequence, i.e. in the sequence (a)-(b)-(c), (a)-(c)-(b) or (b)-(a)-(c), wherein (b) and/or (c) can be covalently conjugated either from the N-terminus to the C-terminus or from the C-Terminus to the N-terminus or conjugated via a functional group within the polypeptide (e.g. via a functional group in a lysine, arginine, aspartic acid, glutamic acid, asparagine, glutamine, serine, threonine, tyrosine, tryptophan or histidine residue, especially via the ε-ammonium group of a lysine residue). Of course, the β-glucan can be coupled to one or more of each of the components (b) and (c), preferably by the methods disclosed herein. Preferably, these components are conjugated by linkers, especially by linkers between all at least three components. Preferred linkers are disclosed herein, such as a cysteine residue or a linker comprising a cysteine or glycine residue, a linker resulting from hydrazide-mediated coupling, from coupling via heterobifunctional linkers, such as BMPH, MPBH, EMCH or KMUH, from imidazole mediated coupling, from reductive amination, from carbodiimide coupling a —NH—NH₂ linker; an NRRA, NRRA-C or NRRA-NH—NH₂ linker, peptidic linkers, such as bi-, tri-, tetra-(or longer)-meric peptide groups, such as CG or CG. In the case of established carrier proteins, especially CRM, CRM197 and KLH, a preferred sequence of the at least three components is (a)-(c)(b), i.e. wherein the β-glucan and the least one B-cell or one T-cell epitope polypeptide is coupled to the carrier protein.

According to another preferred embodiment, the conjugates according to the present invention comprise a T-cell epitope and are free of B-cell epitopes, wherein the conjugate preferably comprises more than one T-cell epitope, especially two, three, four or five T-cell epitopes. This construct is specifically suitable for cancer vaccines. This construct is also specifically suitable for self-antigens, especially autoimmune disease associated self-antigens. The treatment effect of the respective conjugate is associated with a reduction of effector T-cells and the development of regulatory T-cell (T_{reg}-cell) populations which leads to the dampening of the respective disease, e.g. autoimmune disease or allergic disorders, for example as shown for multiple sclerosis. Notably, these T reg cells execute strong bystander immunosuppression and thus improve disease induced by cognate and noncognate autoantigens.

Preferred CLECs to be used as polysaccharide backbones according to the present invention are pustulan or other β-(1,6) glucans (including also synthetic forms of such glucans); others to be used: mannan, β-glucan family members, esp. linear β-(1,3) (S. cerevisiae β-glucan (e.g.: 150 kDa), curdlan) or branched β-(1,3) and β-(1,6) containing glucans, e.g.: laminarin (4,5-7 kDa), scleroglucan, schizophyllan, more preferably linear glucans, (e.g.: β(1,3): S. cerevisiae β-glucan (150 kd), curdlan (75-80 kDa or bigger), β-(1,3)+β-(1,4) lichenan (22-250 kDa) β-(1,6) pustulan (20 kDa). Preferred CLECs according to the present invention are therefore mannan and β-glucans, including linear and branched β-glucans characterized by presence of β-(1,3)-, β-(1,3)+β-(1,4)- and β(-1,6) main chains as well as with attached side chains with β-(1,6) residues, more preferred linear β-glucans containing β-(1,3), β-(1,3)+β-(1,4) and β-(1,6) chains, more preferred linear β-(1,6) β-glucans, especially pustulan, fragments or synthetic variants thereof consisting of multimeric β-(1,6)-glucan saccharides (e.g. 4-mer, 5-mer, 6 mer, 8-mer, 10-mer, 12-mer, 15-mer, 17-mer or 25 mer).

Preferably, the minimum length of the CLECs according to the present invention is a 6-mer, because with smaller polysaccharides oxidation reactions as performed with the present invention are problematic (eventually other coupling mechanisms can be used for such smaller forms and/or terminally linking with addition of reactive forms). CLECs with 6 or more monomer units (i.e. 6-mers and larger-mers) show good dectin binding. Usually, the longer the CLEC, the better the dectin binding. The degree of polymerization (i.e. the amount of single glucose molecules within one glucan entity, DP) of 20-25 (i.e. DP20-25) definitely ascertains good binding and in vivo efficacy (e.g. laminarin is a typical example with a DP of 20-30).

Molecular weight of synthetic CLECs may also be smaller, Accordingly, e.g. as low as 1-2 kDa, whereas preferred molecular weight ranges of glucans and fragments thereof may be from 1 to 250 kDa (e.g. laminarin, lichenan, S. cerevisiae β-glucan, pustulan, curdlan and barley glucans, etc.), preferably from 4.5 to 80 kDa (e.g. laminarin, pustulan, curdlan, low molecular weight lichenan, etc.), especially 4.5 to 30 kDa (e.g. laminarin, pustulan, low MW lichenan, etc.). Mannans are polysaccharides that are linear polymers of the sugar mannose. Plant mannans have β-(1,4) linkages. They are a form of storage polysaccharide. Mannan cell wall polysaccharide found in yeasts have an α-(1,6) linked backbone and α-(1,2) and α-(1,3) linked branches. It is serologically similar to structures found on mammalian glycoproteins.

In order to produce the conjugates according to the present invention, the CLEC, especially pustulan, must be activated (e.g. by using mild periodate mediated oxidation) and the degree of oxidation is important for the immune response. As already disclosed above, practical oxidation ranges are-specifically for pustulan-from about 20 to 260% oxidation. In many cases, the optimal oxidation range is between a low/middle oxidation (i.e. 20-60% oxidation) and a high degree of oxidation (i.e. 140-260% oxidation), i.e. in the range of 60-140% oxidation. Optimization for other CLECs may easily be adapted by a person skilled in the art, e.g. for lichenan more than 200% is necessary to gain a similar amount of aldehyde groups.

Accordingly, the ranges may alternatively also be defined as the reactivity with Schiff's fuchsin reagent which—for the example of pustulan—can be defined as follows: a low/middle oxidation degree at a molar ratio (sugar monomer:periodate) of 0.2-0.6, an optimal range of 0.6-1.4, and a high degree of oxidation of 1.4-2.6, respectively.

In any way, the degree of oxidation should be defined to meet the optimal range for each specific CLEC. Preferably, a linear Bglucan, more preferred a β-(1,6β-glucan, especially pustulan, pustulan fragments or synthetic variants thereof consisting of multimeric β(1,6)-glucan saccharides (e.g. 4-mer, 5-mer, 6 mer, 8-mer, 10-mer, 12-mer, 15-mer, 17-mer or 25-mer) is activated by mild periodate oxidation resulting in cleavage of vicinal OH groups and thus generation of reactive aldehydes. Mild periodate oxidation refers to the use of sodium periodate (NaIO₄), a well-known mild agent for effectively oxidizing vicinal diols in carbohydrate sugars to yield reactive aldehyde groups. The carbon-carbon bond is cleaved between adjacent hydroxyl groups. By altering the amount of periodate used, aldehydes can be stoichiometrically introduced into a smaller or larger number of sugar moieties of a given polysaccharide.

Other exemplary methods for activation of carbohydrates are well known in the art and include cyanylation of hydroxyls (e.g.: by use of organic cyanylating reagents, like 1-cyano-4-(dimethylamino)-pyridinium tetrafluoroborate (CDAP) or N-cyanotriethylammonium tetrafluoroborate (CTEA), reductive amination of carbohydrates or activation and coupling using Carboxylic acid-reactive chemical groups like Carbodiimides.

Activated carbohydrates are then reacted with the polypeptides to be coupled to the activated CLEC and allowed to form a conjugate of the CLEC with the B-cell or a T-cell epitope polypeptide.

Accordingly, the present invention also relates to a method for producing the conjugates according to the present invention, wherein the β-glucan or mannan is activated by oxidation and wherein the activated β-glucan or mannan is contacted with the B-cell and/or the T-cell epitope polypeptide, thereby obtaining a conjugate of the β-glucan or mannan with the B-cell and/or the T-cell epitope polypeptide.

Preferably, the β-glucan or mannan is obtained by periodate oxidation at vicinal hydroxyl groups, as reductive amination, or as cyanylation of hydroxyl groups.

According to a preferred embodiment, the β-glucan or mannan is oxidized to an oxidation degree defined as the reactivity with Schiff's fuchsin-reagent corresponding to an oxidation degree of an equal amount of pustulan oxidized with periodate at a molar ratio of 0.2-2.6 preferably of 0.6-1.4, especially 0.7-1.

Preferably, the conjugate is produced by hydrazone based coupling for conjugating hydrazides to carbonyls (aldehyde) or coupling by using hetero-bifunctional, maleimide-and-hydrazide linkers (e.g.: BMPH (N-β-maleimidopropionic acid hydrazide, MPBH (4-[4-N-maleimidophenyl]butyric acid hydrazide), EMCH (N-[ε-Maleimidocaproic acid) hydrazide) or KMUH (N-[κ-maleimidoundecanoic acid] hydrazide) for conjugating sulfhydryls (e.g.: cysteines) to carbonyls (aldehyde).

The polypeptides to be coupled to the CLECs according to the present invention are or comprise at least one B-cell or at least one T-cell epitope. Preferably, the polypeptide coupled to the CLECs contain a single B- or T-cell epitope (even in the embodiment when more than one kind of polypeptide is coupled to the CLEC polysaccharide backbone). As also shown in the example section, preferred lengths of the polypeptides are from 5 to 29 amino acid residues, preferably from 5 to 25 amino acid residues, more preferred from 7 to 20 amino acid residues, even more preferred from 7 to 15 amino acid residues, especially from 7 to 13 amino acid residues. In this connection it important to note that these length ranges are drawn to the epitope sequences only but do not include linkers, including peptidic linkers, such as cysteine or glycine or bi-, tri-, tetra- (or longer)-meric peptide groups, such as CG or CG, or cleavage sites, such as the cathepsin cleavage site; or combinations thereof (e.g. —NRRAC). Illustrative examples of epitopes have been tested in the example section; it follows from these results that the platform according to the present invention is not limited to any specific polypeptide. Therefore, virtually all possible epitopes are eligible for the present invention, including those epitopes which are already known in the present field and especially those which have already been described to be integrable into a presentation platform (e.g. together with a “classical” carrier molecule or adjuvant).

Epitopes are specifically preferred, if they can be coupled to activated β-glucan based on state-of-the-art coupling methods including hydrazide-mediated coupling, coupling via heterobifunctional linkers (e.g.: BMPH, MPBH, EMCH, KMUH etc.), imidazole mediated coupling, reductive amination, carbodiimide coupling etc. (more to be added). Epitopes used comprise individual peptides, can be contained within peptides or proteins or can be presented as peptide-protein conjugates before coupling to CLECs.

Preferred coupling methods to be used to provide the conjugates according to the present invention are therefore hydrazide coupling or coupling using thioester formation (e.g. maleimide coupling using BMPH (N-β-maleimidopropionic acid hydrazide), MPBH, EMCH, KMUH, especially where pustulan is coupled to the BMPH via hydrazone formation and the polypeptide is coupled via thioester.

In this embodiment, it is preferred to provide the polypeptides with two preferred linkers, such as hydrazide polypeptides/epitopes for hydrazone coupling:

N-terminal coupling of peptide: H₂N—NH—CO—CH₂—CH₂—CO-Polypeptide-COOH; preferably in combination with succinic acid or alternative suitable linkers, e.g. other suitable dicarboxylic acids, especially also glutaric acid used as a spacer/linker; C-terminal coupling (which is the preferred coupling orientation according to the present invention): NH₂-Polypeptide-NH—NH₂.

Alternatively, non-modified polypeptides/epitopes may be applied in the present invention, e.g. polypeptides containing an (extra) cysteine residue or an alternative source for SH groups at either C- or N-terminus for heterobifunctional linker mediated coupling (especially BMPH, MPBH, EMCH, KMUH): NH₂-Cys-Pep-COOH or NH₂-Pep-Cys-COOH.

Preferred B-cell polypeptides to be used according to the present invention are polypeptides with a length of 5 to 19 amino acid residues, preferably 6 to 18 amino acid residues, especially 7 to 15 amino acid residues. The B-cell epitopes are preferably short, linear polypeptides, glycopolypeptides, lipopolypeptides, other post-translationally modified polypeptides (e.g.: phosphorylated, acetylated, nitrated, containing pyroglutamate residues, glycosylated etc.), cyclic polypeptides, etc.

Preferred B-cell epitopes are B-cell epitopes representing self-antigens, B-cell epitopes representing antigens present in neoplastic diseases, B-cell epitopes representing antigens present in allergic, IgE-mediated diseases, B-cell epitopes representing antigens present in autoimmune diseases, B-cell epitopes representing antigens present in infectious diseases, B-cell epitopes representing conformational epitopes, B-cell epitopes representing carbohydrate epitopes, B-cell epitopes immobilized/coupled to polypeptides or proteins forming multivalent B-cell epitope-protein/polypeptide conjugates suitable for CLEC coupling including carrier molecules like CRM197, KLH, tetanus toxoid or other commercially available carrier proteins or carriers known to skilled persons in the field, preferably CRM197 and KLH, most preferred CRM197; non-peptidogenic antigens amenable to coupling to reactive aldehydes present on pustulan/CLECs (including linear polypeptides, polypeptides representing conformational epitopes, mimotopes or polypeptide variants from natural epitopes/sequences, glycopolypeptides, lipopolypeptides, other post-translationally modified peptides (e.g.: phosphorylated, acetylated, containing pyroglutamate residues, etc.), cyclic polypeptides, etc.).

Preferred T-cell polypeptides to be used according to the present invention have a length of 8 to 30 amino acid residues, preferably of 13 to 29 amino acid residues, more preferably of 13 to 28 amino acid residues.

Preferred specificities of the T-cell epitopes to be used in the present invention are short linear peptides suitable or known to be suitable for presentation via MHC I and II (as known to the person skilled in the art), especially MHCII epitopes for CD4 effector T-cells and CD4 Treg cells, MHCI epitopes for cytotoxic T-cell (CD8+) and CD8 Treg cells, for example useful for cancer, autoimmune or infectious diseases) with known efficacy in humans or animals; short linear peptides suitable for presentation via MHC I and II (as known to the person skilled in the art) with a N- or C-terminal addition of a lysosomal protease cleavage site, specifically a Cathepsin protease family member specific site, more specifically a site for cysteine cathepsins like cathepsins B, C, F, H, K, L, O, S, V, X, and W, especially a cathepsin S- or L-, most preferred a Cathepsin L cleavage site fostering efficient endo/lysosomal release of peptides for MHC presentation, especially MHCII with known efficacy in humans or animals. Cathepsin cleavage sites in various proteins have been identified and are well known in the art. This includes disclosures of sequences or methods to identify such sequences: e.g.: Biniossek et al., J. Proteome Res. 2011, 10, 12, 5363-5373; Adams-Cioaba et al., Nature Comm. 2011, 2:197; Ferrall-Fairbanks PROTEIN SCIENCE 2018 VOL 27:714-724; Kleine-Weber et al., Scientific Reports (2018) 8:1659, https://en.wikipedia.org/wiki/Cathepsin_S and others. Specifically, the adaption of peptide sequences using artificial protease cleavage sites as shown in the present invention is based on the surprising effect of these sequence extensions in eliciting more efficient immune responses following dermal application of the CLEC vaccines according to the present invention when the antigens are coupled to CLECs. Vaccines are according to the present invention are taken up by DCs and peptide antigens are subsequently lysosomally processed and presented at MHCs.

Lysosomes are intracellular membrane-bound organelles characterized by an acidic interior and harbor a variety of hydrolytic enzymes including lipases, proteases and glycosidases that participate in cellular catabolismamong the variety of enzymes that lysosomes harbor, cathepsins are a family of lysosomal proteases with a broad spectrum of functions. All cathepsins fall into three different protease families: serine proteases (cathepsins A and G), aspartic proteases (cathepsin D and E) and eleven cysteine cathepsins. In humans eleven cysteine cathepsins are known which also have structures similar to that of papain: cathepsins B, C (J, dipeptidyl peptidase I or DPPI), F, H, K (O2), L, O, S, V (L2), X (P, Y, Z) and W (lymphopain).

Cathepsins exhibit similarities in their cellular localization and biosynthesis with some differences in their expression pattern. Of all the lysosomal proteases, cathepsins L, B, and D are the most abundant with their lysosomal concentrations equivalent to 1 mM. Cathepsins B, H, L, C, X, V, and O are ubiquitously expressed while cathepsins K, S, E, and W show cell or tissue-specific expression. Cathepsin K is expressed in osteoclasts and in epithelial cells. Cathepsins S, E, and W are mainly expressed in immune cells.

Besides their main function in lysosomal protein recycling, cathepsins play significant roles in a variety of physiological processes. Cathepsin S is the major protease involved in MHC II Ag processing and presentation. Cathepsin S null mice show a marked variation in generation of MHC II-bound li fragments and presentation, due to the substantially diminished li degradation in professional APCs where cathepsin S is abundantly expressed. In addition, endocytosis targets exogenous material selectively to cathepsin S in human DCs. Enrichment of MHC II molecules within late endocytic structures has consistently been noted in splenic DCs of cathepsin S-deficient mice as well. Recent studies suggest that both cathepsin B and D are involved, but not essential for MHC II-mediated Ag presentation as well. Cathepsin L also plays a role in a wide variety of cellular processes including antigen processing, tumor invasion and metastasis, bone resorption, and turnover of intracellular and secreted proteins involved in growth regulation. Although commonly recognized as a lysosomal protease, cathepsin L is also secreted. This broad-spectrum protease is potent in degrading several extracellular proteins (laminins, fibronectin, collagens I and IV, elastin, and other structural proteins of basement membranes) as well as serum proteins and cytoplasmic and nuclear proteins.

As a novel means to augment T-cell epitope efficacy in a vaccine, especially a CLEC based vaccine, a N- or C-terminal addition of a lysosomal protease cleavage site is provided as a preferred embodiment of the present invention.

Such cleavage sites according to the present invention may be characterized as follows:

Cathepsin L-like cleavage sites:

The intended Cathepsin L like cleavage site is defined based on protease cleavage site sequences known by the man skilled in the art, specifically also those as disclosed in Biniossek et al. (J. Proteome Res. 2011, 10, 5363-5373) and Adams-Cioaba et al. (Nature Comm. 2011, 2:197). The orientation of the site can be Nor C-terminally, preferred C-terminally. The preferred consensus sequence for C-terminal a Cathepsin L site is consisting of the formula:

Xₙ-X₁-X₂-X₃-X₄-X₅-X₆-X₇-X₈
Xₙ: 3-27 amino acids from the immunogenic peptide
X₁: any amino acid
X₂: any amino acid
X₃: any amino acid
X₄: N/D/A/Q/S/R/G/L; preferred N/D, more preferred N
X₅: F/R/A/K/T/S/E; preferred F or R, more preferred R
X₆: F/R/A/K/V/S/Y; preferred F or R, more preferred R
X₇: any amino acid, preferred A/G/P/F, more preferred A
X₈: cysteine or Linker like NHNH₂
Most preferred sequence: Xₙ-X₁X₂X₃NRRA-Linker

Cathepsin S like cleavage site:

The intended Cathepsin S cleavage site is based on protease cleavage site sequences known by the man skilled in the art, specifically also those as disclosed in Biniossek et al. (J. Proteome Res. 2011, 10, 5363-5373) and in https://en.wikipedia.org/wiki/Cathepsin S and is characterized by the consensus sequence:

Xₙ-X₁-X₂-X₃-X₄-X₅-X₆-X₇-X₈
Where X is characterised by
Xₙ: 3-27 amino acids from the immunogenic peptide
X₁: any amino acid
X₂: any amino acid
X₃: any amino acid, preferred V, L, I, F, W, Y, H, more preferred V
X₄: any amino acid, preferred V, L, I, F, W, Y, H, more preferred V
X₅: K, R, E, D, Q, N, preferably K, R more preferably R
X₆: any amino acid
X₇: any amino acid, preferred A
X₈: preferred A
X₈: cysteine or linker like NHNH₂
Most preferred sequence: Xₙ-X₁X₂VVRAA-Linker

T-cell epitopes contained within proteins where the proteins are suitable for coupling to CLECs including carrier proteins, especially non-toxic cross-reactive material of diphtheria toxin (CRM), especially CRM₁₉₇, KLH, diphtheria toxoid (DT), tetanus toxoid (TT), Haemophilus influenzae protein D (HipD), and the outer membrane protein complex of serogroup B meningococcus (OMPC), recombinant non-toxic form of Pseudomonas aeruginosa exotoxin A (rEPA), flagellin, Escherichia coli heat labile enterotoxin (LT), cholera toxin (CT), mutant toxins (e.g., LTK63 and LTR72), virus-like particles, albumin binding protein, bovine serum albumin, ovalbumin, a synthetic peptide dendrimer e.g. a Multiple antigenic peptide (MAP) or other commercially available carrier proteins, preferably CRM197 and KLH, most preferred CRM197, preferably wherein the ratio of carrier protein to β-glucan in the conjugate is from 1/0.1 to 1/50, preferably 1/0.1 to 1/40, more preferred from 1/0.1 to 1/20, especially from 1/0.1 to 1/10.

According to preferred embodiments of the present invention, the CLEC conjugates according to the present invention comprise conjugated with individual B- and/or T-cell epitopes, (a) CLECS including mixes of B- or r T-cell epitopes, especially these epitope(s) coupled to pustulan; (b) CLECs conjugated with polypeptide-carrier protein conjugates, preferably polypeptide-KLH or polypeptide CRM197 conjugates coupled to pustulan, most preferably, polypeptide-CRM197 conjugates coupled to pustulan; (c) CLECS conjugated with individual B- and T-cell epitopes from self-proteins (cancer) or pathogens (infectious diseases), not the promiscuous MHC/HLA-specific but known disease specific T-cell epitopes; coupled to CLECs, most preferably to pustulan; (d) CLECs coupled individually (“individually” here means that the polypeptide chains are not present as a fusion protein, tandem repeat polypeptide or peptide-protein conjugate but as independent entities; i.e. an independent B-cell epitope-containing polypeptide and an independent T-cell epitope containing polypeptide) with B-cell epitopes and T-cell epitopes which are contained within polypeptides or proteins, e.g. carrier proteins, self-proteins, foreign proteins from pathogens, allergens etc.; (e) CLECs coupled individually (“individually”, again has the same meaning as for (d)) with T-cell epitopes representing linear MHCI and MHCII epitopes or which are contained within proteins, e.g. carrier proteins or target proteins for example for the treatment of neoplastic disease, or autoimmune disease.

In view of these advantageous properties of the conjugates of the present invention, it follows that the conjugates and vaccines according to the present invention are specifically useable for an active anti-Aβ, anti-Tau and/or anti-alpha synuclein vaccine for the treatment and prevention of β-amyloidoses, tauopathies, or synucleopathies, preferably Parkinson's disease (PD), dementia with Lewy bodies (DLB), multiple system atrophy (MSA), Parkinson's disease dementia (PDD), neuroaxonal dystrophies, Alzheimer's Disease (AD), AD with Amygdalar Restricted Lewy Bodies (AD/ALB), dementia in Down syndrome, Pick disease, progressive supranuclear palsy (PSP), corticobasal degeneration, Frontotemporal dementia and parkinsonism linked to chromosome 17 (FTDP-17) and argyrophilic grain disease.

Therefore, the conjugates according to the present invention are specifically useful for the prevention or treatment of diseases, for example in humans, mammals or birds, especially for the treatment and prevention of human diseases. An aspect of the present invention is therefore the use of the present conjugates in the medical field as a medical indication. The present invention relates to the conjugates according to the present invention for use in the treatment or prevention of diseases. The present invention therefore also relates to the use of a conjugate according to the present invention for the manufacture of a medicament for the prevention or treatment of diseases, preferably for the prevention or treatment of infectious diseases, chronic diseases, allergies or autoimmune diseases. Accordingly, the present invention also relates to a method for the prevention or treatment of diseases, preferably for use in the prevention or treatment of infectious diseases, chronic diseases, allergies or autoimmune diseases, wherein an efficient amount of a conjugate according to the present invention is administered to a patient in need thereof.

According to a further aspect, the novel glycoconjugates according to the present invention can be used for the prevention of infectious diseases; with the preferred proviso that the use in the prevention or treatment of diseases caused directly or indirectly by fungi, especially by C. albicans, by providing the predominantly linear β-(1,6)-glucan with a ratio of (1,6)-coupled monosaccharide moieties to non-β-(1,6)-coupled monosaccharide moieties of at least 1:1 as an antigen (eventually coupled to a carrier protein) are excluded. Such diseases are for example microbial infections for example caused by Haemophilus influenzae type b (Hib), Streptococcus pneumoniae, Neisseria meningitidis and Salmonella Typhi or other infectious agents.

According to a further aspect, the present invention also relates to a pharmaceutical composition comprising a conjugate or vaccine as defined above and a pharmaceutically acceptable carrier.

Preferably, the pharmaceutically acceptable carrier is a buffer, preferably a phosphate or TRIS based buffer.

According to a preferred embodiment of the present invention, the pharmaceutical composition is contained in a needle-based delivery system, preferably a syringe, a mini-needle system, a hollow needle system, a solid microneedle system, or a system comprising needle adaptors; an ampoule, needle-free injection systems, preferably a jet injector; a patch, a transdermal patch, a microstructured transdermal system, a microneedle array patch (MAP), preferably a solid MAP (S-MAP), coated MAP (C-MAP) or dissolving MAP (D-MAP); an electrophoresis system, a iontophoresis system, a laser-based system, especially an Erbium YAG laser system; or a gene gun system. The conjugates according to the present invention are not limited to any form of production, storage or delivery state. All traditional and typical forms are therefore adaptable to the present invention. Preferably, the compositions according to the present invention may contain the present conjugates or vaccines in contained as a solution or suspension, deep-frozen solution or suspension; lyophilizate, powder, or granulate.

The present invention is further illustrated by the following examples and figures, however without being restricted thereto.

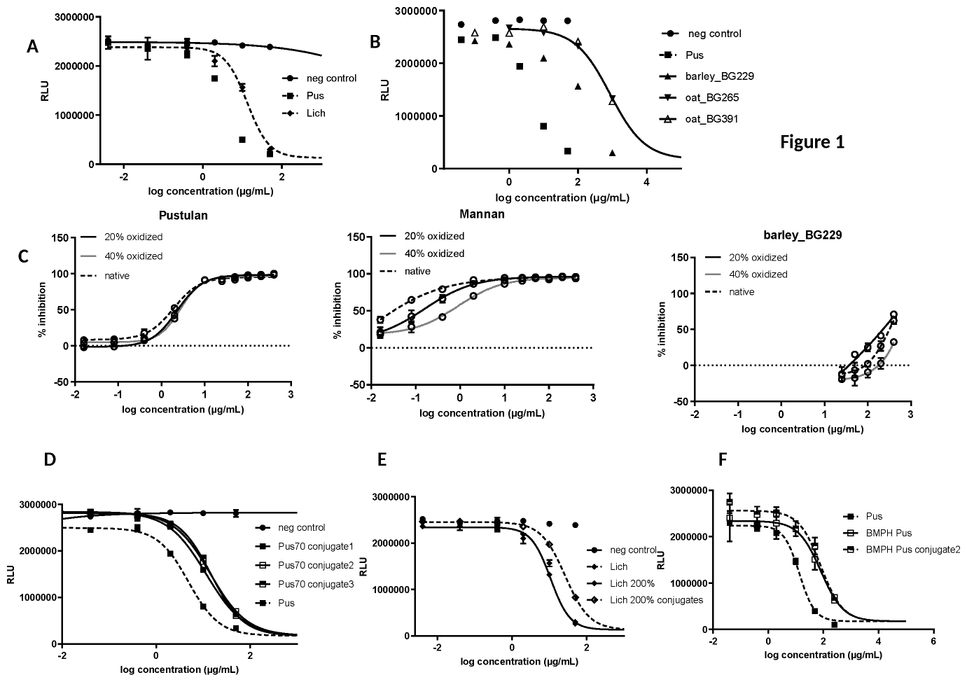

FIG. 1 shows: ConA and DC receptor (i.e. dectin-1) binding activity by CLEC-conjugates in vitro A) higher binding efficacy to dectin-1 is demonstrated for pustulan (Pus) when compared to lichenan (Lich) and B) beta-glucans from oat (oat_BG265, oat_BG391) and barley (Barley_BG229) showed limited binding efficacy in comparison to pustulan. C) different Glucan types (i.e., pustulan, mannan, and barley glucan (229kd)) retain high or intermediate receptor binding activity following glucan oxidation as assessed by competitive binding assays. “20% and 40% oxidized” denotes the oxidation status of glucan moieties used for conjugation. % Inhibition indicates the inhibition of binding of soluble dectin-1 receptor (pustulan and barley_BG229) or ConA (mannan) to plate bound beta-glucan or mannan in the presence of the indicated concentrations of the tested CLEC. D) Pustulan-conjugates and E) Lichenan-conjugates maintain approximately 50% of dectin-1 binding capacity compared to uncoupled beta-glucan, as assessed by competitive binding assay. F) Pustulan-conjugates produced via heterobifunctional linkers maintain high dectin-1 binding efficacy. Data are shown as relative light units (RLU) of a luminometric ELISA. Pus70 Conjugate 1-3 refers to three different CLEC peptide conjugates, respectively (SeqID2, SeqID10 and SeqID16). Pus 70% and Lich 200% refers to pustulan and Lichenan with the respective oxidation status. BMPH Pus refers to activated pustulan. BMPH Conjugate 2 refers to CLEC-SeqID10 conjugate.

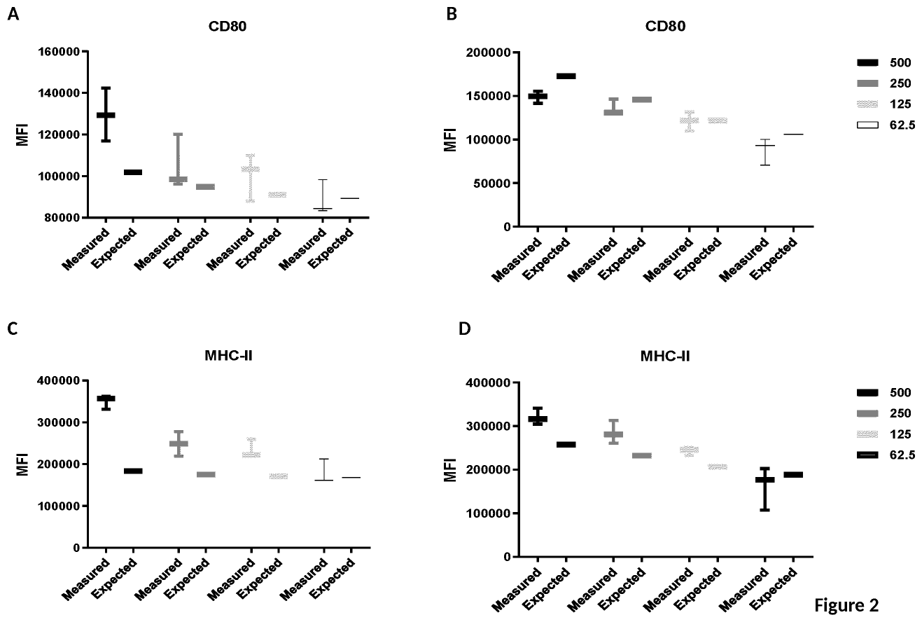

FIG. 2 shows: Flow cytometry analysis of dendritic cell activation by lipopolysaccharide (LPS) and different pustulan preparations. Immature, bone marrow derived mouse dendritic cells (BMDCs) were generated in vitro, using granulocyte-macrophage colony-stimulating factor (GM-CSF). GM-CSF-BMDCs were stimulated with LPS (equivalent dose contained in oxidized pustulan and in pustulanconjugate preparations), SeqID2+SeqID7+pustulan conjugates or oxidized pustulan only for 24 hours. Pustulan-conjugates and pustulan only were used in increasing doses starting at 62.5 μg/mL of the respective sugar (up to 500 μg/mL). DCs were identified based on CD11c/CD11b expression, and the surface expression of CD80 and major histocompatibility complex (MHC) class II by A) and C) SeqID2+SeqID7+pustulan conjugates or B) and D) oxidized pustulan only were measured by flow cytometry. The expression of activation markers was analyzed by CytExpert Software for DCs treated with pustulan-preparations (=measured) and DCs treated with equivalent amounts of LPS (=expected).

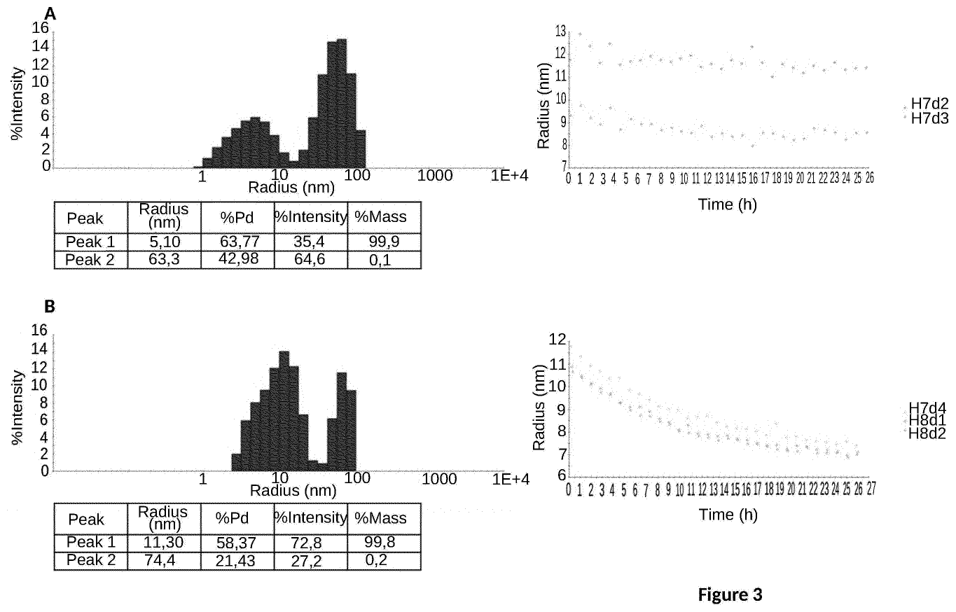

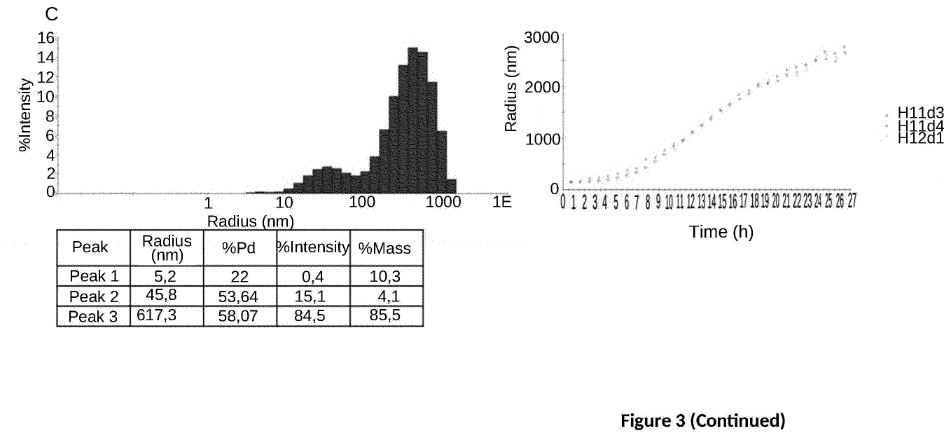

FIG. 3 shows: Particle size determination of CLEC-conjugates by dynamic light scattering (DLS). Particle size has been determined by measuring the random changes in the intensity of light scattered from a suspension or solution by DLS. Regularisation analysis and the corresponding cumulant radius analysis over 24 hours, respectively, are shown for A) SeqID5+SeqID7+Pustulan (80% oxidation status) conjugates, B) SeqID6+CRM+Pustulan conjugates and C) non-modified pustulan.

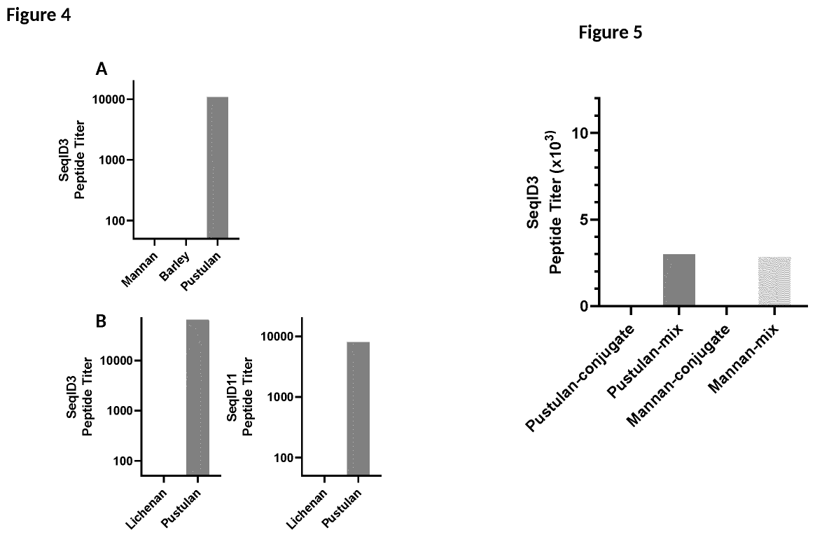

FIG. 4 shows: Comparison of immunogenicity of different CLEC based vaccines. Female BALB/c mice, 8-12 weeks of age, received a total of 3 intradermal vaccinations applied at a 2-week interval. Blood samples have been collected at baseline and after each vaccination to inform on the kinetics of the ensuing immune response. Samples were taken 2 weeks after 3^{rd} application and analysed for A) antipeptide response (SeqID3) of mannan-, barley- and pustulan-based vaccines (SeqID2+SeqID7+CLEC) and B) anti-peptide responses (SeqID3 and SeqID11) of pustulan- and lichenan-based vaccine (SeqID2+SeqID7+CLEC and SeqID10+SeqID7+CLEC).

FIG. 5 shows: Comparative analysis of the immunogenicity of peptide-pustulan conjugates and vaccines consisting of unconjugated peptides and CLECs. Female BALB/c mice, 8-12 weeks of age received a total of 3 intradermal vaccinations applied at a 2-week interval. Blood samples have been collected at baseline and after each vaccination to inform on the kinetics of the ensuing immune response. Samples were taken 2 weeks after 3rd application and analysed for antipeptide responses (SeqID3). Vaccines used: SeqID2+SeqID7+CLEC or mixes of unconjugated SeqID2, SeqID7 and CLEC.

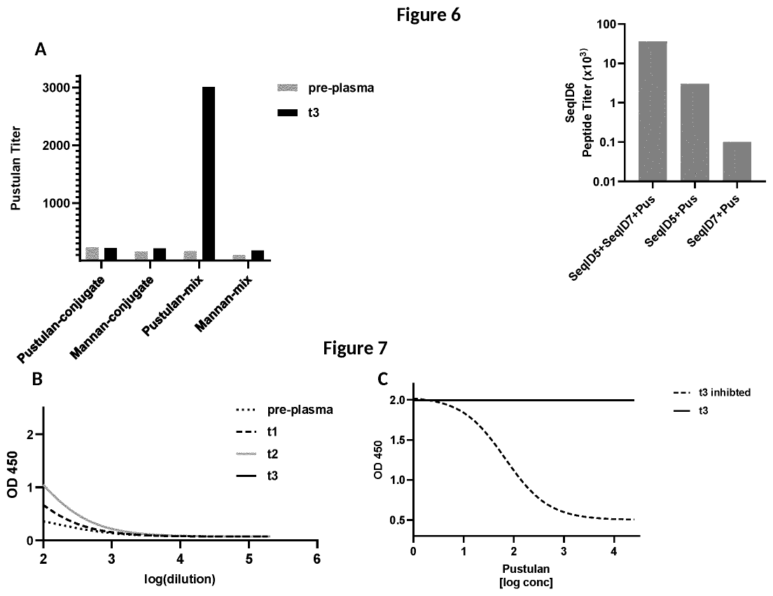

FIG. 6 shows: Comparative analysis of the immunogenicity of pustulan conjugates containing B- and T-cell epitopes to conjugates containing either the respective B-cell or T-cell epitope only. Female BALB/c mice, 8-12 week of age received a total of 3 intradermal vaccinations applied at a 2-week interval. Blood samples have been collected at baseline and after each vaccination to inform on the kinetics of the ensuing immune response. Vaccines used: SeqID5+SeqID7+CLEC or SeqID5+CLEC, and SeqID7+CLEC. Samples were taken 2 weeks after 3rd application and analysed for antipeptide responses (SeqID6).

FIG. 7 shows: Comparative analysis of anti-pustulan antibody responses in mice following repeated immunisation using peptide-pustulan conjugates or vaccines containing the respective non conjugated components Female BALB/c mice, 8-12 weeks of age received a total of 3 intradermal vaccinations applied at a 2-week interval. Blood samples have been collected at baseline and after each vaccination to inform on the kinetics of the ensuing immune response. Pre-plasma and t1-t3 indicates immune responses detectable before (preplasma) or after the first (t1), 2nd (t2) or third (t3) immunization. Samples were taken 2 weeks after 3rd application and analysed for anti-pustulan responses. A) Analysis of the anti-pustulan response elicited by different vaccines. B) Kinetics of the immune response. C) Inhibition ELISA demonstrating the specificity of the ELISA system. Vaccines used: SeqID2+SeqID7+CLEC or mixes of unconjugated SeqID2, SeqID7 and CLEC

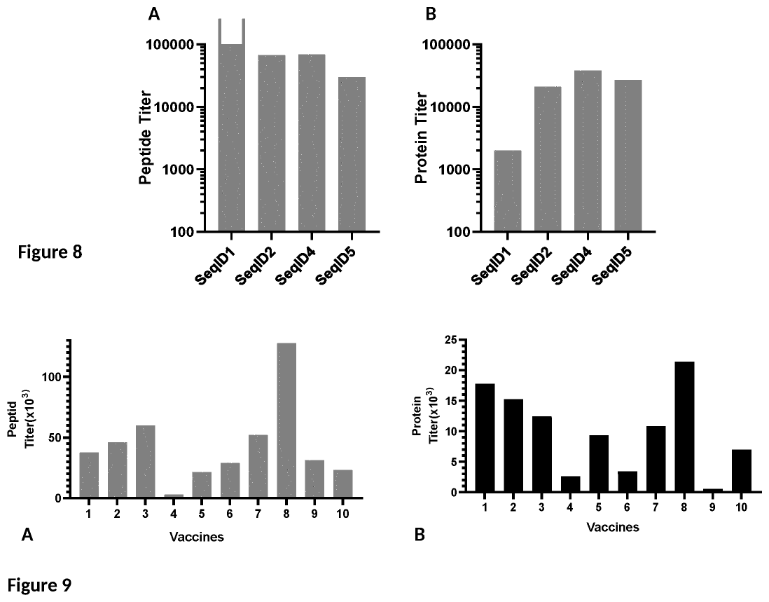

FIG. 8 shows: Comparative analysis of immune responses elicited by CLEC-based vaccines using differential peptide coupling orientation. Female BALB/c mice, 8-12 weeks of age received a total of 3 intradermal vaccinations applied at a 2-week interval. Blood samples have been collected at baseline and after each vaccination to inform on the kinetics of the ensuing immune response. 4 different CLEC-based prototype vaccine candidates (two different peptides either coupled via their C- or N-terminus to pustulan) were tested. Samples were taken 2 weeks after 3rd application and analysed for A) anti-peptide and B) anti-aSyn protein responses. Vaccines used: SeqID1/2/4/5+SeqID7+CLEC

FIG. 9 shows: Comparative analysis of the immunogenicity of CLEC-based vaccines using different promiscuous T-helper cell epi topes. Female BALB/c mice, 8-12 weeks of age received a total of 3 intradermal vaccinations applied at a 2-week interval. Blood samples have been collected at baseline and after each vaccination to inform on the kinetics of the ensuing immune response. Immune responses elicited by 9 different CLEC-based vaccines (Vaccine 1-9) containing the same B-cell epitope and different T-helper epitopes (i.e. SeqID7, SeqID22-29) were evaluated against the respective peptide-KLH conjugate (Vaccine 10), respectively. Samples were taken 2 weeks after 3rd application and analysed for A) antipeptide and B) anti-aSyn protein responses.

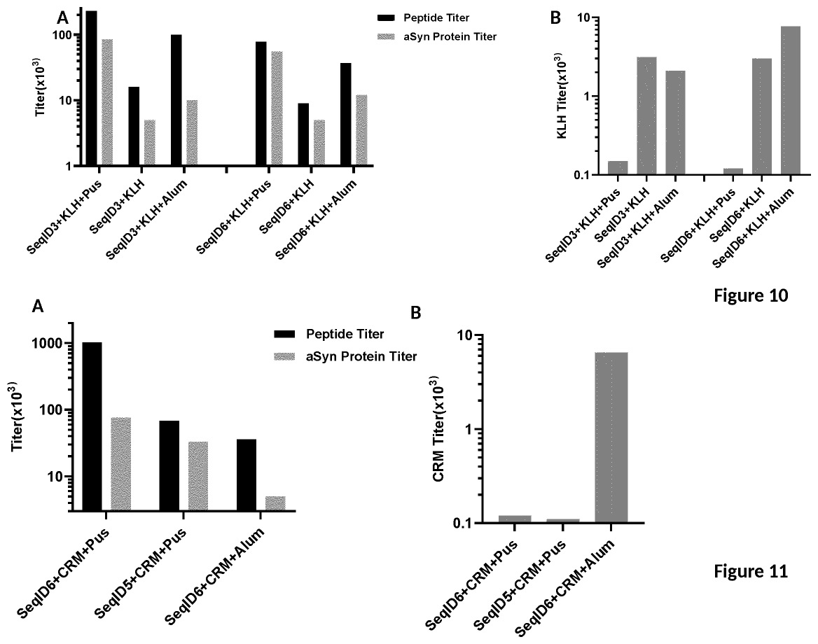

FIG. 10 shows: the Comparative analysis of the target- and carrier protein specific immunogenicity induced by CLEC-based- and conventional peptide-protein conjugate vaccines using the carrier protein KLH as source for T-helper cell epitopes. Female BALB/c mice, 8-12 weeks of age received a total of 3 intradermal or sub cutaneous (s.c.) vaccinations applied at a 2-week interval. Blood samples have been collected at baseline and after each vaccination to inform on the kinetics of the ensuing immune response. Immune reactions elicited by 2 peptide-protein conjugate vaccines using KLH as source for T-helper epitopes in combination with CLEC modifications (SeqID3+KLH+Pustulan and SeqID6+KLH+Pustulan, respectively) were evaluated against reactions induced by conventional peptide-KLH conjugates (i.e. SeqID3+KLH and SeqID6+KLH) either applied with Alum/Alhydrogel s.c. or without additional adjuvant i.d . . . . Samples were taken 2 weeks after 3rd application and analysed for A) anti-peptide and anti-aSyn protein responses and B) anti-KLH responses by ELISA.

FIG. 11 shows: the Comparative analysis of the target- and carrier protein specific immunogenicity induced by CLEC-based- and conventional peptide-protein conjugate vaccines using the carrier protein CRM197 as source for T-helper cell epitopes Female BALB/c mice, 8-12 weeks of age received a total of 3 intradermal or s.c. vaccinations applied at a 2-week interval. Blood samples have been collected at baseline and after each vaccination to inform on the kinetics of the ensuing immune response. 2 different CRM-based vaccine types have been used in this study. SeqID6+CRM+Pus represents a peptide-CRM conjugate which has been subsequently coupled to pustulan whereas SeqID5+CRM+Pus represents a conjugate where the peptide component and the carrier molecule have been coupled to the CLEC individually. Immune reactions induced by both types have been evaluated against the respective conventional peptide-CRM conjugate (i.e. SeqID6+CRM adjuvanted with Alum/Alhydrogel and applied s.c.). Samples were taken 2 weeks after 3rd application and analysed for A) anti-peptide and anti-aSyn protein responses and B) anti-CRM responses by ELISA.

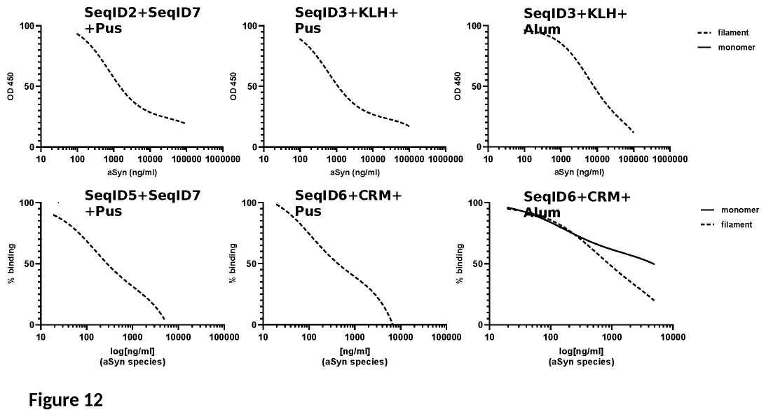

FIG. 12 shows: The comparative analysis of the selectivity of the immune responses elicited by CLEC based vaccines in vivo against two different aSyn forms. Female BALB/c mice, 8-12 weeks of age received a total of 3 intradermal or s.c. vaccinations applied at a 2-week interval. CLEC based vaccine (SeqID2+SeqID7+Pus and SeqID5+SeqID7+Pus; applied i.d.) and alternative CLEC based vaccine (SeqID3+KLH+Pus and SeqID6+CRM+Pus; applied i.d.) were evaluated against conventional peptide-component vaccine (SeqID3+KLH+Alum and SeqID6+CRM+Alum, applied s.c.). Sample were taken 2 weeks after 3rd application and subjected to aSyn selectivity assay (inhibition ELISA). Black line: monomeric aSyn used for inhibition; dashed line: filamentous aSyn used for inhibition.

FIG. 13 shows: a comparative analysis of the avidity of immune responses elicited by CLEC based vaccines. Female BALB/c mice, 8-12 weeks of age received a total of 3 intradermal or s.c. vaccinations applied at a 2-week interval. CLEC based vaccine (SeqID2+SeqID7+Pus and SeqID5+SeqID7+Pus, applied i.d.) and alternative CLEC based vaccine (SeqID3+KLH+Pus and SeqID6+CRM+Pus, applied i.d.) were evaluated against conventional peptide-component vaccine (SeqID3+KLH+Alum and SeqID6+CRM+Alum, applied s.c.). Samples were taken 2 weeks after the second (T2) or two weeks after the third immunization (T3) immunisation and antibody avidity to aSyn was assessed by ELISA based avidity assay.

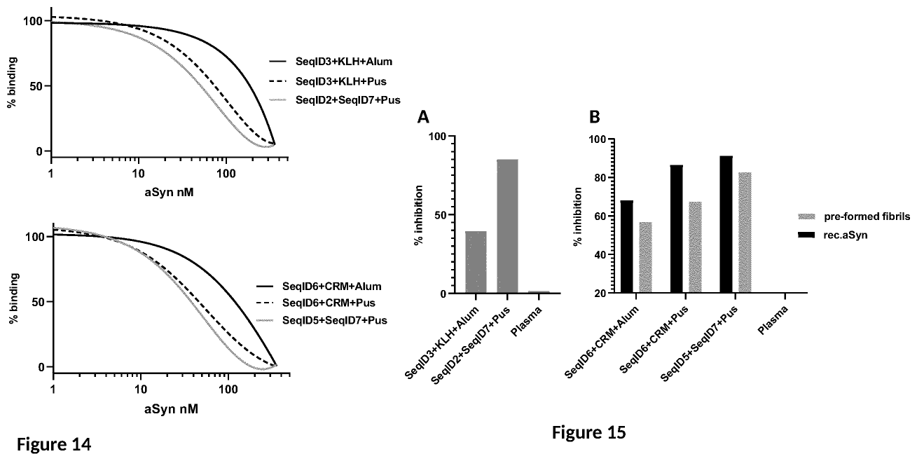

FIG. 14 shows: a comparative analysis of the affinity of immune responses elicited by CLEC based vaccines. Female BALB/c mice, 8-12 weeks of age received a total of 3 intradermal or s.c. vaccinations applied at a 2-week interval. CLEC based vaccine (SeqID2+SeqID7+Pus and SeqID5+SeqID7+Pus, applied i.d.) and alternative CLEC based vaccine (SeqID3+KLH+Pus and SeqID6+CRM+Pus, applied i.d.) were evaluated against conventional peptide-component vaccine (SeqID3+KLH+Alum and SeqID6+CRM+Alum, applied s.c.). Samples were taken 2 weeks after 3rd application and antibody equilibrium dissociation constant (Kd) to aSyn was assessed by aSyn displacement ELISA assay.

FIG. 15 shows: the comparative analysis of in vitro functionality of immune responses elicited by CLEC based vaccines. Female BALB/c mice, 8-12 weeks of age received a total of 3 intradermal and s.c. vaccinations applied at a 2-week interval. Samples were taken 2 weeks after 3rd application and modulation of aSyn aggregation in the presence of aSyn-specific Abs were evaluated by ThT fluorescence assays. A) aSyn was aggregated in the presence of CLEC-vaccine-induced Abs (SeqID2+SeqID7+Pus; applied i.d.), conventional peptide-component-induced Abs (SeqID3+KLH+Alum, applied s.c.) or murine plasma for 0-72 hours. B) aSyn or aSyn with pre-formed fibrils was aggregated in the presence of CLEC-vaccine-induced Abs (SeqID5+SeqID7+Pus and SeqID6+CRM+Pus, both applied i.d.), conventional peptide-component-induced Abs (SeqID6+CRM+Alum, applied s.c.) or murine plasma for 0-92 hours. Kinetic curves were calculated by normalization of ThT fluorescence at to and slope values extracted from linear regression analysis in the exponential growth phase of the ThT kinetic were used to calculate % inhibition of aSyn aggregation.

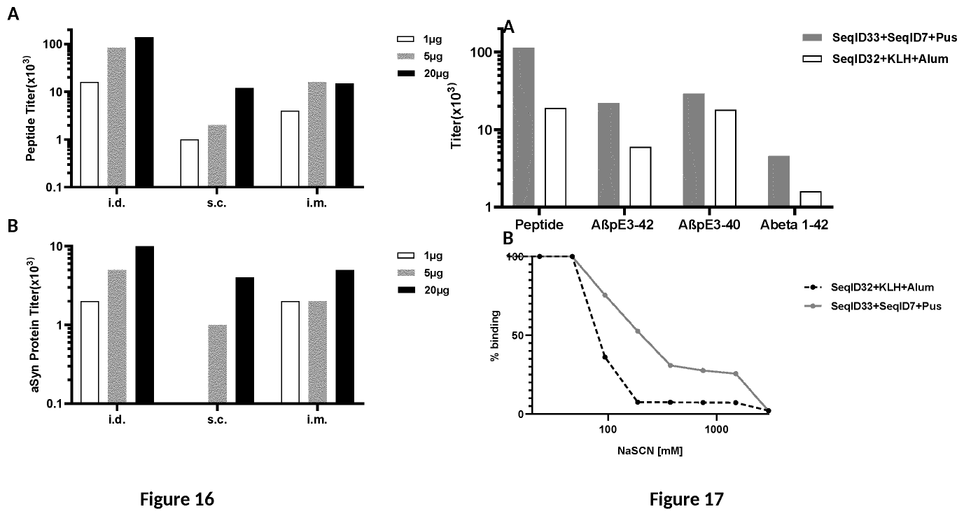

FIG. 16 shows: a comparative analysis of the effects of the route of immunization on immune responses elicited by CLEC based vaccines. Female BALB/c mice, 8-12 weeks of age received a total of 3 vaccinations applied at a 2-week interval. Blood samples have been collected at baseline and after each vaccination to inform on the kinetics of the ensuing immune response. Two alternative routes including sub cutaneous (s.c.) and intra-muscular (i.m.) were compared to intra dermal (i.d.) application for CLEC-based vaccines. Three doses of CLEC based vaccine (SeqID2+SeqID7+Pus) were applied per route. Samples were taken 2 weeks after 3rd application and analysed for A) anti-peptide and B) anti-aSyn protein responses.

FIG. 17 shows: the comparative analysis of immune responses elicited by CLEC vaccines containing B-cell epitopes characterised by post-translationally modified peptides: AB. Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID33+SeqID7+Pus, i.d.) were evaluated against conventional peptide-conjugate-based vaccines (SeqID32+KLH+Alum, s.c.). Samples were taken 2 weeks after 3rd application and analysed for A) anti-peptide, anti-AβpE3-40, anti-AβpE3-42 and anti-Aβ1-42 responses. B) Antibody avidity to AβpE3-42 was assessed by ELISA based avidity assay.

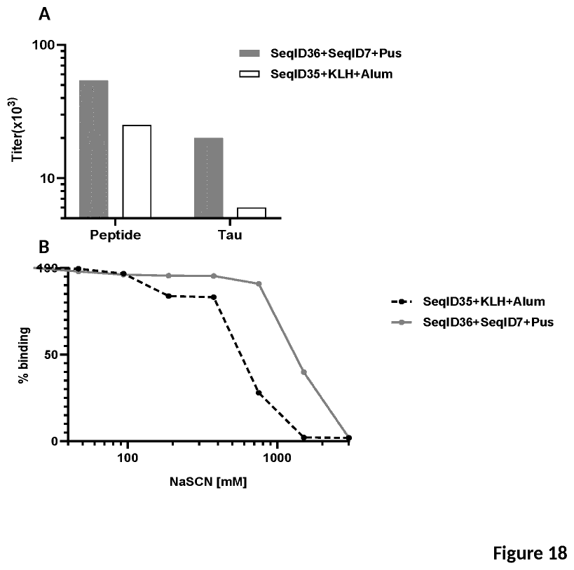

FIG. 18 shows: the comparative analysis of immune responses elicited by CLEC vaccines containing B-cell epitopes derived from intracellular proteins and self-antigens: Tau. Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID36+SeqID7+Pus, i.d.) were evaluated against conventional peptide-component-based vaccines (SeqID35+KLH+Alum, s.c.). Samples were taken 2 weeks after 3rd application and analysed for A) anti-peptide and anti-recombinant Tau 441 protein responses. B) Antibody avidity to SeqID35 was assessed by ELISA based avidity assay.

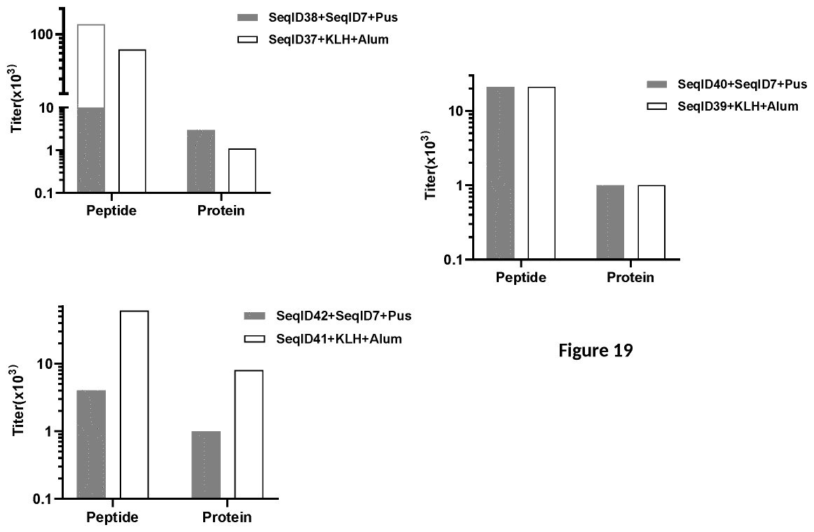

FIG. 19 shows: the comparative analysis of immune responses elicited by CLEC vaccines containing B-cell epitopes derived from secreted proteins, self-antigens, and conformational epitopes: IL23. Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. 3 CLEC-based vaccines (SeqID38/SeqID40/SeqID42 all conjugated with SeqID7 and pustulan, i.d.) were evaluated against conventional peptide-conjugate-based vaccines (SeqID37/SeqID39/SeqID41 conjugated with KLH and Alhydrogel (Alum), s.c.). Samples were taken 2 weeks after 3rd application and analysed for anti-peptide and anti-IL23 protein responses.

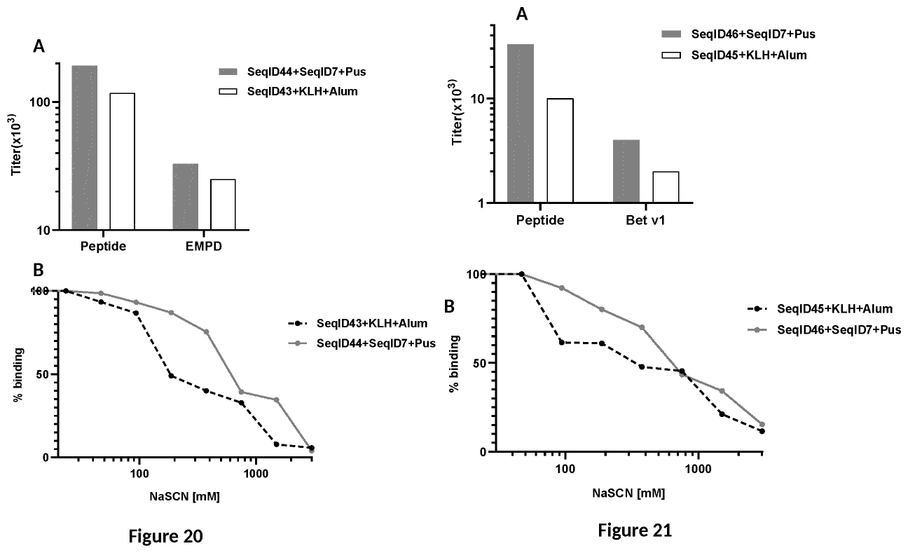

FIG. 20 shows: the comparative analysis of immune responses elicited by CLEC vaccines containing B-cell epitopes derived from self-epitopes present in transmembrane proteins: Extracellular Membrane-Proximal Domain of Membrane-Bound IgE (EMPD). Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID44+SeqID7+Pus, i.d.) were evaluated against conventional peptide-component-based vaccines (SeqID43+KLH+Alum, s.c.). Samples were taken 2 weeks after 3rd application and analysed for A) anti-injected peptide and anti-EMPD peptide responses. B) Antibody avidity to EMPD peptide was assessed by ELISA based avidity assay.

FIG. 21 shows: the comparative analysis of immune responses elicited by CLEC vaccines containing B-cell epitopes derived from allergens, mimotopes and conformational epitopes: Bet v 1. Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID46+SeqID7+Pus, i.d.) were evaluated against conventional peptide-component-based vaccines (SeqID45+KLH+Alum, s.c.). Samples were taken 2 weeks after 3rd application and analysed for A) anti-peptide and anti-Bet v 1 protein responses. B) Antibody avidity to Bet v 1 was assessed by ELISA based avidity assay.

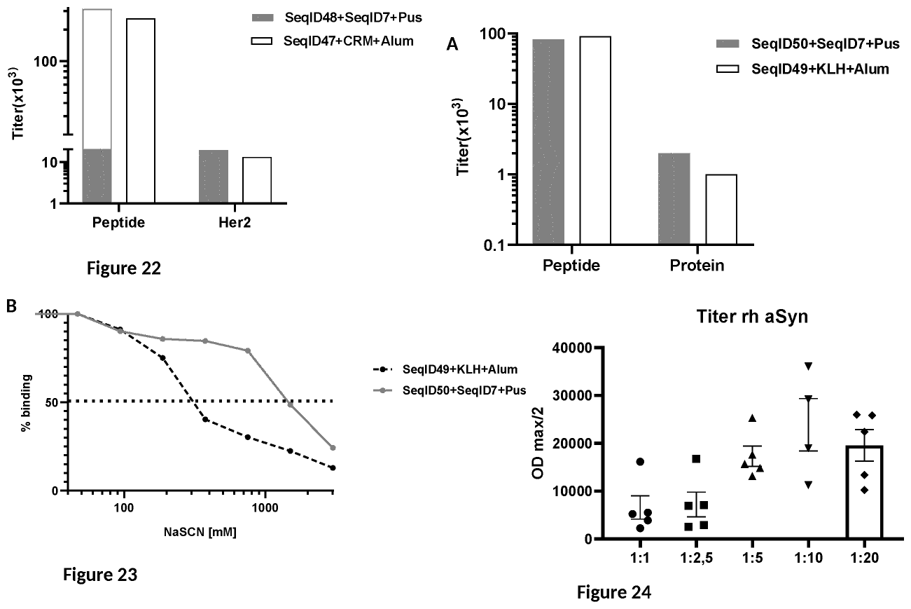

FIG. 22 shows: the comparative analysis of immune responses elicited by CLEC vaccines containing B-cell epitopes present in different forms of cancer/neoplastic disease (i.e. oncogenes): Her2. Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID48+SeqID7+Pus, i.d.) were evaluated against conventional peptide-component-based vaccines (SeqID47+KLH+Alum, s.c.). Samples were taken 2 weeks after 3rd application and analysed for anti-peptide and anti-Her2 protein responses.

FIG. 23 shows: the comparative analysis of immune responses elicited by CLEC vaccines containing B-cell epitopes present in different forms of neo-plastic disease/cancer (i.e. oncogenes): PD1. Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID50+SeqID7+Pus, i.d.) were evaluated against conventional peptide-component-based vaccines (SeqID49+KLH+Alum, s.c.). Samples were taken 2 weeks after 3rd application and analysed for A) anti-peptide and anti-PD1 protein responses. B) Antibody avidity to SeqID49 was assessed by ELISA based avidity assay.

FIG. 24 shows: the Comparative analysis of the target-protein specific immunogenicity induced by CLEC-based-peptide-CRM197 conjugate vaccines using different peptide-CRM197/CLEC ratios Female BALB/c mice, 8-12 weeks of age received a total of 3 intradermal vaccinations applied at a 2-week interval. Blood samples have been collected at baseline and after each vaccination to inform on the kinetics of the ensuing immune response. 5 different peptide-CRM-based vaccines have been used in this study applying different peptide-CRM/Pustulan ratios (w/w). All 5 groups have been immunised using SeqID6+CRM+Pus conjugates. 1:1, 1:2,5, 1:5, 1:10 and 1:20 represent conjugates with a w/w peptide-CRM conjugate/CLEC ratio of 1/1, 1/2,5, 1/5, 1/10 and 1/20. Immune reactions induced have been evaluated using samples taken 2 weeks after 3rd application and analysed for anti-aSyn protein responses by ELISA. Titer determination was based on calculation of ODmax/2.

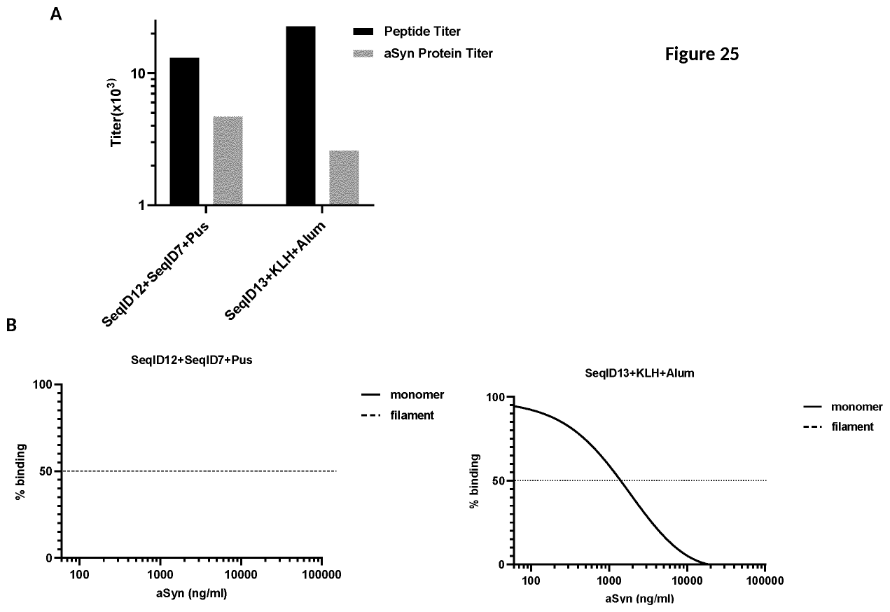

FIG. 25 shows: the comparative analysis of immune responses elicited by CLEC vaccines containing B-cell epitopes from aSyn (aa1-8). Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID12+SeqID7+Pustulan, i.d.) were evaluated against conventional peptide-component conjugate-based vaccines (SeqID13 conjugated with KLH and Alhydrogel (Alum), s.c.). Samples were taken 2 weeks after 3rd application and analysed for A) antipeptide and anti-aSyn protein responses and B) aSyn selectivity (inhibition ELISA). Black line: monomeric aSyn used for inhibition; dashed line: filamentous aSyn used for inhibition.

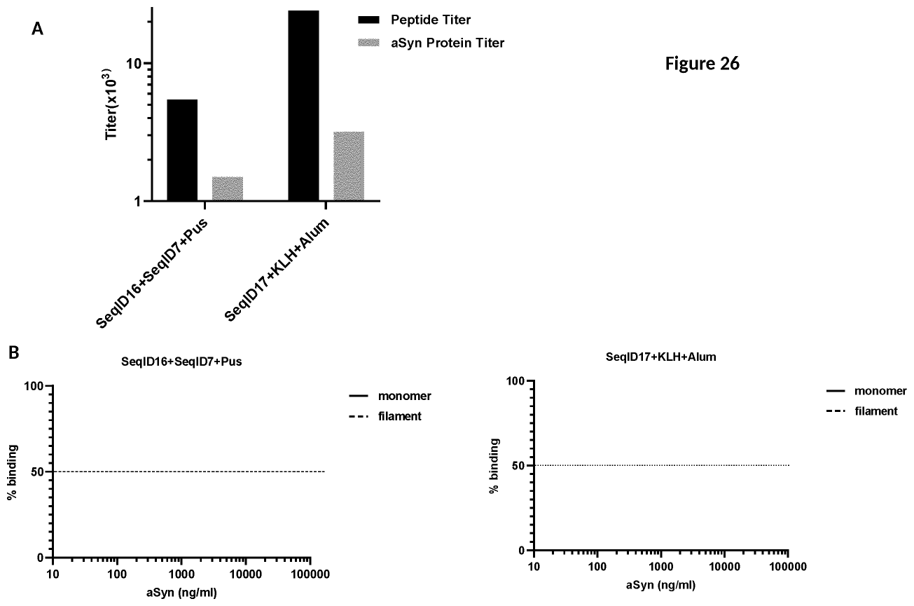

FIG. 26 shows the comparative analysis of immune responses elicited by CLEC vaccines containing B-cell epitopes from aSyn (aa100-108). Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID16+SeqID7 and Pustulan, i.d.) were evaluated against conventional peptide-component conjugate-based vaccines (SeqID17 conjugated with KLH and Alhydrogel (Alum), s.c.). Samples were taken 2 weeks after 3rd application and analysed for A) antipeptide and anti-aSyn protein responses and B) aSyn selectivity (inhibition ELISA). Black line: monomeric aSyn used for inhibition; dashed line: filamentous aSyn used for inhibition.

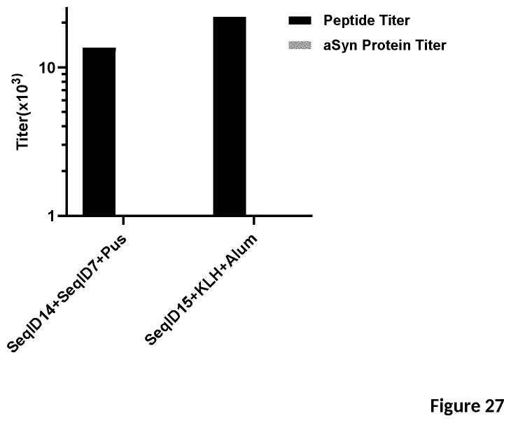

FIG. 27 shows the comparative analysis of immune responses elicited by CLEC vaccines containing B-cell epitopes from aSyn (aa91-97). Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID14+SeqID7 and Pustulan, i.d.) were evaluated against conventional peptide-component conjugate-based vaccines (SeqID15 conjugated with KLH and Alhydrogel (Alum), s.c.). Samples were taken 2 weeks after 3rd application and analysed for anti-peptide and anti-aSyn protein responses.

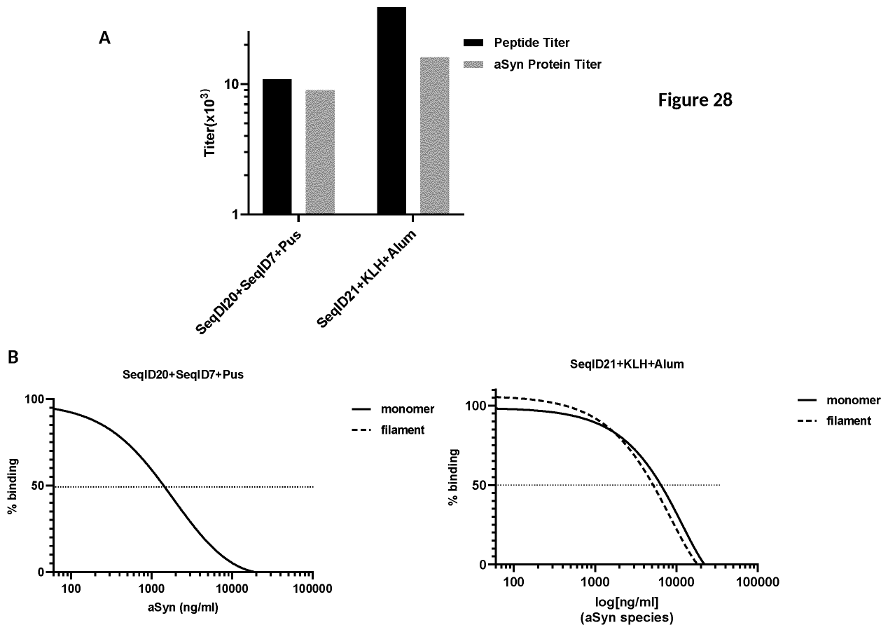

FIG. 28 shows the comparative analysis of immune responses elicited by CLEC vaccines containing B-cell epitopes from aSyn (aa130-140). Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID20+SeqID7 and Pustulan, i.d.) were evaluated against conventional peptide-component conjugate-based vaccines (SeqID21 conjugated with KLH and Alhydrogel (Alum), s.c.). Samples were taken 2 weeks after 3rd application and analysed for A) anti-peptide and anti-aSyn protein responses and B) aSyn selectivity (inhibition ELISA). Black line: monomeric aSyn used for inhibition; dashed line: filamentous aSyn used for inhibition.

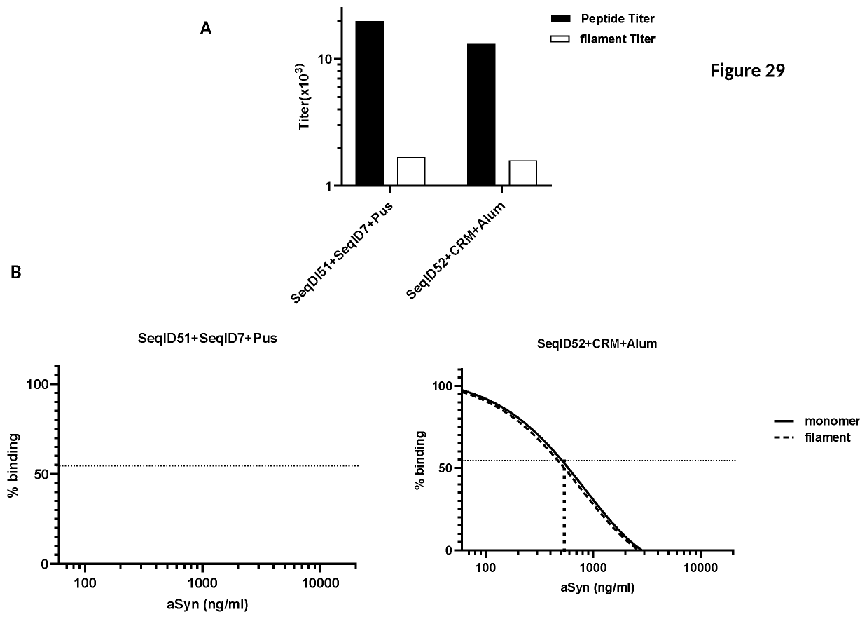

FIG. 29 shows the comparative analysis of immune responses elicited by CLEC vaccines containing B-cell epitopes from aSyn (aa115-122). Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID51+SeqID7 and Pustulan, i.d.) were evaluated against conventional peptide-component conjugate-based vaccines (SeqID52 conjugated with CRM and Alhydrogel (Alum), s.c.). Samples were taken 2 weeks after 3rd application and analysed for A) anti-peptide and anti-aSyn filament responses and B) aSyn selectivity (inhibition ELISA). Black line: monomeric aSyn used for inhibition; dashed line: filamentous aSyn used for inhibition.

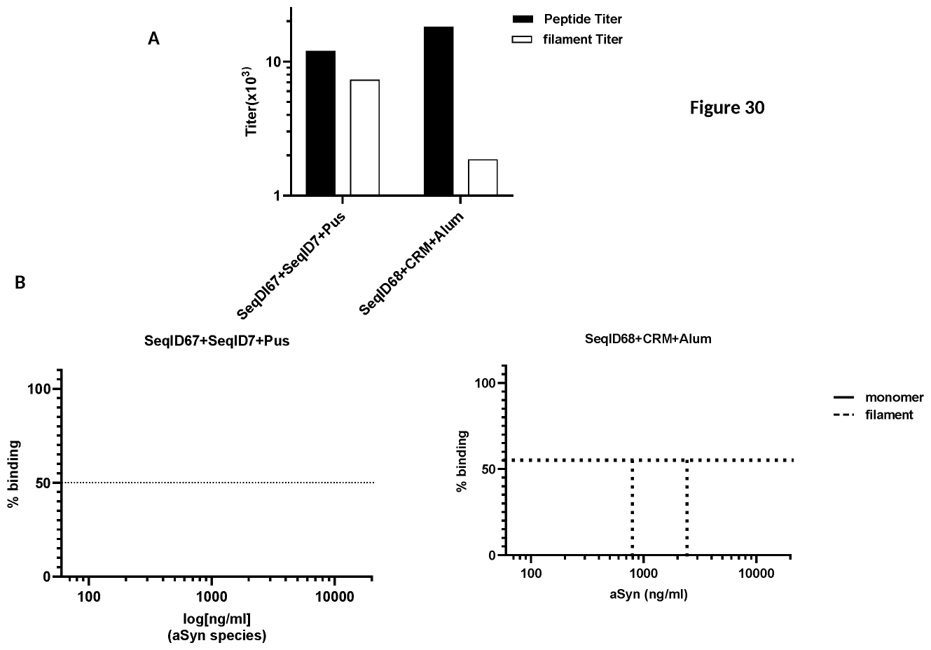

FIG. 30 shows the comparative analysis of immune responses elicited by CLEC vaccines containing B-cell epitopes from aSyn (aa115-124). Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID67+SeqID7 and Pustulan, i.d.) were evaluated against conventional peptide-component conjugate-based vaccines (SeqID68 conjugated with CRM and Alhydrogel (Alum), s.c.). Samples were taken 2 weeks after 3rd application and analysed for A) anti-peptide and anti-aSyn filament responses and B) aSyn selectivity (inhibition ELISA). Black line: monomeric aSyn used for inhibition; dashed line: filamentous aSyn used for inhibition.

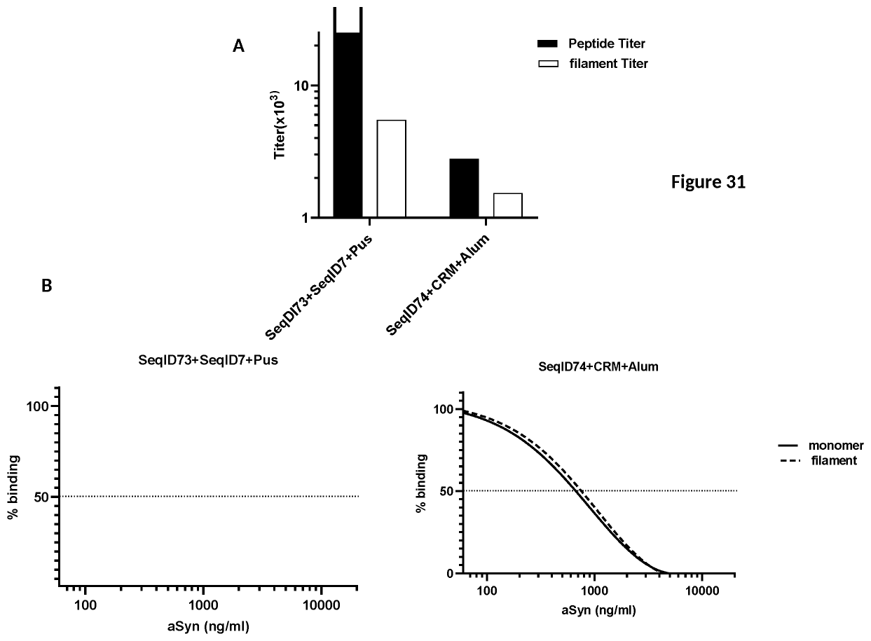

FIG. 31 shows the comparative analysis of immune responses elicited by CLEC vaccines containing B-cell epitopes from aSyn (aa107-113). Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. CLEC-based vaccines (SeqID73+SeqID7 and Pustulan, i.d.) were evaluated against conventional peptide-component conjugate-based vaccines (SeqID74 conjugated with CRM and Alhydrogel (Alum), s.c.). Samples were taken 2 weeks after 3rd application and analysed for A) anti-peptide and anti-aSyn filament responses and B) aSyn selectivity (inhibition ELISA). Black line: monomeric aSyn used for inhibition; dashed line: filamentous aSyn used for inhibition.

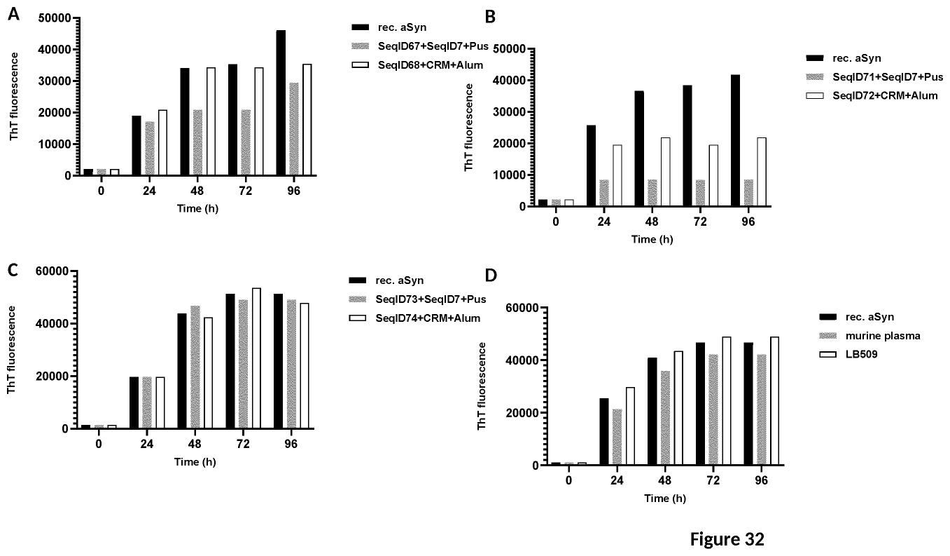

FIG. 32 shows the comparative analysis of in vitro functionality of immune responses elicited by CLEC based vaccines. Female BALB/c mice, 8-12 weeks of age received a total of 3 vaccinations applied at a 2-week interval (i.d. and s.c.). Blood samples have been collected at baseline and after each vaccination to inform on the kinetics of the ensuing immune response. Sample were taken 2 weeks after 3rd application and ThT kinetic measurements (i.e. fibrillar fraction of aSyn) were evaluated in the presence of A-C) CLEC-vaccine induced Abs (SeqID67/71/73+SeqID7 and pustulan, i.d.) or conventional peptide-component-induced Abs (SeqID68/72/74 conjugated with CRM and Alhydrogel (Alum), s.c.), or D) the aSyn specific, monoclonal Ab LB09 or untreated murine plasma.

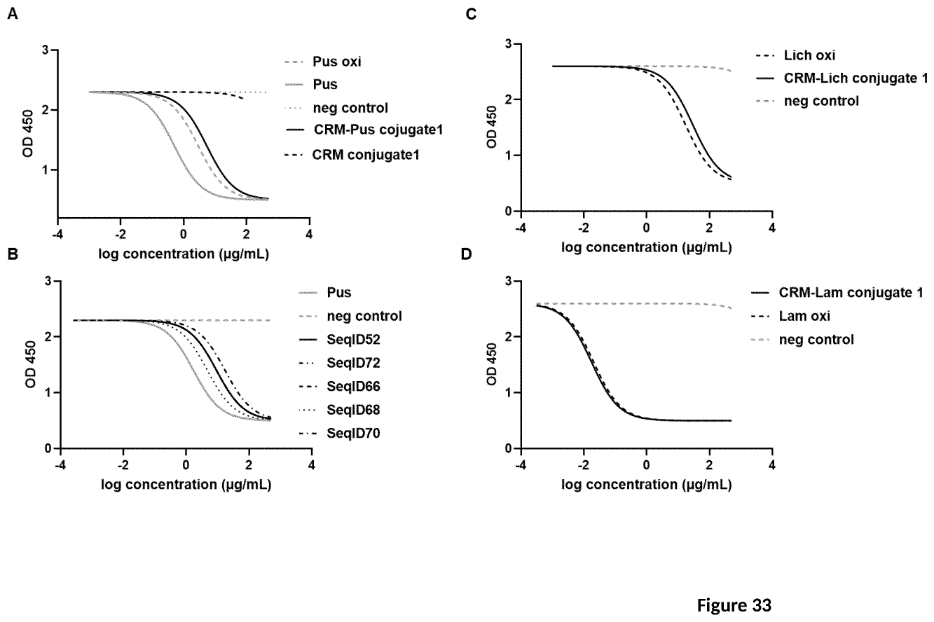

FIG. 33 shows the murine DC receptor (i.e. dectin-1) binding activity by CRM197-CLEC-conjugates in vitro. Comparative analysis of the dectin-1 binding ability determined by ELISA is shown. A) Pus refers to non-modified pustulan and pus oxi refers to activated pustulan. CRM-pus conjugate 1 refers to the SeqID6+CRM197+pustulan conjugate and CRM conjugate 1 refers to a CRM197+SeqID6 conjugate without β-Glucan modification. Neg control refers to sample without inhibitor B) SeqID52/66/68/70/72 refer to CRM197-pustulan conjugates with indicated B-cell epitopes. C) Lich oxi refers to activated lichenan and CRM-Lich conjugate 1 refers to the SeqID6+CRM197+lichenan conjugate. D) Lam oxi refers to activated laminarin and CRM-Lam conjugate 1 refers to the SeqID6+CRM197+laminarin conjugate.

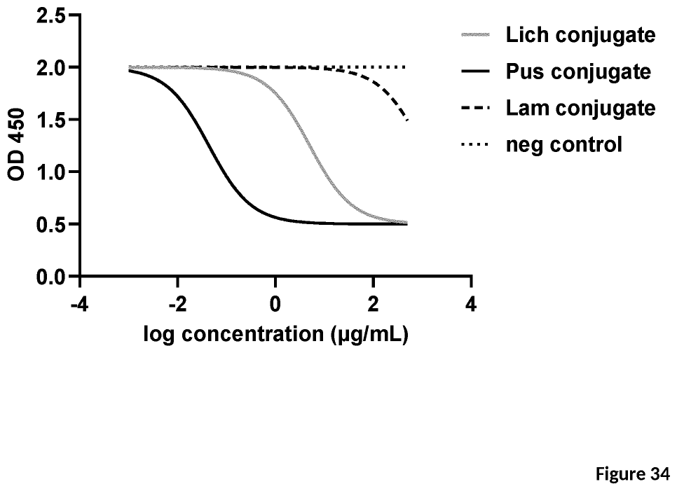

FIG. 34 shows the human DC receptor (i.e. dectin-1) binding activity by CRM197-CLEC-conjugates in vitro. Comparative analysis of the dectin-1 binding ability determined by ELISA is shown. Lich conjugate refers to the SeqID6+CRM197+lichenan conjugate, Pus conjugate refers to the SeqID6+CRM197+pustulan conjugate and Lam conjugate refers to the SeqID6+CRM197+laminarin conjugate. Neg control refers to sample without inhibitor.

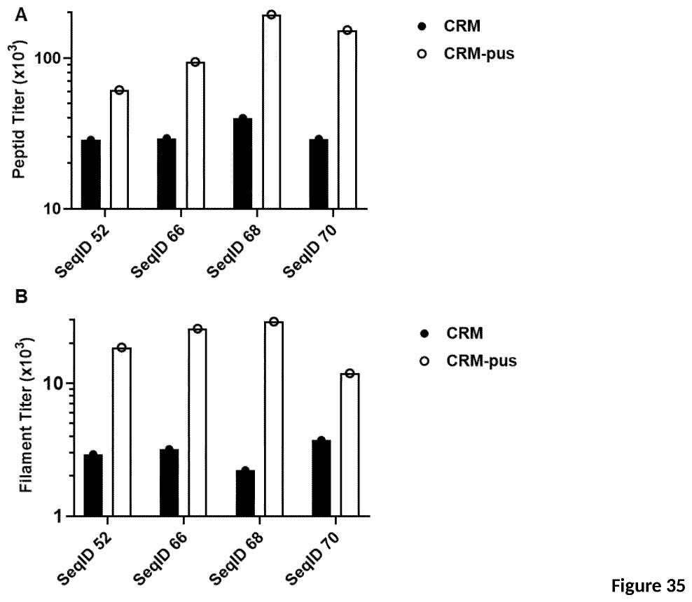

FIG. 35 shows the comparison of immunogenicity of different CRM-pustulan based vaccines. Female BALB/c mice, 8-12 weeks of age, received a total of 3 intradermal vaccinations applied at a 2-week interval. Blood samples have been collected at baseline and after each vaccination to inform on the kinetics of the ensuing immune response. Samples were taken 2 weeks after 3rd application and analysed for A) antipeptide response B) anti-aggregated aSyn filament responses.

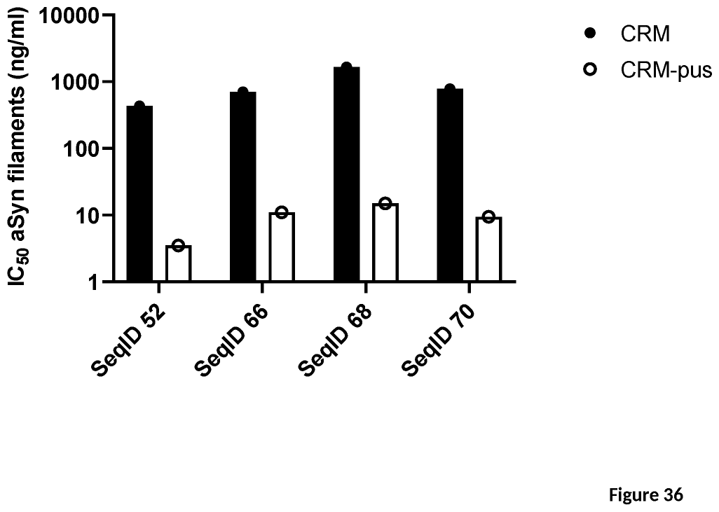

FIG. 36 shows the comparative analysis of the selectivity of the immune responses elicited by peptide+CRM+pustulan based vaccines in vivo against aSyn filaments. Female BALB/c mice, 8-12 weeks of age received a total of 3 intradermal or s.c. vaccinations applied at a 2-week interval. CRM-pustulan based vaccine were evaluated against conventional CRM vaccine. Sample were taken 2 weeks after 3rd application and subjected to aSyn selectivity assay (inhibition ELISA). IC50 values of antibodies inhibited with increasing doses of aSyn filaments are shown.

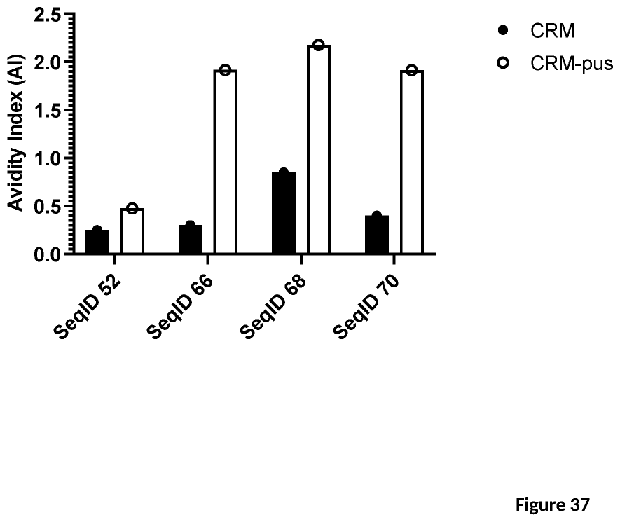

FIG. 37 shows the avidity of antibodies induced by peptide+CRM197+pustulan vaccines. Stability of aSyn-antibody complexes induced by peptide+CRM197+pustulan- or peptide+CRM197 vaccines after challenging with different concentrations of the chaotropic agent sodium thiocyanate (NaSCN) and the determined avidity indexes are shown.

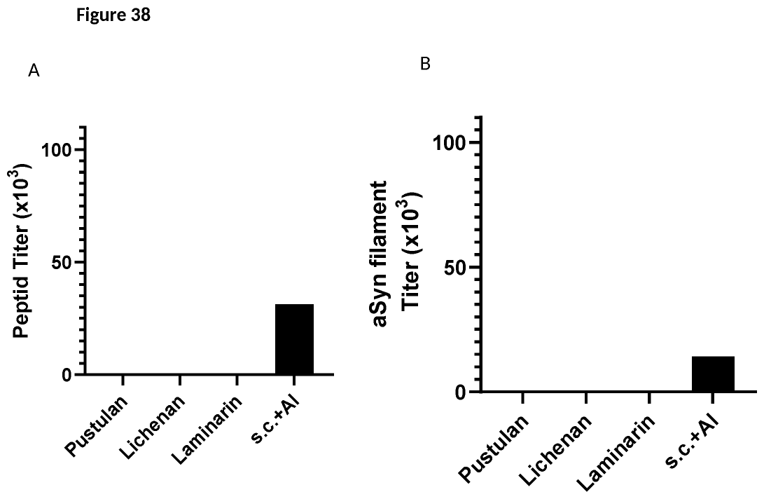

FIG. 38 shows the comparison of immunogenicity of different CLEC based vaccines. Female BALB/c mice, 8-12 weeks of age, received a total of 3 intradermal vaccinations applied at a 2-week interval. Blood samples have been collected at baseline and after each vaccination to inform on the kinetics of the ensuing immune response. Samples were taken 2 weeks after 3rd application and analysed for anti-SeqID6 peptide response (A) and anti aSyn Filament response (B) induced by the peptide+carrier+glucan-based vaccines or the non-CLEC modified, vaccine adjuvanted with Alum; dose: 20 μg peptide equivalent/injection; pustulan indicates SeqID6+CRM+pustulan, lichenan indicates SeqID6+CRM+lichenan, laminarin indicates SeqID6+CRM+laminarin, and s.c.+Alum indicates non-CLEC modified, vaccine SeqID6+CRM adjuvanted with Alum

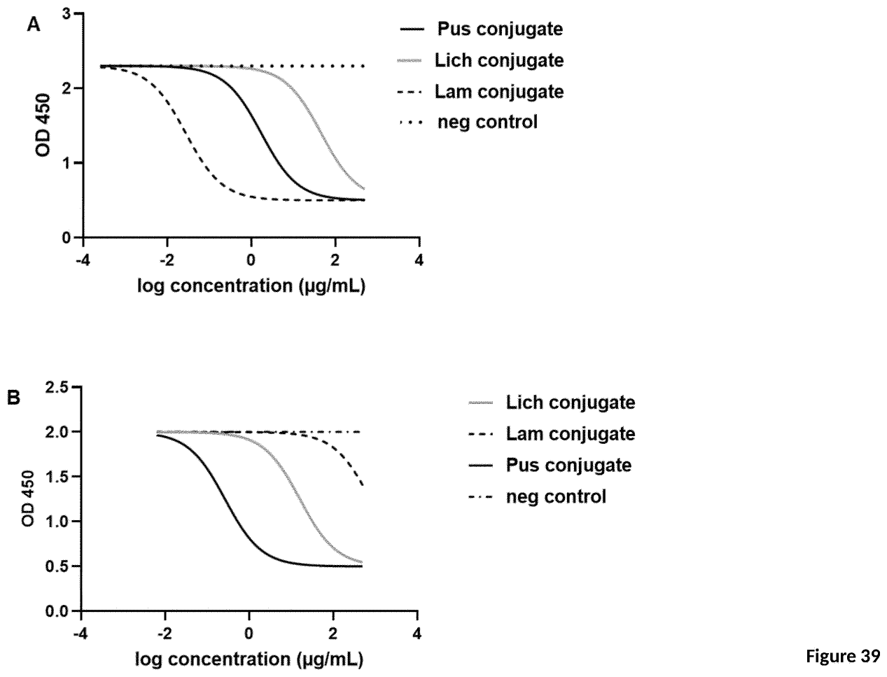

FIG. 39 shows the murine (A) and human (B) DC receptor (i.e. dectin-1) binding activity by peptide-CLEC-conjugates in vitro. Comparative analysis of the dectin-1 binding ability determined by ELISA is shown. Lich conjugate refers to the SeqID5+SeqID7+lichenan conjugate, Pus conjugate refers to the SeqID5+SeqID7+pustulan conjugate and Lam conjugate refers to the SeqID5+SeqID7+laminarin conjugate. Neg control refers to sample without inhibitor.

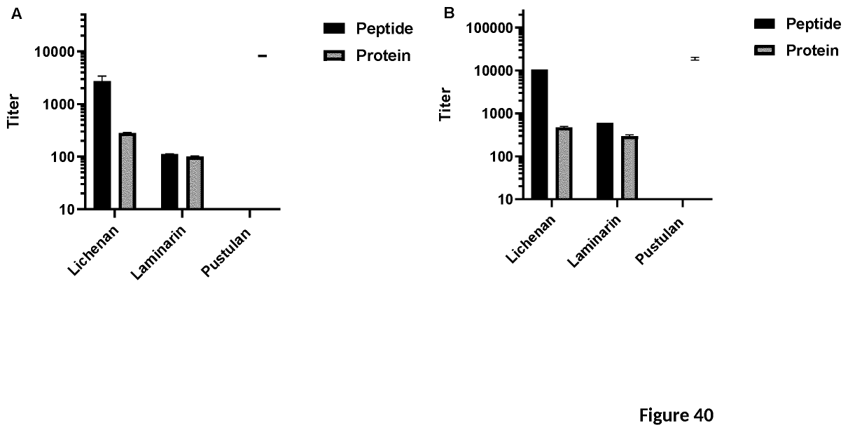

FIG. 40 shows the comparison of immunogenicity of different CLEC based vaccines. Female BALB/c mice, 8-12 weeks of age, received a total of 3 intradermal vaccinations applied at a 2-week interval. Blood samples have been collected at baseline and after each vaccination to inform on the kinetics of the ensuing immune response. Samples were taken 2 weeks after 3rd application and analysed for anti-peptide response (SeqID6, indicated as peptide) and anti aSyn response (indicated as protein) induced by the peptide-glucan-based vaccines (i.e.: SeqID5+SeqID7+CLEC, dose: 5 μg and 20 μg/injection; lichenan indicates SeqID5+SeqID7+lichenan; laminarin indicates SeqID5+SeqID7+laminarin and pustulan indicates SeqID5+SeqID7+pustulan)

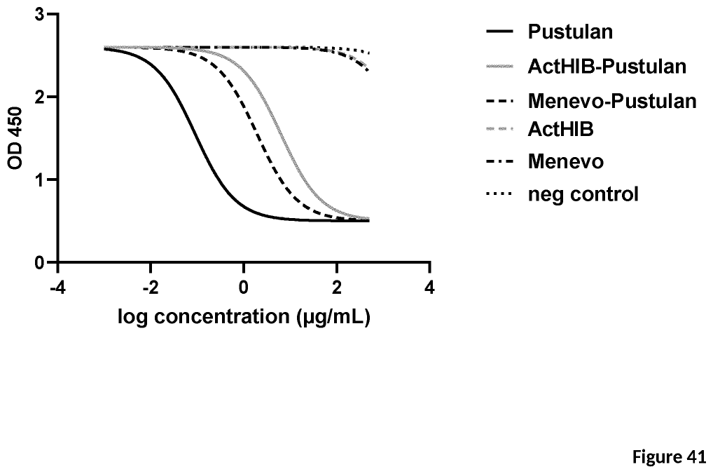

FIG. 41 shows the DC receptor (i.e. dectin-1) binding activity by glycoconjugate-pustulan-conjugates in vitro. Both CLEC modified vaccines, the oligosaccharide+CRM197+pustulan- and the polysaccharide+TT+pustulan-conjugates maintain high dectin-1 binding efficacy. Comparative analysis of the dectin-1 binding ability determined by ELISA is shown. Act-Pus refers to the Haemophilus influenzae type b capsular polysaccharide (polyribosyl-ribitol-phosphate, PRP) Tetanus Toxoid (TT) conjugate ActHIB® modified with pustulan, Act refers to ActHIB® conjugate vaccine without β-Glucan modification, Men refers to the Neisseria meningitidis oligosaccharide (A, C, W135, and Y) containing CRM197 conjugate vaccine Menveo® without β-glucan modification, Men-Pus refers to Menveo® vaccine modified with pustulan, and pus oxi refers to activated pustulan used for modification.

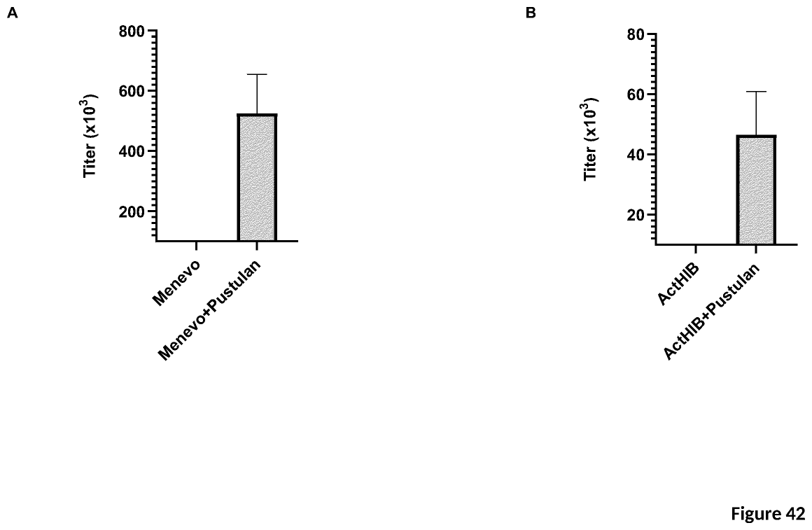

FIG. 42 shows the comparison of immunogenicity of different CLEC based glycoconjugate vaccines. Female BALB/c mice, 8-12 weeks of age, received a total of 3 i.d./i.m. vaccinations applied at a 2-week interval. Blood samples have been collected at baseline and after each vaccination to inform on the kinetics of the ensuing immune response. Samples were taken 2 weeks after 3rd application and analysed for anti-vaccine response induced by oligo/polysaccharide-carrier-glucan-based or non-glucan modified conjugate vaccines. A) shows responses induced by Menveo® conjugated to pustulan (Menveo®+Pustulan): N. meningitidis (A, C, W135, Y)+CRM197+pustulan (80%), or non-modified Menveo®: N. meningitidis (A, C, W135, Y)+CRM197, (dose: 5 μg); B) shows responses induced by ActHIB® conjugated to pustulan (ActHIB®+pustulan): H. influenzae (b) PRP+TT+pustulan (80%), or non-modified ActHIB® H. influenzae (b) PRP+TT (dose: 2 μg)

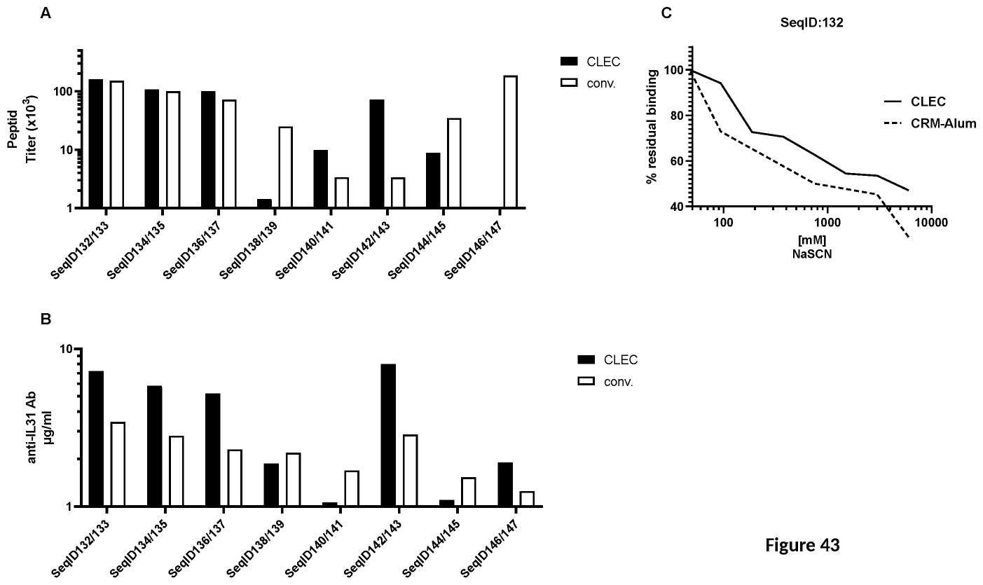

FIG. 43 shows: Comparative analysis of the immunogenicity of CLEC-based vaccines using different IL31 peptide epitopes. Female BALB/c mice, 8-12 weeks of age received a total of 3 intradermal vaccinations applied at a 2-week interval. Blood samples have been collected at baseline and after each vaccination to inform on the kinetics of the ensuing immune response. Immune responses elicited by 8 different CLEC-based vaccines (SeqID132+SeqID7+pustulan; SeqID134+SeqID7+pustulan; SeqID136+SeqID7+pustulan; SeqID138+SeqID7+pustulan; SeqID140+SeqID7+pustulan; SeqID142+SeqID7+pustulan; SeqID144+SeqID7+pustulan; and SeqID146+SeqID7+pustulan) were evaluated against the respective peptide-CRM197 conjugates adjuvanted with Alum (i.e. SeqID133+CRM197; SeqID135+CRM197; SeqID137+CRM197; SeqID139+CRM197; SeqID141+CRM197; SeqID143+CRM197; SeqID145+CRM197; and SeqID147+CRM197), respectively. Samples were taken 2 weeks after 3rd application and analysed for A) anti-peptide and B) anti-IL31 protein responses. C) shows the avidity of antibodies induced by SeqID132+SeqID7+pustulan or SeqID133+CRM vaccines determined by challenging with different concentrations of the chaotropic agent sodium thiocyanate (NaSCN).

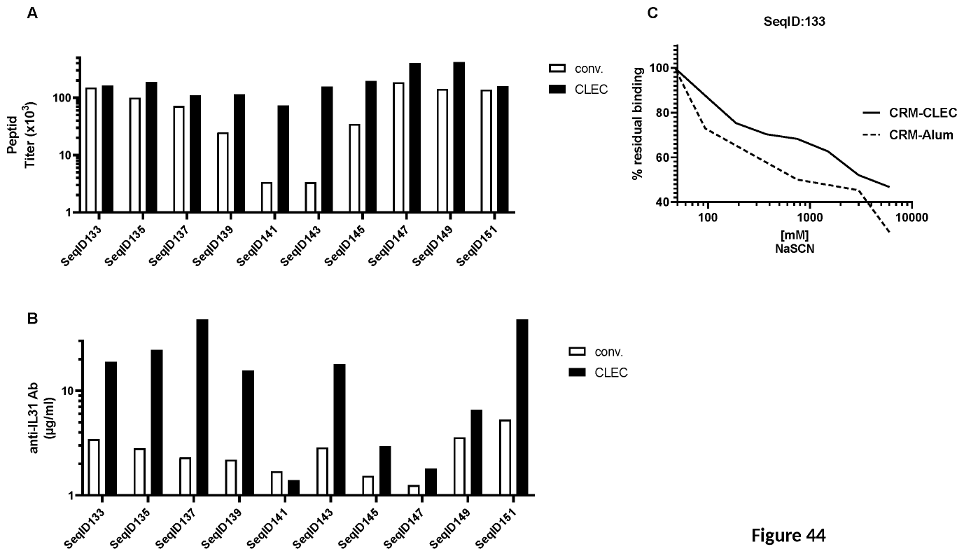

FIG. 44 shows: Comparative analysis of the immunogenicity of CLEC-based vaccines using different IL31 peptide epitopes Female BALB/c mice, 8-12 weeks of age received a total of 3 intradermal vaccinations applied at a 2-week interval. Blood samples have been collected at baseline and after each vaccination to inform on the kinetics of the ensuing immune response. Immune responses elicited by 10 different CLEC-based vaccines (SeSeqID135+CRM197+pustulan; Se-qID133+CRM197+pustulan; qID137+CRM197+pustulan; SeqID139+CRM197+pustulan; SeqID141+CRM197+pustulan; SeqID143+CRM197+pustulan; SeqID145+CRM197+pustulan; SeqID147+CRM197+pustulan; SeqID149+CRM197+pustulan; and SeqID151+CRM197+pustulan) were evaluated against the respective non modified peptide-CRM197 conjugates adjuvanted with Alum (i.e. SeqID133+CRM197; SeqID135+CRM197; SeqID137+CRM197; SeqID139+CRM197; SeqID141+CRM197; SeqID143+CRM197; SeqID145+CRM197; SeqID147+CRM197; Se-qID149+CRM197; and SeqID151+CRM197), respectively. Samples were taken 2 weeks after 3rd application and analysed for A) anti-peptide and B) anti-IL31 protein responses. C) shows the avidity of antibodies induced by SeqID133+CRM197+pustulan or SeqID133+CRM vaccines determined by challenging with different concentrations of the chaotropic agent sodium thiocyanate (NaSCN).

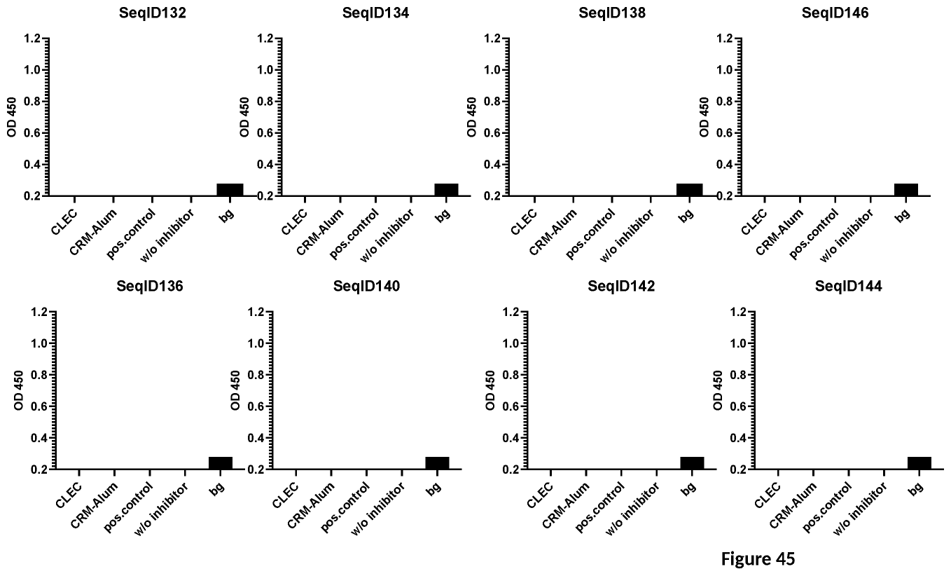

FIG. 45 shows inhibition of IL31 signaling by IL31 peptide-CLEC vaccine induced anti IL31 antibodies Inhibition of human IL-31 signaling by vaccine induced antibodies was assessed in human A549 cells (ATCC, Virginia, USA). Vaccine induced antibodies used were obtained from animals undergoing repeated immunization using IL31-peptide+SeqID7+pustulan conjugates (CLEC; IL31 peptides: SeqID132, SeqID134, SeqID136, SeqID138, SeqID140, SeqID142, SeqID144, SeqID146) as well as conventional IL31-peptide+CRM conjugates adjuvanted with Alum (CRMAlum; IL31 peptides: SeqID133, SeqID135, SeqID137, SeqID139, SeqID141, SeqID143, SeqID145, SeqID147). Pos.control: commercially available anti IL31 blocking Ab; w/o inhibitor: IL31 stimulation only, bg: background without IL31 stimulation.

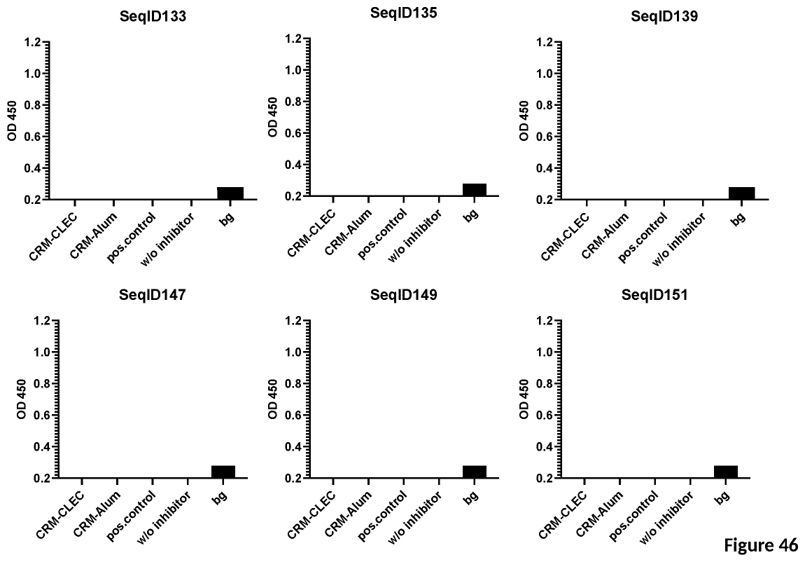

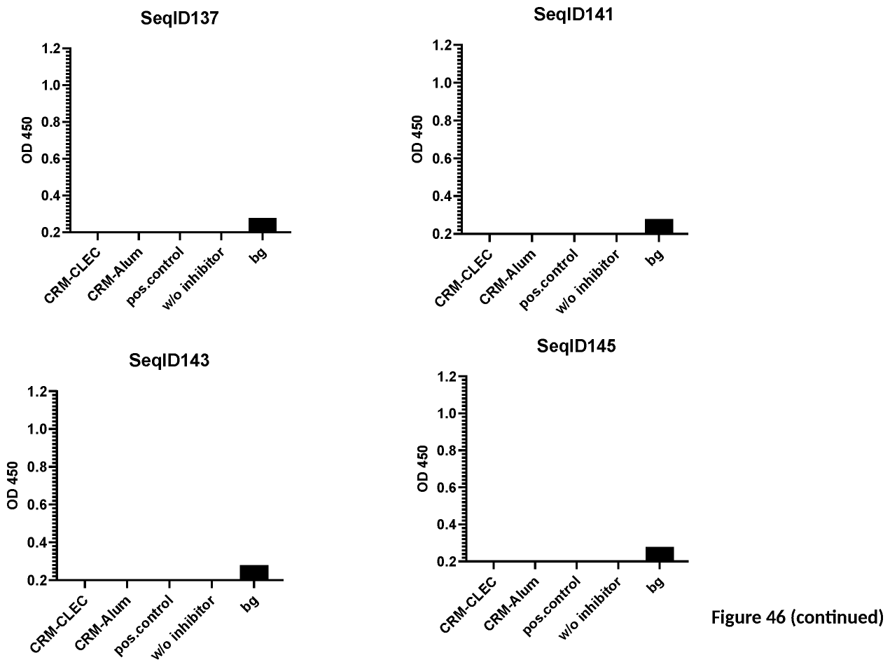

FIG. 46 shows inhibition of IL31 signaling by IL31 peptide-carrier-CLEC vaccine induced anti IL31 antibodies Inhibition of human IL-31 signaling by vaccine induced antibodies was assessed in human A549 cells (ATCC, Virginia, USA). Vaccine induced antibodies used were obtained from animals undergoing repeated immunization using IL31-peptide+CRM197+pustulan conjugates (CRM-CLEC; IL31 peptides: SeqID133, SeqID135, SeqID137, SeqID139, SeqID141, SeqID143, SeqID145, SeqID147, SeqID149, SeqID 151) as well as conventional IL31-peptide+CRM conjugates adjuvanted with Alum (CRM-Alum; IL31 peptides: SeqID133, SeqID135, SeqID137, SeqID139, SeqID141, SeqID143, SeqID145, SeqID147, SeqID149, SeqID 151). Pos.control: commercially available anti IL31 blocking Ab; w/o inhibitor: IL31 stimulation only, bg: background without IL31 stimulation.

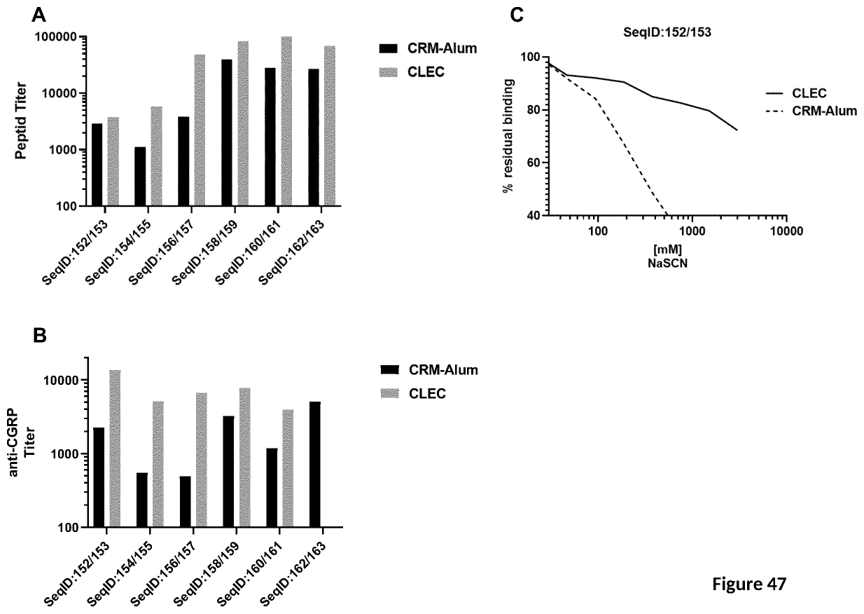

FIG. 47 shows: Comparative analysis of the immunogenicity of CLEC-based vaccines using different CGRP peptide epitopes Female BALB/c mice, 8-12 weeks of age received a total of 3 intradermal vaccinations applied at a 2-week interval. Blood samples have been collected at baseline and after each vaccination to inform on the kinetics of the ensuing immune response. Immune responses elicited by 6 different CLEC-based vaccines (SeqID152+SeqID7+pustulan; SeqID154+SeqID7+pustulan; SeqID156+SeqID7+pustulan; SeqID158+SeqID7+pustulan; SeqID160+SeqID7+pustulan; and SeqID162+SeqID7+pustulan) were evaluated against the respective peptide-CRM197 conjugates adjuvanted with Alum (i.e. SeqID153+CRM197; SeqID155+CRM197; SeqID157+CRM197; SeqID159+CRM197; SeqID161+CRM197; and SeqID163+CRM197), respectively. Samples were taken 2 weeks after 3rd application and analysed for A) antipeptide and B) anti-CGRP protein responses. C) shows the avidity of antibodies induced by SeqID152+SeqID7+pustulan or SeqID153+CRM vaccines determined by challenging with different concentrations of the chaotropic agent sodium thiocyanate (NaSCN).

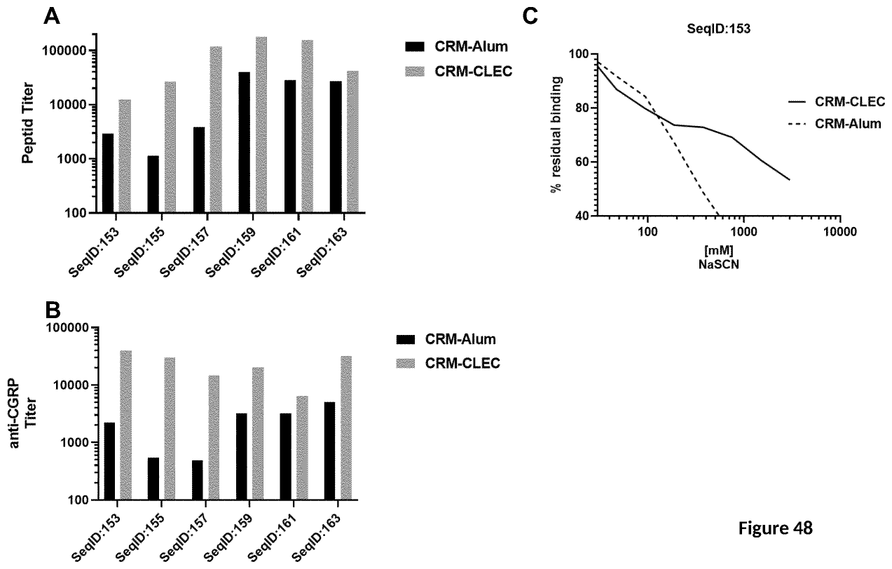

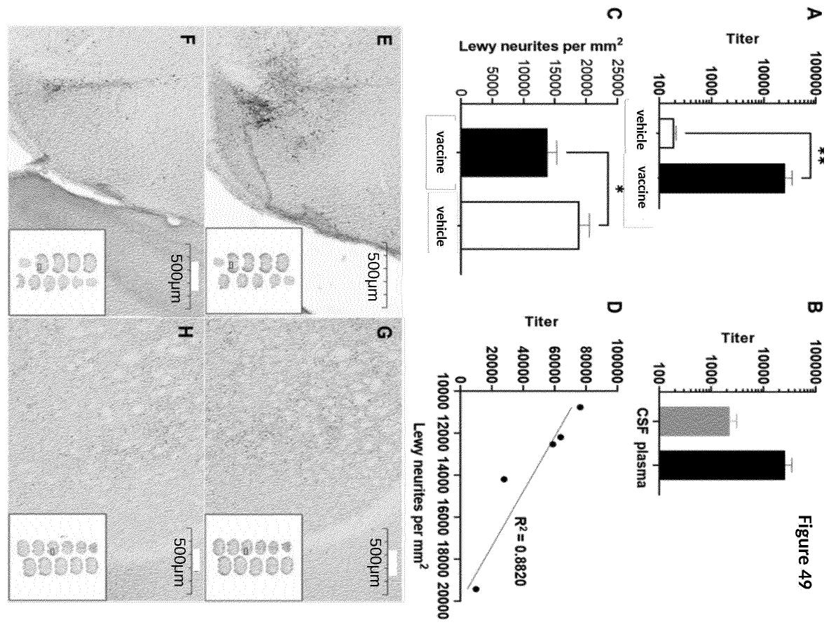

FIG. 48 shows: Comparative analysis of the immunogenicity of CLEC-based vaccines using different CGRP peptide epitopes Female BALB/c mice, 8-12 weeks of age received a total of 3 intradermal vaccinations applied at a 2-week interval. Blood samples have been collected at baseline and after each vaccination to inform on the kinetics of the ensuing immune response. Immune responses elicited by 6 different CLEC-based vaccines (SeSeqID155+CRM197+pustulan; qID153+CRM197+pustulan; SeSeqID159+CRM197+pustulan; qID157+CRM197+pustulan; SeqID161+CRM197+pustulan; and SeqID163+CRM197+pustulan) were evaluated against the respective non modified peptide+CRM197 conjugates adjuvanted with Alum (i.e. SeqID153+CRM197; SeqID155+CRM197; SeqID157+CRM197; SeqID159+CRM197; SeqID161+CRM197; and SeqID163+CRM197), respectively. Samples were taken 2 weeks after 3rd application and analysed for A) anti-peptide and B) anti-CGRP protein responses. C) shows the avidity of antibodies induced by SeqID153+CRM197+pustulan or SeqID153+CRM vaccines determined by challenging with different concentrations of the chaotropic agent sodium thiocyanate (NaSCN).

FIG. 49 shows SeqID5+SeqID7+pustulan vaccine induced antibodies inhibit aSyn aggregation in a PFF model in vivo. C57BL/6 mice stereotactically injected in the right substantia nigra with recombinant aSyn PFFs were immunized four times using SeqID5+SeqID7+pustulan vaccine (vaccine) or unconjugated CLEC (vehicle) as control starting on the day of PFF inoculation. Plasma was collected after the third immunization. Brains, plasma and CSF were harvested 126 days post PFF inoculation. Plasma titer of antibodies specific for the peptide used for immunization (A) collected two weeks after the third immunization at day 126. (B) Comparison of CSF and plasma titers of antibodies specific for the B-cell peptide of the vaccine at day 126. (C) Analysis of phosphor-S129 aSyn-positive aggregates over all brain areas in SeqID5+SeqID7+pustulan vaccinated and CLEC treated mice. (D) Correlation between antibody response and the level of synucleinopathy in vaccine recipients (r=−0.9391; CI (95%) −0.9961 to −0.3318, p=0.0179, and R2=0.882). (E-H) Representative pSer129 aSyn staining in the injected brain hemisphere at the level of (E, F) the substantia nigra and (G, H) the striatum. (E, G) vehicle treated mice and (F, H) vaccine treated mice following PFF injection. Error bars indicate the mean±SEM of n=5-9 animals per group. Statistical differences were evaluated by an unpaired t-test; **p<0.01; *p<0.05.

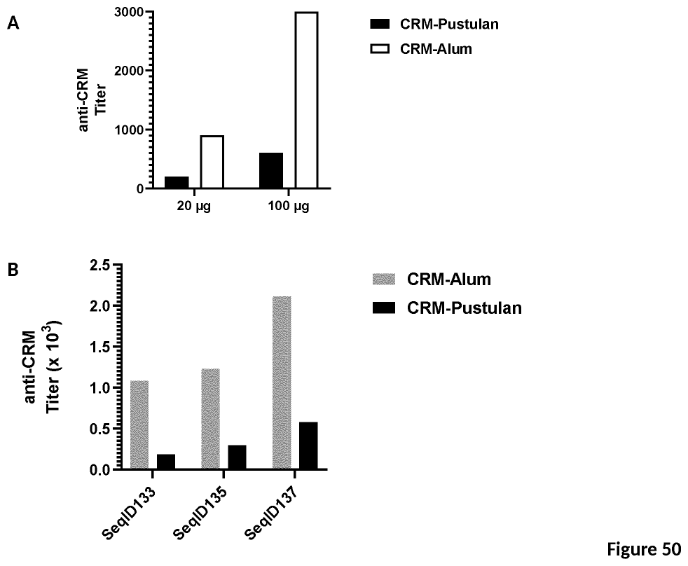

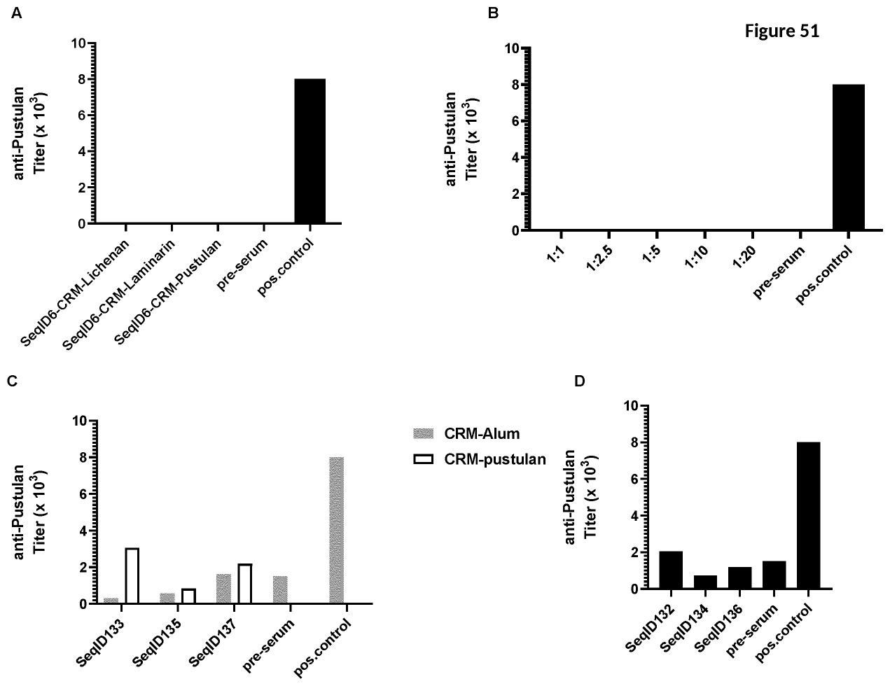

FIG. 50 shows the analysis of carrier-specific immunogenicity of Peptide+CLEC and Peptide+CRM+CLEC conjugates Female BALB/c mice, 8-12 weeks of age received a total of 3 intradermal/s.c. vaccinations applied at a 2-week interval. Blood samples have been collected at baseline and after each vaccination to inform on the kinetics of the ensuing immune response. Immune responses elicited by 4 different CLEC-based vaccines (CRM-Pustulan; i.e. SeqID6+CRM197+pustulan; SeqID133+CRM197+pustulan; SeqID135+CRM197+pustulan; and SeqID137+CRM197+pustulan) were evaluated against the respective peptide-CRM197 conjugates adjuvanted with Alum (CRM-Alum; i.e. SeqID6+CRM197; SeqID133+CRM197; SeqID135+CRM197; and SeqID137+CRM197), respectively. Samples were taken 2 weeks after 3rd application and analysed for A) SeqID6+CRM197+Pustulan induced and B) SeqID133+CRM197+pustulan; SeqID135+CRM197+pustulan; and SeqID137+CRM197+pustulan induced anti-CRM responses in vivo.

FIG. 51 shows the analysis of CLEC-specific immunogenicity of Peptide+CLEC and Peptide+CRM+CLEC conjugates Female BALB/c mice, 8-12 weeks of age received a total of 3 intradermal vaccinations applied at a 2-week interval. Blood samples have been collected at baseline and after each vaccination to inform on the kinetics of the ensuing immune response. Immune responses elicited by 14 different CLEC-based vaccines were evaluated. Samples were taken 2 weeks after 3rd application and analysed for anti-pustulan responses in vivo; A) samples: SeqID6+CRM197+pustulan, SeqID6+CRM197+lichenan; SeqID6+CRM197+laminarin B) samples SeqID6+CRM197+pustulan; pustulan coupled at indicated conjugate/pustulan ratios (w/w); C) samples: SeqID133+CRM197+pustulan; SeqID135+CRM197+pustulan; and SeqID137+CRM197+pustulan; D) SeqID132+SeqID7+pustulan; SeqID134+SeqID7+pustulan; and SeqID136+SeqID7+pustulan; pre-serum: samples obtained prior to immunisation; pos. control: samples from animals immunized with non-oxidized Pustulan only.

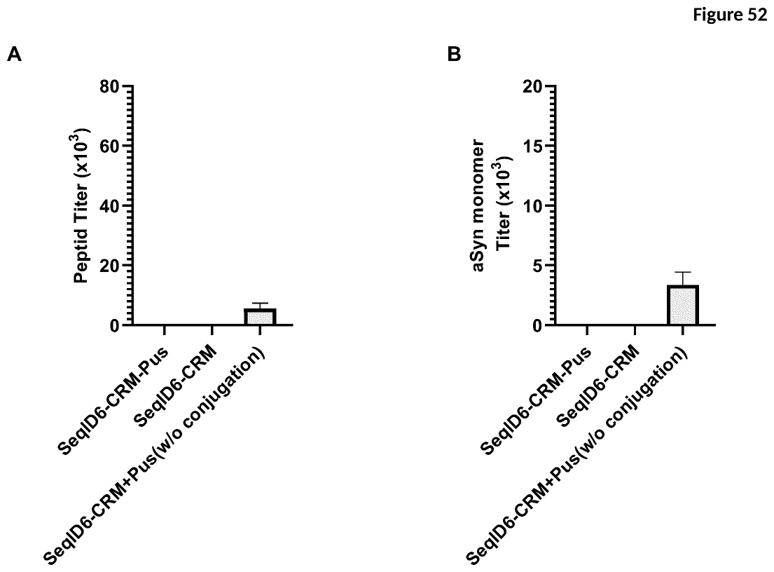

FIG. 52 shows: Analysis of the immunogenicity of peptide-carrier-glucan conjugates and vaccines consisting of peptide-carrier conjugates and unconjugated glucan. Female BALB/c mice, 8-12 weeks of age received a total of 3 intradermal vaccinations applied at a 2-week interval. Blood samples have been collected at baseline and after each vaccination to inform on the kinetics of the ensuing immune response. Samples were taken 2 weeks after 3rd application and analysed for anti-SeqID6 peptide (A) and anti aSyn monomer (B) responses. Vaccines used: SeqID6+CRM197+pustulan, SeqID6+CRM197 mixed with non-oxidized pustulan and non-CLEC modified, non-adjuvanted SeqID6+CRM197.

### EXAMPLES

### Material and Methods

### 1) Oxidation of CLEC/Glucan-Backbone

For formation of vaccine conjugates, polysaccharides, especially also CLEC/β-glucans need to be chemically modified to generate reactive groups that can be used to link proteins/peptides. Two commonly used methods for polysaccharide activation are periodate oxidation at vicinal hydroxyls as well as cyanylation of hydroxyls. Further methods of activation of polysaccharides are possible and well known in the art. Examples shown in the present example section rely on mild periodate oxidation.

Depending on their solubility, CLECs and β-glucans (e.g. mannan, lichenan, pustulan or β-glucan from barley) are oxidized using periodate oxidation in aqueous solution or DMSO.

The degree of oxidation is predetermined based on adding the periodate solution at a molar ratio (periodate:sugar subunit; 100%=1 Mol periodate per Mol sugar monomers) of 1:5 (i.e., 20% oxidation) to 2, 6:1 (260% oxidation degree).

Briefly, sodium periodate is added to a molar ratio of 1:5 to 2,6:1 (periodate: sugar subunit, corresponding to 20% and 260% oxidation degree) to open furanose rings of the β-glucans between vicinal diols leaving two aldehyde groups as substrate for the subsequent coupling reactions. 10% (v/v) 2-propanol is added as radical scavenger. The reaction is incubated for 4 h at room temperature on an orbital shaker (1000 rpm) in the dark. Subsequently, oxidized glucans are dialysed 3 times against water using Slide-A-Lyzer™ (Thermo Scientific) or Pur-A-Lyzer™ (Sigma Aldrich) cassettes with a 20 kDa cutoff to remove sodium (per) iodate and low molecular weight glucan impurities. Dialysed glucans can be directly subjected to the peptide conjugation reaction or stored at −20° C. or lyophilized and stored at 4° C. for further use.

### 2) Conjugation of WISIT Vaccines

2a. Via Hydrazone Formation

Polypeptides contain a hydrazide group at the N- or C-terminus for aldehyde coupling. In the case that coupling orientation is intended via the N-terminus of the selected peptide to the aldehyde groups of the glucan moieties the peptide is designed to contain a suitable linker/spacer, e.g. succinic acid. Alternatively, also intact proteins (e.g.: CRM197) have been used for glucan coupling.

Typical examples for such peptides: N-terminal coupling of peptides: H₂N—NH—CO—CH₂—CH₂—CO-Polypeptide-COOH; C-terminal coupling: NH₂-Polypeptide-NH—NH₂.

For coupling, activated glucan solution (i.e., oxidized pustulan) is stirred with the dissolved hydrazide modified peptides or intact proteins (e.g.: CRM197) in coupling buffer (depending on the isoelectric point of the peptide either sodium acetate buffer at pH 5.4, or DMEDA at neutral pH (6.8) are used). The free hydrazide group within the peptides reacts with the aldehyde group to a hydrazone bond forming the final conjugate. For proteins, coupling to activated glucan is based on reaction of the amino group of the Lysine residues present to reactive aldehydes on the glucan moieties in the presence of sodium cyanoborohydride.

Subsequently, the conjugate is reduced by addition of sodium borohydride in borate buffer (pH 8.5). This step reduces the hydrazine bond to a stable secondary amine and converts unreacted aldehyde groups in the sugar backbone into primary alcohols. Carbohydrate concentration in conjugates was estimated using anthrone method and peptide concentration was estimated by UV spectroscopy or determined by amino acid analysis.

2b. Coupling Using Heterobifunctional Linkers

The second conjugation technique applied relies on heterobifunctional linkers (e.g.: BMPH (N-β-maleimidopropionic acid hydrazide, MPBH (4-[4-N-maleimidophenyl] butyric acid hydrazide), EMCH (N-[ε-Maleimidocaproic acid) hydrazide) or KMUH (N-[κ-maleimidoundecanoic acid] hydrazide) short, maleimide-and-hydrazide crosslinkers for conjugating sulfhydryls (cysteines) to carbonyls (aldehyde)).

Polypeptides contain a cysteine (Cys) at the N- or C-terminus for maleimide coupling. Typical examples for such peptides: N-terminal coupling of peptides: Cys-Peptide-COOH; C-terminal coupling: NH₂-Pept-Cys-COOH.

For coupling, activated glucan solution (i.e., oxidized pustulan) is reacted overnight with BMPH (ratios used 1:1 ratio (w/w) to 2:1 ratio BMPH:pustulan) and subsequently dialysed 3× with PBS. BMPH-activated glucan is then mixed with the dissolved Cys-modified polypeptides in coupling buffer (e.g. phosphate-buffered saline, PBS). The maleimide group reacts with sulfhydryl groups from the peptides to form stable thioether linkages and together with the hydrazone formed between linker and reactive aldehydes results in stable conjugates. Carbohydrate concentration in conjugates was estimated using anthrone method and polypeptide concentration was determined by amino acid analysis or Ellmann's assay using Ellman's reagent (5, 5′-dithio-bis-(2-nitrobenzoic acid), DTNB). DTNB reacts with sulfhydryl groups to yield a colored product, providing a reliable method to measure reduced cysteines and other free sulfhydryls in solution by spectrophotometric measurement (λmax=412 nm; ε=14,150/M·cm).

### 2c) Polypeptide KLH/CRM Conjugation

Polypeptides (containing N- or C-terminal Cys residues, see above) were coupled to the carrier CRM-197 (e.g.: EcoCRM, Fina Biosolutions) or KLH (Sigma Aldrich) by using the heterobifunctional crosslinking agents GMBS or SMCC (Thermo Fisher). Briefly, CRM-197/KLH was mixed with an excess of GMBS or SMCC (acc. to manufacturer's protocol) at room temperature to allow for activation, followed by removal of excess GMBS by desalting column centrifugation. Excess peptide was then added to the activated carrier for coupling (buffer: 200 mM Na-phosphate (pH=6, 8)) and subsequently dialysed 3× with PBS. Coupling efficacy/peptide content was assessed using an Ellmann assay (Ellmann reagent: 5,5′-dithiobis-(2-nitrobenzoic acid) used for quantitating free sulfhydryl groups in solution). The polypeptide CRM-197/KLH conjugate was further formulated with Alum (Alhydrogel® adjuvant 2%) and applied to animals subcutaneously. Identical amounts of conjugated polypeptides were injected per mouse when the CRM-197/KLH vaccines were compared to other vaccines according to the present invention.

### 2d) Gluco-Neoconjugate Formation Using Polypeptide, KLH/CRM197 and Glucan

Polypeptide-KLH and polypeptide-CRM197 conjugates, produced as described in 2c), were also coupled to activated glucans at different Polypeptide-KLH and polypeptide-CRM197 to Glucan ratios (i.e. 1/1 (w/w), 1/2 (w/w), 1/5 (w/w), 1/10 (w/w) and 1/20 (w/w), respectively). Following polypeptide conjugate formation, Pep-KLH or Pep-CRM conjugates are reduced using Dithiothreitol (DTT). Reduced carrier-conjugates are coupled to activated glucans in the presence of an excess of heterobifunctional linker BMPH. Coupling is achieved via the maleimide group of BMPH to sulfhydryl residues of the reduced KLH or CRM197 conjugate forming a stable thioether bond and of aldehyde groups in the glycan with the hydrazide group of BMPH. After 2 hours at room temperature, the generated hydrazones are reduced to stable secondary amines by overnight incubation with sodium cyanoborohydride. Subsequently, gluco-neoconjugates are dialysed 3 times against PBS or water using Slide-ALyzer™ (Thermo Scientific) or Pur-A-Lyzer™ (Sigma Aldrich) cassettes to remove low molecular weight impurities (see also: Example 23).

3) Determination of Biological Activity of CLEC-Conjugates in vitro

Biological activity of mannan and glucan conjugates in vitro was analyzed by ELISA using a soluble murine Fc-dectin-1a receptor (InvivoGen) or ConA as described in Korotchenko et al., 2020. Briefly, ELISA plates are coated with a reference glucan (CLRagonists, CLECs), e.g.: pustulan, lichenan or mannan, and are reacted with fluorescently labeled ConA (for mannan) or soluble murine Fc-dectin-1a receptor (for pustulan and other β-D-glucans), which can be detected by a HRP-labeled secondary antibody. The oxidized carbohydrates as well as the gluconeoconjugates are tested in a competitive ELISA (increasing concentration of CLECs or conjugates are added to the soluble receptors used for the assay to reduce receptor binding to coated CLECs) to demonstrate their functionality. IC50 values are used to determine biological activity (i.e.: binding efficacy to soluble receptors in comparison to non-oxidised, non-conjugated ligands).

### 4) Activation Analysis Using Bone Marrow-Derived Dendritic Cells

Bone marrow-derived dendritic cells (BMDCs) were harvested from mouse femur and tibia and incubated with 20 ng/ml murine GM-CSF (Immunotools), as described in Korotchenko et al., 2020 with minor changes. Effects of various conjugates as well as of positive controls (=LPS) on CD80 and MHCII expression were assessed by FACS analysis on CD11c⁺MHCII⁺CD11b^{int} GM-CSF-derived DCs (GMDCs).

### 5) Determination of the Hydrodynamic Radius

The hydrodynamic radius of conjugates was analyzed by dynamic light scattering (DLS). Briefly, samples (i.e., conjugates) were centrifuged at 10,000 g for 15 minutes (Merck Millipore, Ultrafree-MC-VV Durapore PVDF). All sample wells were sealed with silica oil to prevent evaporation and data was collected sequentially for approximately 24 hours. All measurements were performed with a WYATT DynaPro PlateReader-II at 25° C. in a 1536 well plate (1536W SensoPlate, Greiner Bio-One). Samples were measured in triplicate. All measurements were filtered for a baseline value of 1.00±0.005 so only curves that returned to values between 0.995 and 1.005 were considered for further analysis (e.g., cumulants radius and regularization analysis). Analysis of samples was performed according to https://www.wyatt.com/library/application-notes/bytechnique/dls.html, and by DYNAMICS User's Guide (M1406 Rev. C, version 7.6.0), Technical Notes TN2004 and TN2005 (all on: www.Wyatt.com)

### 6) Animal Experiments

Female BALB/c mice, n=5 mice per group, were immunized either with different CLEC conjugates (i.d., i.m., s.c.), with peptide-CRM-197/KLH conjugates (i.d.) or peptide-CRM-197/KLH conjugates adsorbed to Alum (s.c.) as well as with respective controls (e.g. unconjugated CLEC, mixture of CLEC and peptides, etc.). Animals were vaccinated 3 times in bi-weekly intervals and blood samples were taken regularly one day before each vaccination and two weeks after the last application unless differently indicated.

### 7) Quantification of Vaccine Induced Antibodies in Murine Plasma Using ELISA

Whole blood was collected from mice using heparin as anticoagulant and plasma was obtained by centrifugation. Plasma samples were stored at −80° C. To detect anti-target specific antibodies, ELISA plates (Nunc Maxisorb) were coated with peptide-BSA conjugates or recombinant proteins/fragments (usual concentration 1 μg/ml) using 50 mM sodium carbonate buffer, overnight at 4° C. All anti-polypeptide ELISA used in the examples provided are performed using Pep-BSA conjugates (e.g., SeqID3 (Sequence: DQPVLPD) with a C-terminal C for coupling to maleimide activated BSA; nomenclature: Pep1c (DOPVLPD-C, SeqID 3) is used as bait for anti-Pep1 specific responses elicited by Pep1b (SeqID2; DQPVLPD-(NH—NH₂)) and Pep1c-containing conjugate vaccines). Plates were blocked with 1% bovine serum albumin (BSA) and plasma samples were serially diluted in the plates. Detection of target specific antibodies was performed with biotinylated anti-mouse IgG (Southern Biotech) and subsequent colour reaction using Streptavidin-POD (Roche) and TMB. EC₅₀ values were calculated using GraphPad Prism software (Graph Pad Prism www.graphpad.com/scientific-software/prism/) following non-linear regression analysis (four-parameter logistic fit function).

| Target protein | Antibody |
|---|---|
| Alpha synuclein recombinant | Anti-alpha synuclein 115-121 AB |
| (Anaspec) | (LB509) (Biolegend) |
| Alpha synucuclein monomer | Anti-alpha synuclein 115-121 AB |
| (Abcam) | (LB509) (Biolegend) |
| Alpha synuclein filament | Anti-alpha synuclein 115-121 AB |
| (Abcam) | (LB509) (Biolegend) |
| Amyloid beta 1-40 (Biolegend) | Anti-Amyloid beta 1-16 AB (6E10) |
| | (Biolegend) |
| Amyloid beta 1-42 (Biolegend) | Anti-Amyloid beta 1-16 AB (6E10) |
| | (Biolegend) |
| [Pyr3] Amyloid beta 3-40 | |
| (Anaspec) | |
| [Pyr3] Amyloid beta 3-42 | |
| (Anaspec) | |
| Tau 441 recombinant (Anaspec) | |
| PD1 recombinant (Abcam) | |
| Erb2/Her2 recombinant (R&D | |
| Systems) | |
| Bet v1 recombinant | |
| Extra Membrane Proximal Domain | |
| (EMPD) recombinant | |
| Pustulan (Biozol) | |
| KLH (Sigma) | Anti-KLH AB (Sigma) |
| CRM197 (FinaBiosolution) | Anti-Diphtheria AB (Abcam) |

8) Characterization of Binding Preference of aSyn Specific Antibodies by Inhibition ELISA

ELISA plates (Nunc Maxisorb) were coated either with aSyn monomers (Abcam) or aSyn filaments (Abcam) and blocked with 1% bovine serum albumin (BSA). The control antibodies and plasma samples were incubated with serially diluted aSyn monomers or aSyn filaments in low-binding ELISA plates. Next, the pre-incubated antibodies/plasma samples were added to the monomer/filament-coated plates and detection of binding was performed with biotinylated anti-mouse IgG (Southern Biotech) and subsequent colour reaction using Streptavidin-POD (Roche) and TMB. log IC50 values were calculated as the concentration of either monomeric or filamentous aSyn needed to quench half of the ELISA signal and were used as an estimate of the Abs selectivity for the investigated antigen. log IC50 values were calculated using GraphPad Prism software (Graph Pad Prism www.graphpad.com/scientific-software/prism/) following non-linear regression analysis (four-parameter logistic fit function).

9) Quantification of aSyn Aggregation

The protein aggregation assay in the automated format was carried out in a reaction volume of 0.1 ml in black, flat-bottomed 96-well plates at continuous orbital shaking in an GENIOS Microplate Reader (Tecan, Austria). The kinetics was monitored by top reading of fluorescence intensity every 20 minutes using 450-nm excitation and 505-nm emission filters. Fibril formation in the absence and presence of antibodies (antibody/protein molar ratio varied from 6×10⁻⁵ to 3×10⁻³) was initiated by shaking the aSyn solution, at a concentration of 0.3 mg/ml (20.8 UM), in 10 mM HEPES buffer (pH 7.5), 100 mM NaCl, 5 μM ThT, and 25 μg/ml heparin sulfate at 37° C. in the plate reader (Tecan, Austria).

In addition, fibril formation in the absence and presence of antibodies was also initiated by the presence of pre-formed fibrils. In brief, aSyn preformed fibrils (1 μM) were aggregated in the presence of activated aSyn monomers (10 μM) and 10 μM ThT in 100 μl PBS for 0-24 hours.

For data analysis, the mean of the negative control samples, i.e., the background fluorescence of ThT was calculated and divided from each sample at the given time point, e.g., in Microsoft Excel. To compare different conditions/inhibitors in the aggregation assay, each sample was normalized to the fluorescence reading determined at the beginning of the assay and set to 1. (t0=1).

To evaluate kinetic curves, a Michaelis Menten kinetic model was applied: Km (substrate concentration that yield a half-maximal velocity) and Vmax (maximum velocity) values of each condition were calculated using GraphPad Prism software, following enzyme kinetics analysis (Michaelis-Menten).

To compare different conditions/inhibitors in the aggregation assay, the slope value in the exponential growth phase of the ThT kinetic was calculated using GraphPad Prism software following linear regression.

### 10) Determination of Affinity and Avidity

For determination of Ab avidity, a variation of the standard ELISA assay was used where replicate wells containing antibody bound to the different antigens of the respective examples were exposed to increasing concentrations of chaotropic thiocyanate ions. Resistance to thiocyanate elution was used as the measure of avidity and an index (avidity index) representing 50% of effective antibody binding was used to compare different sera. In brief, plasma was diluted 1/500 in PBS and dispended on coated and blocked ELISA plates (Nunc Maxisorb). After incubation for 1h, sodium thiocyanate (NaSCN, SIGMA; in PBS) was added to the samples at concentrations of 0.25 to 3 M. Plates were incubated at room temperature for 15 min prior to washing, detection and subsequent colour reaction using Streptavidin-POD (Roche) and TMB. The absorbance readings in the absence of NaSCN were assumed to represent effective total binding of specific antibody (100% binding), and subsequent absorbance readings in the presence of increasing concentrations of NaSCN were converted to the appropriate percentage of the total bound antibody. The data were fitted to a graph of (% binding) vs. (log) concentration of NaSCN and by linear regression analysis the avidity index, representing the concentration of NaSCN required to reduce the initial optical density by 50% was estimated. Data were rejected as if the correlation coefficient for the line-fitting was below 0.88.

For determination of k_{D} values (binding affinity) towards aSyn filaments, displacement ELISAs which allow a simple determination of the k_{D} value of the complex formed by an Ab and its competitive ligand were used. In brief, equal concentration of Abs were incubated with increasing concentrations of free aSyn filaments prior to measurement of free antibody titer on plates with immobilized aSyn filaments. The relative binding of Abs is expressed as a percentage of maximum binding observed in the assay for each sample; the competition reactions with aSyn filaments (5 μg/ml) were defined as representing 0% binding (unspecific binding), and reactions without competition are taken to indicate 100% (maximum) of binding in the displacement curves.

Analysis of the competition binding curves was performed according to the one-site models using the computer-assisted curve fitting software from GraphPad.

### 11) Induction of Synucleinopathy in Mice

For induction of synucleinopathy, nine-week-old male C57BL/6 mice were stereotactically injected at the level of the right substantia nigra, with preformed polymorph fibrils (PFFs; i.e., preformed sonicated t-polymorph aSyn fibrils 1B). PFFs were prepared and validated as described in Sci. Adv. 2020, 6, eabc4364, doi: 10.1126/sciadv.abc4364; DOI: 10.1126/sciadv.abc4364. Briefly, each animal received a unilateral injection of 2 μL PFFs 1B solution (concentration: 2.5 mg/ml) into the region immediately above the right substantia nigra (coordinates from bregma: −2.9 AP, ±1.3 L and −4.5 DV) at a flow rate of 0.4 μL/min [Sci. Adv. 2020, 6, eabc4364, doi: 10.1126/sciadv.abc4364; DOI: 10.1126/sciadv.abc4364] and the needle has been left in place for 5 min before being slowly withdrawn from the brain.

Starting at the same day as the inoculation, animals received three i.d. immunizations, once every two weeks (i.e., weeks 0, 2, 4), with either CLEC-based vaccine (n=5) or non-conjugated CLEC (n=10) as control, followed by a boost immunization in week 10. Upon termination of the study (day 126) cerebrospinal fluid (CSF) was collected by cisterna magna puncture and brains were carefully removed and fixed in paraformaldehyde (PFA; 4%). Coronal serial sections of the entire brain (from rostral cerebral cortex anterior to striatum to the medulla—i.e., bregma −6.72 mm) using a cryostat at 50 μm intervals were collected and processed for immunohistochemistry.

### 12) Immunohistochemistry (IHC)

IHC staining of phosphor-S129 aSyn (pS129 aSyn) on coronal serial sections was performed as previously described [Sci. Adv. 2020, 6, eabc4364, doi: 10.1126/sciadv.abc4364; DOI: 10.1126/sciadv.abc4364]. The monoclonal rabbit anti-ps129 aSyn antibody EP1536Y (ab51253, Abcam) was used, followed by an incubation with labelled poly-mer-HRP anti-rabbit (Dako EnVision+TM Kit, K4011). Visualization of pS129 aSyn staining was done with Dako DAB (K3468) and sections were counterstained with Nissl stain. The actual number of pS129 aSyn aggregates per structure (cerebral cortex, striatum, thalamus, substantia nigra and brainstem) and the total number of pS129 aSyn aggregates were assessed using whole section acquisition by Panoramic Scan II (3DHISTECH, Hungary) further processed with ad-hoc developed QuPath algorithm.

### Example 1: Determination of Biological Activity of CLEC-Conjugates In Vitro

PAMPs like CLECs are recognized by PRRs present in APCs. Binding of CLECs to their cognate PRRs (e.g.: dectin-1 for β-glucans) is required to control adaptive immunity at various levels, e.g., by inducing downstream carbohydrate-specific signaling and cell activation, maturation and migration of cells to draining lymph nodes or through crosstalk with other PRRs. To provide a novel vaccine platform technology as proposed in this application, it is therefore crucial that the CLECs used are retaining their PRR binding ability, which demonstrates biological activity of the CLEC selected as well as of the CLEC based conjugate.

Along these lines and to ensure that 1) the structure of CLECs was not destroyed during mild periodate oxidation and 2) that the polysaccharide remained biologically active after coupling, binding to dectin-1 was assessed by ELISA. First, several different CLECs have been oxidized by mild periodate oxidation to produce the reactive sugar backbone of the proposed vaccines. These CLECs include: mannan, pustulan (20 kDa), lichenan (245 kDa), barley β-glucan (229 kDa), Oat β-glucan (295 kDa) and Oat β-glucan (391 kDa). Subsequently, vaccine conjugates have been produced by hydrazone coupling using different B-cell epitope peptides (SeqID2, SeqID10, SeqID16) and SeqID7 as T-helper epitope peptide, all containing a C-terminal hydrazide linker for coupling. In addition, also a peptide-pustulan conjugate produced by coupling SeqID10 via the heterobifunctional linker BMPH has been used.

Non-oxidized and oxidized CLECs as well as CLEC-based novel conjugates have then been assessed for their biological activity using a competitive ELISA system based on competitive binding of a soluble murine Fc-dectin-1a receptor (InvivoGen) or ConA as described in Korotchenko et al. 2020.

### Results:

Different CLECs tested display differential efficacy of PRR binding. In a series of ELISA experiments the dectin-1 ligands pustulan, lichenan, barley β-glucan, oat β-glucan, have been assessed for their binding efficacy to dectin-1. Ensuing experiments revealed that the median molecular weight (20 kDa), linear β-(1,6) linked β-D-glucan pustulan was surprisingly exerting significantly higher binding efficacy (ca. 3-fold) to dectin-1 than the larger, high molecular weight, linear β-(1,3) β-(1,4)-β-D glucan lichenan (ca 245 kDa) (see FIG. 1).

This difference was even more pronounced when comparing pustulan to other linear, β-(1,3) β-(1,4)-β-D glucans from oat and barley (barley β-glucan (229 kDa), oat β-glucan: 265 and 391kd) which displayed only limited binding efficacy in comparison to pustulan (e.g.: ca. 30-fold lower for barley β-glucan (229 kDa)).

Mild periodate oxidation of selected CLECs leads to a reduction in dectin-1 binding. Oxidation of mannan reduced its binding capacity to the lectin ConA to a similar extent as the reduction described for oxidized pustulan-dectin-1 binding following periodate oxidation. Similarly, oxidation of glucans leads to a similar proportional reduction in PRR binding (see FIG. 1A).

Importantly, conjugate formation also resulted in reduction of PRR binding capacity of the peptide-CLEC conjugates compared to unconjugated CLECs, as shown for mannan-containing conjugate as well as for different pustulan, lichenan or barley and oat-Bglucan conjugates tested (see FIG. 1B).

The experiments revealed that pustulan, despite its smaller size and the absence of β-(1,3) glycosidic bonds (please note: β-(1,3) containing glucans are described as optimal ligand for dectin-1) the linear β-(1,6) linked β-D-glucan pustulan was exerting highest binding efficacy, irrespective of oxidation or conjugation. For example, pustulan containing conjugates retain an approx. 3-fold higher binding than lichenan based constructs.

With respect to IC50 values, the binding results according to FIG. 1 show binding of the various constructs to the soluble murine Fc-dectin-1a receptor. The IC50 value obtained are (FIG. 1):
Oat β-glucan 265:860 μg/ml
Oat β-glucan 391:820 μg/ml
Barley β-glucan 229:145 μg/ml
Lichenan (FIG. 1E): 13 μg/ml
Lichenan 200% conjugate (FIG. 1E): 27 μg/ml (i.e. about half of unconjugated lichenan)
Pustulan: 3,5 μg/ml (FIG. 1B) and 5 μg/ml (FIG. 1D) (at least 30-fold stronger binding than the 145 μg/ml for Barley Bglucan 229)
Pustulan conjugates (FIG. 1D): 11, 14 und 15 μg/ml (i.e. about half of unconjugated pustulan)
Pustulan BMPH-Conjugate (FIG. 1F): 80 μg/ml (peptide was coupled with a heterobifunctional linker to pustulan).

FIGS. 1A and 1B further demonstrate that conjugation of peptides via hydrazone formation or via heterobifunctional linkers is equally suitable for WISIT conjugates as both types of conjugates are retaining high dectin-1 binding efficacy.

### Example 2: Determination of DC Activation Following Pustulan Exposure In Vitro

An important function of the vaccines proposed is their capacity to activate DCs following PRR binding and uptake. To demonstrate that CLEC based conjugates are not only binding to PRRs but also exert biological function in their target cells, i.e. DCs, a DC activation experiment has been performed.

First, murine bone marrow cells were incubated with mGM-CSF to generate BMDCs according to published protocols. These GM-CSF DCs were then exposed to either the peptide-glucan conjugate P SeqID2+SeqID7+pustulan or to equivalent amounts of oxidized but unconjugated sugar. In each case, conjugates/sugars were titrated from 500 μg to 62.5 μg/mL of the respective sugar. For comparison, the strong activator LPS has been used as control starting at a concentration of 2 ng/ml. Importantly, pustulan preparations used for oxidation and conjugate formation also contain small amounts of LPS. Thus, the equivalent dose of LPS was used to normalize the effects. DCs were then assessed for expression of markers for DC activation and maturation using FACS analysis including CD80 and MHCII.

### Results:

GM-CSF DCs stimulated in vitro with SeqId2-SeqID7-pustulan conjugates revealed a significantly increased expression of CD80 and MHCII (see FIG. 2). Levels were significantly higher than the effects observed by equivalent doses of LPS contained within the conjugate preparations. In contrast, equivalent amounts of oxidized but unconjugated sugar led to a slight reduction in CD80 expression as would have been expected from the LPS level in the preparation and a significantly less pronounced induction of MHCII as compared to the pustulan conjugates.

In summary, the up-regulation of MHC-II is indicative of DC activation. In addition, CD80 is upregulated more than would be expected by the same amount of LPS which strongly indicates that pustulan conjugates contribute significantly to the maturation and activation of DCs (beyond the effect explained by LPS exposure alone). Thus, examples 1 and 2 clearly demonstrate biological activity of the pustulan vaccines.

### Example 3: Particle Size Determination by DLS

Individual experiments analysing the particle size/hydrodynamic radius of different glucan conjugates have been performed.

For DLS analysis, different peptide-glucan, and peptide-carrier-glucan conjugates have been analysed and compared to nonconjugated pustulan, respectively. All analyses were performed in triplicates with a WYATT DynaPro PlateReader-II. Results obtained indicate a particle size distribution with a maximum in the low nm spectrum for all conjugates tested.

### Conjugates Tested:

| B-cell epitope | T-cell epitope | CLEC |
|---|---|---|
| SeqID2 | SeqID7 | Pustulan (80%) |
| SeqID3 | CRM197 | Pustulan (80%) |
| na | na | Non oxidized |
| | | Pustulan |

### Results:

Current analysis indicates an average main particle hydrodynamic radius (HDR) of ca. 5 nm for the peptide-pustulan conjugate SeqID2+SeqID7+pustulan used in this assay. A minor second peak detectable at ca. 60 nm indicates a very small number of aggregates present in the formulation (see FIG. 3A). Most of the conjugate preparation however seems to be present as monomeric form. This prevalence of monomeric rather than cross-linked or aggregated conjugates is also strongly supported by the fact that monomeric pustulan (ca. 20 kDa) is detectable at approx. 5 nm (as shown in the control samples see also FIG. 3C), which also supports the prevalence of monomeric pustulan conjugates (given a HDR of ˜5 nm for monomeric pustulan). As shown by Cumulants radius analysis over 24 h, the conjugates are also stable for their HDR and do not tend to aggregate again supporting the prevalence of monomeric conjugates.

To characterize vaccines based on peptide-carrier-glucan conjugates a SeqID6+CRM197 conjugate which has been additionally conjugated to pustulan was analysed. Again, DLS analysis revealed an average HDR of 11 nm and a second minor peak of ca. 75 nm again indicating the presence of a small number of aggregates (see FIG. 3B). The slight increase to 11 nm is most likely reflecting the increase of the MW of the resulting conjugate as CRM197 is around 60 kDa in size. No significant aggregation or cross linking of CRM conjugates can be detected and cumulants radius analysis over 24h also shows that the conjugates are stable for their HDR and do not tend to aggregate. Again, DLS analysis of this alternative type of CLEC based vaccines is supporting the prevalence of monomeric conjugates.

Control samples (i.e., non-oxidised pustulan) showed a much larger HDR with an average of ca. 600 nm as well as two additional smaller peaks at 5 nm and 46 nm, respectively (see FIG. 3C). Pustulan monomers have a HDR of ca. 5 nm, which fits well with the assumed MW of 20 kD, larger aggregates can be readily detected, and the majority of the glucan is present as large, high MW particles. Importantly, cumulants radius analysis over 24h also shows that, in contrast to pustulan conjugates, non-conjugated pustulan tends to strongly aggregate over time leading to the prevalent formation of large particles, consistent with various literature reports.

Example graphs for these two conjugates and non-oxidized pustulan controls are depicted in FIG. 3.

The results obtained in this example further demonstrate the so far unique characteristics of CLEC based conjugates as compared to examples well-known in the field (e.g.: Wang et al., 2019, Jin et al., 2018) with displaying small (i.e., 5-11 nm), prevalently monomeric sugar-based nanoparticles with far less than 150 nm HDR, a size which is generally considered a preferable size for immune-therapeutically active conjugate vaccines. This is mainly due to the PRR binding and activation characteristics of larger particulates including also whole glucan particles. Larger particulates (>150 nm up to 2-4 μm) are known to interact more efficiently with their receptors and can initiate DC signalling, -activation, -maturation and -migration to draining lymph-nodes whereas small, even soluble PRR-ligands are believed to be able to bind to their receptor but to block subsequent DC activation (Goodridge et al., 2011). These data, together with data described in Examples 1, 2 and 3 as well as other examples provided below however for the first time demonstrate that small, soluble peptide-based gluconeo-conjugates building on a monomeric β-glucan, e.g.: the linear β(1,6)-β-D glucan pustulan, as backbone can effectively bind to the PRR (dectin-1), activate the respective APC (as exemplified by GM-CSF DCs) and display very high biological activity and immunogenicity in skin specific manner also surpassing the effects of classical conjugate vaccines significantly.

### Example 4: In Vivo Comparison of Different CLEC-Based Vaccines

CLEC based vaccines which were able to bind to their DC receptor (e.g.: dectin-1 or ConA) were tested for their ability to induce a robust and specific immune response following repeated application in n=5 Balb/c mice/group. Typical experiments were performed applying 5 μg net peptide content of B-cell epitope peptides per dose.

In a first set of experiments three different CLECs were compared. In this experiment the aSynuclein derived peptide SeqID2 or the amyloid β 42 (Aβ42) derived peptide SeqID10 and the promiscuous T-helper cell epitope SeqID7 were coupled via C-terminal hydrazide linkers to oxidized pustulan (20% degree of oxidation), mannan (20% degree of oxidation) or barley β-glucan (229 kDa, 20% degree of oxidation).

### Vaccine Used:

| B-cell epitope | T-cell epitope | CLEC |
|---|---|---|
| SeqID2 | SeqID7 | Pustulan (20%) |
| SeqID2 | SeqID7 | Mannan (20% |
| SeqID2 | SeqID7 | barley β-glucan |
| | | (229 kDa, 20%) |
| SeqID2 | SeqID7 | Pustulan (80%) |
| SeqID2 | SeqID7 | Lichenan |
| | | (245 kDa, 200% |
| SeqID10 | SeqID7 | Pustulan (80%) |
| SeqID10 | SeqID7 | Lichenan |
| | | (245 kDa, 200% |

Animals (female Balb/c mice) were vaccinated 3 times in biweekly intervals (route: i.d.) and the ensuing immune response directed against the injected peptides (i.e., SeqID2 and SeqID10, respectively) was analyzed using murine plasma taken two weeks after the third immunization.

### Results:

As shown in FIG. 4A, all three CLEC vaccines (SeqID2+SeqID7+mannan, SeqID2+SeqID7+pustulan (linear β(1,6) β-glucan) and barley SeqID2+SeqID7+β-glucan (229 kDa) were able to induce a detectable immune response. Interestingly, immunization using the vaccine based on barley high molecular weight β-glucan did only induce a very low anti-peptide response (ODmax/2 titer ca 1/100). In contrast, pustulan based conjugates could induce a significantly higher response with average titers of ca 1/11000. Mannan based conjugates showed a ca. 7× lower immunogenicity as compared to pustulan-based conjugates as average titers following immunization reached around 1/1500 in this experiment.

FIG. 4B displays results from a second set of experiments comparing immunogenicity of two different variants of glucan-based conjugates using either aSynuclein derived peptide SeqID2 or the amyloid B 42 (Aβ42) derived peptide SeqID10 as B-cell epitopes and the T-cell epitope SeqID7. The first variant was again relying on pustulan as CLEC for conjugation, the second variant was produced using the linear β-(1,3) β-(1,4)-β-D glucan lichenan (ca 245 kDa). As shown in FIG. 4B, both variants could induce high titer immune responses against the injected peptides (i.e. SeqID2/3 (SeqID3=SeqID2 adapted for BSA conjugation) and SeqID10/11 (SeqID11=SeqID10 adapted for BSA conjugation)). Peptide lichenan conjugates however showed a significantly lower immunogenicity than peptide pustulan conjugates in these experiments (4-8x higher anti-peptide titers at 5 μg dose) which is also in line with lower dectin-1 binding ability as shown in example 1. This demonstrates that dectin-1 binding efficacy in vitro can be directly linked to in vivo immunogenicity and biological activity of the vaccines. This leads to the identification of pustulan or fragments thereof (i.e. linear β(1,6)-β-D glucans) as most efficacious glucan variant as proposed in this application. Vaccines are also functional with different peptides demonstrating the platform potential of this vaccine type.

### Example 5: In Vivo Comparison of Peptide Pustulan Conjugates to Unconjugated Peptide Vaccines

To assess whether conjugation of CLECs to peptide immunogens is required for the induction of superior immunogenicity of the vaccines according to the present invention a set of experiments was initiated comparing two conjugates, (SeqID2+SeqID7+pustulan or SeqID2+SeqID7+mannan) to vaccine preparations containing all components as a mix without conjugation (i.e., SeqID2 and SeqID7 plus either non-oxidised pustulan or mannan, respectively). Again, n=5 female Balb/c mice were immunized i.d. three times in biweekly intervals and the ensuing immune response directed against the injected peptides (i.e., SeqID3) was analyzed using murine plasma taken two weeks after the third immunization.

### Vaccine Used:

| B-cell epitope | T-cell epitope | CLEC | Conjugation |
|---|---|---|---|
| SeqID2 | SeqID7 | Pustulan (20%) | yes |
| SeqID2) | SeqID7 | Mannan (20%) | yes |
| SeqID2 | SeqID7 | Pustulan | Non |
| | | (non- | conjugated; |
| | | activated) | mixing |
| SeqID2 | SeqID7 | Mannan | Non |
| | | (non- | conjugated; |
| | | activated) | mixing |

### Results:

FIG. 5 shows the comparison of anti-peptide (SeqID3) specific immune responses detectable following three immunizations. SeqID2+SeqID7+pustulan conjugates (20% oxidation) were able to induce 4 times higher immune responses as reported for the mix of unconjugated peptides SeqID2, SeqID7 and non-oxidized pustulan (i.e., 1/12000 vs. 1/3000) in this experiment. Similarly, also SeqID2+SeqID7+mannan conjugates (20% oxidation) were more efficient in inducing peptide specific immune responses as application of a mix of the components (1/7000 vs. 1/4000; 1, 75-fold increase). These data show that conjugation of peptide immunogens to activated CLECs is required to induce a strong and sustainable immune response in vivo.

### Example 6: In Vivo Comparison of SeqID5+SeqID7+Pustulan and SeqID2+ or Seq-7+Pustulan Conjugates

To assess whether CLECs based vaccines require B-cell and T-cell epitopes for the induction of sustainable anti-B-cell epitope specific immune responses in vivo a set of experiments was initiated comparing three conjugates, SeqID5+SeqID7+pustulan, SeqID5+pustulan and SeqID7+pustulan. n=5 female Balb/c mice were immunized i.d. three times in biweekly intervals and the ensuing immune response directed against the injected peptides (i.e., SeqID6) was analysed using murine plasma taken two weeks after the third immunization.

| B-cell epitope | T-cell epitope | CLEC |
|---|---|---|
| SeqID5 | SeqID7 | Pustulan (80%) |
| SeqID5 | n.a. | Pustulan (80%) |
| n.a. | SeqID7 | Pustulan (80%) |

### Results:

As shown in FIG. 6, SeqID5+SeqID7+pustulan conjugates (80% oxidation) is able to induce a high and highly specific immune response directed against the injected peptide moiety (i.e., the aSynuclein derived peptide SeqID6) reaching average titers of 1/36000 in these experiments. Peptide-pustulan conjugates containing either SeqID5 or SeqID7 alone coupled to pustulan via hydrazone coupling could induce either a 12-fold lower immune response in the case of SeqID5-pustulan (1/3000) or no SeqID6 specific immune response (for SeqID7-pustulan conjugates, titer <1/100, below detection limit) following three biweekly immunizations (route: i.d.).

These data show that conjugation of peptide immunogens to activated CLECs is required to induce a strong and sustainable immune response in vivo. It however also demonstrates that pustulan conjugation to individual short B-cell epitopes in the absence of T-cell epitopes (e.g.: SeqID5 alone) allows for induction of T-cell independent B-cell responses in vivo, albeit at significantly lower efficacy as reported for T- and B-cell epitope containing CLEC conjugates.

### Example 7: In Vivo Analysis of Anti-Pustulan/Glucan Immune Responses Following Peptide-Pustulan Immunisation

Analysis of anti-CLEC antibodies is important on two levels for the novelty and efficacy of the proposed CLEC-vaccines according to the present invention:
1) β-glucans are major constituents of the cell wall of various fungi, lichens and plants conferring to the cell wall its typical strength opposing intracellular osmotic pressure. β-glucans are therefore also considered typical microbial pathogen-associated molecular patterns (PAMP) s and a major target for high titer circulating natural Abs in healthy human subjects. PAMPs are common and relatively invariant molecular structures shared by many pathogens, which are powerful activators of the immune system. (Chiani et al. Vaccine 27 (2009) 513-519, Noss et al. Int Arch Allergy Immunol 2012; 157:98-108, Dong et al. J Immunol 2014; 192:1302-1312, Ishibashi et al. FEMS Immunology and Medical Microbiology 44 (2005) 99-109, Harada et al. Biol Pharm Bull. 2003 August; 26(8):1225-8). IgG to -β-(1,3)- and -β-(1,6)-glucans can be found in normal human sera and β-(1,6)-glucans appear to be much more potent antigens than β-(1-3) variants. In addition, β-(1→6)β-glucan moiety has been identified as one of the typical microbial PAMPs, which acts as a focal point of recognition and attack for immunological malignancy surveillance, as well as defense against microbial invasion. Pustulan, the preferred glucan-backbone for the CLEC conjugates according to the present invention, is constituted from linear β-(1-6)-β-glucan moieties and it has been reported by several research groups that anti-pustulan immune responses can be detected in plasma from naïve, non-pustulan immunized human subjects. It is thus crucial to investigate the potential of CLEC based vaccines on activating anti-pustulan immunoreactivity. Anti-β-glucan antibodies could interact with peptide-pustulan specifically in vivo and could lead to the quick elimination by forming antigen-antibody complexes and thereby precluding induction of efficient immune reactions. Alternatively, induction/boosting of anti-pustulan antibody response following immunization could also foster immunogenicity as a potential cross-presentation of CLEC conjugates by anti-pustulan specific IgG antibodies and uptake into APCs could also increase the efficacy of vaccines applied.

No formal studies have been published investigating the presence of anti-pustulan antibodies in naïve mice. However, Ishibashi et al. and Harada et al. could demonstrate the existence of β-glucan IgGs to soluble scleroglucan/β-glucan (i.e., 1,3/1,6-beta-glucans) in sera of naive DBA/2 mice.
2) as previously reported (e.g.: Torosantucci et al, Bromuro et al., Donadei et al., Liao et al.) CLEC-protein conjugates, e.g. CRM197-coupled to laminarin, Curdlan or synthetic β(1,3) β-D glucans, were acting as strong immunogens not only inducing high anti-CRM197 titers but also high anti-glucan titers combined with protection from antifungal infection. Thus, previous attempts using such conjugates have been directed at using CLECs as bona fide disease/fungal infection specific immunogens instead of using it as a carrier and immunologically inert backbone as proposed in this application.

Along these lines, an extensive analysis of plasma samples of naïve and peptide-CLEC conjugate immunized Balb/c mice (n=5/group) for the presence of anti-pustulan antibodies prior to immunization and following repeated immunizations, respectively, was initiated.

### Vaccine Used:

| B-cell epitope | T-cell epitope | CLEC | Conjugation |
|---|---|---|---|
| SeqID2 | SeqID7 | Pustulan | yes |
| | | (20%) | |
| SeqID2 | SeqID7 | Mannan | yes |
| | | (20%) | |
| SeqID2 | SeqID7 | Pustulan | Non |
| | | (non- | conjugated; |
| | | activated) | mixing |
| SeqID2 | SeqID7 | Mannan | Non |
| | | (non- | conjugated; |
| | | activated) | mixing |

### Results:

Therefore, samples from animals undergoing peptide-pustulan (SeqID2+SeqID7+pustulan (20%), and peptide-mannan (SeqID2+SeqID7+mannan (20%) immunisations were analysed (all vaccines: 4 μg of aSyn targeting peptide/dose). For control purposes also animals undergoing application of vaccines consisting of non-conjugated peptides and non-oxidised CLECs were used (i.e., SeqID2+SeqID7+non-ox. pustulan, SeqID2+SeqID7+non-ox. mannan). As shown in FIG. 7A, the Balb/c animals analysed showed a pre-existing low level immune response directed against pustulan/β(1,6)-β-D glucan. Both CLEC vaccines tested (SeqID2+SeqID7+pustulan (20%), and SeqID2+SeqID7+mannan (20%)) failed in inducing a strong de novo immune responses directed against the glucan backbone in vivo. In contrast, repeated application of unconjugated, non-oxidised pustulan present in the control group (receiving a mix of all three components) led to the induction of a strong anti-glucan immune response by boosting antibody levels against pustulan 18, 5 times (compared to pre-immune plasma). Mannan containing conjugates or mixes were unable to induce anti-pustulan titers indicating specificity of the anti-glucan response detected. Kinetic analysis of anti-pustulan antibody titers showed a steady increase over time with a strong increase after the third immunization in animals undergoing immunization using non-conjugated and non-oxidized pustulan (see FIG. 7B). A competition ELISA using increasing amounts of native pustulan also demonstrated the specificity of the antibody response detectable in the group receiving a mix of components (FIG. 7C).

In summary, these analyses could demonstrate that: despite presence of a low-level auto-reactivity against pustulan (IgG) in naïve Balb/c mice, no/very low vaccination dependent increase of anti-pustulan immunoreactivity is induced by immunization using various CLEC conjugates. Therefore, the CLECs as used as peptide-CLEC conjugates according to this invention are indeed immunologically inert using the novel vaccine design according to the present invention. This is in strong contrast to previously published results and therefore constitutes a surprising and inventive novel characteristic of the carbohydrate backbone (e.g. the β-glucans or mannans, especially the pustulan backbone) according to the present invention.

In addition, pre-existing anti-pustulan-responses do not seem to preclude immune reactions to the peptide component of WISIT vaccines as the injected peptide responses for both experiments revealed high anti-peptide titers.

### Example 8: Analysis of Immunogenicity of Glucan Conjugates with N- or C-Terminally Coupled Peptide Immunogens

To assess whether linker orientation used for coupling would interfere with immunogenicity of the vaccines, 4 different vaccine candidates were produced: In this experiment the aSynuclein derived peptides SeqID1/2 and SeqID4/5 were coupled either via N- or C-terminal hydrazide linkers to oxidized pustulan (80%). In addition, each of the 4 vaccines was carrying the promiscuous T-helper cell epitope SeqID7 coupled via C-terminal hydrazide linkers to the CLEC backbone.

### Vaccine Used:

| B-cell epitope | T-cell epitope | CLEC |
|---|---|---|
| SeqI1 | SeqID7 | Pustulan (80%) |
| SeqID2 | SeqID7 | Pustulan (80%) |
| SeqID4 | SeqID7 | Pustulan (80%) |
| SeqID5 | SeqID7 | Pustulan (80%) |

weekly intervals (route: i.d.) and the ensuing immune response directed against the injected peptide (i.e., SeqID3 and SeqID6) as well as against the target protein, i.e. recombinant aSynuclein was analyzed using murine plasma taken two weeks after the third immunization.

### Results:

As shown in FIG. 8, all 4 CLEC vaccines using either N- or C-terminally coupled B-cell epitopes were able to induce a strong and highly specific immune response against both, the injected peptide moieties (FIG. 8A), and the target protein: aSynuclein (FIG. 8B). Interestingly, coupling orientation was differentially affecting immunogenicity. For example, the N-terminally coupled SeqID1+SeqID7+CLEC vaccine was inducing a 7-fold lower anti-injected peptide response and a 10-fold lower immune response directed against rec. aSyn as the C-terminally coupled SeqID2+SeqID7+CLEC, respectively. In contrast C-terminally coupled SeqID5+SeqID7+CLEC vaccine was inducing an approx. 4-fold lower injected peptide response but an equal anti-aSyn response as compared to N-terminally coupled SeqID4+SeqID7+CLEC vaccine, respectively. Thus, coupling orientation can be changed based on peptide specific characteristics without affecting the development of high titer immune responses.

However, as shown in FIG. 8, by variation of the coupling orientation of the immunogenic peptide the specificity of the ensuing response against the target protein can be significantly increased and can thus be used to create novel and unprecedented immune responses:
e.g.: SeqID1 vaccination leads to a 4, 5-fold higher response against the peptide as compared to the target protein which is comparable to the 3,3-fold higher anti-peptide response as compared to the protein induced by the SeqID2 vaccine.

In contrast SeqID4 vaccine induces a 1, 7-fold higher response against the peptide as compared to the protein, whereas the SeqID5 vaccine could reverse this ratio leading to a 2.5-fold higher protein specific response as compared to the injected peptide response detectable.

In summary, these data clearly show that vaccines using either coupling direction are biologically active and are suitable for this application. It is also shown that coupling orientation can be used to elect specifically preferred and unprecedently active vaccines depending on the peptide and target to be addressed.

### Example 9: Analysis of Immunogenicity of CLEC Conjugates Using Different T-Helper Cell Epitopes

In this example immunogenicity of CLEC based vaccines containing the nonnatural pan DR epitope (PADRE containing an artificial Cathepsin cleavage site, SeqID7) were compared to other well-known T-helper cell epitopes. For this purpose, several promiscuous epitopes have been selected which have either been adapted using a novel, artificially included Cathepsin L cleavage site for efficient endo/lysomal release following receptor mediated uptake in APCs/DCs or left unchanged. The epitopes selected include:

| | | | Cathepsin |
|---|---|---|---|
| | | | L cleavage |
| Peptide | Sequence | Epitope | site |
| SeqID7 | AKFVAAWTLKAAANRRA-(NH-NH2) | PADRE | + |
| SeqID22 | AKFVAAWTLKAAA-(NH-NH2) | PADRE | − |
| SeqID23 | KAAAVKAAFWTAL-NRRA-(NH-NH2) | artificial | + |
| SeqID24 | DSETADNLEKTVAALSILPGHGC-(NH-NH2) | Diphtheria | − |
| SeqID25 | DSETADNLEKTVAALSILPGHGCNRRA-(NH-NH2) | Diphtheria | + |
| SeqID26 | ISITEIKGVIVHRIETILF-(NH-NH2) | Measles | − |
| | | Virus fusion | |
| | | protein | |
| SeqID27 | ISITEIKGVIVHRIETILENRRA-(NH-NH2) | Measles | + |
| | | Virus fusion | |
| | | protein | |
| SeqID28 | ISQAVHAAHAEINEAGR-(NH-NH2) | Chicken OVA (323-339) | − |
| SeqID29 | ISQAVHAAHAEINEAGRNRRA-(NH-NH2) | Chicken OVA (323-339) | + |

To assess whether peptide vaccines carrying these T-helper cell epitope peptides could mount high immune reactions following repeated immunization and could induce immune reactions which were superior to conventional conjugate vaccines, 10 different vaccine candidates were tested:

In this experiment the aSyn derived peptide SeqID2 was either used as peptide-CLEC vaccine (i.e.: SeqID2, in combination with the different T-helper cell epitopes coupled via C-terminal hydrazide linkers to oxidized pustulan (80%); or a conventional peptide-conjugate was produced using SeqID3 containing a C-terminal cysteine for coupling to GMBS activated KLH.

### Vaccine Used:

| | | T-cell | | | |
|---|---|---|---|---|---|
| | B-cell | epitope/ | | | |
| Vaccine | epitope | carrier | CLEC | adjuvant | Route |
| 1 | SeqID2 | SeqID7 | Pustulan | n.a | i.d. |
| | | | (80%) | | |
| 2 | SeqID2 | SeqID22) | Pustulan | n.a | i.d. |
| | | | (80%) | | |
| 3 | SeqID2 | SeqID23 | Pustulan | n.a | i.d. |
| | | | (80%) | | |
| 4 | SeqID2 | SeqID24 | Pustulan | n.a | i.d. |
| | | | (80%) | | |
| 5 | SeqID2 | SeqID25 | Pustulan | n.a | i.d. |
| | | | (80%) | | |
| 6 | SeqID2 | SeqID26 | Pustulan | n.a | i.d. |
| | | | (80%) | | |
| 7 | SeqID2 | SeqID27 | Pustulan | n.a | i.d. |
| | | | (80%) | | |
| 8 | SeqID2 | SeqID28 | Pustulan | n.a | i.d. |
| | | | (80%) | | |
| 9 | SeqID2 | SeqID29 | Pustulan | n.a | i.d. |
| | | | (80%) | | |
| 10 | SeqID3 | KLH | n.a | Alhydrogel | s.c. |

Animals (female Balb/c mice) were vaccinated 3 times in biweekly: intervals (all vaccines: 5 μg of aSyn targeting peptide/dose; route: i.d. for the CLEC based vaccine and s.c. for the KLH based vaccine (adjuvanted with Alhydrogel) and the ensuing immune response directed against the injected peptide (i.e., SeqID3) as well as against the target protein, i.e., recombinant human aSynuclein has been analysed using murine plasma taken two weeks after the third immunization.

### Results:

As shown in FIG. 9, all 9 CLEC vaccines using different T-helper-cell epitopes and the KLH conjugate were able to induce a strong and specific immune response against both, the injected peptide moieties (SeqID3, FIG. 9A) and the target protein: recombinant aSynuclein (FIG. 9B).

All T-helper epitopes could induce anti-peptide titers similar or superior to the conventional SeqID3+KLH conjugate. Vaccine 1 (containing SeqID2 and SeqID7 coupled to pustulan) for example could induce a 60% higher response as the KLH control, whereas Vaccine 8 (containing SeqID28, a well well-known T-helper epitope specifically suitable for application in Balb/c animals, SeqID2 and pustulan) could induce a 5,5-fold higher response than the control. Even the promiscuous T-helper epitope SeqID24 (derived from Diphteria Toxin, a weak T-helper epitope for Balb/c animals disclosed in WO 2019/21355 A1) was able to induce a sustainable immune response, albeit weaker than the KLH control.

Similarly, all T-helper epitopes could induce anti-protein titers similar or superior to the conventional SeqID3-KLH conjugate. Importantly, Vaccine 1 (containing SeqID2 and SeqID7 coupled to pustulan) for example could induce a 2, 5-fold higher response as the KLH control, and Vaccine 8 (containing SeqID28, a well well-known T-helper epitope specifically suitable for application in Balb/c animals, SeqID2 and pustulan) could induce a 3-fold higher response than the control again supporting the fact that CLEC based vaccines according to this invention can induce superior anti-target responses.

The example also shows that introduction of an additional Cathepsin L cleavage site to the well described T-helper epitopes leads to more efficient induction of immune responses as compared to conventional vaccines and to CLEC based vaccines devoid of this artificial sequence.

For example, SeqID25, the modified variant of the weak T-helper epitope SeqID24 containing the cleavage site, was able to induce a 7,5-fold higher anti-peptide and a 3,6-fold higher anti-protein response as compared to the unmodified peptide (vaccine 5 vs. vaccine 4). In addition, this alteration also led to a 40% increase in anti-protein titers as compared to the KLH control. SeqID27, the Cathepsin L cleavage site modified variant of SeqID26 (an epitope derived from Measles virus fusion protein, disclosed in in WO 2019/21355 A1) could also significantly augment titers with a 1,8-fold increase in anti-peptide and a 3,2-fold increase in anti-protein titers as compared to the SeqID26-CLEC vaccine (i.e., vaccine 7 vs. vaccine 6). Vaccine 7 was also inducing a 2,2-fold higher anti-peptide response and a 1,6-fold higher anti-protein response as the KLH control. SeqID7 based CLEC vaccines are also inducing superior anti-protein titers (20% increase) as compared to non-modified variants (e.g.: SeqID22) and both peptides lead to an approximate doubling of anti-SeqID2 peptide and anti-aSyn titers as compared to the KLH control, respectively.

Addition of the Cathepsin cleavage site leads to formation of a peptide variant with an additional N (e.g.: at the C-terminus released upon cleavage. E.g.: SeqID22, PADRE, is released as AKFVAAWTLKAAA whereas SeqID7, modified PADRE is released as AKFVAAWTLKAAA-N. This N could also negatively impact further processing and MHCII presentation and could thus lower efficacy of the respective peptide. This phenomenon can be seen at the example of the very strong OVA derived epitopes SeqID28 and SeqID29. The unmodified peptide is inducing very high immune responses whereas the modified variant pep17 induces a 75% reduced anti-peptide and a 98% reduced anti-protein titer as compared to the unmodified variant.

### Example 10: Analysis of Immunogenicity of CLEC Conjugates Using Carrier Proteins as T-Helper Cell Epitopes: KLH

In this example immunogenicity of CLEC based conjugate vaccines containing the well-known carrier protein KLH was compared to conventional KLH vaccines. For this purpose, two aSyn derived epitopes (SeqID3 and SeqID6) have been selected which have been coupled to GMBS activated KLH. Subsequently, Pep-KLH conjugates have been coupled to reactive aldehydes of oxidized pustulan using the BPMH crosslinker to form CLEC based conjugate vaccines with KLH as source for T-helper cell epitopes to induce a sustainable immune response.

| | T-cell | | | |
|---|---|---|---|---|
| B-cell | epitope/ | | | |
| epitope | carrier | CLEC | adjuvant | Route |
| SeqID3 | KLH | Pustulan | n.a | i.d. |
| | | (140%) | | |
| SeqID3 | KLH | n.a | n.a | i.d |
| SeqID3 | KLH | n.a | Alhydrogel | s.c. |
| SeqID6 | KLH | Pustulan | n.a | i.d. |
| | | (80%) | | |
| SeqID6 | KLH | n.a | n.a | i.d. |
| SeqID6 | KLH | n.a | Alhydrogel | s.c. |

Animals (female Balb/c mice) were vaccinated 3 times in bi-weekly intervals (all vaccines: 20 μg of aSyn targeting peptide/dose; route: i.d. for the CLEC based vaccine and for non-adjuvanted KLH based vaccine and s.c. for the KLH based vaccine adjuvanted with Alhydrogel) and the ensuing immune response directed against the injected peptide (i.e. SeqID3 and SeqID6) as well as against the target protein, i.e. recombinant human aSynuclein has been analysed using murine plasma taken two weeks after the third immunization.

### Results:

As shown in FIG. 10A, all 6 vaccines using KLH as source for T-helper epitopes were able to induce a strong and specific immune response against both, the injected peptide moieties (SeqID3 and SeqID6) and the target protein: recombinant aSynuclein.

CLEC modification of the KLH conjugates lead to a highly superior immune response using both peptides, SeqID3 and SeqID6, respectively. SeqID3+KLH+pustulan was able to induce 2, 3 times higher anti-peptide responses as Alhydrogel adjuvanted SeqID3+KLH and a 14 times higher response as obtained following i.d. application of non-adjuvanted SeqID3+KLH. Similarly, also anti-protein titers were 8, 5-fold increased (compared to Alhydrogel adjuvanted SeqID3+KLH) and 17 times as compared to non-adjuvanted material. SeqID6+KLH+pustulan was also 2 (inj. peptide) to 4, 6 times (alpha synuclein) more effective than adjuvanted SeqID6+KLH and 8, 7 (inj. peptide) and 11 times (alpha synuclein) more immunogenic than the non-adjuvanted SeqID6+KLH vaccine, respectively.

Besides a general increase in immunogenicity of CLEC modified vaccines, the results also show that CLEC modification according to this invention leads to a significant increase in the relative amount of antibodies induced which are binding to the target molecule, i.e., the protein thereby increasing target specificity of the ensuing immune response significantly. Accordingly, the relative amount of antibodies detecting alpha synuclein (i.e., the ratio of total anti-injected peptide titers compared to anti-alpha synuclein specific titers) is 3, 7 times higher for SeqID3+KLH+pustulan induced responses as compared to adjuvanted SeqID3+KLH and 2, 2 times higher in the case of SeqID6+KLH+pustulan as compared to adjuvanted conjugates.

In a second set of experiments, the same vaccines used (all vaccines: 5 μg of aSyn targeting peptide/dose; route: i.d. for the CLEC based vaccine and s.c. for the KLH based vaccine adjuvanted with Alhydrogel) were compared for their ability to induce anti-carrier specific antibody responses. As expected, conventional SeqID3+ and SeqID6+KLH based vaccines were able to induce high anti-KLH titers (SeqID3+KLH: 1/2100 and SeqID6+KLH: 1/7700) whereas the CLEC based SeqID3+KLH+pustulan and SeqID6+KLH+pustulan vaccines were basically unable to induce sustainable anti-carrier antibodies. The titers obtained were close to the detection limit with 1/150 for SeqID3+KLH+pustulan and less than 1/100 for SeqID6+KLH+pustulan respectively thus creating a novel, yet undescribed optimization strategy for peptide-conjugate vaccines to increase target specific titers while reducing unwanted anti-carrier responses.

### Example 11: Analysis of Immunogenicity of CLEC Conjugates Using Carrier Proteins as T-Helper Cell Epitopes: CRM197

In this example immunogenicity of CLEC based conjugate vaccines containing the well-known carrier protein CRM197 was compared to conventional CRM197 vaccines. For this purpose, the alpha synuclein derived epitope SeqID6 has been coupled to maleimide activated CRM197. Subsequently, SeqID6+CRM197 conjugate has been coupled to activated pustulan using the heterobifunctional linker BPMH to form CLEC based conjugate vaccines with CRM197 as source for T-helper cell epitopes to induce a sustainable immune response. Alternatively, SeqID5-(NH—NH2; SeqID5) and CRM197 have been coupled to activated pustulan, independently. This was done by reaction of the hydrazide at the C-terminus of SeqID5 and via Lysins present in CRM197 to reactive aldehydes on activated pustulan.

### Vaccines Used:

| | T-cell | | | | |
|---|---|---|---|---|---|
| B-cell | epitope/ | | CLEC | | |
| epitope | carrier | CLEC | coupling | adjuvant | Route |
| SeqID6 | CRM197 | Pustulan | as conjugate | n.a | i.d. |
| | | (80%) | | | |
| SeqID5 | CRM197 | Pustulan | independent | n.a | i.d. |
| | | (80%) | | | |
| SeqID6 | CRM197 | n.a | n.a | Alhydrogel | s.c. |

Animals (female Balb/c mice) were vaccinated 3 times in bi-weekly weekly intervals (all vaccines: 20 μg of alpha synuclein targeting peptide/dose; route: i.d. for the CLEC based vaccines and and s.c. for the CRM197 based vaccine adjuvanted with Alhydrogel) and the ensuing immune response directed against the injected peptide (i.e., SeqID6) as well as against the target protein, i.e. recombinant human alpha synuclein as well as alpha synuclein filament has been analysed using murine plasma taken two weeks after the third immunization.

### Results:

As shown in FIG. 11A, all 3 vaccines using CRM197 as source for T-helper epitopes were able to induce a strong and specific immune response against both, the injected peptide moieties (SeqID6) and the target protein: recombinant alpha synuclein.

Again, CLEC modification of the CRM197 conjugates led to a highly superior immune response. SeqID6+CRM197+pustulan was able to induce 28 times higher anti-peptide responses as Alhydrogel adjuvanted SeqID6+CRM197. Similarly, also anti-protein titers against recombinant alpha synuclein were 15-fold increased (compared to Alhydrogel adjuvanted SeqID6+CRM197) and titers against the aggregated form of aSyn, aSyn filaments, was 11-fold increased. The vaccine produced by independently coupling SeqID5 and CRM197 to pustulan was also inducing 1, 7 times higher inj. peptide titers as conventional Alhydrogel adjuvanted SeqID6+CRM197. Reactivity to recombinant aSyn was also increased 6, 6 times and anti-filament responses were increased by a factor of 4,25, respectively.

Comparison of anti-carrier specific antibody responses revealed that conventional SeqID6+CRM197 based vaccines were able to induce high anti-CRM197 titers (1/6600) whereas the CLEC based SeqID6+CRM197+pustulan vaccine was basically unable to induce sustainable anti-carrier antibodies. The titers obtained were close to the detection limit with less than 1/100 for SeqID6+CRM197+pustulan respectively.

Thus, the experiments show that CLEC modification of conventional peptide-protein conjugates impairs development of an anti-carrier response significantly and leads to a strongly enhanced target specificity of the ensuing immune response providing a novel unprecedented strategy to optimize current state of the art conjugate vaccines building on carrier proteins like KLH, CRM197 or others.

Independent coupling of CRM197 and SeqID6 to pustulan leads to sustainable response against the B-cell epitopes present on CRM197, although at a lower rate as detectable for conventional, non-CLEC modified conjugates (Titer ca. 1/400). This shows that the CLEC backbone according to the current invention is also suitable to provide B-cell epitopes from CLEC coupled immunogenic proteins for use as vaccine.

### Example 12: Analysis of Selectivity of Immune Responses Elicited by CLEC Based Vaccines In Vivo

Aggregation of the presynaptic protein aSyn has been implicated as major pathologic culprit in synucleinopathies like Parkinson's disease whereas monomeric, non-aggregated aSyn has important neuronal functions. It is thus believed to be crucial for treatment of synucleinopathies, for example by active or passive immunotherapy, to reduce/remove aggregated aSyn without affecting the available pool of non-aggregated molecules present.

To further characterize the immune responses elicited by CLEC based vaccines containing the aSyn targeting peptides SeqID2 and SeqID3 and SeqID5 and SeqID6 as compared to conventional peptide-carrier vaccines (i.e., SeqID3+KLH and SeqID6+CRM197) a set of experiments was performed analysing the selectivity of the ensuing immune response elicited towards two different forms of the presynaptic protein aSyn: non aggregated, mainly monomeric aSyn as well as aggregated aSyn filaments.

### Vaccines Used:

| | T-cell | | | |
|---|---|---|---|---|
| B-cell | epitope/ | | | |
| epitope | carrier | CLEC | adjuvant | Route |
| SeqID2 | SeqID7 | Pustulan | n.a | i.d. |
| | | (80%) | | |
| SeqID3 | KLH | Pustulan | n.a | i.d. |
| | | (80%) | | |
| SeqID3 | KLH | n.a | Alhydrogel | s.c. |
| SeqID5 | SeqID7 | Pustulan | n.a | i.d. |
| | | (80%) | | |
| SeqID6 | CRM197 | Pustulan | n.a | i.d. |
| | | (80%) | | |
| SeqID6 | CRM197 | n.a | Alhydrogel | s.c. |

Animals (female Balb/c mice) were vaccinated 3 times in bi-weekly weekly intervals (all vaccines: 20 μg of aSyn targeting peptide/dose; route: i.d. for the CLEC based vaccines and and s.c. for the KLH and CRM197 based vaccine adjuvanted with Alhydrogel) and the ensuing immune response against the target protein, i.e., recombinant human alpha Synuclein as well as aSyn filament has been analysed using murine plasma taken two weeks after the third immunization. The plasma samples were subjected to an aSyn specific inhibition ELISA and IC50 values were determined.

### Results:

Briefly, all CLEC based conjugates used in this experiment demonstrate superior immunogenicity and aSynuclein aggregate specific target selectivity as compared to the conventional peptide-conjugate vaccines (i.e., SeqID3+KLH and SeqID6+CRM, see FIG. 12).

Conventional peptide conjugate vaccines can induce an antibody response with slightly increased selectivity for aSyn aggregates (i.e., filaments) as compared to monomeric/recombinant aSyn. SeqID3+KLH adjuvanted with Alhydrogel was mounting an immune response with 9-fold higher selectivity for aSyn aggregates as compared to recombinant aSyn. SeqID6+CRM197 adjuvanted with Alhydrogel was inducing a less selective immune response reaching 3,5-fold more selective binding directed towards aggregates as compared to mainly monomeric, recombinant aSyn.

In contrast, antibodies induced by CLEC based peptide conjugate vaccines were characterized by several fold more selective binding as compared to KLH or CRM197 conjugate vaccines. The SeqID2+SeqID7+pustulan and SeqID5+SeqID7+pustulan induced plasma shows an approx. 97-fold (i.e. 14× higher than the comparator vaccine SeqID3+KLH, Alhydrogel) and 50-fold higher aggregate selectivity (i.e. 14x higher than the comparator vaccine SeqID6+CRM, Alhydrogel). SeqID3+KLH+pustulan and SeqID6+CRM197+pustulan were similarly selective reaching 40—(i.e. 5 fold higher than SeqID3+KLH) and 50-fold (i.e. 14 times higher than SeqID6+CRM) higher selectivity for aSyn aggregates respectively.

Thus, the experiments show that CLEC modification of peptide conjugates as well as of peptide-protein conjugates leads to a strongly enhanced target specificity of the ensuing immune response providing a novel unprecedented strategy to optimize current state of the art conjugate vaccines.

### Example 13: Analysis of Avidity and Affinity of Immune Responses Elicited by CLEC Based Vaccines

To further characterize the immune responses elicited by CLEC based vaccines containing the aSyn targeting peptides SeqID2 and SeqID3 and SeqID5 and SeqID6 as compared to conventional peptide-carrier vaccines (i.e., SeqID3+KLH and SeqID6+CRM197) a set of experiments was performed analysing the avidity and affinity of the antibodies elicited towards aSyn.

### Vaccines Used:

| | T-cell | | | |
|---|---|---|---|---|
| B-cell | epitope/ | | | |
| epitope | carrier | CLEC | adjuvant | Route |
| SeqID2 | SeqID7 | Pustulan | n.a | i.d. |
| | | (80%) | | |
| SeqID3 | KLH | Pustulan | n.a | i.d. |
| | | (80%) | | |
| SeqID3 | KLH | n.a | Alhydrogel | s.c. |
| SeqID5 | SeqID7 | Pustulan | n.a | i.d. |
| | | (80%) | | |
| SeqID6 | CRM197 | Pustulan | n.a | i.d. |
| | | (80%) | | |
| SeqID6 | CRM197 | n.a | Alhydrogel | s.c. |

Animals (female Balb/c mice) were vaccinated 3 times in biweekly intervals (all vaccines: 20 μg of aSyn targeting peptide/dose; route: i.d. for the CLEC based vaccines and s.c. for the KLH and CRM197 based vaccine adjuvanted with Alhydrogel) and the ensuing immune response against the target protein, i.e., recombinant human aSyn as well as aSyn filament has been analysed using murine plasma taken two weeks after each immunization. To determine avidity of the induced Abs towards recombinant aSyn, a variation of the standard ELISA assay was used where replicate wells containing antibody bound to antigens were exposed to increasing concentrations of chaotropic thiocyanate ions. Resistance to thiocyanate elution was used as the measure of avidity and an index (avidity index) representing 50% of effective antibody binding was used to compare plasma samples (both between treatment groups and between time points).

In addition, the k_{D} value for aSyn filaments (antibody affinity toward aSyn filaments) of the antibodies 2 weeks after the last immunization was determined as well based on an aSyn competition ELISA.

### Results:

As shown in FIG. 13, conventional SeqID3+KLH conjugate (adjuvanted with Alhydrogel) showed only limited avidity maturation towards aSyn binding when comparing immune samples obtained two weeks after the second (T2) or two weeks after the third immunization (avidity maturation (AM, comparing IC₅₀ values for T2 and T3 samples: 1, 1)). In contrast CLEC based vaccines like SeqID2+SeqID7+pustulan could induce a strong maturation of the anti-aSyn response as indicated by an AI of 2,2 associated with a strong increase in avidity of T3 samples towards aSynuclein. Samples obtained from animals undergoing SeqID3+KLH+pustulan immunisation also showed a significantly higher avidity and a slightly increased maturation as compared to SeqID3+KLH alone.

Similarly, avidity of the immune response elicited against aSyn proteins was also significantly higher for SeqID5+SeqID7+pustulan and SeqID6+CRM197+pustulan vaccine induced antibodies as compared to the SeqID6+CRM197 benchmark vaccine (analysed at T3; i.e. 3-3,8 times higher chaotropic salt levels were required to reduce binding) and affinity maturation was also increased comparing T2 and T3 values, respectively. SeqID6+CRM197 did not lead to an increase in avidity towards aSyn comparing T2 and T3 whereas the two CLEC based vaccines lead to a strong increase in aSyn specific binding comparing T2 and T3.

Experiments quantifying the aSyn filament k_{D} for the immune response elicited by CLEC based vaccines as well as conventional benchmark vaccines revealed a highly significant increase in the overall affinity of antibodies induced by CLEC based vaccines for aSyn (see FIG. 14). SeqID2+SeqID7+pustulan and SeqID3+KLH+pustulan conjugates showed a 6-9-fold higher affinity (i.e., Kd: 110 nM and 160 nM compared to a k_{D} of 1 mM) than the benchmark vaccine SeqID3+KLH adjuvanted with Alhydrogel. SeqID5+SeqID7+pustulan and SeqID6+CRM+pustulan conjugates are displaying 12-15 times better Kd values as the benchmark control SeqID6+CRM197, adjuvanted with Alhydrogel (i.e., Kd: 50 AM and 60 M compared to a k_{D} of 750 nM).

The experiments therefore show that CLEC modification of peptide conjugates as well as of peptide-protein conjugates leads to a strongly enhanced target specificity and affinity of the ensuing immune response providing a novel unprecedented strategy to optimize current state of the art conjugate vaccines.

### Example 14: Analysis of In Vitro Functionality of Immune Responses Elicited by CLEC Based Vaccines

To analyse whether aSyn specific antibodies elicited by CLEC based vaccines (containing aSyn targeting peptides SeqID2/3 and SeqID5/6) are biologically active a set of experiments was performed analysing the capacity of antibodies to inhibit aSyn aggregation in vitro.

### Vaccines Used:

| | T-cell | | | |
|---|---|---|---|---|
| B-cell | epitope/ | | | |
| epitope | carrier | CLEC | adjuvant | Route |
| SeqId2 | SeqID7 | Pustulan | n.a | i.d. |
| | | (80%) | | |
| SeqID3 | KLH | n.a | Alhydrogel | s.c. |
| SeqID5 | SeqID7 | Pustulan | n.a | i.d. |
| | | (80%) | | |
| SeqID6 | CRM197 | Pustulan | n.a | i.d. |
| | | (80%) | | |
| SeqID6 | CRM197 | n.a | Alhydrogel | s.c. |

Animals (female Balb/c mice) were vaccinated 3 times in biweekly intervals (all vaccines: 20 μg of aSyn targeting peptide/dose; route: i.d. for the CLEC based vaccines and s.c. for the KLH and CRM197 based vaccine adjuvanted with Alhydrogel). Samples of murine plasma taken two weeks after each immunization as well as respective control samples (e.g.: non aSyn binding antibodies or pre-immune plasma obtained before immunization) have been analyzed for in vitro aggregation inhibition capacity.

### Results:

As shown in FIG. 15A, control antibodies or plasma taken from animals prior to immunization had no significant effects on the aggregation kinetics of aSyn confirming the specificity of the assay. Conventional SeqID3+KLH conjugate (adjuvanted with Alhydrogel) induced Abs were able to reduce aSyn aggregation significantly as indicated by a 40% decreased slope value (aSyn monomer only: 100%; KLH. 60%). The SeqID2+SeqID7+pustulan vaccine induced Abs strongly inhibited aSyn aggregation as indicated by an 85% decreased slope value (aSyn monomer only: 100%; CLEC: 15%) in this assay indicating a significantly higher inhibition capacity as compared to classical vaccine induced Abs.

SeqID5-SeqID7-pustulan and SeqID6+CRM+pustulan based vaccine induced antibodies show 86-92% inhibition of the formation of aggregates starting with rec. aSyn (low content of aggregates) and 67-82% inhibition of the formation of aggregates starting with preformed fibrils (=bona fide aggregates) as compared to 68% and 57% for the benchmark vaccine SeqID6+CRM, Alhydrogel induced antibodies (see FIG. 15B).

### Example 15: Analysis of the Effects of the Route of Immunization on Immune Responses Elicited by CLEC Based Vaccines

A series of immunisations has been performed to compare i.d. administration to alternative routes including sub cutaneous (s.c.) and intra-muscular (i.m.).

### Vaccines Used:

| | T-cell | | | |
|---|---|---|---|---|
| B-cell | epitope/ | | | |
| epitope | carrier | CLEC | adjuvant | Route |
| SeqID2 | SeqID7 | Pustulan | n.a | i.d. |
| | | (80%) | | |
| SeqID2 | SeqID7 | Pustulan | n.a | s.c. |
| | | (80%) | | |
| SeqID2 | SeqID7 | Pustulan | n.a | i.m |
| | | (80%) | | |

Animals (female Balb/c mice) were vaccinated 3 times in biweekly intervals (all vaccines: 1 μg, 5 μg and 20 μg of aSyn targeting peptide/dose) and the ensuing immune response against the injected peptide and the target protein, i.e., recombinant human aSynuclein as well as aSyn filament has been analysed using murine plasma taken two weeks after the third immunization.

### Results:

Tables 1 and 2 and FIG. 16 show that SeqID2+SeqID7+pustulan vaccines applied via i.m. or s.c. routes could induce high immune responses against both, injected peptide (FIG. 16A) and anti-aSyn responses (FIG. 16B). Maximum titers reached were significantly lower than those following i.d. application at all doses tested. S.c. application showed a similar dose response behaviour as i.d. whereas i.m. did not show significant differences between 5 and 20 μg indicating a saturation at these doses/application volumes reached. Similar results were obtained for reactivity against monomeric as well as aggregated aSyn, respectively. These results demonstrate the high selectivity of the CLEC backbone as presented in this invention for application in skin as opposed to other routes/tissues.

TABLE
anti-SeqID2/3 induced antibody response following
WISIT vaccine application via different routes

| | 1 μg | 5 μg | 20 μg |
|---|---|---|---|
| i.d. | 16.000 | 83.000 | 140.000 |
| s.c. | 1000 | 2.000 | 12.000 |
| i.m. | 4.000 | 16.000 | 15.000 |

TABLE
anti-aSyn induced antibody response following
WISIT vaccine application via different routes

| | 1 μg | 5 μg | 20 μg |
|---|---|---|---|
| i.d. | 2.000 | 5.000 | 10.000 |
| s.c. | 100 | 1.000 | 4.000 |
| i.m. | 2.000 | 2.000 | 5.000 |

### Example 16: Analysis of B-Cell Epitopes Using Post-Translationally Modified Peptides: Aβ

To assess whether peptide vaccines carrying peptides characterized by posttranslational modifications (e.g.: including phosphorylation, acetylation, or pyroglutamate modification of amino acids) could mount high immune reactions following repeated immunization and could induce immune reactions which were superior to conventional conjugate vaccines, 2 different vaccine candidates were tested:

In this experiment an Amyloid Beta (Aβ) 40/42 derived peptide, carrying an N-terminal pyroglutamate amino acid as example for post translational modifications, was either used as peptide-CLEC vaccine (i.e.: SeqID33, in combination with the promiscuous T-cell epitope SeqID7 was coupled via C-terminal hydrazide linkers to oxidized pustulan (80%); or a conventional peptide-conjugate was produced using SeqID32 containing a C-terminal cysteine for coupling to GMBS activated KLH.

### Vaccine Used:

| B-cell | T-cell epitope/ | | | |
|---|---|---|---|---|
| epitope | carrier | CLEC | adjuvant | Route |
| SeqID33 | SeqID7 | Pustulan | n.a | i.d. |
| | | (80%) | | |
| SeqID32 | KLH | n.a | Alhydrogel | s.c. |

Animals (female Balb/c mice) were vaccinated 3 times in biweekly intervals (route: i.d. for the CLEC based vaccine and s.c. for the KLH based vaccine (adjuvanted with Alhydrogel) and the ensuing immune response directed against the injected polypeptide (i.e., SeqID32/33) as well as against the target proteins, i.e. recombinant Aβ(pE) 3-40 and Aβ(pE) 3-42 has been analysed using murine plasma taken two weeks after the third immunization.

### Results:

As shown in FIG. 17, both vaccines were able to induce a strong and specific immune response against both, the injected peptide moieties and the target proteins: Aβ1-40/42, Aβ(pE) 3-40 and Aβ(pE) 3-42. As compared to SeqID32+KLH, adjuvanted with Alhydrogel, the SeqID33+SeqID7+CLEC based vaccine however showed a 6-fold higher immune response directed against the injected peptide moiety and most importantly also a 3, 7-fold higher immune response directed against the target protein/peptide AβpE3-42 as well as a 1,6 times higher response against the Aβ variant AβpE3-40. Surprisingly, both vaccines tested also induced a response against Aβ1-42 showing an unexpected extension of immunogenicity from pyroglutamate (pE)-modified truncated Aβ forms (i.e. AβpE3-42 and AβpE3-40) to intact, non-modified forms of this amyloidogenic and pathologic molecule (i.e. Aβ1-40/42) thereby also extending the potential therapeutic activity of such vaccines. Again, the SeqID33+SeqID7+CLEC based vaccine showed a several-fold higher immune response (3 fold) directed against this non-modified form of Aβ as compared to SeqID32+KLH, adjuvanted with Alhydrogel, showing the superior immunogenicity of CLEC based vaccines.

In addition, analysis of avidity using resistance to thiocyanate elution (NaSCN) revealed a 2, 6-fold higher avidity towards AβpE3-42 for SeqID33+SeqID7+CLEC induced antibodies as compared to SeqID32+KLH induced antibodies.

Thus, CLEC based vaccines are highly suitable for use of posttranslationally modified peptides as immunogens (irrespective whether they constitute self-antigens (as SeqID32/33) or foreign target structures) and that such epitopes can induce superior immune responses when administered as CLEC based vaccine conferring higher immune responses as well as a higher target specific responses as conventional vaccines.

In addition, this example also provides clear evidence that CLEC based vaccines using epitopes of target proteins present in amyloidosis including Alzheimer's disease, dementia with Lewy bodies or down syndrome are inducing surprisingly more specific immune responses than state of the art vaccines.

### Example 17: Analysis of B-Cell Epitopes of Intracellular Proteins and Self-Antigens: Tau

In order to assess whether peptide vaccines carrying peptides derived from intracellular proteins (irrespective whether they constitute self-antigens (as SeqID32/SeqID33 or SeqID35/36) or foreign target structures) which can undergo extensive modification (e.g. hyperphosphorylation, truncation and aggregation) could mount high immune reactions following repeated immunization and could induce immune reactions which were superior to conventional conjugate vaccines, 2 different vaccine candidates were tested:

In this experiment a Tau derived peptide was either used as peptide-CLEC vaccine (i.e.: SeqID 35, in combination with the pan-T-cell epitope SeqID7 was coupled via C-terminal hydrazide linkers to oxidized pustulan (80%); or a conventional peptide-conjugate was produced using SeqID36 containing a C-terminal cysteine for coupling to GMBS activated KLH.

### Vaccine Used:

| | T-cell epitope/ | | | |
|---|---|---|---|---|
| B-cell epitope | carrier | CLEC | adjuvant | Route |
| SeqID36 | SeqID7 | Pustulan | n.a | i.d. |
| | | (80%) | | |
| SeqID35 | KLH | n.a | Alhydrogel | s.c. |

Animals (female Balb/c mice) were vaccinated 3 times in biweekly intervals (route: i.d. for the CLEC based vaccine and s.c. for the KLH based vaccine (adjuvanted with Alhydrogel) and the ensuing immune response directed against the injected peptide (i.e., SeqID35/36) as well as against the target protein, i.e., recombinant human Tau441 has been analysed using murine plasma taken two weeks after the third immunization. SeqID35/36 is a well-known and effective Tau epitope, spanning aa 294-305 in human 4R tau 441, and has been elected as functional and effective epitope in EP2758433.

### Results:

As shown in FIG. 18, both vaccines were able to induce a strong and specific immune response against both, the injected peptide moieties and the target protein: Tau 441. As compared to SeqID35+KLH, adjuvanted with Alhydrogel (preferred Tau targeting vaccine conjugate according to EP2758433), the SeqID36+SeqID7+CLEC based vaccine however showed a 2,3-fold higher immune response directed against the injected peptide moiety and most importantly also a 3,3-fold higher immune response directed against the target protein Tau441.

In addition, analysis of avidity using resistance to thiocyanate elution (NaSCN) revealed a 2, 3-fold higher avidity towards SeqID35 for SeqID36+SeqID7+CLEC induced antibodies as compared to SeqID35+KLH induced antibodies, respectively.

Thus, CLEC based vaccines are highly suitable for use of epitopes directed against intracellular proteins as immunogens conferring higher immune responses as well as a higher target specific responses as conventional vaccines.

In addition, this example also provides clear evidence that CLEC based vaccines using epitopes of target proteins present in Tauopathies and Alzheimer's disease are inducing surprisingly superior and more specific immune responses against self-epitopes present in Tau as current state of the art Tau targeting vaccines. Tauopathies are neurodegenerative disorders characterized by the deposition of abnormal tau protein in the brain. The spectrum of tau pathologies expands beyond the traditionally discussed disease forms like Pick disease, progressive supranuclear palsy, corticobasal degeneration, and argyrophilic grain disease. It also includes globular glial tauopathies, primary age-related tauopathy, which includes neurofibrillary tangle dementia, chronic traumatic encephalopathy (CTE), and aging-related tau astrogliopathy.

### Example 18: Analysis of B-Cell Epitopes of Secreted Proteins, Self-Antigens, and Conformational Epitopes: IL23

To assess whether peptide vaccines carrying peptides derived from secreted proteins (irrespective whether they constitute self-antigens (as SeqID37 to SeqID42) or foreign target structures) could mount high immune reactions following repeated immunization and could induce immune reactions which were superior to conventional conjugate vaccines, 6 different vaccine candidates were tested:

In this experiment three different IL23 derived peptides were either used as peptide-CLEC vaccine (i.e.: SeqID38, SeqID40, SeqID42 in combination with the pan-T-cell epitope SeqID7, coupled via C-terminal hydrazide linkers to oxidized pustulan (80%); or as conventional peptide-conjugates, produced using SeqID37, SeqID39, and SeqID41 containing a C-terminal cysteine for coupling to GMBS activated KLH.

### Vaccines Used:

| | T-cell epitope/ | | | |
|---|---|---|---|---|
| B-cell epitope | carrier | CLEC | adjuvant | Route |
| SeqID38 | SeqID7 | Pustulan | n.a | i.d. |
| | | (80%) | | |
| SeqID37 | KLH | n.a | Alhydrogel | s.c. |
| SeqID40 | SeqID7 | Pustulan | n.a | i.d. |
| | | (80%) | | |
| SeqID39 | KLH | n.a | Alhydrogel | s.c. |
| SeqID42 | SeqID7 | Pustulan | n.a | i.d. |
| | | (80%) | | |
| SeqID41 | KLH | n.a | Alhydrogel | s.c. |

Animals (female Balb/c mice) were vaccinated 3 times in bi-weekly weekly intervals (route: i.d. for the CLEC based vaccine and s.c. for the KLH based vaccine (adjuvanted with Alhydrogel) and the ensuing immune response directed against the injected peptide (i.e., SeqID37, SeqID39 and SeqID41) as well as against the target protein, i.e., recombinant human IL23 has been analysed using murine plasma taken two weeks after the third immunization.

### Results:

As shown in FIG. 19, all 6 vaccines were able to induce a strong and specific immune response against both, the injected peptide moieties and the target protein: IL23 (p12/p40). As compared to SeqID37+KLH, adjuvanted with Alhydrogel, the SeqID38+SeqID7+CLEC based vaccine however showed a 2-fold higher immune response directed against the injected peptide moiety and most importantly also a 3-fold higher immune response directed against the target protein IL23. SeqID37/SeqID38 represents a conformational epitope on the D1 domain of the p40 subunit of both IL-12 and IL-23. It reflects the epitope of the fully human monoclonal antibody Ustekinumab that binds specifically to IL-12/IL23p40 and neutralizes human IL-12 and IL-23 bioactivity (Luo et al. J Mol Biol 2010 Oct. 8; 402 (5): 797-812.).

In contrast, SeqID39+KLH, adjuvanted with Alhydrogel, and the SeqID40+SeqID7+CLEC based vaccine induced similar responses directed against the injected peptide moiety and against the target protein IL23. Interestingly, SeqID39/SeqID40 is a peptide spanning the linear epitope aa38-46 in the p40 subunit of IL12 and IL23, respectively (Guan et al., 2009).

In contrast to SeqID38 and SeqID40 which either showed comparable or highly superior anti-target responses using CLEC backbones, SeqID41/42 did not show the same characteristics supporting the fact that selected peptide immunogens are suitable for the surprising effects provided in these examples. SeqID41/42 is a peptide spanning the linear epitope aa144-154 in the p19 subunit of IL23, respectively. SeqID41+KLH, adjuvanted with Alhydrogel, was inducing a 15-fold higher response directed against the injected peptide moiety and an 8-fold higher response directed against the target protein IL23 than the SeqID42+SeqID7+CLEC vaccine used in this experiment.

In summary, CLEC based vaccines are highly suitable for use of epitopes directed against secreted proteins including signaling molecules or Cyto/Chemokines as immunogens conferring higher immune responses as well as a higher target specific responses as conventional vaccines.

In addition, this example also provides clear evidence that CLEC based vaccines are suitable for use of conformational epitopes and that conformational epitopes can induce superior immune responses when administered as CLEC based vaccine.

This example also provides results demonstrating that CLEC based immunogens using epitopes of IL12/IL23 are inducing surprisingly more specific immune responses than state of the art vaccines against these self-epitopes. Therefore, such vaccines can be used for the treatment of IL12/IL23 associated autoimmune inflammatory diseases including: Psoriasis, chronic inflammatory bowel disease, rheumatoid arthritis

### Example 19: Analysis of B-Cell Epitopes of Self-Epitopes Present in Transmembrane Proteins: Extracellular Membrane-Proximal Domain of Membrane-Bound IgE (EMPD)

In this experiment a peptide derived from human EMPD has been used. SeqID43/SeqID44 constitutes an epitope disclosed in WO2017/005851 A1 and was either used as peptide-CLEC vaccine (i.e.: SeqID44 in combination with the pan-T-cell epitope SeqID7, coupled via C-terminal hydrazide linkers to oxidized pustulan (80%); or as conventional peptide-conjugates, produced using SeqID43 containing a C-terminal cysteine for coupling to GMBS activated KLH.

### Vaccines Used:

| | T-cell epitope/ | | | |
|---|---|---|---|---|
| B-cell epitope | carrier | CLEC | adjuvant | Route |
| SeqID44 | SeqID7 | Pustulan | n.a | i.d. |
| | | (80%) | | |
| SeqID43 | KLH | n.a | Alhydrogel | s.c. |

Animals (female Balb/c mice) were vaccinated 3 times in biweekly intervals (route: i.d. for the CLEC based vaccine and s.c. for the KLH based vaccine (adjuvanted with Alhydrogel) and the ensuing immune response directed against the injected peptide (i.e., SeqID43) as well as against a 41 aa fragment of the target protein region, EMPD (disclosed in WO2017/005851 A1 as suitable surrogate for protein recognition) has been analysed using murine plasma taken two weeks after the third immunization.

### Results:

As shown in FIG. 20, both vaccines were able to induce a strong and specific immune response against both, the injected peptide moieties and the EMPD protein-fragment.

As compared to SeqID43+KLH, adjuvanted with Alhydrogel, the SeqID44+SeqID7+CLEC based vaccine however showed an approx. 60% higher immune response directed against the injected peptide moiety and most importantly also an approx. 30% higher immune response directed against the target protein fragment.

In addition, analysis of avidity using resistance to thiocyanate elution (NaSCN) revealed a 3,8-fold higher avidity towards the EMPD peptide for SeqID44+SeqID7+CLEC induced antibodies as compared to SeqID43+KLH induced antibodies, respectively.

In summary, CLEC based vaccines are highly suitable for use of epitopes directed against transmembrane proteins including Extracellular Membrane-Proximal Domain of Membrane-Bound IgE (EMPD) conferring higher immune responses as well as a higher target specific responses as conventional vaccines.

Therefore, it is evident that the CLEC based vaccines according to the present invention can be preferably used for active anti-EMPD vaccination for the treatment and prevention of IgE related diseases. IgE-related disease include allergic diseases such as seasonal, food, pollen, mold spores, poison plants, medication/drug, insect-, scorpion- or spider-venom, latex or dust allergies, pet allergies, allergic asthma bronchiale, non-allergic asthma, Churg-Strauss Syndrome, allergic rhinitis and -conjunctivitis, atopic dermatitis, nasal polyposis' Kimura's disease, contact dermatitis to adhesives, antimicrobials, fragrances, hair dye, metals, rubber components, topical medicaments, rosins, waxes, polishes, cement t and leather, chronic rhinosinusitis, atopic eczema, autoimmune diseases where IgE plays a role (“autoallergies”), chronic (idiopathic) and autoimmune urticaria, cholinergic urticaria, mastocytosis, especially cutaneous mastocytosis, allergic bronchopulmonary aspergillosis, chronic or recurrent idiopathic angioedema, interstitial cystitis, anaphylaxis, especially idiopathic and exercise-induced anaphylaxis, immunotherapy, eosinophil-associated diseases such as eosinophilic asthma, eosinophilic gastroenteritis, eosinophilic otitis media and eosinophilic oesophagitis (see e.g. Holgate World Allergy Organization Journal 2014, 7:17, U.S. Pat. No. 8,741,294 B2, Usatine Am Fam Physician. 2010 Aug. 1; 82 (3): 249-55.). Furthermore, the vaccines according to the present invention are used for the treatment of lymphomas or the prevention of sensibilisation side effects of an anti-acidic treatment, especially for gastric or duodenal ulcer or reflux. For the present invention, the term “IgE-related disease” includes or is used synonymously to the terms “IgE-dependent disease” or “IgE-mediated disease”.

### Example 20: Analysis of B-Cell Epitopes of Allergens Mimotopes and Conformational Epitopes: Bet v 1

To assess whether peptide vaccines carrying peptides derived from foreign proteins/allergens could mount high immune reactions following repeated immunization and could induce immune reactions which were superior to conventional conjugate vaccines, 2 different vaccine candidates were tested:

In this experiment peptide SeqID45/SeqID46 derived from the well described major white birch (Betula verrucosa) pollen antigen, Bet v 1, has been used. SeqID45/SeqID46 constitutes a mimotope of the native sequence of Bet v 1 (Immunol Lett. 2009 Jan. 29; 122 (1): 68-75.). In addition, the authors also showed that antibodies induced by such a mimotope bind to two different regions within Bet v 1, amino acids 9-22 and 104-113. Thus, the mimotope SeqID45/SeqID46 is also an example for a conformational epitope.

SeqID45/SeqID46 was either used as peptide-CLEC vaccine (i.e.: SeqID46 in combination with the pan-T-cell epitope SeqID7, coupled via C-terminal hydrazide linkers to oxidized pustulan (80%); or as conventional peptide-conjugate, produced using SeqID45 containing a C-terminal cysteine for coupling to GMBS activated KLH.

Vaccines used:

| | T-cell epitope/ | | | |
|---|---|---|---|---|
| B-cell epitope | carrier | CLEC | adjuvant | Route |
| SeqID46 | SeqID7 | Pustulan | n.a | i.d. |
| | | (80%) | | |
| SeqID45 | KLH | n.a | Alhydrogel | s.c. |

Animals (female Balb/c mice) were vaccinated 3 times in bi-weekly weekly intervals (route: i.d. for the CLEC based vaccine and s.c. for the KLH based vaccine (adjuvanted with Alhydrogel) and the ensuing immune response directed against the injected peptide (i.e. SeqID45) as well as against the target protein, i.e. recombinant BetvI has been analysed using murine plasma taken two weeks after the third immunization.

### Results:

As shown in FIG. 21, both vaccines were able to induce a strong and specific immune response against both, the injected peptide moieties and the target protein: Bet v 1.

As compared to SeqID45+KLH, adjuvanted with Alhydrogel, the SeqID46+SeqID7+CLEC based vaccine however showed a 3, 3-fold higher immune response directed against the injected peptide moiety and most importantly also a 2-fold higher immune response directed against the target protein Bet v 1.

In addition, analysis of avidity using resistance to thiocyanate elution (NaSCN) revealed a 1, 9-fold higher avidity towards recombinant BetvI for SeqID46+SeqID7+CLEC induced antibodies as compared to SeqID45+KLH induced antibodies, respectively.

In summary, CLEC based vaccines are highly suitable for use of allergen epitopes including Bet v 1 conferring higher immune responses as well as a higher target specific responses as conventional vaccines. In addition, this example also provides clear evidence that CLEC based vaccines are suitable for use of mimotopes and conformational epitopes and that such mimotopes and conformational epitopes can induce superior immune responses when administered as CLEC based vaccine. Therefore, such vaccines can be used for the treatment of allergic diseases such as hay fever, seasonal-, food-, pollen-, mold spores-, poison plants-, medication/drug-, insect-, scorpion- or spider-venom, latex- or dust allergies, pet allergies, allergic asthma bronchiale, allergic rhinitis and -conjunctivitis, atopic dermatitis, contact dermatitis to adhesives, antimicrobials, fragrances, hair dye, metals, rubber components, topical medicaments, rosins, waxes, polishes, cement and leather, chronic rhinosinusitis, atopic eczema, autoimmune diseases where IgE plays a role (“autoallergies”), chronic (idiopathic) and autoimmune urticaria, anaphylaxis, especially idiopathic and exercise-induced anaphylaxis.

### Example 21: Analysis of B-Cell Epitopes Present in Different Forms of Cancer/Neoplastic Disease (i.e. Oncogenes): Her2

To assess whether peptide vaccines carrying peptides derived from cancer-associated antigens/oncogenes could mount high immune reactions following repeated immunization and could induce immune reactions which were superior to conventional conjugate vaccines, 2 different vaccine candidates were tested:

In this experiment peptide SeqID47/SeqID48 derived from the well described member of the human epidermal growth factor receptor (HER/EGFR/ERBB) family, Her2, has been used.

SeqID47/SeqID48 constitutes an epitope of the native sequence of the extracellular domain of human Her2: aa position 610-623. The epitope SeqID47/SeqID48 has been disclosed as powerful antigen by Wagner et al., 2007 and Tobias et al., 2017, present in conventional conjugate vaccines like peptide-Tetanus Toxoid and peptide-CRM197 conjugates, respectively.

SeqID47/SeqID48 was either used as peptide-CLEC vaccine (i.e.: SeqID48 in combination with the pan-T-cell epitope SeqID7, coupled via C-terminal hydrazide linkers to oxidized pustulan (80%); or as conventional peptide-conjugate, produced using SeqID47 containing a C-terminal cysteine (part of the native sequence) for coupling to maleimide activated CRM197.

### Vaccines Used:

| | T-cell epitope/ | | | |
|---|---|---|---|---|
| B-cell epitope | carrier | CLEC | adjuvant | Route |
| SeqID48 | SeqID7 | Pustulan | n.a | i.d. |
| | | (80%) | | |
| SeqID47 | CRM197 | n.a | Alhydrogel | s.c. |

Animals (female Balb/c mice) were vaccinated 3 times in biweekly intervals (route: i.d. for the CLEC based vaccine and s.c. for the CRM197 based vaccine (adjuvanted with Alhydrogel) and the ensuing immune response directed against the injected peptide (i.e., SeqID47) as well as against the target protein, i.e. recombinant human Her2 has been analysed using murine plasma taken two weeks after the third immunization.

### Results:

As shown in FIG. 22, both vaccines were able to induce a strong and specific immune response against both, the injected peptide moieties and the target protein: Her2.

As compared to SeqID47+CRM197, adjuvanted with Alhydrogel, the SeqID48+SeqID7+CLEC based vaccine however showed a 23% higher immune response directed against the injected peptide moiety and most importantly also a 30% higher immune response directed against the target protein Her2.

In summary, CLEC based vaccines are highly suitable for use as cancer vaccine conferring higher immune responses as well as a higher target specific responses as conventional vaccines, e.g. CRM197 based conjugate vaccines. Therefore, it is evident that such vaccines can be used for the treatment of neoplastic diseases.

### Example 22: Analysis of B-Cell Epitopes Present in Different Forms of Neoplastic Disease/Cancer (i.e. Oncogenes): PD1

To assess whether peptide vaccines carrying peptides derived from cancer-associated antigens/oncogenes could mount high immune reactions following repeated immunization and could induce immune reactions which were superior to conventional conjugate vaccines, different vaccine candidates targeting the Programmed cell death protein 1 (PD1) were tested:

PD-1 is an immune checkpoint and guards against autoimmunity through two mechanisms. 1) it promotes apoptosis (programmed cell death) of antigen-specific T-cells in lymph nodes. 2) it reduces apoptosis in regulatory T-cells (anti-inflammatory, suppressive T-cells). Downregulation of immune checkpoint activity, e.g.: PD1 signaling (by blocking PD1 activation) is a recently developed strategy to activate the immune system to attack tumors and is currently used to treat certain types of cancer. Suitable B-cell epitopes and prototype vaccines for targeting human PD1 have been disclosed previously.

In this experiment peptide SeqID49/SeqID50 derived from human PD1, aa position 92-110, has been used as B-cell epitope. The epitope has been disclosed as powerful antigen by Kaumaya et al. (ONCOIMMUNOLOGY 2020, VOL. 9, NO. 1, e1818437), present in a fusion-peptide based vaccine where the PD1 epitope was linked to a measles virus fusion peptide (MVF) amino acid (aa 288-302) via a four amino acid residue (GPSL) emulsified in the adjuvant Montanide ISA 720VG to induce antibodies that block PD-1 signaling.

SeqID49/SeqID50 was either used as peptide-CLEC vaccine (i.e.: SeqID50 in combination with the pan-T-cell epitope SeqID7, coupled via C-terminal hydrazide linkers to oxidized pustulan (80%); or as conventional peptide-conjugate, produced using SeqID49 containing a C-terminal cysteine (part of the native sequence) for coupling to maleimide activated KLH.

### Vaccines Used:

| | T-cell epitope/ | | | |
|---|---|---|---|---|
| B-cell epitope | carrier | CLEC | adjuvant | Route |
| SeqID50 | SeqID7 | Pustulan | n.a | i.d. |
| | | (80%) | | |
| SeqID49 | KLH | n.a | Alhydrogel | s.c. |

Animals (female Balb/c mice) were vaccinated 3 times in biweekly intervals (route: i.d. for the CLEC based vaccine and s.c. for the KLH based vaccine (adjuvanted with Alhydrogel) and the ensuing immune response directed against the injected peptide (i.e., SeqID49) as well as against the target protein, i.e. recombinant human PD1 has been analysed using murine plasma taken two weeks after the third immunization.

### Results:

As shown in FIG. 23, both vaccines were able to induce a strong and specific immune response against both, the injected peptide moieties and the target protein: PD1.

As compared to SeqID49+KLH, adjuvanted with Alhydrogel, the SeqID50+SeqID7+CLEC based vaccine however showed a similarly high immune response directed against the injected peptide moiety but most importantly also a 2-fold higher immune response directed against the target protein PD1 indicating a different development of antibodies specifically detecting the target protein of choice as compared to conventional vaccines.

In addition, analysis of avidity using resistance to thiocyanate elution (NaSCN) revealed a 4, 5-fold higher avidity towards SeqID49 for SeqID50+SeqID7+CLEC induced antibodies as compared to SeqID49+KLH induced antibodies, respectively.

In summary, CLEC based vaccines are highly suitable for use as cancer vaccine, especially also by targeting immune checkpoints like the PD-PDL1/2 system or CTLA4, conferring higher immune responses, especially also higher target specific responses as conventional vaccines, e.g., KLH based conjugate vaccines. Therefore, it is also evident that such vaccines can be used for the treatment of neoplastic diseases.

### Example 23: Analysis of Immunogenicity of CLEC Conjugates Using Carrier Proteins as T-Helper Cell Epitopes: Different Conjugate/CLEC Ratios

In this example immunogenicity of CLEC based conjugate vaccines containing the well-known carrier protein CRM197 using different peptide-CRM/CLEC ratios was compared. For this purpose, the aSyn derived epitope SeqID6 has been coupled to maleimide activated CRM197. Subsequently, SeqID6+CRM197 conjugate has been coupled to activated pustulan at different w/w ratios using the heterobifunctional linker BPMH to form CLEC based conjugate vaccines with CRM197 as source for T-helper cell epitopes to induce a sustainable immune response.

### Vaccines Used:

| B-cell | T-cell | | Ratio | | |
|---|---|---|---|---|---|
| epitope | epitope/carrier | CLEC | (w/w) | adjuvant | Route |
| SeqID6 | CRM197 | Pustulan | 1/1 | | |
| | | (80%) | | n.a | i.d. |
| SeqID6 | CRM197 | Pustulan | 1/2.5 | n.a | i.d. |
| | | (80%) | | | |
| SeqID6 | CRM197 | Pustulan | 1/5 | n.a | i.d. |
| | | (80%) | | | |
| SeqID6 | CRM197 | Pustulan | 1/10 | n.a | i.d. |
| | | (80%) | | | |
| SeqID6 | CRM197 | Pustulan | 1/20 | n.a | i.d. |
| | | (80%) | | | |

Animals (female Balb/c mice) were vaccinated 3 times in bi-weekly weekly intervals (all vaccines: 5 μg of aSyn targeting peptide/dose; route: i.d. for the CLEC based vaccines) and the ensuing immune response directed against the injected peptide (i.e., SeqID6) as well as against the target protein, i.e. recombinant human aSynuclein as well as aSyn filament has been analysed using murine plasma taken two weeks after the third immunization.

### Results:

As shown in FIG. 24, all 5 vaccines using CRM197 as source for T-helper epitopes were able to induce a strong and specific immune response against both, the injected peptide moieties (SeqID6) and the target protein: recombinant aSynuclein.

CLEC modification of the CRM197 conjugates led to a highly efficient immune response with all w/w Conjugate/CLEC ratios tested. SeqID6-CRM197-pustulan (w/w 1/10) was delivering highest anti-aSyn specific immune responses as compared to the other variants tested. Thus, SeqID6+CRM197 conjugates with medium/high Conjugate/CLEC ratios are especially suited for inducing optimal immune responses (e.g.: 1/5, 1/10 and 1/20).

Thus, the experiments show that CLEC modification of conventional peptide-protein conjugates leads to a strong target specificity of the ensuing immune response providing a novel unprecedented strategy to optimize current state of the art conjugate vaccines building on carrier proteins like KLH, CRM197 or others.

### Example 24: Analysis of Immunogenicity of CLEC Conjugates and Peptide Conjugates Using Carrier Proteins as T-Helper Cell Epitopes-aSyn N-Terminus (Aa1-10)

In this example we assessed whether CLEC based conjugate vaccines according to the present invention are able to induce superior immune responses against aSyn aggregates as compared to respective peptide conjugates using state of the art carrier proteins as source for T-cell epitopes.

Therefore, a set of experiments was initiated comparing two conjugates, both containing an epitope suggested to be suitable as aSyn targeting epitope. Experiments could demonstrate the immune response elicited against injected peptide and aSyn protein as well as the selectivity of the ensuing immune response elicited towards two different forms of the presynaptic protein aSyn: non aggregated, mainly monomeric aSyn as well as aggregated aSyn filaments.

For example, Weihofen et al (Neurobiology of Disease 124 (2019) 276-288, aa1-10 as epitope of Cinpanemab) and WO2016/062720 (aa1-8 as epitope in a VLP based immunotherapeutic) suggest the N-terminal aSyn sequence derived from position aa1-10 as a potentially suitable epitope for aSyn targeting immunotherapy. To assess whether CLEC modification indeed results in a superior immune response, we therefore compared a CLEC based vaccine containing aSyn sequence aa1-8 (SeqID12+SeqID7+Pustulan) with the respective conventional peptide-KLH vaccine (SeqID13+KLH adjuvanted with Alum).

### Vaccines Used:

| B-cell | T-cell epitope/ | | | |
|---|---|---|---|---|
| epitope | carrier | CLEC | adjuvant | Route |
| SeqID12 | SeqID7 | Pustulan | n.a | i.d. |
| | | (80%) | | |
| SeqID13 | KLH | na | Alum | i.d. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein by ELISA and EC50 values were determined. In addition, to assess selectivity of the immune response, the plasma samples were subjected to an aSyn specific inhibition ELISA and expressed as percentage of maximum binding.

### Results:

The aSyn N-terminus targeting CLEC based conjugate vaccine used in this experiment (SeqID12+SeqID7+Pus) demonstrates superior immunogenicity against aSyn protein as compared to the conventional peptide-conjugate vaccines (i.e., SeqID13+KLH see FIG. 25A). The CLEC based vaccine induces a 1,8-fold increase in anti aSyn titers and a concomitant 3-fold increase in the ratio of the anti-peptide to anti protein response as the comparator group. This strongly supports the teaching of this invention, that CLEC modification leads to a superior immune response as similar conventional vaccines.

In addition, the conventional peptide KLH conjugate vaccine induces an antibody response with strongly increased selectivity (ca. 10-fold) for aSyn monomers as compared to aggregates (i.e., filaments, see FIG. 25B). In contrast to this finding and very surprisingly, the CLEC based conjugate leads to a completely different selectivity: SeqID12+SeqID7+Pustulan induces antibodies with a significantly, ca. 10-fold higher selectivity for aSyn aggregates as compared to recombinant aSyn thereby changing the profile of the antibodies induced completely (see FIG. 25B).

Thus, the experiments show that conventional peptide vaccines applying aSyn aa1-8 are less suitable to mount an efficient and selective immune response in vivo suggesting that this epitope is not suitable for aggregate selective immunotherapy. Importantly, the results also demonstrate that CLEC modification of peptide conjugates leads to a strongly enhanced target specificity of the ensuing immune response as well as a change in selectivity towards aggregates which is therefore providing a novel unprecedented conjugate vaccine targeting aSyn.

### Example 25: Analysis of Immunogenicity of CLEC Conjugates and Peptide Conjugates Using Carrier Proteins as T-Helper Cell Epitopes aSyn aa100-108

Here, a set of experiments was initiated comparing two conjugates, both containing an epitope suggested to be suitable as aSyn targeting epitope by analysing the immune response elicited against injected peptide and aSyn protein as well as the selectivity of the ensuing immune response elicited towards two different forms of the presynaptic protein aSyn: non aggregated, mainly monomeric aSyn as well as aggregated aSyn filaments.

For example, WO 2011/020133 and WO2016/062720 suggest the aSyn sequence derived from position aa100-108/109 (either as native sequence or mimotope, i.e. 100-108) as a potentially suitable epitope for aSyn targeting immunotherapy. To assess whether CLEC modification indeed results in a superior immune response using this epitope region we therefore compared a CLEC based vaccine containing aSyn aa100-108 (SeqID16+SeqID7+Pustulan) with the respective conventional peptide-KLH vaccine (SeqID17+KLH adjuvanted with Alum).

| B-cell | T-cell epitope/ | | | |
|---|---|---|---|---|
| epitope | carrier | CLEC | adjuvant | Route |
| SeqID16 | SeqID7 | Pustulan | n.a | i.d. |
| | | (80%) | | |
| SeqID17 | KLH | na | Alum | i.d. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein by ELISA and EC50 values were determined. In addition, to assess selectivity of the immune response, the plasma samples were subjected to an aSyn specific inhibition ELISA and expressed as percentage of maximum binding.

### Results:

The aSyn targeting CLEC based conjugate vaccine used in this experiment (SeqID16+SeqID7+Pus) demonstrates an overall very low anti aSyn protein response, also lower as compared to the conventional peptide-conjugate vaccines (i.e., SeqID17+KLH see FIG. 26A). The conventional vaccine induces an immune response characterized by a 2, 1-fold increase in anti aSyn titers but at the same time a 2-fold decrease in the ratio of the anti-peptide/anti protein titers as the CLEC based vaccine. The latter finding supports the teaching of this invention, that CLEC modification leads to a superior anti target protein response, even in the case of overall lower immunogenicity as similar conventional vaccines.

In addition, both vaccines, the conventional peptide conjugate and the CLEC based vaccine, are less preferred to induce an aggregate selective immune response see FIG. 26B). Thus, the experiments provided show that CLEC-based and conventional peptide vaccines targeting the region aa100-108 are less suitable to mount an efficient and selective immune response in vivo suggesting that this epitope may not be the optimal choice for aggregate selective immunotherapy according to this invention.

### Example 26: Analysis of Immunogenicity of CLEC Conjugates and Peptide Conjugates Using Carrier Proteins as T-Helper Cell Epitopes aSyn aa91-100

In this example, a set of experiments was initiated comparing two conjugates, both containing an epitope suggested to be suitable as aSyn targeting epitope by analysing the immune response elicited against injected peptide and aSyn protein.

For example, US 2014/0377271 A1 suggests that the epitope aa91-99 acts as an autoepitope in PD patients and should therefore constitute a potentially suitable epitope for aSyn targeting immunotherapy. To assess whether CLEC modification indeed results in a superior immune response applying this epitope we therefore compared a CLEC based vaccine containing aSyn aa91-100 (SeqID14+SeqID7+Pustulan) with the respective conventional peptide-KLH vaccine (SeqID15+KLH adjuvanted with Alum).

### Vaccines Used:

| B-cell | T-cell epitope/ | | | |
|---|---|---|---|---|
| epitope | carrier | CLEC | adjuvant | Route |
| SeqID14 | SeqID7 | Pustulan | n.a | i.d. |
| | | (80%) | | |
| SeqID15 | KLH | na | Alum | i.d. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein aSyn by ELISA and EC50 values were determined.

### Results:

Surprisingly, both vaccines induced relevant anti peptide titers but were less successful to induce detectable anti aSyn protein titers (see FIG. 27). Thus, the experiments provided show that CLEC-based and conventional peptide vaccines targeting the region aa91-100 are less suitable to mount an efficient and selective immune response in vivo suggesting that this epitope may not be the optimal choice for aggregate selective immunotherapy according to this invention.

### Example 27: Analysis of Immunogenicity of CLEC Conjugates and Peptide Conjugates Using Carrier Proteins as T-Helper Cell Epitopes-aSyn C-Terminal Region Aa131-140

In this example, a set of experiments was initiated comparing two conjugates, both containing an epitope suggested to be suitable as aSyn targeting epitope by analysing the immune response elicited against injected peptide and aSyn protein. In addition, the selectivity of the ensuing immune response elicited towards two different forms of the presynaptic protein aSyn was assessed.

For example, US 2015/0232524 and WO2016/062720 suggest the C-terminal aSyn sequence derived from positions aa-126-140 and 131-140 as potentially suitable epitope for aSyn targeting immunotherapy. To assess whether CLEC modification indeed results in a superior immune response we therefore compared a CLEC based vaccine containing aSyn aa131-140 (SeqID20+SeqID7+Pustulan) with the respective conventional peptide-KLH vaccine (SeqID21+KLH adjuvanted with Alum).

### Vaccines Used:

| B-cell | T-cell epitope/ | | | |
|---|---|---|---|---|
| epitope | carrier | CLEC | adjuvant | Route |
| SeqID20 | SeqID7 | Pustulan | n.a | i.d. |
| | | (80%) | | |
| SeqID21 | KLH | na | Alum | i.d. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein by ELISA and EC50 values were determined. In addition, to assess selectivity of the immune response, the plasma samples were subjected to an aSyn specific inhibition ELISA and expressed as percentage of maximum binding.

### Results:

The aSyn targeting CLEC based conjugate vaccine used in this experiment (SeqID20+SeqID7+Pus) demonstrates an overall lower anti aSyn protein response as compared to the conventional peptide-conjugate vaccines (i.e., SeqID21+KLH see FIG. 28A). The conventional vaccine induces an immune response characterized by a 1,8-fold increase in anti aSyn titers but a 45% decrease in the ratio of the anti-peptide/anti protein titers as the CLEC based vaccine. The latter finding supports the teaching of this invention, that CLEC modification leads to a superior anti target protein response, even in the case of overall lower immunogenicity as similar conventional vaccines.

In addition, the conventional peptide conjugate are less suitable to induce an aggregate selective immune response (see FIG. 27B). In contrast, the CLEC based vaccine elicits antibodies with an approx. 10-fold increased selectivity for monomeric aSyn at the expense of aggregated aSyn (see FIG. 28B). Thus, the experiments provided show that CLEC-based and conventional peptide vaccines targeting the region aa131-140 are less preferred to mount an efficient and selective immune response towards aggregated aSyn in vivo suggesting that this epitope may not be the optimal choice for aggregate selective immunotherapy according to this invention.

### Example 28: Analysis of Immunogenicity of CLEC Conjugates and Peptide Conjugates Using Carrier Proteins as T-Helper Cell Epitopes-aSyn C-Terminal Region Aa103-135

In this example we assessed whether CLEC based conjugate vaccines according to the present invention can induce superior immune responses against aSyn as compared to respective peptide conjugates using state of the art carrier proteins as source for T-cell epitopes.

Therefore, a set of experiments was initiated comparing several conjugates, derived from an epitope region suggested to be suitable as aSyn targeting epitope. These experiments could demonstrate the immune response elicited against injected peptide and aSyn protein as well as the selectivity of the ensuing immune response elicited towards two different forms of the presynaptic protein aSyn: non aggregated, mainly monomeric aSyn as well as aggregated aSyn filaments.

Several studies suggest that the C-terminal aSyn sequence derived from position aa103-135 is a potentially suitable epitope for aSyn targeting either immunotherapy as source for autoepitopes, for peptides containing the original sequence or mimotopes thereof. To assess whether CLEC modification indeed results in a superior immune response using this region within aSyn we therefore compared several CLEC based vaccines (using peptides within region 107-126) with the respective conventional peptide-CRM vaccines (adjuvanted with Alum).

### Vaccines Used:

| B-cell | T-cell | | | |
|---|---|---|---|---|
| epitope | epitope/carrier | CLEC | adjuvant | Route |
| SeqID56 | SeqID7 | Pustulan | n.a | i.d. |
| | | (80%) | | |
| SeqID58 | CRM | na | Alum | s.c. |
| SeqID53 | SeqID7 | Pustulan | n.a | i.d. |
| | | (80%) | | |
| SeqID55 | CRM | na | Alum | S.c. |
| SeqID51 | SeqID7 | Pustulan | n.a | i.d. |
| | | (80%) | | |
| SeqID52 | CRM | na | Alum | s.c. |
| SeqID65 | SeqID7 | Pustulan | n.a | i.d. |
| | | (80%) | | |
| SeqID66 | CRM | na | Alum | S.c. |
| SeqID67 | SeqID7 | Pustulan | n.a | i.d. |
| | | (80%) | | |
| SeqID68 | CRM | na | Alum | S.c. |
| SeqID69 | SeqID7 | Pustulan | n.a | i.d. |
| | | (80%) | | |
| SeqID70 | CRM | na | Alum | S.c. |
| SeqID71 | SeqID7 | Pustulan | n.a | i.d. |
| | | (80%) | | |
| SeqID72 | CRM | na | Alum | s.c. |

Animals (female Balb/c mice) were vaccinated 3 times in biweekly intervals (all vaccines: 5 μg of aSyn targeting peptide/dose; route: i.d. for the CLEC based vaccines and s.c. for the KLH based vaccine adjuvanted with Alhydrogel) and the ensuing immune response against the injected peptide and the target protein by ELISA and EC50 values were determined. In addition, to assess selectivity of the immune response, the plasma samples were subjected to an aSyn specific inhibition ELISA and expressed as percentage of maximum binding.

### Results:

TABLE 1
immune response elicited by vaccines covering aa107-126

| | Peptide Titer | aSyn filament Titer |
|---|---|---|
| Vaccine | (x1000) | (x1000) |
| SeqDI56 + SeqID7 + Pus | 0.1 | 0.1 |
| SeqID58 + CRM + Alum | 9.781 | 0.1 |
| SeqDI53 + SeqID7 + Pus | 0.1 | 0.1 |
| SeqID55 + CRM + Alum | 17.647 | 0.8 |
| SeqDI51 + SeqID7 + Pus | 19. 694 | 1.687 |
| SeqID52 + CRM + Alum | 13.115 | 1.594 |
| SeqDI65 + SeqID7 + Pus | 9.04 | 3.256 |
| SeqID66 + CRM + Alum | 7.984 | 1.108 |
| SeqDI67 + SeqID7 + Pus | 12.016 | 7.335 |
| SeqID68 + CRM + Alum | 18.126 | 1.864 |
| SeqDI69 + SeqID7 + Pus | 28.125 | 3.523 |
| SeqID70 + CRM + Alum | 37.88 | 1.054 |
| SeqDI71 + SeqID7 + Pus | 7.783 | 5.788 |
| SeqID72 + CRM + Alum | 14.603 | 2.429 |

CLEC based as well as CRM based vaccines containing 5- and 6 mer peptides both were less suitable to induce high anti aSyn filament titers in this experiment. The aSyn C-terminus targeting CLEC based conjugate vaccines (7- to 12-mer peptides) used in this experiment (see table 1 and FIGS. 29A, 30A, and 31A) all demonstrate superior immunogenicity against aSyn filaments as compared to the conventional peptide-conjugate vaccines (see table 1, up to 4-fold increase). This strongly supports the teaching of this invention, that CLEC modification leads to a superior immune response as similar conventional vaccines using epitopes derived from 103-135, especially 107-126.

Analysis of selectivity for aggregated aSyn further supports this teaching. As shown in FIGS. 29B and 30B, CLEC vaccines containing epitopes derived from sequence aa115-126 are surprisingly effective in eliciting highly aggregate selective immune responses. As shown in FIG. 29B, the CLEC based vaccine SeqID51+SeqID7+Pus, containing an 8-mer aSyn targeting epitope, induces antibodies with a 10-fold higher selectivity for aSyn aggregates whereas the respective conventional vaccine (SeqID52+CRM+Alum) fails to induce aggregate selective antibodies. Similarly, CLEC based vaccine SeqID67+SeqID7+Pus containing a 10 mer aSyn targeting epitope, induces an approx. 10-fold higher selectivity for aSyn aggregates as compared to monomers whereas the respective conventional vaccine (SeqID68+CRM+Alum) elicits antibodies which are more selective, ca. 3-fold, for monomers as compared to aggregates (see FIG. 30B).

Analysis of selectivity for vaccines containing the epitope aa107-114 (SeqID73+SeqID7+Pus and SeqID74+CRM+Alum, see FIG. 31B) surprisingly showed that, despite the presence of high anti-aSyn filament titers (i.e. superior immunogenicity) induced by the CLEC-based vaccine, neither CLEC-nor conventional vaccines could induce aggregate selective antibodies indicating that only highly selected peptide sequences within aa103-135 are suitable as immunotherapeutics targeting aggregated aSyn specifically.

It may also be noted, as shown before (see FIGS. 26-28), that epitopes derived from aa91-100, aa100-108 and aa131-140 are all less suitable as potential immunotherapeutic regions for targeting aggregated aSyn specifically.

### Example 29: Analysis of In Vitro Functionality of Immune Responses Elicited by CLEC Based Vaccines

To analyse whether aSyn specific antibodies elicited by CLEC based vaccines (containing aSyn targeting peptides from epitope region aa103-135) are biologically active a set of experiments was performed analysing the capacity of antibodies to inhibit aSyn aggregation in vitro.

### Vaccines Used:

| | T-cell | | | |
|---|---|---|---|---|
| B-cell | epitope/ | | | |
| epitope | carrier | CLEC | adjuvant | Route |
| SeqID67 | SeqID7 | Pustulan | n.a | i.d. |
| | | (80%) | | |
| SeqID68 | CRM | n.a | Alhydrogel | s.c. |
| SeqID71 | SeqID7 | Pustulan | n.a | i.d. |
| | | (80%) | | |
| SeqID72 | CRM197 | n.a | Alhydrogel | i.d. |
| SeqID73 | SeqID7 | Pustulan | n.a | i.d. |
| | | (80%) | | |
| SeqID74 | CRM197 | n.a | Alhydrogel | s.c. |

Animals (female Balb/c mice) were vaccinated 3 times in biweekly weekly intervals (all vaccines: 20 μg of aSyn targeting peptide/dose; route: i.d. for the CLEC based vaccines and s.c. for the CRM197 based vaccines adjuvanted with Alhydrogel). Samples of murine plasma taken two weeks after each immunization as well as respective control samples (e.g.: the aSyn binding antibody LB509, epitope aa115-122, or pre-immune plasma obtained before immunization) have been analyzed for in vitro aggregation inhibition capacity.

### Results:

As shown in FIG. 32D, plasma taken from animals prior to immunization had no significant effects on the aggregation kinetics of aSyn confirming the specificity of the assay.

The SeqID67+SeqID7+pustulan vaccine (containing a 10-mer aSyn derived peptide) induced Abs strongly inhibited aSyn aggregation as indicated by an 40% decreased aggregation in this assay over time whereas the respective CRM conjugate vaccine was showing only minimal effects indicating a significantly higher inhibition capacity as compared to classical vaccine induced Abs (FIG. 32A). A similar result could be achieved analysing antibodies induced by SeqID71+SeqID7+pustulan (containing a 12-mer aSyn derived peptide), which could reduce aggregation even more strongly (70-80% inhibition) and could surpass inhibition capacity of antibodies induced by conventional CRM vaccine (SeqID72+CRM+Alhydrogel) by 2 to 2.5-fold. FIG. 32C shows that the antibodies induced by CLEC based and conventional vaccines building on epitope aa107-114 ((containing an 8-mer aSyn derived peptide) in contrast failed to inhibit aSyn aggregation.

As shown in FIG. 32D, the aSyn specific antibody LB509 fails to inhibit aSyn aggregation. In the contrary, a slight increase in aggregation can be detected in this analysis.

This is a highly surprising effect considering the teachings in this invention (see example 14 and FIG. 15 analysing epitopes derived of aSyn sequence aa115-126 as well, especially also aa115-121), as well as table 1 and FIGS. 29-32 describing vaccines covering epitopes 115-126) as the monoclonal LB509 (epitope aa: 115-122) known to bind to different forms of aSyn (Jakes et al. Neurosci. Lett. 1999 Jul. 2; 269(1):13-6) and shares the same epitope as biologically effective vaccines according to this invention (see FIG. 32D). It follows that the biologically superior effects obtained with CLEC based vaccines are indeed surprising and are showing that highly selected peptide sequences contained within aa115-126 are preferred within the region aa103-135 as immunotherapeutics targeting aggregated aSyn specifically.

### Example 30: Determination of Biological Activity of Peptide-CRM197-CLEC-Conjugates In Vitro Towards Murine Dectin-1 Receptor

In a series of ELISA experiments conjugates containing the dectin-1 ligands pustulan, lichenan and laminarin have been assessed for their binding efficacy to murine dectin-1. Biological activity of the peptide+CRM197+CLEC conjugates is represented by their PRR binding ability. Along these lines and to ensure that the structure of CLEC (pustulan, lichenan, laminarin) remained biologically active after coupling, binding to murine dectin-1 was assessed. Non-oxidized and oxidized pustulan, lichenan and laminarin as well as CRM conjugate vaccine and peptide+CRM197+CLEC-based novel conjugates have then been assessed for their biological activity using a competitive ELISA system based on competitive binding of a soluble murine Fc-dectin-1a receptor (InvivoGen) as described in Korotchenko et al. 2020.

### Results:

Ensuing experiments revealed that the median molecular weight (20 kDa), linear β-(1,6) linked β-D-glucan pustulan and the linear β(1-3)-glucan with β(1-6)-linkages laminarin exert significantly higher binding efficacy (ca. 10-fold) to murine dectin-1 than the larger, high molecular weight, linear β-(1,3)-(1,4)-β-D glucan lichenan (ca 245 kDa) (see FIG. 33).

As shown in FIG. 33A the dectin-1 ligand pustulan, oxidized pustulan, SeqID6+CRM-conjugate (CRM-conjugate 1) and SeqID6+CRM+pustulan conjugate (CRM-Pus conjugate 1) have been assessed for their binding efficacy to murine dectin-1 by ELISA analysis. Ensuing experiments revealed that the peptide+CRM197+pustulan conjugate displays similar binding efficacy to murine dectin-1 as oxidized pustulan. In contrast, the conventional CRM-conjugate 1 displays no specific murine dectin-1 binding. High binding efficacy to murine dectin-1 is also shown as well by 5 novel CRM-pustulan conjugates (SeqID52/66/68/70/72) (FIG. 33B). Ensuing experiments revealed that peptide-CRM197-pustulan conjugates with different B-cell epitopes, ranging from 7-mer B-cell epitope (SeqID6+CRM+Pus; FIG. 33A) to 12-mer B-cell epitope (SeqID71+CRM+Pus; FIG. 1B) display similar binding efficacy to murine dectin-1 as oxidized pustulan. As shown in FIG. 33C, the high molecular weight (ca 22-245 kDa) linear, β-(1,3) β-(1,4)-β-D glucan lichenan, exerts lower binding efficacy, irrespective of oxidation or conjugation, than the linear β-(1,6) linked β-D-glucan pustulan based constructs. For example, pustulan containing CRM197-peptide conjugates retain an approx. 10-fold higher binding than lichenan based constructs. High binding efficacy to murine dectin-1 is also shown by the linear β(1-3)-glucan with β(1-6)linkages laminarin (FIG. 33D). Ensuing experiments revealed that the peptide+CRM197+laminarin conjugate displays similar binding efficacy to murine dectin-1, irrespective of oxidation or conjugation, as for pustulan based constructs.

The experiment revealed that peptide+CRM197+CLEC conjugates demonstrate biological activity towards dendritic cells via binding to dectin-1 in the murine system.

### Example 31: Determination of Biological Activity of Peptide-CRM197-CLEC-Conjugates In Vitro Towards Human Dectin-1 Receptor

In a series of ELISA experiments the dectin-1 ligands pustulan, lichenan and laminarin have been assessed for their binding efficacy to human dectin-1. Biological activity of the peptide+CRM197+CLEC conjugates is represented by their PRR binding ability. Along these lines and to ensure that the structure of CLEC (pustulan, lichenan, laminarin) remained biologically active after coupling, binding to human dectin-1 was assessed by competitive ELISA system based on competitive binding of a soluble human Fc-dectin-1a receptor (InvivoGen).

### Results:

As shown in FIG. 34, the SeqID6+CRM-conjugate coupled to either lichenan (Lich conjugate), pustulan (Pus conjugate) or laminarin (Lam conjugate) have been assessed for their binding efficacy to human dectin-1 by ELISA analysis.

Ensuing experiments revealed that peptide+CRM197+pustulan vaccines exert significantly higher binding efficacy (ca. 30-fold) to human dectin-1 than vaccine conjugated to Lichenan (see FIG. 34). In contrast, peptide+CRM197+laminarin vaccines display weak binding to human Dectin-1.

### Example 32: In Vivo Comparison of Different Peptide+CRM197+Pustulan-Based Vaccines

Novel CRM197-pustulan based vaccines with different B-cell epitopes, ranging from 8 mer to 11 mer, which were able to bind to their DC receptor (e.g.: dectin-1) were tested for their ability to induce a robust and specific immune response following repeated application in n=5 Balb/c mice/group. Typical experiments were performed applying 5 μg net peptide content of B-cell epitope peptides per dose.

In this experiment the aSyn derived peptide SeqID52+CRM197 and SeqID66/68/70+CRM conjugates, were coupled to oxidized pustulan. Animals (female Balb/c mice) were vaccinated 3 times in biweekly intervals (route: i.d.) with either ß-Glucan-modified or unmodified Peptide-CRM conjugates and the ensuing immune response directed against the injected peptides (i.e., SeqID52/66/68/70, respectively) and against aggregated aSyn filaments was analyzed using murine plasma taken two weeks after the third immunization.

### Vaccine Used:

| B-cell | T-cell | | | |
|---|---|---|---|---|
| epitope | epitope/carrier | CLEC | adjuvant | Route |
| SeqID52 | CRM | Pustulan | n.a | i.d. |
| | | (80%) | | |
| SeqID66 | CRM | Pustulan | n.a | i.d. |
| | | (80%) | | |
| SeqID68 | CRM | Pustulan | n.a | i.d. |
| | | (80%) | | |
| SeqID70 | CRM | Pustulan | n.a | i.d. |
| | | (80%) | | |
| SeqID52 | CRM | n.a. | Alhydrogel | s.c. |
| SeqID66 | CRM | n.a | Alhydrogel | s.c. |
| SeqID68 | CRM | n.a | Alhydrogel | s.c. |
| SeqID70 | CRM | n.a. | Alhydrogel | s.c. |

### Results:

As shown in FIG. 35A, all 4 CRM-pustulan based vaccines (SeqID52/66/68/70/72) were able to induce significantly higher responses against both, the injected peptide moieties (e.g.: SeqID52/66/68/70) and against aggregated aSyn filaments when compared to unmodified peptide-CRM-based vaccines adjuvanted with Alhydrogel.

Peptide+CRM+pustulan based conjugates could induced 2-5x higher titers against the respective peptide (highest titers of 1/190.000) and 3-13x higher titers against aSyn filaments (highest titers of 1/29.000) as unmodified peptide-CRM-based vaccines.

### Example 33: Analysis of Selectivity of Immune Responses Elicited by Peptide+CRM+Pustulan Based Vaccines In Vivo

To further characterize the immune responses elicited by peptide+CRM197+pustulan based vaccines containing different B-cell epitopes as compared to conventional peptide+CRM197 vaccines, a set of experiments was performed analysing the selectivity of the ensuing immune response elicited towards aggregated aSyn filaments.

Animals (female Balb/c mice) were vaccinated 3 times in biweekly intervals (all vaccines: 5 μg of aSyn targeting peptide/dose; route: i.d. for the 4 peptide+CRM197+CLEC based vaccines (SeqID52/SeqID66/68/70-CRM197-pus) and s.c. for the 4 peptide+CRM197 based vaccines (SeqID52/SeqID66/68/70-CRM197 adjuvanted with Alhydrogel) and the ensuing immune response against the target protein, i.e., recombinant human alpha Synuclein as well as aSyn filament has been analysed using murine plasma taken two weeks after the third immunization. The plasma samples were subjected to an aSyn specific inhibition ELISA and IC50 values were determined.

### Vaccine Used:

| B-cell | T-cell | | | |
|---|---|---|---|---|
| epitope | epitope/carrier | CLEC | adjuvant | Route |
| SeqID52 | CRM | Pustulan | n.a | i.d. |
| | | (80%) | | |
| SeqID66 | CRM | Pustulan | n.a | i.d. |
| | | (80%) | | |
| SeqID68 | CRM | Pustulan | n.a | i.d. |
| | | (80%) | | |
| SeqID70 | CRM | Pustulan | n.a | i.d. |
| | | (80%) | | |
| SeqID52 | CRM | n.a. | Alhydrogel | s.c. |
| SeqID66 | CRM | n.a | Alhydrogel | s.c. |
| SeqID68 | CRM | n.a | Alhydrogel | s.c. |
| SeqID70 | CRM | n.a. | Alhydrogel | s.c. |

### Results:

Briefly, all CLEC based conjugates used in this experiment demonstrate superior aSyn aggregate specific target selectivity, as determined by a much lower IC50 value against aSyn filaments, as compared to the conventional peptide-CRM197 conjugate vaccines (see FIG. 36).

All 4 conventional peptide-CRM197 conjugate vaccines tested in this experiment induced antibodies demonstrating a very weak selectivity towards aSyn filaments, shown by very high IC50 values with 400-1.700 ng/ml.

In contrast, all antibodies induced by novel peptide+CRM197+pustulan based conjugate vaccines were characterized by much lower IC50 values for aSyn filaments ranging from 3.5-15 ng/ml.

Thus, the experiments show that CLEC modification of CRM197 conjugates leads to a strongly enhanced target specificity of the ensuing immune response, regardless of the epitope used, providing a novel unprecedented strategy to optimize current state of the art conjugate vaccines.

### Example 34: Analysis of Avidity of Immune Responses Elicited by Peptide+CRM197+Pustulan Based Vaccines

To further characterize the immune responses elicited by peptide-CRM197-pustulan based vaccines containing different B-cell epitopes as compared to conventional peptide-CRM197 vaccines a set of experiments was performed analysing the avidity of the antibodies elicited towards aSyn filaments.

Animals (female Balb/c mice) were vaccinated 3 times in biweekly intervals (all vaccines: 5 μg of aSyn targeting peptide/dose; route: i.d. for the CLEC based vaccines (SeqID52/66/68/70+CRM197+pustulan) and s.c. for CRM197 based vaccines adjuvanted with Alhydrogel (SeqID52/66/68/70-CRM197) and the ensuing immune response against the target protein, i.e., aSyn filament has been analysed using murine plasma taken two weeks after each immunization. To determine avidity of the induced Abs towards aSyn filaments, a variation of the standard ELISA assay was used where replicate wells containing antibody bound to antigens were exposed to increasing concentrations of chaotropic thiocyanate ions. Resistance to thiocyanate elution was used as the measure of avidity and an index (avidity index) representing 50% of effective antibody binding was used to compare plasma samples.

### Vaccine Used:

| B-cell | T-cell | | | |
|---|---|---|---|---|
| epitope | epitope/carrier | CLEC | adjuvant | Route |
| SeqID52 | CRM197 | Pustulan | n.a | i.d. |
| | | (80%) | | |
| SeqID66 | CRM197 | Pustulan | n.a | i.d. |
| | | (80%) | | |
| SeqID68 | CRM197 | Pustulan | n.a | i.d. |
| | | (80%) | | |
| SeqID70 | CRM197 | Pustulan | n.a | i.d. |
| | | (80%) | | |
| SeqID52 | CRM197 | n.a. | Alhydrogel | s.c. |
| SeqID66 | CRM197 | n.a | Alhydrogel | s.c. |
| SeqID68 | CRM197 | n.a | Alhydrogel | s.c. |
| SeqID70 | CRM197 | n.a | Alhydrogel | s.c. |

### Results:

As shown in FIG. 37, all tested conventional peptide-CRM197 conjugate (adjuvanted with Alhydrogel) induced antibodies showed only limited binding strength towards aSyn filaments as demonstrated by very low avidity indexes ranging from 0.25 to 0,85. In contrast, all novel peptide+CRM197+pustulan based vaccines induced antibodies showed significantly higher binding strength towards aSyn filaments with AIs ranging from 0.5-2.2.

The experiments therefore show that CLEC modification of peptide-CRM197 conjugates leads to a strongly enhanced target specific immune response (titer), as well as to a strongly enhanced target specificity and affinity of the induced antibody response regardless of the epitope used, providing a novel unprecedented strategy to optimize current state of the art protein-conjugate vaccines, including CRM197.

### Example 35: In Vivo Comparison of Different Peptide+CRM197+CLEC-Based Vaccines

The aSyn derived peptide SeqID6+CRM197 conjugates coupled either to pustulan, lichenan, or laminarin are tested for their ability to induce a robust and specific immune response following repeated application in n=5 Balb/c mice/group. Typical experiments are performed applying 5 μg net peptide content of B-cell epitope peptides per dose. Animals (female Balb/c mice) are vaccinated 3 times in biweekly intervals (route: i.d.) and the ensuing immune response directed against the injected peptides (i.e., SeqID6) and against aggregated aSyn filaments is analyzed using murine plasma taken two weeks after the third immunization.

### Vaccine Used:

| B-cell | T-cell | | | |
|---|---|---|---|---|
| epitope | epitope/carrier | CLEC | adjuvant | Route |
| SeqID6 | CRM197 | Pustulan | n.a | i.d. |
| | | (80%) | | |
| SeqID6 | CRM197 | Lichenan | n.a | i.d. |
| | | (200%) | | |
| SeqID6 | CRM197 | Laminarin | n.a | i.d. |
| | | (200%) | | |
| SeqID6 | CRM197 | n.a | Alhydrogel | s.c. |

### Results:

Vaccines tested can induce significant immune responses against the injected peptide (e.g. SeqID6) as well as against aggregated aSyn filaments following repeated immunization in mice. Peptide+CRM+pustulan based conjugates induce high titers against the respective peptide and high titers against aSyn filaments compared to conventional peptide-CRM-based vaccines and to peptide-CRM-based vaccines conjugated to laminarin or lichenan (see FIG. 38).

Specifically, SeqID+CRM197+pustulan induces 1.6 fold higher titers directed against the injected peptide SeqID6 as compared to SeqID6+CRM197+lichenan and 12 fold higher titers as compared to SeqID6+CRM197+laminarin. SeqID6+CRM197+Lichenan could induce 7.5 fold higher titers as compared to SeqID6+CRM197+Laminarin, respectively.

Similarly, SeqID+CRM197+Pustulan induces 3,1 fold higher titers directed against aSyn aggregates (filaments) as compared to SeqID6+CRM197+lichenan, 7.6 fold higher titers as compared to SeqID6+CRM197+laminarin and 6 fold higher titers as compared to non-CLEC modified SeqID6+CRM197 adjuvanted with Alum. SeqID6+CRM197+Lichenan could induce 2.4 fold higher titers as compared to SeqID6+CRM197+Laminarin and 2 fold higher titers as compared to non-CLEC modified SeqID6+CRM197 adjuvanted with Alum, respectively. CLEC modification of peptide-CRM197 conjugates are providing a novel unprecedented strategy to optimize current state of the art protein-conjugate vaccines, including CRM197.

### Example 36: Determination of Biological Activity of Peptide-CLEC-Conjugates In Vitro Towards Murine and Human Dectin-1 Receptor

In a series of ELISA experiments the dectin-1 ligands pustulan, lichenan and laminarin have been assessed for their binding efficacy to murine and human dectin-1. Biological activity of the peptide-CLEC conjugates is represented by their PRR binding ability. Along these lines and to ensure that the structure of CLEC (pustulan, lichenan, laminarin) remained biologically active after coupling, binding to murine and human dectin-1 was assessed by competitive ELISA system based on competitive binding of a soluble murine and human Fc-dectin-1a receptor (InvivoGen).

### Results:

As shown in FIG. 39, the SeqID5+SeqID7+CLEC conjugates coupled to either lichenan (Lich conjugate), pustulan (Pus conjugate) or laminarin (Lam conjugate) have been assessed for their binding efficacy to murine and human dectin-1 by ELISA analysis.

Ensuing experiments revealed that peptide-pustulan vaccines exert significantly higher binding efficacy to murine and human dectin-1 than vaccines conjugated to lichenan (see FIG. 39A+B). In contrast, the peptide-laminarin vaccine display very high binding to murine dectin1 (FIG. 39A) but only exerts weak binding to human Dectin-1 (FIG. 39B).

### Example 37: In Vivo Comparison of Different Peptide-CLEC-Based Vaccines

CLEC based vaccines which were able to bind to murine and/or human dectin-1 were tested for their ability to induce a robust and specific immune response following repeated application in n=5 Balb/c mice/group. Typical experiments were performed applying 5 μg net peptide content of B-cell epitope peptides per dose.

In this experiment the aSynuclein derived peptide SeqID5 and the promiscuous T-helper cell epitope SeqID7 were coupled via C-terminal hydrazide linkers to oxidized Pustulan, Lichenan or Laminarin.

### Vaccine Used:

| B-cell epitope | T-cell epitope | CLEC |
|---|---|---|
| SeqID5 | SeqID7 | Pustulan |
| SeqID5 | SeqID7 | Lichenan |
| SeqID5 | SeqID7 | Laminarin |

Animals (female Balb/c mice) were vaccinated 3 times in biweekly intervals (dose: 5 μg and 20 μg; route: i.d.) and the ensuing immune response directed against the injected peptides (i.e., SeqID6) was analyzed using murine plasma taken two weeks after the third immunization.

### Results:

As shown in FIG. 40A (dose: 5 μg SeqID5 peptide equivalent) and B (dose: 20 μg SeqID5 peptide equivalent), all three CLEC vaccines (SeqID5+SeqID7+pustulan, SeqID5+SeqID7+lichenan and SeqID5+SeqID7+laminarin were able to induce a detectable immune response in a dose dependent way. Interestingly, immunization using the vaccine based on laminarin did only induce a very low antipeptide and anti aSyn response irrespective of the dose applied. In contrast, pustulan based conjugates could induce a significantly higher response. Lichenan based conjugates showed a lower immunogenicity as compared to pustulan-based conjugates but could induce higher titers as the laminarin based conjugate in this experiment.

This demonstrates that dectin-1 binding efficacy in vitro, especially also to human dectin-1, can be directly linked to in vivo immunogenicity and biological activity of the vaccines. This leads to the identification of pustulan or fragments thereof (i.e. linear β(1,6)-β-D glucans) as most efficacious glucan variant as proposed in this application.

### Example 38: Determination of Biological Activity of CLEC Modified Oligo/Polysaccharide+CRM197 and Oligo/Polysaccharide+TT-Glycoconjugates In Vitro

Biological activity of the oligo/polysaccharide+CRM197+pustulan and oligo/polysaccharide+TT+pustulan conjugates is represented by their PRR binding ability. In this example two commercially available conjugates have been either coupled to pustulan or left non-modified and have been analysed: i) the Neisseria meningitidis oligosaccharide (A, C, W135, and Y) containing CRM197 conjugate vaccine Menveo® and ii) the Haemophilus influenzae type b capsular polysaccharide (polyribosyl-ribitol-phosphate, PRP) Tetanus Toxoid (TT) conjugate ActHIB®

To ensure that the structure of pustulan remained biologically active after coupling to Menveo® and ActHIB®, binding to dectin-1 was assessed using a competitive ELISA system based on competitive binding of a soluble murine Fc-dectin-1a receptor (InvivoGen) as described in Korotchenko et al. 2020. Non-modified and pustulan-modified CRM197 and TT conjugate vaccines have then been assessed for their biological activity and compared to relevant controls.

### Results:

In an ELISA experiment the oxidized dectin-1 ligand pustulan, the Haemophilus influenzae type b capsular polysaccharide (polyribosyl-ribitol-phosphate, PRP) Tetanus Toxoid (TT) conjugate ActHIB® modified with pustulan or left unmodified, and the Neisseria meningitidis oligosaccharide (A, C, W135, and Y) containing CRM197 conjugate vaccine Menveo® with and without β-Glucan modification have been assessed for their binding efficacy to dectin-1. Ensuing experiments (FIG. 41) revealed that CRM-pustulan and TT-pustulan conjugates display similar binding efficacy to dectin-1 as oxidized pustulan. In contrast, the conventional non modified CRM- and TT-conjugates displays no specific dectin-1 binding.

The experiment revealed that oligo/polysaccharide-CRM197/TT-pustulan conjugates demonstrate biological activity towards dendritic cells via binding to dectin-1.

### Example 39: In Vivo Comparison of Different Oligo/Polysaccharide+CRM197+Pustulan-Based Vaccines and Oligo/Polysaccharide+TT+Pustulan-Based Vaccines

The Haemophilus influenzae type b capsular polysaccharide (polyribosyl-ribitol-phosphate, PRP) Tetanus Toxoid (TT) conjugate ActHIB® and the Neisseria meningitidis oligosaccharide (A, C, W135, and Y) containing CRM197 conjugate vaccine Menveo®, are coupled to oxidized pustulan and tested for their ability to induce a robust and specific immune response following repeated application in n=5 Balb/c mice/group.

In this experiment animals (female Balb/c mice) were vaccinated 3 times in biweekly intervals with either β-Glucan-modified (route: i.d.,) or unmodified conjugates (route i.m.;) and the ensuing immune response directed against ActHIB® and Menveo® is analyzed using murine plasma taken two weeks after the third immunization.

### Vaccine Used:

| B-cell | T-cell | | | |
|---|---|---|---|---|
| epitope | epitope/carrier | CLEC | adjuvant | Route |
| N. meningitidis | CRM197 | Pustulan | n.a | i.d. |
| (A, C, W135, Y) | | (80%) | | |
| N. meningitidis | CRM197 | n.a. | n.a | i.m. |
| (A, C, W135, Y) | | | | |
| H. influenzae | TT | Pustulan | n.a | i.d. |
| (b) PRP | | (80%) | | |
| H. influenzae | TT | n.a. | n.a | i.m. |
| (b) PRP | | | | |

### Results:

As shown in FIG. 42, all vaccines tested could induce significant immune responses against the immunizing conjugates following repeated immunization in mice.

CLEC modified Menveo® and ActHIB® treated animals showed 2,4-fold and 1,4-fold higher anti conjugate responses as non-modified vaccines indicating an improvement of immunogenicity of oligo/polysaccharide-carrier vaccines. These results also demonstrate that CLEC modification of existing, clinically validated oligo/polysaccharide-carrier vaccines according to the current invention improves immunogenicity of said vaccines.

Furthermore, the examples provided show that peptide- and oligo/polysaccharide-CRM/TT-βGlucan vaccines are functional in vivo and suitable as novel vaccine compositions for the treatment of infectious diseases according to the present invention.

### Example 40: Analysis of B-Cell Epitopes of Secreted Proteins, Self-Antigens, and Conformational Epitopes: IL31

To assess whether peptide vaccines carrying peptides derived from secreted proteins (irrespective whether they constitute self-antigens (as SeqID132 to SeqID147) or foreign target structures) can mount high immune reactions following repeated immunization and can induce immune reactions which are superior to conventional conjugate vaccines, 8 different vaccine candidates were tested:

In this experiment 8 different IL31 derived peptides were either used as peptide-CLEC vaccine i.e.: SeqID132; SeqID134; SeqID136; SeqID138; SeqID140; SeqID142; SeqID144; and SeqID146 in combination with the pan-T-cell epitope SeqID7, coupled via C-terminal hydrazide linkers to oxidized pustulan (80%); or as conventional peptide-conjugates, produced using SeqID133, SeqID135, SeqID137, SeqID139, SeqID141 SeqID143; SeqID145; and SeqID147; containing a C-terminal cysteine for coupling to GMBS activated CRM197.

### Vaccines Used:

| B-cell | T-cell | | | |
|---|---|---|---|---|
| epitope | epitope/carrier | CLEC | adjuvant | Route |
| SeqID132 | SeqID7 | Pustulan | n.a | i.d. |
| | | (80%) | | |
| SeqID133 | CRM197 | n.a | Alhydrogel | s.c. |
| SeqID134 | SeqID7 | Pustulan | n.a | i.d. |
| | | (80%) | | |
| SeqID135 | CRM197 | n.a | Alhydrogel | s.c. |
| SeqID136 | SeqID7 | Pustulan | n.a | i.d. |
| | | (80%) | | |
| SeqID137 | CRM197 | n.a | Alhydrogel | s.c. |
| SeqID138 | SeqID7 | Pustulan | n.a | i.d. |
| | | (80%) | | |
| SeqID139 | CRM197 | n.a | Alhydrogel | s.c. |
| SeqID140 | SeqID7 | Pustulan | n.a | i.d. |
| | | (80%) | | |
| SeqID141 | CRM197 | n.a | Alhydrogel | s.c. |
| SeqID142 | SeqID7 | Pustulan | n.a | i.d. |
| | | (80%) | | |
| SeqID143 | CRM197 | n.a | Alhydrogel | s.c. |
| SeqID144 | SeqID7 | Pustulan | n.a | i.d. |
| | | (80%) | | |
| SeqID145 | CRM197 | n.a | Alhydrogel | s.c. |
| SeqID146 | SeqID7 | Pustulan | n.a | i.d. |
| | | (80%) | | |
| SeqID147 | CRM197 | n.a | Alhydrogel | s.c. |

Animals (female Balb/c mice) were vaccinated 3 times in biweekly weekly intervals (route: i.d. for the CLEC based vaccine and s.c. for the CRM based vaccine (adjuvanted with Alhydrogel) and the ensuing immune response directed against the injected peptide (i.e., SeqID133, SeqID135, SeqID137, SeqID139, SeqID141 SeqID143; SeqID145; and SeqID147) as well as against the target protein, i. e., recombinant human IL31 was analysed using murine plasma taken two weeks after the third immunization.

### Results:

As shown in FIG. 43, vaccines were able to induce a strong and specific immune response against both, the injected peptide moieties (FIG. 43A) and the target protein: human IL31 (FIG. 43 B).

As compared to CRM197 peptide conjugate vaccines, adjuvanted with Alhydrogel, peptide-CLEC based vaccines of this example however showed a similar or significantly higher immune response directed against the injected peptide moiety and most importantly also against the full-length target, human IL31.

In addition, analysis of avidity using resistance to thiocyanate elution (NaSCN) revealed a significantly higher avidity towards full length human IL31 for peptide-CLEC induced antibodies as compared to peptide-CRM197 induced antibodies, respectively (see FIG. 43C; example: comparison of SeqID132+SeqID7+Pustulan and SeqID133+CRM Alum induced antibodies).

In summary, CLEC based vaccines tested were highly suitable for use of epitopes directed against secreted proteins including signaling molecules or Cyto/Chemokines, especially human IL31, as immunogens conferring high immune responses as well as high target specific responses compared to conventional vaccines.

This example also provides results demonstrating that CLEC based immunogens using epitopes of human IL31 were surprisingly inducing immune responses with higher affinity as compared to state of the art vaccines against these self-epitopes.

Therefore, it is evident that the CLEC based vaccines according to the present invention can be preferably used for active anti-IL31 immunization.

### Example 41: Analysis of Immunogenicity of IL31 Targeting CLEC Conjugates Using Carrier Proteins as T-Helper Cell Epitopes: CRM197

In this example immunogenicity of CLEC based conjugate vaccines containing the well-known carrier protein CRM197 was compared to conventional CRM197 vaccines. For this purpose, the human IL31 derived epitopes SeqID133, SeqID135, SeqID137, SeqID139, SeqID141 SeqID143; SeqID145; SeqID147; SeqID149 and SeqID151 were coupled to maleimide activated CRM197. Subsequently, the CRM197 conjugates were coupled to activated pustulan using the heterobifunctional linker BPMH to form CLEC based conjugate vaccines with CRM197 as source for T-helper cell epitopes to induce a sustainable immune response.

### Vaccines Used:

| B-cell | T-cell | | | |
|---|---|---|---|---|
| epitope | epitope/carrier | CLEC | adjuvant | Route |
| SeqID133 | CRM197 | Pustulan | n.a | i.d. |
| | | (80%) | | |
| SeqID133 | CRM197 | n.a | Alhydrogel | s.c. |
| SeqID135 | CRM197 | Pustulan | n.a | i.d. |
| | | (80%) | | |
| SeqID135 | CRM197 | n.a | Alhydrogel | s.c. |
| SeqID137 | CRM1 97 | Pustulan | n.a | i.d. |
| | | (80%) | | |
| SeqID137 | CRM197 | n.a | Alhydrogel | s.c. |
| SeqID1391 | CRM197 | Pustulan | n.a | i.d. |
| | | (80%) | | |
| SeqID139 | CRM197 | n.a | Alhydrogel | s.c. |
| SeqID141 | CRM197 | Pustulan | n.a | i.d. |
| | | (80%) | | |
| SeqID141 | CRM197 | n.a | Alhydrogel | s.c. |
| SeqID143 | CRM197 | Pustulan | n.a | i.d. |
| | | (80%) | | |
| SeqID143 | CRM197 | n.a | Alhydrogel | s.c. |
| SeqID145 | CRM197 | Pustulan | n.a | i.d. |
| | | (80%) | | |
| SeqID145 | CRM197 | n.a | Alhydrogel | s.c. |
| SeqID147 | CRM197 | Pustulan | n.a | i.d. |
| | | (80%) | | |
| SeqID147 | CRM197 | n.a | Alhydrogel | s.c. |
| SeqID149 | CRM197 | Pustulan | n.a | i.d. |
| | | (80%) | | |
| SeqID149 | CRM197 | n.a | Alhydrogel | s.c. |
| SeqID151 | CRM197 | Pustulan | n.a | i.d. |
| | | (80%) | | |
| SeqID151 | CRM197 | n.a | Alhydrogel | s.c. |

Animals (female Balb/c mice) were vaccinated 3 times in biweekly weekly intervals (all vaccines: 5 μg of IL31 targeting peptide/dose; route: i.d. for the CLEC based vaccines and s.c. for the CRM197 based vaccine adjuvanted with Alhydrogel) and the ensuing immune response directed against the injected peptide (i.e., SeqID133, SeqID135, SeqID137, SeqID139, SeqID141 SeqID143; SeqID145; SeqID147; SeqID149 and SeqID151) as well as against the full length IL31 was analysed using murine plasma taken two weeks after the third immunization.

### Results:

IL31-peptide+CRM197 based vaccines induced a strong and specific immune response against both, the injected peptide moieties (FIG. 44A) and the target protein: human IL31 (FIG. 44B).

CLEC modification of the IL31 targeting CRM197 conjugates led to a similar or significantly higher immune response against the immunizing peptide as compared to non-CLEC modified Alhydrogel adjuvanted conventional CRM197 based vaccines. Importantly, target specific anti-full length IL31 titers elicited by non-CLEC modified, Alhydrogel adjuvanted conventional CRM197 based vaccines were either similar (SeqID141+CRM and SeqID147+CRM) or 2-9 fold lower than CLEC modified vaccines.

In addition, analysis of avidity using resistance to thiocyanate elution (NaSCN) revealed a significantly higher avidity towards full length human IL31 for IL31-peptide+CRM197+CLEC induced antibodies as compared to IL31-peptide+CRM197 induced antibodies, respectively (see FIG. 44C, example: comparison of SeqID133+CRM+Pustulan and SeqID133+CRM Alum induced antibodies).

Thus, the experiments showed that CLEC modification of conventional peptide-protein conjugates led to a strongly enhanced target specificity of the ensuing immune response providing a novel unprecedented strategy to optimize current state of the art conjugate vaccines building on carrier proteins like KLH, CRM197 or others.

This example also provides results demonstrating that CLEC based immunogens using epitopes of human IL31 were surprisingly inducing immune responses with higher titer and affinity as compared to state-of-the-art vaccines against IL31.

Therefore, it is evident that the CLEC based vaccines according to the present invention can be preferably used for active anti-IL31 immunization.

### Example 42: Inhibition of IL31 Signaling by WISIT Vaccine Induced Anti IL31 Antibodies

To investigate the inhibition of native IL-31 signalling by WISIT vaccine induced and conventional CRM197 vaccine induced antibodies, A549 cells, human adenocarcinomic alveolar basal epithelial cells (ATCC, Virginia, USA), were treated with different vaccine induced antibodies (1000 ng/ml) followed by addition of human IL-31. Vaccine induced antibodies used were obtained from animals undergoing repeated immunization described in examples 40 and 41. All samples were applied at an anti IL31 antibody concentration of 1000 ng/ml. Controls include an IL31 blocking antibody (Immunogen against E. coli-derived recombinant human IL-31 Ser24-Thr164 Accession #Q6EBC2, at a concentration of 1000 ng/ml) used as positive control as well as murine plasma without the inhibitory antibody as negative control in this assay.

After incubation for 20 minutes, cells were lysed and the phosphorylation of STAT3 was analysed with a PathScan Phospho-Stat3 (Tyr705) Sandwich ELISA Kit (Cell Signaling Technologies, Danvers, MA, USA).

### Results:

Conventional peptide+carrier, peptide+CLEC and peptide+carrier+CLEC vaccine induced antibodies were able to exert a specific inhibition of IL31 signaling using this cell based in vitro assay (FIGS. 45 and 46), demonstrating their ability to modify effects exerted by IL31 activity (i.e. demonstrate biological activity and therapeutic potential).

CLEC modification of the IL31 targeting vaccines (both types, peptide-conjugate as well as peptide-CRM-conjugates) surprisingly led to immune responses with similar or significantly higher inhibitory capacity as compared to state of the art, non-CLEC modified Alhydrogel adjuvanted conventional CRM197 based vaccines.

FIG. 45 summarizes analysis of inhibitory capacity of antibodies induced by IL31-peptide+SeqID7+pustulan conjugates (IL31 peptides: SeqID132, SeqID134, SeqID136, SeqID138, SeqID140, SeqID142, SeqID144, SeqID146) as well as conventional IL31-peptide+CRM conjugates (IL31 peptides: SeqID133, SeqID135, SeqID137, SeqID139, SeqID141, SeqID143, SeqID145, SeqID147).

FIG. 46 summarizes analysis of inhibitory capacity of antibodies induced by IL31-peptide+CRM+pustulan conjugates (IL31 peptides: SeqID133, SeqID135, SeqID137, SeqID139, SeqID141, SeqID143, SeqID145, SeqID147, SeqID149, SeqID 151) as well as conventional IL31-peptide+CRM conjugates adjuvanted with Alum (IL31 peptides: SeqID133, SeqID135, SeqID137, SeqID139, SeqID141, SeqID143, SeqID145, SeqID147, SeqID149, SeqID 151), respectively.

Therefore, it is evident that the CLEC based vaccines according to the present invention can be preferably used for active anti-IL31 immunization. Such vaccines can therefore be used for the treatment and prevention of IL31 related diseases and autoimmune inflammatory diseases. The analysis of the inhibitory capacity of vaccine induced Abs also revealed that the immunogenic peptides SeqID132/133, SeqID 134/135, SeqID138/139, SeqID146/147, SeqID148/149 and SeqID 150/151 induce more efficient antibodies (both in inhibiting IL31 activity as well as compared to conventional, vaccine induced antibodies) and are thus highly suitable whereas SeqID136/137, SeqID140/141, SeqID142/143 and SeqID144/145 are less suitable.

### Example 43: Analysis of B-Cell Epitopes of Secreted Proteins, Self-Antigens, and Conformational Epitopes: CGRP

To assess whether peptide vaccines carrying peptides derived from secreted proteins (irrespective whether they constitute self-antigens or foreign target structures) can mount high immune reactions following repeated immunization and can induce immune reactions which are superior to conventional conjugate vaccines, different vaccine candidates were tested:

In this experiment different CGRP (calcitonin-gene related peptide) derived peptides were either used as peptide+CLEC vaccine i.e.: SeqID152, SeqID154, SeqID156, SeqID158, SeqID160 and SeqID162 in combination with the pan-T-cell epitope SeqID7, coupled via C-terminal hydrazide linkers to oxidized pustulan (80%); or as conventional peptide+CRM conjugates, produced using SeqID153 SeqID157 SeqID159 SeqID161 and SeqID163 containing a C-terminal cysteine for coupling to GMBS activated CRM197.

### Vaccines Used:

| B-cell | T-cell | | | |
|---|---|---|---|---|
| epitope | epitope/carrier | CLEC | adjuvant | Route |
| SeqID152 | SeqID7 | Pustulan | n.a | i.d. |
| | | (80%) | | |
| SeqID153 | CRM197 | n.a | Alhydrogel | s.c. |
| SeqID154 | SeqID7 | Pustulan | n.a | i.d. |
| | | (80%) | | |
| SeqID155 | CRM197 | n.a | Alhydrogel | s.c. |
| SeqID156 | SeqID7 | Pustulan | n.a | i.d. |
| | | (80%) | | |
| SeqID157 | CRM197 | n.a | Alhydrogel | s.c. |
| SeqID158 | SeqID7 | Pustulan | n.a | i.d. |
| | | (80%) | | |
| SeqID159 | CRM197 | n.a | Alhydrogel | s.c. |
| SeqID160 | SeqID7 | Pustulan | n.a | i.d. |
| | | (80%) | | |
| SeqID161 | CRM197 | n.a | Alhydrogel | s.c. |
| SeqID162 | SeqID7 | Pustulan | n.a | i.d. |
| | | (80%) | | |
| SeqID163 | CRM197 | n.a | Alhydrogel | s.c. |

Animals (female Balb/c mice) were vaccinated 3 times in biweekly intervals (route: i.d. for the CLEC based vaccine and s.c. for the CRM based vaccine (adjuvanted with Alhydrogel) and the ensuing immune response directed against the injected peptide (i.e., SeqID153, SeqID155, SeqID157, SeqID159, SeqID161 and SeqID163) as well as against the target protein, i.e., recombinant human CGRP was analysed using murine plasma taken two weeks after the third immunization.

### Results:

Vaccines tested were able to induce a strong and specific immune response against both, the injected peptide moieties and the target protein: human CGRP (FIG. 47).

As compared to CRM197 peptide conjugate vaccines, adjuvanted with Alhydrogel, the peptide-CLEC based vaccines of this example showed a similar or significantly higher immune response directed against the injected peptide moieties (FIG. 47A) and most importantly also against the full-length target, human CGRP (FIG. 47B).

In addition, analysis of avidity using resistance to thiocyanate elution (NaSCN) revealed a significantly higher avidity towards full length human CGRP for peptide+CLEC induced antibodies compared to peptide+CRM197 induced antibodies, respectively (FIG. 47C).

In summary, the CLEC based vaccines tested are highly suitable for use of epitopes directed against secreted proteins including signaling molecules or Cyto/Chemokines, especially human CGPR, as immunogens conferring high immune responses as well as high target specific responses as compared to conventional vaccines.

This example also provides results demonstrating that CLEC based immunogens using epitopes of human CGPR are surprisingly inducing immune responses with higher affinity compared to state of the art vaccines against these self-epitopes.

Therefore, it is evident that the CLEC based vaccines according to the present invention can be preferably used for active anti-CGRP immunization. Such vaccines can therefore be used for the treatment of CGRP associated diseases including: episodic and chronic migraine and cluster headache, hyperalgesia, hyperalgesia in dysfunctional pain states, such as for example rheumatoid arthritis, osteoarthritis, visceral pain hypersensitivity syndromes, fibromyalgia, inflammatory bowel syndrome, neuropathic pain, chronic inflammatory pain and headaches.

### Example 44: Analysis of Immunogenicity of CGRP Targeting CLEC Conjugates Using Carrier Proteins as T-Helper Cell Epitopes: CRM197

In this example immunogenicity of CLEC based conjugate vaccines containing the well-known carrier protein CRM197 was compared to conventional peptide+CRM197 vaccines. For this purpose, the human CGRP derived epitopes SeqID153, SeqID155, SeqID157, SeqID159, SeqID161 and SeqID163 were coupled to maleimide activated CRM197. Subsequently, the CRM197 conjugates were coupled to activated pustulan using the heterobifunctional linker BPMH to form CLEC based conjugate vaccines with CRM197 as source for T-helper cell epitopes to induce a sustainable immune response.

| B-cell | T-cell | | | |
|---|---|---|---|---|
| epitope | epitope/carrier | CLEC | adjuvant | Route |
| SeqID153 | CRM197 | Pustulan | n.a | i.d. |
| | | (80%) | | |
| SeqID153 | CRM197 | n.a | Alhydrogel | s.c. |
| SeqID155 | CRM197 | Pustulan | n.a | i.d. |
| | | (80%) | | |
| SeqID155 | CRM197 | n.a | Alhydrogel | s.c. |
| SeqID157 | CRM197 | Pustulan | n.a | i.d. |
| | | (80%) | | |
| SeqID157 | CRM197 | n.a | Alhydrogel | s.c. |
| SeqID159 | CRM197 | Pustulan | n.a | i.d. |
| | | (80%) | | |
| SeqID159 | CRM197 | n.a | Alhydrogel | s.c. |
| SeqID161 | CRM197 | Pustulan | n.a | i.d. |
| | | (80%) | | |
| SeqID161 | CRM197 | n.a | Alhydrogel | s.c. |
| SeqID163 | CRM197 | Pustulan | n.a | i.d. |
| | | (80%) | | |
| SeqID163 | CRM197 | n.a | Alhydrogel | s.c. |

Animals (female Balb/c mice) were vaccinated 3 times in biweekly intervals (all vaccines: 5 μg of CGRP targeting peptide/dose; route: i.d. for the CLEC based vaccines and s.c. for the CRM197 based vaccine adjuvanted with Alhydrogel) and the ensuing immune response directed against the injected peptide (i.e., SeqID153, SeqID155, SeqID157, SeqID159, SeqID161 and SeqID163) as well as against the full length CGRP was analysed using murine plasma taken two weeks after the third immunization.

### Results:

CRM197 based vaccines could induce a strong and specific immune response against both, the injected peptide moieties and the target protein: human CGRP (FIG. 48).

CLEC modification of the CGRP targeting CRM197 conjugates led to induction of a similar or higher immune response compared to non-CLEC modified, Alhydrogel adjuvanted conventional CRM197 based vaccines for both, anti-immunizing peptide (FIG. 48A) and anti-full length CGRP responses (FIG. 48B).

In addition, analysis of avidity using resistance to thiocyanate elution (NaSCN) reveals a significantly higher avidity towards full length human CGRP for CGRP-peptide+CRM197+CLEC induced antibodies as compared to CGRP-peptide+CRM197 induced antibodies, respectively (FIG. 48C).

Thus, the experiments showed that CLEC modification of conventional peptide-protein conjugates led to a high target specificity of the ensuing immune response providing a novel unprecedented strategy to optimize current state of the art conjugate vaccines building on carrier proteins like KLH, CRM197 or others.

This example also provided results demonstrating that CLEC based immunogens using epitopes of human CGPR were surprisingly inducing immune responses with higher titer and affinity compared to state-of-the-art vaccines against CGRP.

Therefore, it is evident that the CLEC based vaccines according to the present invention can be preferably used for active anti-CGRP immunization. Such vaccines can therefore be used for the treatment of CGPR associated diseases including episodic and chronic migraine and cluster headache, hyperalgesia, hyperalgesia in dysfunctional pain states, such as for example rheumatoid arthritis, osteoarthritis, visceral pain hypersensitivity syndromes, fibromyalgia, inflammatory bowel syndrome, neuropathic pain, chronic inflammatory pain and headaches.

### Example 45: Analysis of In Vivo Functionality of Immune Responses Elicited by CLEC Based Vaccines

To determine if aSyn specific antibodies elicited by CLEC based vaccines were able to inhibit aSyn fibril formation in vivo, a proof-of-concept experiment was initiated using an established seeding model for synucleinopathies [Sci. Adv. 2020, 6, eabc4364, doi: 10.1126/sciadv.abc4364; DOI: 10.1126/sciadv.abc4364].

### Vaccines Used:

| B-cell | T-cell | | | |
|---|---|---|---|---|
| epitope | epitope/carrier | CLEC | adjuvant | Route |
| SeqID5 | SeqID7 | Pustulan | n.a | i.d. |
| | | (80%) | | |
| na | na | Pustulan | n.a | i.d. |

In this model, C57BL/6 mice were stereotactically injected with α-syn pre-formed fibrils (PFFs) at the level of the right substantia nigra, subsequently causing widespread synucleinopathy, characterized specifically by phosphosynuclein immunopositive Lewy-like neurites and intracytoplasmic aggregates along anatomical connections. Animals were immunized four times at weeks 0, 2, 4 and 10 with SeqID5+SeqID7+pustulan vaccine, or nonconjugated CLEC as control, starting the first immunization on the day of PFF inoculation. 126 days post PFF injection, animals were sacrificed, and brains were analyzed for the presence of phospho-S129 aSyn-positive aggregates in selected brain areas including cerebral cortex, striatum, thalamus, substantia nigra and brainstem.

### Results:

Analysis of the ensuing immune response was performed using plasma and CSF obtained at the time of sacrifice. High antibody titers against the injected peptide were detected in plasma of SeqID5+SeqID7+pustulan vaccine treated animals. In contrast, no signal above background could be detected in the CLEC-only treated group (FIG. 49). Analysis of the anti-peptide titer in CSF also showed a high level of SeqID5+SeqID7+pustulan vaccine induced antibodies, whereas no signal above background was detectable for the vehicle treated animals (FIG. 49). Immunohistochemistry of brain sections showed high numbers of phospho-S129 aSyn-positive aggregates throughout all analyzed areas in the vehicle treated group indicating a strong propagation of aSyn pathology. In contrast, synucleinopathy was significantly reduced in SeqID5+SeqID7+pustulan vaccinated mice (FIG. 49). Of note, there was a strong and significant reciprocal correlation between the strength of the antibody response and the level of synucleinopathy in vaccine recipients (FIG. 49).

### Example 46: Analysis of Immunogenicity of Peptide+CRM+CLEC Conjugates

In this example carrier specific immunogenicity of CLEC based conjugate vaccines was compared to conventional carrier vaccines.

For this purpose, the alpha synuclein derived epitope SeqID6 or the IL31 derived epitopes SeqID133, SeqID135 and SeqID137 have been coupled to maleimide activated CRM197. Subsequently, Peptide-CRM197 conjugates have been coupled to activated pustulan using the heterobifunctional linker BPMH to form CLEC based conjugate vaccines with CRM197 as source for T-helper cell epitopes to induce a sustainable immune response.

### Vaccines Used:

| B-cell | T-cell | | | |
|---|---|---|---|---|
| epitope | epitope/carrier | CLEC | adjuvant | Route |
| SeqID6 | CRM197 | Pustulan | n.a | i.d. |
| | | (80%) | | |
| SeqID6 | CRM197 | n.a | Alhydrogel | s.c. |
| SeqID133 | CRM197 | Pustulan | n.a | i.d. |
| | | (80%) | | |
| SeqID133 | CRM197 | n.a | Alhydrogel | s.c. |
| SeqID135 | CRM197 | Pustulan | n.a | i.d. |
| | | (80%) | | |
| SeqID135 | CRM197 | n.a | Alhydrogel | s.c. |
| SeqID137 | CRM197 | Pustulan | n.a | i.d. |
| | | (80%) | | |
| SeqID137 | CRM197 | n.a | Alhydrogel | s.c. |

Animals (female Balb/c mice) were vaccinated 3 times in biweekly intervals and the ensuing immune response directed against the carrier protein CRM197 has been analysed using murine plasma taken two weeks after the third immunization. Dose of SeqID6 containing vaccines: 20 μg and 100 μg of alpha synuclein targeting peptide/dose; route: i.d. for the CLEC based vaccines and s.c. for the CRM197 based vaccine adjuvanted with Alhydrogel (FIG. 50A). Dose used for SeqID133-, SeqID135- and SeqID137-containing vaccines: 5 μg IL31 targeting peptide/dose; route: i.d. for the CLEC based vaccines and and s.c. for the CRM197 based vaccine adjuvanted with Alhydrogel

### Results:

Comparison of anti-carrier specific antibody responses revealed that conventional SeqID6+CRM197 based vaccines were able to dose dependently induce high anti-CRM197 titers. In contrast, CLEC based SeqID6+CRM197+pustulan vaccines used were inducing significantly lower anti CRM responses following repeated immunization using 20 μg and 100 μg doses (reduction: 4.5-5 fold; FIG. 50A).

Similarly, non-CLEC-modified SeqID133-, SeqID135- and SeqID137+CRM197 based vaccines, used at a dose of 5 μg IL31 targeting peptide/dose, were inducing 3.7-5.8 fold higher anti-CRM197 titers than CLEC modified peptide-CRM conjugates, respectively (FIG. 50B).

Thus, the experiments show that covalent CLEC modification of conventional peptide-protein conjugates impairs development of an anti-carrier response significantly, providing a novel unprecedented strategy to optimize current state of the art conjugate vaccines building on carrier proteins like KLH, CRM197 or others.

### Example 47: In Vivo Analysis of Anti-Pustulan/Glucan Immune Responses Following Immunisation

As already discussed in example 7, analysis of anti-CLEC antibodies induced by CLEC-based immunogens is important on two levels for the novelty and efficacy of the proposed CLEC-vaccines according to the present invention.

Along these lines, an extensive analysis of anti-pustulan antibodies in plasma samples of naïve, peptide+CLEC and peptide+CRM+CLEC conjugate immunized Balb/c mice (n=5/group) prior to immunization and following repeated immunizations was performed.

| B-cell | T-cell | | | Ratio |
|---|---|---|---|---|
| epitope | epitope | CLEC | Adjuvant | (w/w) |
| n.a. | n.a. | Pustulan | n.a. | n.a. |
| SeqID6 | CRM197 | Pustulan | n.a | 1/1 |
| | | (80%) | | |
| SeqID6 | CRM197 | Pustulan | n.a | 1/2.5 |
| | | (80%) | | |
| SeqID6 | CRM197 | Pustulan | n.a | 1/5 |
| | | (80%) | | |
| SeqID6 | CRM197 | Pustulan | n.a | 1/10 |
| | | (80%) | | |
| SeqID6 | CRM197 | Pustulan | n.a | 1/20 |
| | | (80%) | | |
| SeqID6 | CRM197 | Lichenan | n.a | |
| | | (200%) | | |
| SeqID6 | CRM197 | Laminarin | n.a | |
| | | (200%) | | |
| SeqID132 | SeqID7 | Pustulan | n.a. | |
| | | (80%) | | |
| SeqID133 | CRM197 | Pustulan | n.a. | |
| | | (80%) | | |
| SeqID133 | CRM197 | n.a | Alhydrogel | |
| SeqID134 | SeqID7 | Pustulan | n.a. | |
| | | (80%) | | |
| SeqID135 | CRM197 | Pustulan | n.a. | |
| | | (80%) | | |
| SeqID135 | CRM197 | n.a | Alhydrogel | |
| SeqID136 | SeqID7 | Pustulan | n.a. | |
| | | (80%) | | |
| SeqID137 | CRM197 | Pustulan | n.a. | |
| | | (80%) | | |
| SeqID137 | CRM197 | n.a | Alhydrogel | |

### Results:

4 different types of samples were analysed in this example:

FIG. 51A shows the anti-pustulan immunoreactivity from samples obtained from animals undergoing repeated SeqID6+CRM+pustulan, SeqID6+CRM+lichenan or SeqID6+CRM+laminarin immunizations (all vaccines: 20 μg of aSyn targeting peptide/dose). FIG. 51B shows the anti-pustulan immunoreactivity from samples obtained from animals undergoing repeated SeqID6+CRM+pustulan immunisations using vaccines containing different w/w peptide+CRM conjugate/CLEC ratios: i.e. conjugate/CLEC ratios of 1/1, 1/2,5, 1/5, 1/10 and 1/20 (all vaccines: 5 μg of aSyn targeting peptide/dose). FIG. 51C shows the anti-pustulan immunoreactivity from samples obtained from animals undergoing repeated SeqID133+CRM+pustulan, SeqID135+CRM+pustulan or SeqID137+CRM+pustulan immunizations (all vaccines: 5 μg of IL31 targeting peptide/dose). FIG. 51D shows the anti-pustulan immunoreactivity from samples obtained from animals undergoing repeated SeqID132+SeqID7+pustulan, SeqID134+SeqID7+pustulan or SeqID136+SeqID7+pustulan immunizations (all vaccines: 5 μg of IL31 targeting peptide/dose).

For control purposes samples obtained from animals prior to immunization as well as from non-oxidised CLEC treated animals were used. In addition, samples obtained from animals undergoing application of vaccines consisting of non-CLEC modified peptide+CRM-conjugates (SeqID133+CRM, SeqID135+CRM or SeqID137+CRM, adjuvanted with Alum) were included in this analysis as well.

As shown in FIG. 51, Balb/c animals analysed showed a preexisting low level immune response directed against glucans/pustulan/β(1,6)-β-D glucan.

All CLEC vaccines tested (peptide+CLEC and peptide+CRM+CLEC conjugates) failed in significantly increasing pre-existing anti glucan responses or de novo inducing high immune responses directed against the glucan backbone in vivo (all samples tested: <2x of pre-immune levels; average: 0.8+/−0.5-fold change).

In contrast, repeated application of unconjugated, non-oxidized pustulan present in the control group led to the induction of a strong anti-glucan immune response by boosting antibody levels against pustulan >5 times (compared to pre-immune plasma). NonCLEC modified peptide+CRM conjugates and lichenan- and laminarin-containing conjugates were unable to induce anti-pustulan titers above pre-immune levels indicating specificity of the anti-glucan response detected.

In summary, these analyses could demonstrate that: despite presence of a low-level, pre-existing auto-reactivity against pustulan (IgG) in naïve Balb/c mice, no/very low vaccination dependent change of anti-pustulan immunoreactivity is detected following immunization using various CLEC conjugates. This is indicating a significant lowering of Glucan immunogenicity applying the novel vaccine design according to the present invention. This is in strong contrast to previously published results and therefore constitutes a surprising and inventive novel characteristic of the carbohydrate backbone (e.g. the β-glucans, especially the pustulan backbone) according to the present invention.

In addition, pre-existing anti-pustulan-responses do not seem to preclude immune reactions to the peptide component of WISIT vaccines as the injected peptide responses for all experiments revealed high anti-peptide titers.

### Example 48: In Vivo Comparison of the Effect of Glucan Conjugation on Immunogenicity of Peptide+Carrier Vaccines

To assess whether conjugation of CLECs to peptide+carrier immunogens is required for the induction of superior immunogenicity of the vaccines according to the present invention, a set of experiments was initiated comparing three vaccine preparations: a peptide+carrier conjugate modified covalently with β-Glucan, a vaccine preparation containing a mix of the peptide+carrier conjugate and the β-Glucan without conjugation and a non-modified, non-Alum adjuvanted peptide+carrier vaccine.

Again, n=5 female Balb/c mice were immunized i.d. three times in biweekly intervals and the ensuing immune response directed against the injected peptide and aSyn filament (i.e., SeqID6) was analyzed using murine plasma taken two weeks after the third immunization.

### Vaccine Used:

| B-cell epitope | Carrier | CLEC | CLEC-Conjugation |
|---|---|---|---|
| SeqID6 | CRM197 | Pustulan (80%) | yes |
| SeqID6 | CRM197 | Pustulan (non-ox.) | No; mixing only |
| SeqID6 | CRM197 | n.a., no adjuvant | no |

### Results:

FIG. 52 shows the comparison of anti-peptide (SeqID6) and anti aSyn monomer specific immune responses detectable following three immunizations. SeqID6+CRM197+pustulan conjugates were able to induce approx. 10 times higher immune responses against the injected peptide (FIG. 52A) and 4-fold higher anti aSyn titers (FIG. 52B) as reported for the mix of SeqID6+CRM197 and non-oxidized pustulan as well as approx. 10 fold higher aSyn titers as those for SeqID6+CRM197 (w/o adjuvant) in this experiment. Interestingly, mixing of SeqID6+CRM197 and non-oxidized pustulan did not result in a significantly different immune response as conventional SeqID6+CRM197.

These data show that conjugation of peptide-carrier immunogens to activated CLECs according to the current invention is required to induce a superior immune response in vivo.

B-cell epitope sequences disclosed in the examples were as follows:

| SeqID | Sequence | Paternal Protein |
|---|---|---|
| SeqID1 | (H2N-NH-CO-CH2-CH2-CO)-DQPVLPD | Alpha Synuclein |
| SeqID2 | DQPVLPD-(NH-NH2) | Alpha Synuclein |
| SeqID3 | C-DQPVLPD | Alpha Synuclein |
| SeqID4 | (H2N-NH-CO-CH2-CH2-CO)-DMPVDPD | Alpha Synuclein |
| SeqID5 | DMPVDPD-(NH-NH2) | Alpha Synuclein |
| SeqID6 | C-DMPVDPD | Alpha Synuclein |
| SeqID10 | DAEFRH-(NH-NH2) | Aβ |
| SeqID11 | DAEFRH-C | Aβ |
| SeqID12 | MDVFMKGL-(NH-NH2) | Alpha Synuclein |
| SeqID13 | MDVFMKGL-C | Alpha Synuclein |
| SeqID14 | ATGFVKKDQL-(NH-NH2) | Alpha Synuclein |
| SeqID15 | ATGFVKKDQL-C | Alpha Synuclein |
| SeqID16 | LGKNEEGAP-(NH-NH2) | Alpha Synuclein |
| SeqID20 | EGYQDYEPEA-(NH-NH2) | Alpha Synuclein |
| SeqID21 | EGYQDYEPEA-C | Alpha Synuclein |
| SeqID32 | p (E) FRHDS-C | Aβ |
| SeqID33 | p (E) FRHDS-(NH-NH2) | Aβ |
| SeqID35 | KDNIKHVPGGGS-C | Tau |
| SeqID36 | KDNIKHVPGGGS-(NH-NH2) | Tau |
| SeqID37 | WDPGMLQSELIQKEFGDY-C | IL12/23 |
| SeqID38 | WDPGMLQSELIQKEFGDY-(NH-NH2) | IL12/23 |
| SeqID39 | TPDAPGETV-C | IL12/23 |
| SeqID40 | TPDAPGETV-(NH-NH2) | IL12/23 |
| SeqID41 | TQQIPSLSPSQ-C | IL12/23 |
| SeqID42 | TQQIPSLSPSQ-(NH-NH2) | IL12/23 |
| SeqID43 | QQQGLPRAAGG-C | EMPD |
| SeqID44 | QQQGLPRAAGG-(NH-NH2) | EMPD |
| SeqID45 | QQFLSVRAL-C | Bet v1 |
| SeqID46 | QQFLSVRAL-(NH-NH2) | Bet v2 |
| SeqID47 | YMPIWKFPDEEGAC | Her2 |
| SeqID48 | YMPIWKFPDEEGAC-(NH-NH2) | Her2 |
| SeqID49 | GAISLAPKAQIKESLRAEL-C | PD1 |
| SeqID50 | GAISLAPKAQIKESLRAEL-(NH-NH2) | PD1 |
| SeqID51 | DMPVDPDN-NH-NH2 | Alpha Synuclein |
| SeqID52 | CDMPVDPDN | Alpha Synuclein |
| SeqID53 | DMPVDP-(NH-NH2) | Alpha Synuclein |
| SeqID55 | C-DMPVDP | Alpha Synuclein |
| SeqID56 | DMPVD-(NH-NH2) | Alpha Synuclein |
| SeqID58 | C-DMPVD | Alpha Synuclein |
| SeqID65 | DMPVDPDNE-(NH-NH2) | Alpha Synuclein |
| SeqID66 | DMPVDPDNE-C | Alpha Synuclein |
| SeqID67 | DMPVDPDNEA-(NH-NH2) | Alpha Synuclein |
| SeqID68 | DMPVDPDNEA-C | Alpha Synuclein |
| SeqID69 | DMPVDPDNEAY-(NH-NH2) | Alpha Synuclein |
| SeqID70 | DMPVDPDNEAY-C | Alpha Synuclein |
| SeqID71 | DMPVDPDNEAYE-(NH-NH2) | Alpha Synuclein |
| SeqID72 | DMPVDPDNEAYE-C | Alpha Synuclein |
| SeqID73 | APQEGILE-(NH-NH2) | Alpha Synuclein |
| SeqID74 | APQEGILE-C | Alpha Synuclein |
| SeqID132 | SKMLLKDVEEEKG-NHNH2 | IL31 |
| SeqID133 | SKMLLKDVEEEKG-C | IL31 |
| SeqID134 | EELQSLSK-NHNH2 | IL31 |
| SeqID135 | EELQSLSK-C; | IL31 |
| SeqID136 | KGVLVS-NHNH2 | IL31 |
| SeqID137 | KGVLVS-C | IL31 |
| SeqID138 | SVIDEIIEHLDKLI-NHNH2 | IL31 |
| SeqID139 | SVIDEIIEHLDKLI-C | IL31 |
| SeqID140 | SPAIRAYLKTIRQLDNKSVI-NHNH2 | IL31 |
| SeqID141 | SPAIRAYLKTIRQLDNKSVI-C | IL31 |
| SeqID142 | HERKRFILT-NHNH2 | IL31 |
| SeqID143 | HERKRFILT-C | IL31 |
| SeqID144 | HESKRFILT-NHNH2 | IL31 |
| SeqID145 | HESKRFILT-C | IL31 |
| SeqID146 | SVPTDTHERKRF-NHNH2 | IL31 |
| SeqID147 | SVPTDTHERKRF-C | IL31 |
| SeqID148 | SVPTDTHESKRE-NHNH2 | IL31 |
| SeqID149 | SVPTDTHESKRF-C | IL31 |
| SeqID150 | KRFILTISQQFS-NHNH2 | IL31 |
| SeqID151 | KRFILTISQQFS-C | IL31 |
| SeqID152 | RLAGLLSR-NHNH2 | CGRP |
| SeqID153 | RLAGLLSR-C | CGRP |
| SeqID154 | RLAGLLSRSGGVVKN-NHNH2 | CGRP |
| SeqID155 | RLAGLLSRSGGVVKN-C | CGRP |
| SeqID156 | RSGGVVKN-NHNH2 | CGRP |
| SeqID157 | RSGGVVKN-C | CGRP |
| SeqID158 | NNFVPTNVGSKAF-NHNH2 | CGRP |
| SeqID159 | NNFVPTNVGSKAF-C | CGRP |
| SeqID160 | VPTNVGSKAF-NHNH2 | CGRP |
| SeqID161 | VPTNVGSKAF-C | CGRP |
| SeqID162 | NVGSKAF-NHNH2 | CGRP |
| SeqID163 | NVGSKAF-C | CGRP |

Based on this general disclosure of the present invention and these examples, the following preferred embodiments of the present invention are disclosed:
1. A conjugate consisting of or comprising at least a β-glucan or a mannan and at least a B-cell or T-cell epitope polypeptide, wherein the β-glucan or mannan is covalently conjugated to the B-cell and/or T-cell epitope polypeptide to form a conjugate of the β-glucan or mannan and the B-cell and/or T-cell epitope polypeptide.
2. A conjugate according to embodiment 1, wherein the β-glucan is a predominantly linear β-(1,6)-glucan with a ratio of (1,6)coupled monosaccharide moieties to non-β-(1,6)-coupled monosaccharide moieties of at least 1:1, preferably at least 2:1, more preferred, at least 5:1, especially at least 10:1.
3. A conjugate according to embodiment 1 or 2, wherein the Bglucan is a dectin-1 binding β-glucan, preferably pustulan, lichenan, laminarin, curdlan, β-glucan peptide (BGP), schizophyllan, scleroglucan, whole glucan particles (WGP), zymosan, or lentinan, more preferred pustulan, laminarin, lichenan, lentinan, schizophyllan, or scleroglucan, especially pustulan; and/or wherein the β-glucan is a strong dectin-1 binding β-glucan, preferably a β-glucan which binds to the soluble murine Fc-dectin-1a receptor with an IC50 value lower than 10 mg/ml, more preferred with an IC50 value lower than 1 mg/ml, even more preferred with an IC50 value lower than 500 μg/ml, especially with an IC50 value lower than 200 μg/ml, as determined by a competitive ELISA; and/or wherein the conjugates bind to the soluble murine Fc-dectin-1a receptor with an IC50 value lower than 1 mg/ml, more preferred with an IC50 value lower than 500 μg/ml, even more preferred with an IC50 value lower than 200 μg/ml, especially with an IC50 value lower than 100 μg/ml, as determined by a competitive ELISA; and/or
a β-glucan which binds to the soluble human Fc-dectin-1a receptor with an IC50 value lower than 10 mg/ml, more preferred with an IC50 value lower than 1 mg/ml, even more preferred with an IC50 value lower than 500 μg/ml, especially with an IC50 value lower than 200 μg/ml, as determined by a competitive ELISA; and/or
wherein the conjugates bind to the soluble human Fc-dectin-1a receptor with an IC50 value lower than 1 mg/ml, more preferred with an IC50 value lower than 500 μg/ml, even more preferred with an IC50 value lower than 200 μg/ml, especially with an IC50 value lower than 100 μg/ml, as determined by a competitive ELISA.
4. A conjugate according to any of the embodiments 1 to 3, wherein the polypeptides comprise at least one B-cell and at least one T-cell epitope, preferably a B-cell epitope+CRM197 conjugate covalently linked to β-glucan, especially a peptide+CRM197+linear β-(1,6)-glucan or a peptide+CRM197+linear pustulan conjugate.
5. A conjugate according to any one of embodiments 1 to 4, wherein the ratio of β-glucan to B-cell and/or T-cell epitope polypeptide in the conjugate, especially pustulan to peptide ratios, is from 10:1 (w/w) to 0.1:1 (w/w), preferably from 8:1 (w/w) to 2:1 (w/w), especially 4:1 (w/w), with the proviso if the conjugate comprises a carrier protein, the preferred ratio of β-glucan to B-cell-epitope+carrier polypeptide is from 50:1 (w/w), to 0.1:1 (w/w), especially 10:1 to 0.1:1.
6. A conjugate according to any one of embodiments 1 to 5, wherein a B-cell epitope and a pan-specific/promiscuous T-cell epitope is independently coupled to the β-glucan.
7. A conjugate according to any one of embodiments 1 to 6, wherein the B-cell epitope polypeptide has a length of 5 to 20 amino acid residues, preferably of 6 to 19 amino acid residues, especially of 7 to 15 amino acid residues; and/or wherein the T-cell epitope polypeptide has a length of 8 to 30 amino acid residues, preferably of 13 to 29 amino acid residues, especially of 13 to 28 amino acid residues,
wherein the B-cell epitope and/or the T-cell epitope is preferably linked to the β-glucan and/or to a carrier protein by a linker, more preferred a cysteine residue or a linker comprising a cysteine or glycine residue, a linker resulting from hydrazide-mediated coupling, from coupling via heterobifunctional linkers, such as N-β-maleimidopropionic acid hydrazide (BMPH), 4-[4-N-maleimidophenyl]butyric acid hydrazide (MPBH), N-[ε-Maleimidocaproic acid) hydrazide (EMCH) or N-[κ-maleimidoundecanoic acid] hydrazide (KMUH), from imidazole mediated coupling, from reductive amination, from carbodiimide coupling a —NH—NH₂ linker; an NRRA, NRRA-C or NRRA-NH—NH₂ linker, peptidic linkers, such as bi-, tri-, tetra- (or longer)-meric peptide groups, such as CG or CG, or cleavage sites, such as a cathepsin cleavage site; or combinations thereof, especially by a cysteine or NRRA-NH—NH₂ linker;
wherein the T-cell epitope is preferably a polypeptide comprising the amino acid sequence AKFVAAWTLKAAA, optionally linked to a linker, such as a cysteine residue or a linker comprising a cysteine residue, an NRRA, NRRA-C or NRRA-NH—NH₂ linker; or a variant of the amino acid sequence AKFVAAWTLKAAA, wherein the variants include the amino acid sequence AKFVAAWTLKAA, variants wherein the first residue alanine is replaced by an aliphatic amino acid residue, such as glycine, valine, isoleucine and leucine, variants wherein the third residue phenylalanine is replaced with L-cyclohexylalanine, variants wherein the thirteenth amino acid residue alanine is replaced by an aliphatic amino acid residue (e.g. glycine, valine, isoleucine and leucine), variants comprising aminocaproic acid, preferably coupled to the C-terminus of the amino acid sequence AKFVAAWTLKAA, variants with the amino acid sequence AX₁FVAAX₂TLX₃AX₄A, wherein X₁ is selected from the group consisting of W, F, Y, H, D, E, N, Q, I, and K; X₂ is selected from the group consisting of F, N, Y, and W, X₃ is selected from the group consisting of H and K, and X₄ is selected from the group consisting of A, D, and E, with the proviso that the oligopeptide sequence is not AKFVAAWTLKAAA; especially wherein the T-cell epitope is selected from AKFVAAWTLKAAANRRA-(NH—NH₂), AKFVAAWTLKAAAN-C, AKFVAAWTLKAAA-C, AKFVAAWTLKAAANRRA-C, aKXVAAWTLKAAaZC, aKXVAAWTLKAAaZCNRRA, aKXVAAWTLKAAa, aKXVAAWTLKAAaNRRA, aA(X)AAAKTAAAAa, aA(X)AAATLKAAa, aA(X)VAAATLKAAa, aA(X)IAAATLKAAa, aK(X)VAAWTLKAAa, and aKFVAAWTLKAAa, wherein X is L-cyclohexylalanine, Z is aminocaproic acid and a is an aliphatic amino acid residue selected from alanine, glycine, valine, isoleucine and leucine; and/or wherein the T-cell epitope is an alpha synuclein polypeptide selected from the group GKTKEGVLYVGSKTK (aa31-45), KTKEGVLYVGSKTKE (aa32-46), EQVTNVGGAVVTGVT (aa61-75), VTGVTAVAQKTVEGAGNIAAATGFVK (aa71-86), DPDNEAYEMPSE (aa116-130), DNEAYEMPSEEGYQD (aa121-135), and EMPSEEGYQDYEPEA (aa126-140).
8. A conjugate according to any one of embodiments 1 to 7, wherein the conjugate further comprises a carrier protein, preferably non-toxic cross-reactive material of diphtheria toxin (CRM), especially CRM197, KLH, diphtheria toxoid (DT), tetanus toxoid (TT), Haemophilus influenzae protein D (HipD), and the outer membrane protein complex of serogroup B meningococcus (OMPC), recombinant non-toxic form of Pseudomonas aeruginosa exotoxin A (rEPA), flagellin, Escherichia coli heat labile enterotoxin (LT), cholera toxin (CT), mutant toxins (e.g., LTK63 and LTR72), virus-like particles, albumin binding protein, bovine serum albumin, ovalbumin, a synthetic peptide dendrimer e.g. a Multiple antigenic peptide (MAP), especially wherein the ratio of carrier protein to β-glucan or mannan in the conjugate is from 1/0.1 to 1/50, preferably 1/0.1 to 1/40, more preferred from 1/0.1 to 1/20, especially from 1/0.1 to 1/10; with the preferred proviso that if the conjugate comprises a carrier protein, the conjugate comprises at least a further, independently conjugated T-cell or B-cell epitope polypeptide, wherein preferably the conjugate consists or comprises
(a) a β-glucan
(b) at least a B-cell or a T-cell epitope polypeptide, and
(c) a carrier protein,
wherein the three components (a), (b) and (c) are covalently conjugated with each other in the sequence (a)-(b)-(c), (a)-(c)-(b) or (b)-(a)-(c), especially in the sequence (a)-(c)-(b); and wherein preferably all these components (a), (b) and (c) are conjugated by linkers.
9. A conjugate according to any one of embodiments 1 to 8, wherein the polypeptide is or comprises a B-cell or a T-cell epitope polypeptide, preferably wherein the polypeptide is or comprises a B-cell and a T-cell epitope, especially wherein the epitope polypeptide is selected from the group of Tau polypeptides, preferably
Tau2-18, Tau 176-186, Tau 181-210, Tau 200-207, Tau 201-230, Tau 210-218, Tau 213-221, Tau 225-234, Tau 235-246, Tau 251-280, Tau 256-285, Tau 259-288, Tau 275-304, Tau260-264, Tau 267-273, Tau294-305, Tau 298-304, Tau 300-317, Tau 329-335, Tau 361-367, Tau 362-366, Tau379-408, Tau 389-408, Tau 391-408, Tau 393-402, Tau 393-406, Tau393-408, Tau 418-426, Tau 420-426; mimics of the above-mentioned Tau derived polypeptides including mimotopes and peptides containing amino acid substitutions mimicking phosphorylated amino acids including substitution of phosphorylated S by D and phosphorylated T by E, respectively including Tau176-186, Tau200-207, Tau210-218, Tau213-221, Tau225-234, Tau379-408, Tau389-408, Tau391-408, Tau393-402, Tau393-406, Tau418-426, Tau420-426; Tau379-408 with phosphorylated ps396 and pS404, double phosphorylated peptides Tau195-213[ps202/pT205], Tau207-220[pT212/pS214] and Tau224-238[pT231], an N-terminal YGG linker fused to 7-(Tau418-426) or 11-mer (Tau417-427),

| Tau-position | Sequence |
|---|---|
| 195-213 | SGYSSPGSPGTPGSRSRTP |
| 207-220 | GSRSRTPSLPTPPT |
| 224-238 | KKVAVVRTPPKSPSS |
| 393-408 | VYKpSPVVSGDTpSPRHL |
| 379-408 [P-Ser396, 404] | RENAKAKTDHGAEI- |
| | VYKpSPVVSGDTpSPRHL |
| Tau260-264 [P-Ser262] | IGpSTE |
| 294-305 | KDNIKHVPGGGS* |
| 251-280 | PDLKNVKSKIGSTENLKHQPGGGKVQIINK |
| 256-285 | VKSKIGSTENLKHQPGGGKVQIINKKLDLS |
| 256-285-pS262 | VKSKIGpSTENLKHQPGGGKVQIINKKLDLS |
| 259-288 | KIGSTENLKHQPGGGKVQIINKKLDLSNVQ |
| 275-304 | VQIINKKLDLSNVQSKCGSKDNIKHVPGGG |
| 201-230-pT217 | GSPGTPGSRSRTPSLPpTPPTREPKKVAVVR |
| 379-408-pS396pS404 | RENAKAKTDHGAEI- |
| | VYKpSPVVSGDTpSPRHL |
| 181-210-pS202pT205 | TPPSSGEPPKSGDRSGYSSPGPSPGpT- |
| | PGSRS |
| 300-317 | VPGGGSVQIVYKPVDLSK |
| 267-273 | KHQPGGG |
| 298-304 | KHVPGGG |
| 329-335 | HHVPGGG |
| 361-367 | THVPGGG |
| 200-207 (pS202/pT205) | PGpSPGpTPG |
| 200-207 (pS202/pT205) | PGDPGEPG |
| phospho mimic | |
| 200-207 (pS202/pT205) | SPGDPGEPG |
| mimic | |
| 210-218 (pT212/pS214) | SRpTPpSLPTP |
| 210-218 (pT212/pS214) | SREPDLPTP |
| mimic | |
| 210-218 (pT212/pS214) | SREPDLP |
| mimic | |
| 213-221 (pT217) | PSLPpTPPTR |
| 213-221 (pS214/pT217) | PpSLPpTPPTR |
| 213-221 (pT217 | PSLPEPPTR |
| phospho mimic | |
| 213-221 (pS214/pT217) | PDLPEPPTR |
| phospho mimic | |
| 393-402 (pSer396) | VYKpSPVVSGD |
| 393-402 (pSer396) | VYKDPVVSG |
| phospho mimic | |
| 393-406 (pSer396, pSer404) | VYKpSPVVSGDTpSPR |
| 393-406 (pSer396, pSer404) | VYKDPVVSGDTDPR |
| phospho mimic | |
| 176-186 (pT181) | PPAPKpTPPSSG |
| 176-186 (pT181) | PPAPKEPPSSG |
| phospho mimic | |
| 225-234 (pT231) | KVAVVRpTPPK |
| 225-234 (pT231) | KVAVVREPPKS |
| phospho mimic | |
| Tau379-408 [P-Ser396, 404] | RENAKAKTDHGAEI- |
| | VYKPSPVVSGDTpSPRHL |
| Tau379-408 [P-Ser396, 404] | RENAKAKTDHGAEI VYKDPVVSGDTDPRHL |
| phospho mimic | |
| 389-408 | GAEIVYKpSPVVSGDTpSPRHL |
| 391-408 | EIVYKpSPVVSGDTpSPRHL |
| 389-408 phospho mimic | GAEIVYKDPVVSGDTDPRHL |
| 391-408 phospho mimic | EIVYKDPVVSGDTDPRHL |
| 418-426 | DMVDpSPQLA |
| 418-426 phospho mimic | DMVDDPQLA |
| 420-426 | VDpSPQLA |
| 420-426 phospho mimic | VDDPQLA |
| 362-366 | HVPGG (LDNITHVPGGGNKKIE) |
| 235-246 | SPSSAKSRLQTA |

;

IL12/23 polypeptides, preferably
FYEKLLGSDIFTGE, FYEKLLGSDIFTGEPSLLPDSP, VAQLHASLLGLSQLLOP, GEPSLLPDSPVAQLHASLLGLSQLLOP, PEGHHWETQQIPSLSPSQP, PSLLPDSP, LPDSPVA, FYEKLLGSDIFTGEPSLLPDSPVAQLHASLLGLSQLLQP, LLPDSP, LLGSDIFTGEPSLLPDSPVAQLHASLLG, FYEKLLGSDIFTGEPSLLPDSPVAQLHASLLG, QPEGHHW, LPDSPVGOLHASLLGLSOLLQ and QCQQLSQKLCTLAWSAHPLV; GHMDLREEGDEETT, LLPDSPVGQLHASLLGLSQ and LLRFKILRSLQAFVAVAARV; aa136-145, aa136-143, aa 136-151, aa137-146, aa144-154, aa144-155 of the IL12/23 p40 subunit; QPEGHHWETQQIPSLS, GHHWETQQIPSLSPSQPWORL, QPEGHHWETQ, TQQIPSLSPSQ, QPEGHHWETQQIPSLSPSQ, QPEGHHWETQQIPSLSPS; aa15-66, aa38-46, aa53-71, aa119-130, aa160-177, aa236-253, aa274-285, aa315-330 of native human IL12/23p40; LLLHKKEDGIWSTDILKDQKEPKNKTFLRCE and KSSRGSSDPQG; aa38-46, aa53-71, aa119-130, aa160-177, aa236-253, aa274-285, aa315-330 of murine IL12/23; IgE polypeptides, preferably
SVNPGLAGGSAQSQRAPDRVL, HSGQQQGLPRAAGGSVPHPR; AVSVNPGLAGGSAQSQRAPDRVLCHSGQQQGLPRAAGGSVP, QQQGLPRAAGG, QQLGLPRAAGG, QQQGLPRAAEG, QQLGLPRAAEG, QQQGLPRAAG, QQLGLPRAAG, QQQGLPRAAE, QQLGLPRAAE, HSGQQQGLPRAAGG, HSGQQLGLPRAAGG, HSGQQQGLPRAAEG, HSGQQLGLPRAAEG, QSQRAPDRVLCHSG, GSAQSQRAPDRVL, and WPGPPELDV;
Her2 polypeptides, preferably
LHCPALVTYNTDTFESMPNPEGRYTFGASCV, ACPYNYLSTDVGSCTLVCPLHNQEVTAEDGTQRCEK, and CPLHNQEVTAEDGTQRCEK; KLLSLIKGVIVHRLEGVE; aa266-296, aa563-598, aa585-598, aa597-626, and aa613-626 of the Her2 sequence; AVLDNGDPLNNTTPVTGA, LKGGVLIQRNPQLC, YNTDTFESMPNPEGRYTEGAS, PESFDGDPASNTAPLQPEQLQ, PHQALLHTANRPEDE, CRVLQGLPREYVNARHC, YMPIWKFPDEEGAC; PESFDGDPASNTAPLQPC, RVLQGLPREYVNARHC, YMPIWKFPDEEGAC, PESFDGDPASNTAPLOP, YMPIWKFPDEEGAC, PESFDGDPASNTAPLQPRVLQGLPREYVNARHSLPYMPIWKFPDEEGAC, RVLQGLPREYVNARHSPESFDGDPASNTAPLQPYMPIWKFPDEEGAC; C-QMWAPQWGPD-C, C-KLYWADGELT-C, C-VDYHYEGTIT-C, C-QMWAPQWGPD-C, C-KLYWADGELT-C, C-KLYWADGEFT-C, C-VDYHYEGTIT-C, C-VDYHYEGAIT-C; RLVPVGLERGTVDWV, TRWQKGLALGSGDMA, QVSHWVSGLAEGSFG, LSHTSGRVEGSVSLL, LDSTSLAGGPYEAIE, HVVMNWMREEFVEEF, SWASGMAVGSVSFEE. QVSHWVSGLAEGSFG and LSHTSGRVEGSVSLL; RSLTEILKGGVLIQRNPQLC, VLIQRNPQLCYQDTILWKDI, YQDTILWKDIFHKNNQLALT, FHKNNQLALTLIDTNRSRAC, LIDTNRSRACHPCSMPCKGS, HPCSMPCKGSRCWGESSEDC, RCWGESSEDCQSLTRTVCAG, QSLTRTVCAGGCARCKGPLP, GCARCKGPLPTDCCHEQCAA, TDCCHEQCAAGCTGPKHSDC, GCTGPKHSDCLACLHENHSG, LACLHENHSGICELHCPALV, ICELHCPALVTYNTDTFESM, TYNTDTFESMPNPEGRYTFG, PNPEGRYTFGASCVTACPYN, GASCVTACPYNYLSTDVGS, PYNYLSTDVGSCTLVCPLHNQE, TLVCPLHNQEVTAEDGTQR, VTAEDGTQRCEKCSKPCARV, EKCSKPCARVCYGLGMEHLR, YGLGMEHLREVRAVTSANI, EVRAVTSANIQEFAGCKKI; KKIFGSLAF, GSLAFLPES, FAGCKKIFGS, SLAFLPESFD, FAGCKKIFGSLAFLPESFD, QEFAGCKKIFGSLAFLPESEDGD, SLAFLPESFD, especially YMPIWKFPDEEGAC;
PD1, PDL1 and CTLA-4 polypeptides, preferably GAISLAPKAQIKESLRAEL, PGWFLDSPDRPWNPP, FLDSPDRPWNPPTFS, SPDRPWNPPTFSPA, ISLHPKAKIEESPGA, and FMTYWHLLNAFTVTVPKDL, especially GAISLAPKAQIKESLRAEL;
Aβ polypeptides, preferably
native human Aβ1-40 and/or Aβ1-42 or a polypeptide fragment with aa1-6, aa1-7, aa1-8, aa1-9, aa1-10, aa1-11, aa1-12, aa1-13, aa1-14, aa1-15, aa1-21, aa2-7, aa2-8, aa2-9, aa2-10, aa3-8, aa3-9, aa3-10, aa pE3-8, aa pE3-9, aa pE3-10, aa11-16, aa11-17, aa11-18, aa11-19, aa12-19, aa13-19, aa14-19, aa14-20, aa14-21, aa14-22, aa14-23, aa30-40, aa31-40, aa32-40, aa33-40, aa34-40, aa30-42, aa37-42 of the Aβ1-42 sequence; NYSLDKIIVDYNLQSKITLP, LINSTKIYSYFPSVISKVNQ, LEYIPEITLPVIAALSIAES; cyclised Aβ1-14; DKELRI, DKELRID, DKELRIDS, DKELRIDSG, DKELRIDSGY, SWEFRT, SWEFRTD, SWEFRTDS, SWEFRTDSG, SWEFRTDSGY, TLHEFRH, TLHEFKH, THTDFRH, THTDFKH, AEFKHD, AEFKHG, SEFRHD, SEFRHG, SEFKHD, SEFKHG, ILFRHG, ILFRHD, ILFKHG, ILFKHD, IRWDTP, IRYDAPL, IRYDMAG;
IL31 polypeptides, preferably
native human IL31 (Genbank: AAS86448.1), native canine IL31 (Genbank: BAH97742.1), native feline IL31 (UNIPROT: A0A2I2UKP7), native equine IL31 (UNIPROT F7AHG9) or any peptide sequence which has at least 70, 75, 80, 85, 90 or 95% sequence identity to any of the foregoing, IL31 protein derived polypeptide selected from mimics of the above-mentioned IL31 derived polypeptides including mimotopes and peptides containing amino acid substitutions,
for human IL31: peptides derived from sequences aa98-145, aa87-150, aa105-113, aa85-115, aa84-114, aa86-117, aa87-116; or fragments thereof and peptides SDDVQKIVEELQSLSKMLLKDVEEEKGVLVSQNYTL; DVQKIVEELQSLSKMLLKDV, EELQSLSK and DVOK, LDNKSVIDEIIEHLDKLIFQDA; and DEIIEH, TDTHECKRFILTISQQFSECMDLALKS, TDTHESKRF, TDTHERKRF HESKRF, HERKRF, HECKRF; SDDVOKIVEELQ, VOKIVEELQSLS, IVEELQSLSKML, ELQSLSKMLLKD, SLSKMLLKDVEE, KMLLKDVEEEKG, LKDVEEEKGVLV, VEEEKGVLVSQN, EKGVLVSQNYTL, LDNKSVIDEIIE, KSVIDEIIEHLD IDEIIEHLDKLI, IIEHLDKLIFQD, HLDKLIFQDAPE, KLIFQDAPETNI, FQDAPETNISVP APETNISVPTDT, TNISVPTDTHEC, SVPTDTHESKRF, TDTHECKRFILT, TDTHESKRFILT, TDTHERKRFILT, HECKRFILTISQ, HESKRFILTISQ, HERKRFILTISQ, KRFILTISQQFS, ILTISQQFSECM, ILTISQOFSESM, ILTISQQFSERM, ISQQFSECMDLA, ISQQFSESMDLA, ISQQFSERMDLA, QFSECMDLALKS, QFSESMDLALKS, QFSERMDLALKS, SKMLLKDVEEEKG, EELQSLSK, KGVLVS, SPAIRAYLKTIRQLDNKSVIDEIIEHLDKLI, DEIIEHLDK, SVIDEIIEHLDKLI, SPAIRAYLKTIRQLDNKSVI, TDTHECKRF, HECKRFILT, HERKRFILT, HESKRFILT, SVPTDTHECKRF, SVPTDTHESKRF, and SVPTDTHERKRF for canine IL31: peptides consisting of aa97-144, aa97-133, aa97-122, aa97-114, aa90-110, aa90-144, aa86-144, aa97-149, aa90-149, aa86-149, aa 124-135 or fragments thereof and peptides: SDVRKIILELQPLSRGLLEDYQKKETGV, DVRKIILELQPLSRGLLEDY ELQPLSR LSDKNIIDKIIEQLDKLKFQHE, LSDKNIIDKIIEQLDKLKFQ, KLKFQHE, LSDKNI, LDKL, LSDKN, ADTFECKSFILTILQQFSACLESVEKS and ADNFERKNF
for feline IL31: aa124-135 of a feline IL-31 sequence and peptides SDVRKIILELRPMSKGLLQDYVSKEIGL and DVRKIILELRPMSKGLLQDY, LSDKNTIDKIIEQLDKLKFORE, ADNFERKNFILAVLOOFSACLEHVLOS and ADNFERKNF
for equine IL31: aa118-129 of an equine IL-31 sequence and peptides: LQPKEIQAIIVELQNLSKKLLDDY, EIQAIIVELQNLSKKLLDDY, SLNNDKSLYIIEQLDKLNFQ and TDNFERKRFILTILRWESNCLEHRAQ CGRP polypeptides, preferably:
native human CGRP alpha (ACDTATCVTHRLAGLLSRSGGVVKNNFVPTNVGSKAF); aa83-119 of calcitonin isoform alpha-CGRP preproprotein, accession number NP_001365879.1 or aa82-228 of native human CGRP beta (ACNTATCVTHRLAGLLSRSGGMVKSNFVPTNVGSKAF); aa82-118 of calcitonin gene-related peptide 2 precursor, accession number NP_000719.1 or its precursor molecules (NP_001365879.1 and NP_000719.1), preferably sequences selected from aa8-35, aa11-37, aa1-20 or fragments thereof and sequences ACDTATCVTH; ACDTATCVTHRLAGL; ACDTATCVTHRLAGLLSR; ACDTATCVTHRLAGLLSRSG; ACDTATCVTHRLAGLLSRSGGVVKN; TATCVTHRLAGLL; ATCVTHRLAGLLSR; RLAGLLSR; RLAGLLSRSGGVVKN; RSGGVVKN; RLAGLLSRSGGVVKNNFVPT; RLAGLLSRSGGVVKNNFVPTNVG; RLAGLLSRSGGVVKNNFVPTNVGSK; RLAGLLSRSGGVVKNNFVPTNVGSKAF; LLSRSGGVVKNNFVPTNVGSKAF; RSGGVVKNNFVPTNVGSKAF; GGVVKNNFVPTNVGSKAF; VVKNNFVPTNVGSKAF; NNFVPTNVGSKAF; VPTNVGSKAF; NVGSKAF; GSKAF

Allergen epitope polypeptides, preferably: polypeptides derived from native allergens, allergen protein derived polypeptide selected from mimics of the above-mentioned allergen derived polypeptides including mimotopes, constrained peptides, peptides containing amino acid substitutions and conformational epitopes see Table A and B

Preferably selected from:

| Allergen source | Allergens | UNIPROT |
|---|---|---|
| Alnus glutinosa | Aln g 1 | P38948 |
| Alternaria alternata | Alt a 1 | P79085 |
| | Alt a 3 | P78983 |
| | Alt a 4 | Q00002 |
| | Alt a 5 | P42037 |
| | Alt a 6 | Q9HDT3 |
| | Alt a 7 | P42058 |
| | Alt a 8 | P0C0Y4 |
| | Alt a 10 | P42041 |
| | Alt a 12 | P49148 |
| | Alt a 13 | Q6R4B4 |
| Ambrosia artemisiifolia | Amb a 1 | P27761 |
| | Amb a 1.05 | P27762 |
| | Amb a 3 | P00304 |
| | Amb a 5 | P02878 |
| | Amb a 6 | O04004 |
| | Amb a 7 | no full sequence available |
| | Amb a 8 | Q2KN24 |
| | Amb a 9 | Q2KN27 |
| | Amb a 10 | Q2KN25 |
| Apis mellifera | Api m 1 | P00630 |
| | Api m 2 | Q08169 |
| | Api m 3 | Q4TUB9 |
| | Api m 4 | O1501 |
| | Api m 5 | B2D0J4 |
| | Api m 6 | Q27SJ8 |
| | Api m 7 | Q8MQS8 |
| | Api m 8 | B2D0J5 |
| | Api m 9 | C9WMM5 |
| | Api m 10 | Q1HHN7 |
| | Api m 11 | B3GM11 |
| | Api m 12 | Q868N5 |
| Apium graveolens | Api g 1 | P49372 |
| | Api g 2 | E6Y8S8 |
| | Api g 3 | P92919 |
| | Api g 4 | Q9XF37 |
| | Api g 5 | P81943 |
| Arachis hypogae | Ara h 1 | P43238 |
| | Ara h 2 | Q6PSU2-1 |
| | Ara h 3 | O82580 |
| | Ara h 3.02 | Q9SQH7 |
| | Ara h 5 | Q9SQI9 |
| | Ara h 6 | Q647G9 |
| | Ara h 7 | Q9SQH1 |
| | Ara h 8 | Q6VT83 |
| Betula verrucosa | Bet v 1 | P15494 |
| | Bet v 2 | P25816 |
| | Bet v 3 | P43187 |
| | Bet v 4 | Q39419 |
| | Bet v 6 | O65002 |
| | Bet v 7 | P81531 |
| Canis familiaris | Can f 1 | O18873 |
| | Can f 2 | O18874 |
| | Can f 3 | P49822 |
| | Can f 4 | D7PBH4 |
| | Can £ 5 | P09582 |
| | Can f 6 | H2B3G5 |
| | Can f 7 | Q28895 |
| | Can f 8 | F1PHB6 |
| Carpinus betulus | Car b 1 | P38949 |
| Castanea sativa | Cas s 1 | B7TWE3 |
| | Cas s 5 | Q42428 |
| | Cas s 8 | sequence unclear |
| Cladosporium herbarum | Cla h 2 | no full sequence available |
| | CIa h 5 | P42039 |
| | CIa h 6 | P42040 |
| | CIa h 7 | P42059 |
| | CIa h 8 | P0C0Y5 |
| | CIa h 9 | B7ZK61 |
| | CIa h 10 | P40108 |
| | CIa h 12 | P50344 |
| Corylus avellana | Cor a 1 | Q08407 |
| | Cor a 2 | Q9AXH5 |
| | Cor a 6 | A0A0U1VZC8 |
| | Cor a 8 | Q9ATH2 |
| | Cor a 9 | Q8W1C2 |
| | Cor a 10 | Q9FSY7 |
| | Cor a 11 | Q8S4P9 |
| Cryptomeria japonica | Cry j 1 | P18632 |
| | Cry j 2 | P43212 |
| Cyprinus carpio | Cyp c 1 | Q8UUS3 |
| Daucus carota | Dau c 1 | O04298 |
| | Dau c 4 | Q8SAE6 |
| Dermatophagoides | Der p 1 | P08176 (variant) |
| pteronyssinus | Der p 2 | P49278 |
| | Der p 3 | P39675 |
| | Der p 4 | Q9Y197 |
| | Der p 5 | P14004 |
| | Der p 6 | P49277 |
| | Der p 7 | P49273 |
| | Der p 8 | P46419 |
| | Der p 9 | Q7Z163 |
| | Der p 10 | O18416 |
| | Der p 11 | Q6Y2F9 |
| | Der p 14 | Q8NONO |
| | Der p 20 | B2ZSY4 |
| | Der p 21 | Q2L7C5 |
| | Der p39 | XP_027203190.1 |
| Fagus sylvatica | Fag s 1 | B7TWE6 |
| Felis domesticus | Fel d 1 | P30438 (chain 1), |
| | | P30440 (chain2) |
| | Fel d 2 | P49064 |
| | Fel d 3 | Q8WNR9 |
| | Fel d 4 | Q5VFH6 |
| | Fel d 5 | no full sequence available |
| | Fel d 6 | no full sequence available |
| | Fel d 7 | E5D2Z5 |
| | Fel d 8 | F6K0R4 |
| Hevea brasiliensis | Hev b 1 | P15252 |
| | Hev b 2 | P52407 |
| | Hey b 3 | O82803 |
| | Hev b 4 | Q6T4P0 |
| | Hev b 5 | Q39967 |
| | Hev b 6 | P02877 |
| | Hev b 7.01 | O04008 |
| | Hev b 7.02 | O65811 |
| | Hev b 8 | O65812 |
| | Hev b 9 | Q9LEJ0 |
| | Hev b 10 | P35017 |
| | Hev b 11 | Q949H3 |
| | Hev b 12 | Q8RYA8 |
| | Hev b 13 | Q7Y1X1 |
| Juniperus ashei | Jun a 1 | P81294 |
| | Jun a 2 | Q9FY19 |
| | Jun a 3 | P81295 |
| Malus domestica | Mal d 1 | P43211 |
| | Mal d 2 | Q9FSG7 |
| | Mal d 3.0101w | Q5J026 |
| | Mal d 4 | Q9XF42 |
| Quercus alba | Que a 1 | B6RQS1 |
| Phleum pratense | PhI p 1 | Q40967 |
| | PhI p 2 | P43214 |
| | PhI p 4 | Q5ZQK5 |
| | PhI p 5 | Q40960 |
| | PhI p 6 | P43215 |
| | PhI p 7 | O82040 |
| | PhI p 11 | Q8H6L7 |
| | PhI p 12 | P35079 |
| | PhI p 13 | Q9XG86 |
| Polistes annularis | Pol a 1 | Q9U6W0 |
| | Pol a 5 | Q05109 |
| Polistes dominulus | Pol d 1 | Q6Q252 |
| | Pol d 4 | Q7Z269 |
| | Pol d 5 | Q68KJ8 |
| Polistes exclamans | Pol e 1 | no full sequence available |
| | Pol e 4 | no full sequence available |
| | Pol e 5 | Q68KJ9 |
| Polistes fuscatus | Pol f 5 | P35780 |
| Polistes gallicus | Pol g 1 | P83542 |
| | Pol g 5 | P83377 |
| Polistes metricus | Pol m 5 | P35780 |

and/or selected from:

| Allergens | UNIPROT | EPITOPES / MIMOTOPES |
|---|---|---|
| Amb a 1 | P27761 | GMLATVAFNTFTDNVDQR, AFNKFTDNVDQR, MPRCRFGF, |
| | | WRTQNDVLENG TFTDNVDQRMPRCRH RVVELMDWTVLH, |
| | | GSAMTWGMLAAE, SYNIIATGIHPV, TMVATGLMPVLI, |
| | | QDFDDIL, CLFSQGNRC, VANLKVGV, NPGGLSAAPAGS, |
| | | RHASTLLGRHG, EAAESMWRVASG, QNRLNSNGNNGGSQ, |
| | | DDDLQHQFDDQD |
| Amb a 3 | P00304 | CDIKDPIRLEPGGPD, EVWREEAYHACDIKD, GKVYLVGG- |
| | | PELGGWK, LGGWKLQSDPRAYAL, NETTGEDSVAEVWRE, PGG- |
| | | PDRFTLLTPGSH, PGGPDRFTLLTPGSH, QFKTTDVLWENFTTG, |
| | | RAYALWSARQQFKTT, TPGSHFICTKDQKFV |
| Alt a1 | P79085 | MISTSRK, QKRNTIT, KISEFYGRKP, VATATLPNYC, |
| | | YSCGENSFMD, YYNSLGENIK |
| Ara h 1 | P43238 | REREREEDWRQPREDWRRPS, RTRGRQPGDYDDDRRQPRRE, |
| | | DDDRRQPRREEGGRWGPAGP, TTNQRSPPGERTRGRQPGDY, |
| | | QPREDWRRPSHQQPRKIRPE, GREGEQEWGTPGSHVREETS, |
| | | PVNTPGQFEDFFPASSRDQS, AGGEQEERGQRRWSTRSSEN, RE- |
| | | GEPDLSNNFGKLFEVKPD, NASSELHLLGFGINAENNHR, |
| | | IDQIEKQAKDLAFPGSGEQV, LAFPGSGEQVEKLIKNQKES |
| | | AKSSPYQKKT, QEPDDLKQKA, LEYDPRLUYD, GERTRGRQPG, |
| | | PGDYDDDRRQ, PRREEGGRWG, REREEDWRQP, EDWRRPSHQQ, |
| | | QPKKIRPEGR, TPGQFEDFFP, SYLQEFSRNT, FNAEFNEIRR, |
| | | EQEERGQRRW, DITNPINLRE, NNFGKLFEVK, GTGNLELVAV, |
| | | RRYTARLKEG, ELHLLGEGIN, HRI FLAGDKD, IDOIEKOAKD, |
| | | KDLAFPGSGE, KESHFVSARP, PEKESPEKED |
| Ara h 2 | Q6PSU2-1 | ARQQWELQGDRRCQSQLERA, ERDPYSPSQDPYSPSPYDRR, |
| | | RRCQSQLERANLRPCEQHLM, RDPYSPSPYDRRGAGSSQHQ, GRD- |
| | | PYSPSQDPYSPSQDPDR, PYSPSQDPDRRDPYSPSPYD, |
| | | QKIQRDEDSYERDPYSPSQD, HASARQQWEL, QWELQGDRRC, |
| | | DRRCQSQLER, LRPCEQHLMQ, KIQRDEDSYE, YERDPYSPSQ, |
| | | SQDPYSPSPY, DRLQGRQQEQ, KRELRNLPQQ, QRCDLDVESG, |
| | | HASARQQWEL, ERDPYSPSQDPYSPS, RRCQSQLER, |
| | | CDLEVESGGRDRY, ERDPYSPSQDPYSPS, |
| | | NLRPCEQHLMQKIQRD, PQRCDLE, RQQEQQFKRELRNLPQQ, |
| | | SDRLQGRQQ, RRCQSQLERANLRPCEQHLMQKIQRDEDSYGRD- |
| | | PYSPSQDPY |
| Ara h 3 | 082580 | IETWNPNNQEFECAG, GNIFSGFTPEFLEQA, VTVRGGL- |
| | | RILSPDRK, DEDEYEYDEEDRG, EDEYEYDEEDRRRGRGSRGR, |
| | | NIFSGFTPEFLEQAFQVDDR, ESEEEGAIVTVRGGLRILSP, |
| | | SGFTPEFLEQAFQVDDRQIV, EEGAIVTVRGGLRILSPDRK, |
| | | TYEEPAQQGRRYQSQRPPRR, RRADEEEEYDEDEYEYDEED, |
| | | NHEQEFLRYQQQSRQSRRRS, QEFLRYQQQSRQSRRRSLPY, |
| | | QEERE FSPRGQHSRRERAGQ |
| Ara h 6 | Q647G9 | CDELNEMENTQR, CEALQQIMENQCD, CNFRAPQRCDLDV, |
| | | KPCEQHIMQRI, YDSYDIR, KRELRMLPQQ, MRRERGRGGDSSSS |
| Bet v 1 | P15494 | KAEQVKASKEMGETLLRAVESYLLAHSDAYN, GPGTIK- |
| | | KISFPEGFPFKYVKDRVDEVDHTN, DGGSILKISNKYHTKGDHEV- |
| | | KAEQVKASKE, LFPKVAPQAISSVENIEGNGGPGTIKKISF, |
| | | MGETLLRAVESYLL, MGVFNYETETTSVIPAARLFKAFI, |
| | | VDHTNFKYNYSVIEGGPIGDTLEKISNEIK, ISFPEGEPEK, |
| | | FILDGDNLFPKVAPQAISSVE, NIEGNGGPGTIKK, DGDNLF- |
| | | PKVA, ILDGDNLFPKVAPQAI, SKEMGETLLRAVESYLLAHSD, |
| | | EVDHTNFKYNYSVIEG, GGPIGDTLEKISNEIK, GPGTIKKISE, |
| | | EQVKASKEMGETLLRAVESYLLA, GDNLFPKVAPQAIS, |
| | | ISFPEGFPFKY, ISFPEGFPFKYVKD, aa 32-37, aa 42-50, |
| | | aa 138-153. COQFLSVRALC, QQFLSVRALC, CQQFLSVRAL |
| Cry j 1 | P18632 | DALTLRTATNIW, DGDALTLRTATN, DGRGAQVYIGNG, |
| | | NATPQLTKNAGV, NGGPCVFIKRVS, NGNATPQLTKNA, |
| | | NSDDDPVNPAPG, VENGNATPQLTK, NAGVLTCSLSK |
| Cry j 2 | P43212 | KWVNGREI, GQCKWVNGREICNDRDRPTA, GRENSRAEVSYV- |
| | | HVNGAKFI, PGNKKFVVNNLFENGPCQPH, SHIIYENVEMIN- |
| | | SENPILINQFYCT, YCTSASACQNORSAVQIQDV, TYKNIRGTSA- |
| | | TAAAIQLKCS, AEVSYVHVNGAK |
| Der p 1 | P08176 | KGIPNTKAP, DMFQIGKYG, GIREVWPAG, SSMGAYWGG, |
| | (variant) | KGTTGVRNT, CQIYPPNANKIREALAQ, GYSNAQGVDYWI, |
| | | NOSLDLAEQELVDCASQHGC, VRNSWDTNWGDNGY, |
| | | EQSYPCWLSGTPSTP, TPLCDYAAARVGACG, KEQLPTSYPPER- |
| | | AGW, NCLSSDEPLHIRWCQ, EALSEEEWPRYTSHP, |
| | | SCDATQRAQGRCS, SCDSSQKKOGRCS, SCDESRRRQGRCS, |
| | | KGIPNTKAP, DMFQIGKYG, GIREVWPAG, SSMGAYWGG, |
| | | KGTTGVRNT, CKGIPNTKAP, CDMFQIGKYG, CGIREVWPAG, |
| | | CSSMGAYWGG, CKGTTGVRNT, CKGIPNTKAPC, |
| | | CDMFQIGKYGC, CGIREVWPAGC, CSSMGAYWGGC, KGTT- |
| | | GVRNTC, KGIPNTKAPC, DMFQIGKYGC, GIREVWPAGC, |
| | | SSMGAYWGGC, KGTTGVRNTC, |
| Der p 2 | P49278 | FVVEYTKKW, SWWNLPQIG, KGITTKWMA, AGISYTKTW, |
| | | DQVDVKDCANHEIKK, VPGIDPNACHYMKCK, APKSEN- |
| | | VVVTVKVMGDNGVLACAIATHAKIRD, CHGSEP- |
| | | CIIHRGKPFQLEAVFEANQNSKTAK, DQVDVKDCANHEIK- |
| | | KVLVPGCHGSEPCIIHRGK, EANQNSKTAKIEIKASIEG- |
| | | LEVDVPGIDPNAC, |
| | | EVDVPGIDPNACHYMKCPLVKGQQYDIKYTWIVPKIAPKSEN, |
| | | FVVEYTKKW, SWWNLPQIG, KGITTKWMA, AGISYTKTW, |
| | | FVVEYTKKWC, SWWNLPQIGC, KGITTKWMAC, AGISYTKTWC, |
| | | CFVVEYTKKWC, CSWWNLPQIGC, CKGITTKWMAC, CAG- |
| | | ISYTKTWC, CFVVEYTKKW, CSWWNLPQIG, CKGITTKWMA, |
| | | CAGISYTKTW |
| Der p39 | XP_ | QEELREAFRMY, TSALREILRAL, NDELDEMIAEI |
| | 027203190.1 | |
| Fel d 1 | P30438 | KALPVVLENARILKNCVDAKMTEEDKE, |
| | (chain | KRDVDLELTGTPDEYVEQVAQYKALPV, DAKMTEEDKENALS, |
| | 1), | EICPAVKRDVDLFLTG, EPERTAMKKIQDCY, FA- |
| | P30440 | VANGNELLLDLS, KKIQDCYVENGLIS, LLDKIYTSPLC, |
| | (chain2) | VAQYKALPVVLENA, VKMAETCPIFYDVE, VQNTVEDLKLNTLGR, |
| | | EICPAVKRDVDLELTGTPDEYVEQVAQYK, SSKDCMGEAVQNTVED- |
| | | LKLNTLGR, VKMAITCPI FYDVFFAVANGNELLLDLSLTKVNA, |
| | | CPAVKRDVDLELT, EQVAQYKALPVVLENA KALPVVLENARILNCV |
| | | RILKNCVDAKMTEEDKE KENALSLLDKIYTSPL TAMKKIQDCY |
| | | VENGLI SRVLDGLVMTTISSSK, ENLAYTKALLTG; NNIS- |
| | | VDGLLTS; VIRNELLLTYA; VINDLLLVLA; GDFYPL; |
| | | NDFYPE; LDLNP, FAVANGNELL; LKLNTLGREICPAVKRGVDL; |
| | | YVEQV; ENARILKNCVDAKM; aa 21-31, aa 21-47 |
| PhI p 1 | Q40967 | VRYTTEGGTKTEAEDVIPEGWKADTSYESK, APYHEDLSGHAFGAM, |
| | | HITDDNEEPIAPYHEDLSGHA, NEEPIAPYHEDLSGHAFG, |
| | | CFEIKCTKPEACSGEPVVV, CTK- |
| | | PEACSGEPVVVHITDDNEEP IAPYHFDLSGH, DNEEPIAPYHE, |
| | | EPIAPYHEDLSGH, EQKLRSAGELELQFRRVKC, |
| | | GEPVVVHITDDNEEPIAPYHEDLSGHAFGAMAKKG, |
| | | GNTPIFKSGRGCGSCFEIKCTKPEACSGEPVVVHITDDNEEPIAPY- |
| | | HFDL, GPFTVRYTTEGGTKTE, GTKTEAEDVIPEGWKADTSYESK, |
| | | GYKDVDKPPFSGMTGCGNTPIFKSGRGCGSCFEIKCTKPEACS, |
| | | HITDDNEEPIAPYHE, HITDDNEEPIAPYHEDLSGHAFGA, |
| | | HVEKGSNPNYLALLVKYVNGDGDVVAV, KCTK- |
| | | PEACSGEPVVVHITDDNEEPIAPYHEDLS, KPPFSGMTGCGNT, |
| | | KTEAEDVIPEGWKADTSYESK, LTGPFTVRYTTEGGTKTEAEDVI- |
| | | PEGWKADTSYESK, PIAPYHFD, PIAPYHEDLSGHAFG |
| PhI p 2 | P43214 | VPKVTFTVEKGSNEKHLAVLVKYEGDTMAEVEL, |
| | | VEKGSNEKHLAVLVKYEGDTMAEVELREHGSD, RE- |
| | | HGSDEWVAMTKGEGGVWT FDSEEPLQGPEN, |
| | | FRFLTEKGMKNVEDDVVPEKYTIGATYAPEE, |
| | | CFRFLTEKGMKNVEDDVVPEKYTIGATYAPEE, |
| | | FRFLTEKGMKNVFDDVVPEKYTIGATYAPEEC |
| PhI p 5 | Q40960 | AEEVKVI PAGELQVIEKVDAAFKVAATAANAAPANDK, AT- |
| | | TEEQKLIEKINAGFKAALAAAAGVQPADKYR, FVAT- |
| | | FGAASNKAFAEGLSGEPKGAAESSSKAALTSK, |
| | | ADLGYGPATPAAPAAGYTPATPAAPAEAAPAGK, |
| | | AYKLAYKTAEGATPEAKYDAYVATLSEALRI, EAAFNDAIKAST- |
| | | GGAYESYKFIPALEAAVK, TVATAPEVKYTVFETALKKAITAM- |
| | | SEAQKAAK, SRLGRSSAWV, THWQLGERPD, PSTPGERVRH, |
| | | RGGPDDLTAL, PFWVRGTTW, PSTPGSRQNM, PSTPGDNPLV, |
| | | KFVVNGRWID, KFLVNGRWID, RLTENTEPLL, FTWGGLRDKS, |
| | | ERAGAMERAN, RSVSKEEPGM, KLGKFGAARV, VQDLMKSSGV, |
| | | KLGKFGAARV, CKLGKFGAARVC, CKLGKFGAARV, KLGKF- |
| | | GAARVC, |
| PhI p 6 | P43215 | GKATTEEQKLIEDVNASFRAAMATTANVPPAD, YKT- |
| | | FEAAFTVSSKRNLADAVSKAPQLVPKLDEVYN, |
| | | DAVSKAPQLVPKLDEVYNAAYNAADHAAPEDKY, |
| | | AADHAAPEDKYEAFVLHFSEALRIIAGTPEVHA |

Human PCSK9 polypeptides, preferably
native human PCSK9 or a polypeptide comprising or consisting of amino acid residues aa150 to 170, aa153-162, aa205 to 225, aa211-223, aa368-382, with the amino acid sequence (accession number: Q8NBP7):

MGTVSSRRSW WPLPLLLLLL LLLGPAGARA QEDEDGDYEE
LVLALRSEED GLAEAPEHGT TATFHRCAKD PWRLPGTYVV
VLKEETHLSQ SERTARRLQA QAARRGYLTK ILHVFHGLLP
GFLVKMSGDL LELALKLPHV DYIEEDSSVE AQSIPWNLER
ITPPRYRADE YQPPDGGSLV EVYLLDTSIQ SDHREIEGRV
MVTDFENVPE EDGTRFHRQA SKCDSHGTHL AGVVSGRDAG
VAKGASMRSL RVLNCQGKGT VSGTLIGLEF IRKSQLVQPV
GPLVVLLPLA GGYSRVLNAA CQRLARAGVV LVTAAGNERD
DACLYSPASA PEVITVGATN AQDQPVTLGT LGTNFGRCVD
LFAPGEDIIG ASSDCSTCFV SQSGTSQAAA HVAGIAAMML
SAEPELTLAE LRQRLIHFSA KDVINEAWFP EDQRVLTPNL
VAALPPSTHG AGWQLFCRTV WSAHSGPTRM ATAVARCAPD
EELLSCSSES RSGKRRGERM EAQGGKLVCR AHNAFGGEGV
YAIARCCLLP QANCSVHTAP PAEASMGTRV HCHQQGHVLT
GCSSHWEVED LGTHKPPVLR PRGQPNQCVG HREASIHASC
CHAPGLECKV KEHGIPAPQE QVIVACEEGW TLTGCSALPG
TSHVLGAYAV DNTCVVRSRD VSTTGSTSEG AVTAVAICCR
SRHLAQASQE LQ

and/or PCSK9 protein derived polypeptides selected from mimics of the above-mentioned polypeptides including mimotopes and peptides containing amino acid substitutions,
and/or PCSK9 derived sequences NVPEEDGTRFHRQASK, NVPEEDGTRFHRQASKC, PEEDGTRFHRQASK, CPEEDGTRFHRQASK, PEEDGTRFHRQASKC, AEEDGTRFHRQASK, TEEDGTRFHRQASK, PQEDGTRFHRQASK, PEEDGTRFHRRASK, PEEDGTRFHRKASK, PEEDGTRFHRQASR, PEEDGTRFHRTASK, SIPWNLERITPPR, PEEDGTRFHRQASK, PEEDGTRFHRQA, EEDGTRFHRQASK, EEDGTRFHRQAS, SIPWNLERITP, SIPWNLERITPC, SIPWNLERIT, SIPWNLERITC, LRPRGQPNQC, SRHLAQASQ, SRHLAQASQC, SRSGKRRGER, SRSGKRRGERC, IIGASSDCSTCFVSQ, IIGASSDSSTSFVSQ, IIGASSDSSTSFVSQC, CIGASSDSSTSFVSC, IGASSDSSTSFVSC, CDGTRFHRQASKC, DGTRFHRQASKC, CDGTRFHRQASK, AGRDAGVAKGAC, RDAGVAKC, RDAGVAK, SRHLAQASQLEQC; SRHLAQASQLEQ, GDYEELVLALRC; GDYEELVLALR, LVLALRSEEDC; LVLALRSEED, AKDPWRLPC; AKDPWRLP, AARRGYLTKC, AARRGYLTK, FLVKMSGDLLELALKLPC; FLVKMSGDLLELALKLP, EEDSSVFAQC, EEDSSVFAQ, NVPEEDGTRFHRQASKC, NVPEEDGTRFHRQASK, CKSAQRHFRTGDEEPVN, KSAQRHFRTGDEEPVN,
alpha synuclein polypeptides, preferably
native alpha synuclein or a polypeptide comprising or consisting of amino acid residues 1 to 5, 1 to 8, 1 to 10, 60 to 100, 70 to 140, 85 to 99, 91 to 100, 100 to 108, 102 to 108, 102 to 109, 103 to 129, 103 to 135, 107 to 130, 109 to 126, 110 to 130, 111 to 121, 111 to 135, 115 to 121, 115 to 122, 115 to 123, 115 to 124, 115 to 125, 115 to 126, 118 to 126, 121 to 127, 121 to 140, or 126 to 135, of the amino acid sequence of native human alpha synuclein: MDVFMKGLSK AKEGVVAAAE KTKQGVAEAA GKTKEGVLYV GSKTKEGVVH GVATVAEKTK EQVTNVGGAV VTGVTAVAQK TVEGAGSIAA ATGFVKKDQL GKNEEGAPQE GILEDMPVDP DNEAYEMPSE EGYQDYEPEA (human aSyn (1-140 aa): UNIPROT accession number P37840),
preferably a polypeptide comprising or consisting of amino acid residues 1 to 8, 91 to 100, 100 to 108, 103 to 135, 107 to 130, 110 to 130, 115 to 121, 115 to 122, 115 to 123, 115 to 124, 115 to 125, 115 to 126, 118 to 126, 121 to 127, or 121 to 140; or mimotopes selected from the group DQPVLPD, DOPVLPDN, DOPVLPDNE, DQPVLPDNEA, DQPVLPDNEAY, DQPVLPDNEAYE, DSPVLPDG, DHPVHPDS, DTPVLPDS, DAPVTPDT, DAPVRPDS, and YDRPVQPDR.
10. A conjugate according to any one of embodiments 1 to 8, wherein the conjugate comprises a T-cell epitope and is free of B-cell epitopes, wherein the conjugate preferably comprises more than one T-cell epitope, especially two, three, four or five T-cell epitopes.
11. A conjugate according to any one of embodiments 1 to 10 for use in the prevention or treatment of diseases in humans, mammals or birds, preferably for use in the prevention or treatment of infectious diseases, chronic diseases, allergies or autoimmune diseases, especially in humans; with the preferred proviso that the use in the prevention or treatment of diseases caused directly or indirectly by fungi, especially by C. albicans, are excluded.
12. A conjugate according to any one of embodiments 1 to 11 for use for active anti-Tau protein vaccination against synucleinopathies, Pick disease, progressive supranuclear palsy (PSP), corticobasal degeneration, Frontotemporal dementia and parkinsonism linked to chromosome 17 (FTDP-17) and argyrophilic grain disease; and/or
for use for active immunotherapy for IL12/IL23 related disease and autoimmune inflammatory diseases, especially selected from the group psoriasis, psoriatic arthritis, rheumatoid arthritis, systemic lupus erythematosus, diabetes, preferably type 1 diabetes, atherosclerosis, inflammatory bowel disease (IBD)/M. Crohn, multiple sclerosis, Behcet disease, ankylosing spondylitis, Vogt-Koyanagi-Harada disease, chronic granulomatous disease, hidratenitis suppurtiva, anti-neutrophil cytoplasmic antibodies (ANCA-) associated vasculitides, neurodegenerative diseases, preferably M. Alzheimer or multiple sclerosis, atopic dermatitis, graft-versushost disease, cancer, preferably Oesophagal carcinoma, colorectal carcinoma, lung adenocarcinoma, small cell carcinoma, and squamous cell carcinoma of the oral cavity, especially psoriasis, neurodegenerative diseases or IBD; and/or
for use as active anti-EMPD vaccine for the treatment and prevention of IgE related diseases, preferably allergic diseases such as seasonal, food, pollen, mold spores, poison plants, medication/drug, insect-, scorpion- or spider-venom, latex or dust allergies, pet allergies, allergic asthma bronchiale, non-allergic asthma, Churg-Strauss Syndrome, allergic rhinitis and -conjunctivitis, atopic dermatitis, nasal polyposis, Kimura's disease, contact dermatitis to adhesives, antimicrobials, fragrances, hair dye, metals, rubber components, topical medicaments, rosins, waxes, polishes, cement and leather, chronic rhinosinusitis, atopic eczema, autoimmune diseases where IgE plays a role (“autoallergies”), chronic (idiopathic) and autoimmune urticaria, cholinergic urticaria, mastocytosis, especially cutaneous mastocytosis, allergic bronchopulmonary aspergillosis, chronic or recurrent idiopathic angioedema, interstitial cystitis, anaphylaxis, especially idiopathic and exercise-induced anaphylaxis, immunotherapy, eosinophil-associated diseases such as eosinophilic asthma, eosinophilic gastroenteritis, eosinophilic otitis media and eosinophilic oesophagitis; or for use for the treatment of lymphomas or the prevention of sensibilisation side effects of an anti-acidic treatment, especially for gastric or duodenal ulcer or reflux; and/or
for use for active anti-Human Epidermal Growth Factor Receptor 2 (anti-Her2) vaccination, for the treatment and prevention of Her2 positive neoplastic diseases; and/or
for use in individualized neoantigen specific therapy, preferably with NY-ESO-1, MAGE-A1, MAGE-A3, MAGE-C1, MAGE-C2, MAGE-C3, Survivin, gp100, tyrosinase, CT7, WT1, PSA, PSCA, PSMA, STEAP1, PAP, MUC1, 5 T4, KRAS, or Her2; and/or
for use for active anti-immune checkpoint vaccination for controlling the cancer microenvironment, for the treatment and prevention of neoplastic diseases and for treatment and prevention of T-cell dysfunction in cancer/neoplastic disease (e.g. avoiding exhaustion of CD8 T-cells infiltrating cancer tissues) and chronic degenerative diseases including diseases with reduced T-cell activity like Parkinson's Disease; and/or
for use in familial and sporadic AD, familial and sporadic Aβ cerebral amyloid angiopathies, Hereditary cerebral hemorrhage with amyloidosis (HCHWA), Dementia with Lewy bodies and Dementia in Down syndrome, Retinal ganglion cell degeneration in glaucoma, Inclusion body myositis/myopathy; and/or
for use as active vaccine for the treatment and prevention of synucleopathies, preferably Parkinson's disease (PD), dementia with Lewy bodies (DLB), multiple system atrophy (MSA), Parkinson's disease dementia (PDD), neuroaxonal dystrophies, Alzheimer's Disease with Amygdalar Restricted Lewy Bodies (AD/ALB); and/or
for use as an active vaccine comprising an antigen or neoantigen selected from the group NY-ESO-1, MAGE-A1, MAGE-A3, MAGE-C1, MAGE-C2, MAGE-C3, Survivin, gp100, tyrosinase, CT7, WT1, PSA, PSCA, PSMA, STEAP1, PAP, MUC1, 5 T4, and KRAS; and/or
for use in the treatment or prevention of IL31-related diseases with IL31 protein-derived polypeptides, such as fragments of the IL-31 protein, preferably pruritus-causing allergic diseases, pruritus-causing inflammatory diseases and pruritus-causing auto-immune diseases in mammals, including humans, dogs, cats and horses; atopic dermatitis, prurigo nodularis, psoriasis, cutaneous T-Cell lymphoma (CTCL), and other pruritic disorders, such as uremic pruritus, cholestatic pruritus, bullous pemphigoid and chronic urticaria, allergic contact dermatitis (ACD), dermatomyositis, chronic pruritus of unknown origin (CPUO), primary localized cutaneous amyloidosis (PLCA), mastocytosis, chronic spontaneous urticaria, bullous pemphigoid, dermatitis herpetiformis and other dermatologic conditions including lichen planus, cutaneous amyloidosis, statis dermatitis, scleroderma, itch associated with wound healing and non-pruritic diseases such as allergic asthma, allergic rhinitis, inflammatory bowel disease (IBD), osteoporosis, follicular Lymphoma, Hodgkin lymphoma and chronic myeloid leukemia; especially wherein the anti-IL31 treatment is combined with anti-IL4 and/or anti-IL13 peptide vaccine; and/or
for use in the treatment or prevention of CGRP related diseases with CGRP derived polypeptides, such as fragments of CGRP, preferably episodic and chronic migraine and cluster headache, hyperalgesia, hyperalgesia in dysfunctional pain states, such as for example rheumatoid arthritis, osteoarthritis, visceral pain hypersensitivity syndromes, fibromyalgia, inflammatory bowel syndrome, neuropathic pain, chronic inflammatory pain and headaches; and/or
for use in specific allergen immunotherapy (AIT) for the treatment of IgE mediated type I allergic disease. These diseases include but are not limited to hay fever, seasonal-, food-, pollen-, mold spores-, poison plants-, medication/drug-, insect-, scorpion- or spider-venom, latex- or dust allergies, pet allergies, allergic asthma bronchiale, allergic rhinitis and -conjunctivitis, atopic dermatitis, contact dermatitis to adhesives, antimicrobials, fragrances, hair dye, metals, rubber components, topical medicaments, rosins, waxes, polishes, cement and leather, chronic rhinosinusitis, atopic eczema, autoimmune diseases where IgE plays a role (“autoallergies”), chronic (idiopathic) and autoimmune urticaria, anaphylaxis, especially idiopathic and exercise-induced anaphylaxis; and/or
for use to improve target-specific immune responses from existing vaccines, especially anti-infective vaccines, while inducing no or only very limited CLEC- or carrier protein-specific antibody responses, preferably selected from the group of vaccines Pedvax-HIB®, ActHIB®, Hiberix®, Recombivax HB®, PREHEVBRIO®, Engerix-B, HEPLISAV-B®, Gardasil®, Gardasil 9®, Cervarix®, Menveo®, Menactra®, MenQuadfi®, Prevnar-13®, Prevnar 20®, Pneumovax-23®, Vaxneuvance®, Typhim V®, Typhim VI®, Typherix®, Vi polysaccharide bound to a non-toxic recombinant Pseudomonas aeruginosa exotoxin A, Typbar-TCV®, Shingrix®; and/or
for the prevention of infectious diseases, such asmicrobial infections or viral infections, preferably caused by Haemophilus influenzae type b (Hib), Streptococcus pneumoniae, Neisseria meningitidis and Salmonella Typhi or other infectious agents including those causing Hepatitis A or B, Human Papilloma Virus infections, Influenza, Thyphoid Fever, Measles, Mumps and Rubella. In addition, infections caused by meningococcal group B bacteria, Cytomegalovirus (CMV), Respiratory Syncytial Virus (RSV), Clostridioides Difficile, Extraintestinal Pathogenic Escherichia Coli (Expec), Klebsiella Pneumoniae, Shigella, Staphylococcus Aureus, Plasmodium falciparum, P. vivax, P. ovale, and P. malariae, Coronavirus (SARS-COV, MERS-COV, SARS-COV-2), Ebola Virus, Borrelia burgdorferi, HIV and others; and/or
for use in the treatment or prevention of Proprotein convertase subtilisin/kexin type 9 (PCSK9) related disease including but not limited to hyperlipidemia, hypercholesteremia, atherosclerosis, increased serum level of low-density lipoprotein cholesterol (LDL-C) and cardiovascular events, stroke or various forms of cancer. and/or
for use to induce target-specific immune responses while inducing no or only very limited CLEC- or carrier protein-specific antibody responses; and/or
for the induction of target specific immune responses while inducing no or only very limited CLEC- or carrier-protein specific antibody responses; and/or
for use in diseases with reduced or dysfunctional Treg populations to augment waning/reduced Treg number and activity and thereby reduce autoimmune reactivity of disease specific T-effector cells and dampen autoimmune responses in patients, wherein T-cell epitopes suitable as Treg epitopes, or a combination with Treg inducing agents, such as rapamycin, low-dose IL-2, TNF receptor 2 (TNFR2) agonist, anti-CD20 antibodies (such as rituximab), prednisolone, inosine pranobex, glatiramer acetate, or sodium butyrate; are used; and/or
for use in a treatment for augmenting or preserving T-cell numbers, especially T-effector cell numbers, and T-cell function in a PD patient, which preferably includes a combination of checkpoint inhibitors or vaccines using anti-immune check point inhibitor epitopes to induce an anti-immune checkpoint inhibitor immune response to augment or preserve T-cell numbers, especially T-effector cell numbers and T-cell function in a PD patient, wherein the PD patient is preferably selected by having an overall reduction of CD3+ cells, especially of CD3+CD4+ cells typical for PD patients at all stages of the disease; preferably a patient in H+Y stages 1-4, more preferred H+Y1-3, most preferred H+Y 2-3.
13. A conjugate according to any one of embodiments 1 to 12, wherein the β-glucan or mannan is for use as C-type lectin (CLEC) polysaccharide adjuvant, preferably for enhancing the T-cell response to a given T-cell epitope polypeptide, more preferred wherein the T-cell epitope is a linear T-cell epitope, especially wherein the T-cell epitope is a polypeptide comprising or consisting of the amino acid sequences SeqID7, 8, 22-29, 87-131, GKTKEGVLYVGSKTK, KTKEGVLYVGSKTKE, EQVTNVGGAVVTGVT, VTGVTAVAQKTVEGAGNIAAATGFVK, MPVDPDNEAYEMPSE), DNEAYEMPSEEGYQD, EMPSEEGYQDYEPEA, or combinations thereof.
14. A conjugate according to any one of embodiments 1 to 13 for use in increasing affinity maturation with respect to a specific polypeptide antigen or for inducing an increased immune response with respect to a human self-antigen.
15. A conjugate according to any one of embodiments 1 to 14 further comprising a carrier protein comprising T-cell epitopes for use in reducing or eliminating the B-cell response to the CLEC and/or to the carrier protein and/or in enhancing the T-cell response to the T-cell epitopes of the carrier protein, preferably wherein the carrier protein is non-toxic cross-reactive material of diphtheria toxin (CRM), especially CRM197, KLH, diphtheria toxoid (DT), tetanus toxoid (TT), Haemophilus influenzae protein D (HipD), and the outer membrane protein complex of serogroup B meningococcus (OMPC), recombinant non-toxic form of Pseudomonas aeruginosa exotoxin A (rEPA), flagellin, Escherichia coli heat labile enterotoxin (LT), cholera toxin (CT), mutant toxins (e.g., LTK63 and LTR72), virus-like particles, albumin binding protein, bovine serum albumin, ovalbumin, a synthetic peptide dendrimer e.g. a Multiple antigenic peptide (MAP), preferably wherein the ratio of carrier protein to β-glucan in the conjugate is from 1/0.1 to 1/50, preferably 1/0.1 to 1/40, more preferred from 1/0.1 to 1/20, especially from 1/0.1 to 1/10, especially wherein the T-cell epitope efficacy in a vaccine comprising linear T-cell epitopes is augmented, e.g. by an N- or C-terminal addition of a lysosomal protease cleavage site, such as a Cathepsin L-like cleavage site or an Cathepsin S-like cleavage site, wherein the Cathepsin L-like cleavage site is preferably defined by the following consensus sequence:

Xₙ-X₁-X₂-X₃-X₄-X₅-X₆-X₇-X₈
Xₙ: 3-27 amino acids from the immunogenic peptide
X₁: any amino acid
X₂: any amino acid
X₃: any amino acid
X₄: N/D/A/Q/S/R/G/L; preferred N/D, more preferred N
X₅: F/R/A/K/T/S/E; preferred F or R, more preferred R
X₆: F/R/A/K/V/S/Y; preferred F or R, more preferred R
X₇: any amino acid, preferred A/G/P/F, more preferred A
X₈: cysteine or Linker like NHNH₂,
wherein the most preferred sequence is Xₙ-X₁X₂X₃NRRA-Linker;
and wherein the Cathepsin S-like cleavage site is preferably defined by the following consensus sequence:

Xₙ-X₁-X₂-X₃-X₄-X₅-X₆-X₇-X₈
Xₙ: 3-27 amino acids from the immunogenic peptide
X₁: any amino acid
X₂: any amino acid
X₃: any amino acid, preferred V, L, I, F, W, Y, H, more preferred V
X₄: any amino acid, preferred V, L, I, F, W, Y, H, more preferred V
X₅: K, R, E, D, Q, N, preferably K, R more preferably R
X₆: any amino acid
X₇: any amino acid, preferred A
X₈: preferred A
X₈: cysteine or linker like NHNH₂, wherein the most preferred sequence is Xₙ-X₁X₂VVRAA-Linker.
16. A method for producing a conjugate according to any one of embodiments 1 to 15, wherein the β-glucan or mannan is activated by oxidation and wherein the activated β-glucan or mannan is contacted with the B-cell and/or the T-cell epitope polypeptide, thereby obtaining a conjugate of the β-glucan or mannan with the B-cell and/or the T-cell epitope polypeptide.
17. A method according to embodiment 16, wherein the β-glucan or mannan is obtained by periodate oxidation at vicinal hydroxyl groups, as reductive amination, or as cyanylation of hydroxyl groups.
18. A method according to embodiment 16 or 17, wherein the β-glucan or mannan is oxidized to an oxidation degree defined as the reactivity with Schiff's fuchsin-reagent corresponding to an oxidation degree of an equal amount of pustulan oxidized with periodate at a molar ratio of 0.2-2.6 preferably of 0.6-1.4, especially 0.7-1.
19. A method according to any one of embodiments 16 to 18, wherein the conjugate is produced by hydrazone based coupling for conjugating hydrazides to carbonyls (aldehyde) or coupling by using hetero-bifunctional, maleimide-and-hydrazide linkers (e.g.: BMPH (N-β-maleimidopropionic acid hydrazide, MPBH (4-[4-N-maleimidophenyl]butyric acid hydrazide), EMCH (N-[ε-Maleimidocaproic acid) hydrazide) or KMUH (N-[κ-maleimidoundecanoic acid] hydrazide) for conjugating sulfhydryls (e.g.: cysteines) to carbonyls (aldehyde).
20. A vaccine product designed for vaccinating an individual against a specific antigen, wherein the product comprises a compound comprising a β-glucan or mannan as a C-type lectin (CLEC) polysaccharide adjuvant covalently coupled to the specific antigen.
21. Vaccine product according to embodiment 20, wherein the product comprises a conjugate according to any one of embodiments 1 to 16 or obtainable or obtained by a method according to any one of embodiments 16 to 19.
22. Vaccine product according to embodiment 20 or 21, wherein the antigen comprises at least one B-cell epitope and at least one T-cell epitope, preferably wherein the antigen is a polypeptide comprising one or more B-cell and T-cell epitopes.
23. Vaccine product according to any one of embodiments 20 to 22, wherein the covalently coupled antigen and CLEC polysaccharide adjuvant are present as particles with a size of 1 to 5000 nm, preferably of 1 to 200 nm, especially of 2 to 160 nm, determined as hydrodynamic radius (HDR) by dynamic light scattering (DLS).
24. Vaccine product according to any one of embodiments 20 to 23, wherein the covalently coupled antigen and CLEC polysaccharide adjuvant are present as particles with a size of 1 to 50 nm, preferably of 1 to 25 nm, especially of 2 to 15 nm, determined as HDR by DLS.
25. Vaccine product according to any one of embodiments 20 to 24, wherein the covalently coupled antigen and CLEC polysaccharide adjuvant are present as particles with a size smaller than 100 nm, preferably smaller than 70 nm, especially smaller than 50 nm, determined as HDR by DLS.
26. Pharmaceutical composition comprising a conjugate or vaccine as defined in any one of embodiments 1 to 25 and a pharmaceutically acceptable carrier.
27. Pharmaceutical composition according to embodiment 26, wherein the pharmaceutically acceptable carrier is a buffer, preferably a phosphate or TRIS based buffer.
28. Pharmaceutical composition according to embodiment 26 or 27 contained in a needle-based delivery system, preferably a syringe, a mini-needle system, a hollow needle system, a solid microneedle system, or a system comprising needle adaptors; an ampoule, needle-free injection systems, preferably a jet injector; a patch, a transdermal patch, a microstructured transdermal system, a microneedle array patch (MAP), preferably a solid MAP (S-MAP), coated MAP (C-MAP) or dissolving MAP (D-MAP); an electrophoresis system, a iontophoresis system, a laser-based system, especially an Erbium YAG laser system; or a gene gun system.
29. Pharmaceutical composition according to any one of embodiments 26 to 28, wherein the conjugate or vaccine in contained as a solution or suspension, deep-frozen solution or suspension; lyophilizate, powder, or granulate.
30. Use of a conjugate according to any one of embodiments 1 to 15 for the manufacture of a medicament for the prevention or treatment of diseases, preferably for the prevention or treatment of infectious diseases, chronic diseases, allergies or autoimmune diseases.
31. A method for the prevention or treatment of diseases, preferably for use in the prevention or treatment of infectious diseases, chronic diseases, allergies or autoimmune diseases, wherein an efficient amount of a conjugate according to any one of embodiments 1 to 15 is administered to a patient in need thereof.

1. A conjugate comprising a β-glucan and a B-cell and/or T-cell epitope polypeptide, wherein the β-glucan is covalently conjugated to the B-cell and/or T-cell epitope polypeptide to form a conjugate of the β-glucan and the B-cell and/or T-cell epitope polypeptide, wherein the β-glucan is a predominantly linear β-(1,6)-glucan with a ratio of (1,6)-coupled monosaccharide moieties to non-β-(1,6)-coupled monosaccharide moieties of at least 1:1, preferably at least 2:1, more preferred, at least 5:1, especially at least 10:1.

2. A conjugate according to claim 1, wherein the β-glucan is pustulan.

3. A conjugate according to claim 1, wherein the polypeptides comprise at least one B-cell and at least one T-cell epitope.

4. A conjugate according to claim 1, wherein the ratio of β-glucan to B-cell and/or T-cell epitope polypeptide in the conjugate is from 10:1 (w/w) to 1:1 (w/w), preferably from 8:1 (w/w) to 2:1 (w/w), especially 4:1 (w/w).

5. A conjugate according to claim 1, wherein a B-cell epitope and a pan-specific/promiscuous T-cell epitope is independently coupled to the β-glucan.

6. A conjugate according to claim 1, wherein the B-cell epitope polypeptide has a length of 5 to 20 amino acid residues, preferably of 6 to 19 amino acid residues, especially of 7 to 15 amino acid residues.

7. A conjugate according to claim 1, wherein the T-cell epitope polypeptide has a length of 8 to 30 amino acid residues, preferably of 13 to 29 amino acid residues, especially of 13 to 28 amino acid residues.

8. A conjugate according to claim 1, wherein the conjugate further comprises a carrier protein, preferably non-toxic cross-reactive material of diphtheria toxin (CRM), especially CRM₁₉₇, KLH, diphtheria toxoid (DT), tetanus toxoid (TT), Haemophilus influenzae protein D (HipD), and the outer membrane protein complex of serogroup B meningococcus (OMPC), recombinant non-toxic form of Pseudomonas aeruginosa exotoxin A (rEPA), flagellin, Escherichia coli heat labile enterotoxin (LT), cholera toxin (CT), mutant toxins (e.g., LTK63 and LTR72), virus-like particles, albumin binding protein, bovine serum albumin, ovalbumin, a synthetic peptide dendrimer e.g. a Multiple antigenic peptide (MAP), with the proviso that if the conjugate comprises a carrier protein, the conjugate comprises at least a further, independently conjugated T-cell or B-cell epitope polypeptide, especially wherein the ratio of carrier protein to β-glucan in the conjugate is from 1/0.1 to 1/50, preferably 1/0.1 to 1/40, more preferred from 1/0.1 to 1/20, especially from 1/0.1 to 1/10.

9. A conjugate according to claim 1, wherein the polypeptide is or comprises a B-cell or a T-cell epitope polypeptide, preferably wherein the polypeptide is or comprises a B-cell and a T-cell epitope.

10. A conjugate according to claim 1, wherein the conjugate comprises a T-cell epitope and is free of B-cell epitopes, wherein the conjugate preferably comprises more than one T-cell epitope, especially two, three, four or five T-cell epitopes.

11. A method for prevention or treatment of diseases, preferably for the prevention or treatment of infectious diseases, chronic diseases, allergies or autoimmune diseases comprising administering an effective amount of a conjugate according to claim 1 to a human or mammalian subject in need thereof.

12. A method for the treatment and prevention of β-amyloidoses, tauopathies, or synucleopathies, preferably Parkinson's disease (PD), dementia with Lewy bodies (DLB), multiple system atrophy (MSA), Parkinson's disease dementia (PDD), neuroaxonal dystrophies, Alzheimer's Disease (AD), AD with Amygdalar Restricted Lewy Bodies (AD/ALB), dementia in Down syndrome, Pick disease, progressive supranuclear palsy (PSP), corticobasal degeneration, Frontotemporal dementia and parkinsonism linked to chromosome 17 (FTDP-17) and argyrophilic grain disease comprising administering an effective amount of a conjugate according to claim 1 to a human or mammalian subject in need thereof.

13. A method for producing a conjugate according to claim 1, wherein the β-glucan is activated by oxidation and wherein the activated β-glucan is contacted with the B-cell and/or the T-cell epitope polypeptide, thereby obtaining a conjugate of the β-glucan with the B-cell and/or the T-cell epitope polypeptide.

14. A method according to claim 13, wherein the β-glucan is obtained by periodate oxidation at vicinal hydroxyl groups, as reductive amination, or as cyanylation of hydroxyl groups.

15. A method according to claim 13, wherein the β-glucan is oxidized to an oxidation degree defined as the reactivity with Schiff's fuchsin-reagent corresponding to an oxidation degree of an equal amount of pustulan oxidized with periodate at a molar ratio of 0.2-2.6 preferably of 0.6-1.4, especially 0.7-1.
